(19) United States
(12) Patent Application Publication
(10) Pub. No.: US 2022/0109111 A1
(43) Pub. Date: Apr. 7, 2022

(54) HETEROCYCLIC COMPOUND AND ORGANIC LIGHT-EMITTING DIODE COMPRISING SAME

(71) Applicant: LT MATERIALS CO., LTD., Yongin-City, Gyeonggi-do (KR)

(72) Inventors: Han-Kook OH, Osan-si (KR); Ji-Young KIM, Yangsan-si (KR); Won-Jang JEONG, Hwaseong-si (KR)

(73) Assignee: LT MATERIALS CO., LTD., Yongin-City, Gyeonggi-do (KR)

(21) Appl. No.: 17/288,726

(22) PCT Filed: Nov. 8, 2019
(86) PCT No.: PCT/KR2019/015186

§ 371 Date: Apr. 26, 2021

(30) Foreign Application Priority Data
Nov. 8, 2018 (KR) 10-2018-0136896

(51) Int. Cl. H01L 51/00; C07D 471/04; C09K 11/06; C07F 9/53

(52) U.S. Cl. CPC H01L 51/0072; C07D 471/04; C09K 11/06; H01L 51/0067; H01L 51/5012; C07F 9/5325; H01L 51/0074; H01L 51/0052; H01L 51/0073

(57) ABSTRACT

The present specification relates to a heterocyclic compound represented by Chemical Formula 1, and an organic light emitting device comprising the same.

### TECHNICAL FIELD

This application claims priority to and the benefits of Korean Patent Application No. 10-2018-0136896, filed with the Korean Intellectual Property Office on Nov. 8, 2018, the entire contents of which are incorporated herein by reference.

The present specification relates to a heterocyclic compound, and an organic light emitting device comprising the same.

### BACKGROUND ART

An electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

U.S. Pat. No. 4,356,429

### DISCLOSURE

### Technical Problem

The present disclosure is directed to providing a heterocyclic compound, and an organic light emitting device comprising the same.

### Technical Solution

One embodiment of the present application provides a heterocyclic compound represented by the following Chemical Formula 1.

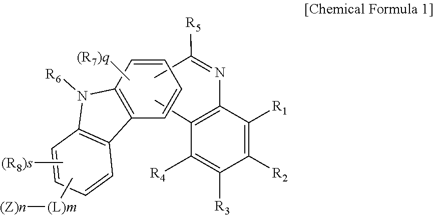

In Chemical Formula 1,

R₁ to R₄, R₇ and R₈ are the same as or different from each other, and each independently selected from the group consisting of hydrogen; deuterium; a substituted or unsubstituted alkyl group; a substituted or unsubstituted aryl group; a substituted or unsubstituted heteroaryl group; —SiRR′R″; —P(═O)RR′; and an amine group unsubstituted or substituted with a substituted or unsubstituted alkyl group, a substituted or unsubstituted aryl group, or a substituted or unsubstituted heteroaryl group, or two or more groups adjacent to each other bond to each other to form a substituted or unsubstituted aromatic hydrocarbon ring or a substituted or unsubstituted heteroring,

R₅ and R₆ are the same as or different from each other, and each independently selected from the group consisting of a substituted or unsubstituted alkyl group; a substituted or unsubstituted aryl group; a substituted or unsubstituted heteroaryl group; and an amine group unsubstituted or substituted with a substituted or unsubstituted alkyl group, a substituted or unsubstituted aryl group, or a substituted or unsubstituted heteroaryl group,

L is a direct bond; a substituted or unsubstituted arylene group; or a substituted or unsubstituted heteroarylene group,

Z is selected from the group consisting of deuterium; —CN; a substituted or unsubstituted alkyl group; a substituted or unsubstituted aryl group; a substituted or unsubstituted heteroaryl group; —SiRR′R″; —P(═O)RR′; and an amine group unsubstituted or substituted with a substituted or unsubstituted alkyl group, a substituted or unsubstituted aryl group, or a substituted or unsubstituted heteroaryl group,

R, R′ and R″ are the same as or different from each other, and each independently hydrogen; a substituted or unsubstituted alkyl group; a substituted or unsubstituted aryl group; or a substituted or unsubstituted heteroaryl group,

m is an integer of 0 to 5,

n is an integer of 1 to 6,

q is an integer of 0 to 2, and

s is an integer of 0 to 3.

Another embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound represented by Chemical Formula 1.

### Advantageous Effects

A compound described in the present specification can be used as a material of an organic material layer of an organic light emitting device. The compound is capable of performing a role of a hole injection material, a hole transfer material, a light emitting material, an electron transfer material, an electron injection material, a charge generation material and the like in an organic light emitting device. Particularly, the compound can be used as a charge generation layer material or an electron transfer layer material of an organic light emitting device.

When using the compound represented by Chemical Formula 1 in an organic material layer, a device driving voltage can be lowered, light efficiency can be enhanced, and device lifetime properties can be enhanced by thermal stability of the compound.

Particularly, the compound represented by Chemical Formula 1 has a substituent of -(L)m-(Z)n on one side benzene ring of the carbazole group, and the π-conjugation structure of the compound of Chemical Formula 1 does not continue from the carbazole group to the fused quinoline group. As a result, the π-conjugation structure of the compound represented by Chemical Formula 1 is disconnected widening a bandgap of a HOMO level and a LUMO level, and the T1 value further increases increasing an effect of locking excitons in a light emitting layer. In addition, by decreasing the HOMO level, holes of the light emitting layer are blocked, and the compound can be used as a compound of a hole blocking layer.

### DESCRIPTION OF DRAWINGS

FIG. 1 to FIG. 4 are diagrams each schematically illustrating a lamination structure of an organic light emitting device according to one embodiment of the present application.

### REFERENCE NUMERAL

100: Substrate
200: Anode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Cathode

### MODE FOR DISCLOSURE

Hereinafter, the present application will be described in detail.

In the present specification, the term “substitution” means a hydrogen atom bonding to a carbon atom of a compound is changed to another substituent, and the position of substitution is not limited as long as it is a position at which the hydrogen atom is substituted, that is, a position at which a substituent can substitute, and when two or more substituents substitute, the two or more substituents may be the same as or different from each other.

In the present specification, “substituted or unsubstituted” means being substituted with one or more substituents selected from the group consisting of C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine, or being unsubstituted, or being substituted with a substituent linking two or more substituents selected from among the substituents illustrated above, or being unsubstituted.

In the present specification, the halogen may be fluorine, chlorine, bromine or iodine.

In the present specification, the alkyl group comprises linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may comprise a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group comprises linear or branched having 2 to 60 carbon atoms, and may be further substituted with other substituents. The number of carbon atoms of the alkenyl group may be from 2 to 60, specifically from 2 to 40 and more specifically from 2 to 20. Specific examples thereof may comprise a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group comprises linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may comprise methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like, but are not limited thereto.

In the present specification, the cycloalkyl group comprises monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may comprise a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group comprises O, S, Se, N or Si as a heteroatom, comprises monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group comprises monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group comprises a spiro group. The number of carbon atoms of the aryl group may be from 6 to 60, specifically from 6 to 40 and more specifically from 6 to 25. Specific examples of the aryl group may comprise a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring thereof, and the like, but are not limited thereto.

In the present specification, the phosphine oxide group is represented by —P(═O)R₁₀₁R₁₀₂, and R₁₀₁ and R₁₀₂ are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the phosphine oxide group may comprise a diphenylphosphine oxide group, a dinaphthylphosphine oxide group and the like, but are not limited thereto.

In the present specification, the silyl group is a substituent comprising Si, having the Si atom directly linked as a radical, and is represented by —SiR₁₀₄R₁₀₅R₁₀₆. R₁₀₄ to R₁₀₆ are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may comprise a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

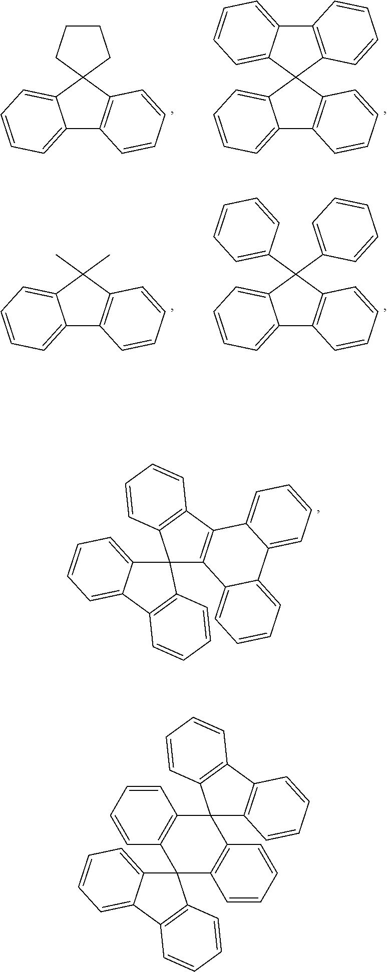

and the like may be included, however, the structure is not limited thereto.

In the present specification, the heteroaryl group comprises O, S, Se, N or Si as a heteroatom, comprises monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may comprise a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a triazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a triazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrobenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may comprise a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. Descriptions on the aryl group provided above may be applied thereto except for those that are each a divalent. In addition, the heteroarylene group means the heteroaryl group having two bonding sites, that is, a divalent group. Descriptions on the heteroaryl group provided above may be applied thereto except for those that are each a divalent.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

One embodiment of the present application provides a compound represented by Chemical Formula 1.

In one embodiment of the present application, Chemical Formula 1 may be represented by any one of the following Chemical Formula 2 to Chemical Formula 7.

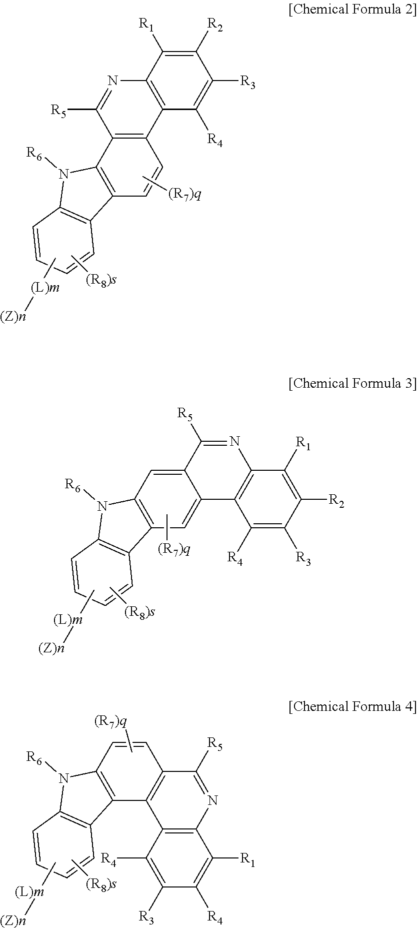

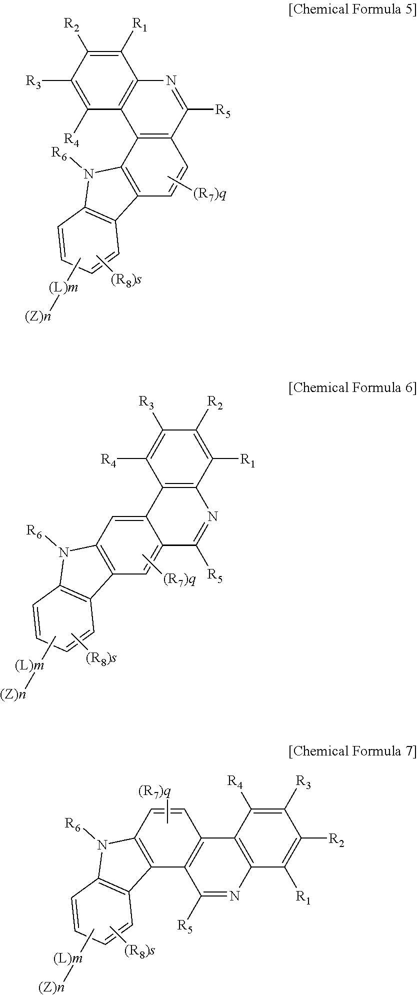

In Chemical Formulae 2 to 7,

R₁ to R₈, L, Z, m, n, s and q have the same definitions as in Chemical Formula 1.

In one embodiment of the present application, Chemical Formula 1 may be represented by any one of the following Chemical Formulae 8 to 11.

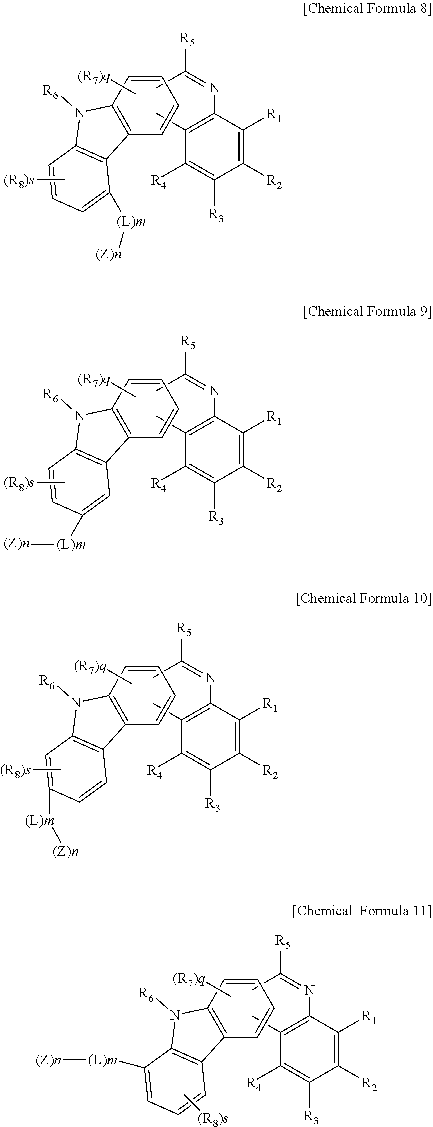

In Chemical Formulae 8 to 11,

R₁ to R₈, L, Z, m, n, s and q have the same definitions as in Chemical Formula 1.

In one embodiment of the present application, R₁ to R₄, R₇ and R₈ are the same as or different from each other, and each independently selected from the group consisting of hydrogen; deuterium; a substituted or unsubstituted alkyl group; a substituted or unsubstituted aryl group; a substituted or unsubstituted heteroaryl group; —SiRR′R″; —P(═O)RR′; and an amine group unsubstituted or substituted with a substituted or unsubstituted alkyl group, a substituted or unsubstituted aryl group, or a substituted or unsubstituted heteroaryl group, or two or more groups adjacent to each other may bond to each other to form a substituted or unsubstituted aromatic hydrocarbon ring or a substituted or unsubstituted heteroring.

In another embodiment, R₁ to R₄, R₇ and R₈ are the same as or different from each other, and may be each independently hydrogen; a substituted or unsubstituted alkyl group; a substituted or unsubstituted aryl group; or a substituted or unsubstituted heteroaryl group.

In another embodiment, R₁ to R₄, R₇ and R₈ are the same as or different from each other, and may be each independently hydrogen; a substituted or unsubstituted C1 to C60 alkyl group; a substituted or unsubstituted C6 to C60 aryl group; or a substituted or unsubstituted C2 to C60 heteroaryl group.

In another embodiment, R₁ to R₄, R₇ and R₈ are the same as or different from each other, and may be each independently hydrogen; a substituted or unsubstituted C1 to C40 alkyl group; a substituted or unsubstituted C6 to C40 aryl group; or a substituted or unsubstituted C2 to C40 heteroaryl group.

In another embodiment, R₁ to R₄, R₇ and R₈ are the same as or different from each other, and may be each independently hydrogen; a C1 to C40 alkyl group; a C6 to C40 aryl group; or a C2 to C40 heteroaryl group.

In another embodiment, R₁ to R₄, R₇ and R₈ may be hydrogen.

In one embodiment of the present application, R₅ and R₆ are the same as or different from each other, and may be each independently selected from the group consisting of a substituted or unsubstituted alkyl group; a substituted or unsubstituted aryl group; a substituted or unsubstituted heteroaryl group; and an amine group unsubstituted or substituted with a substituted or unsubstituted alkyl group, a substituted or unsubstituted aryl group, or a substituted or unsubstituted heteroaryl group.

In another embodiment, R₅ and R₆ are the same as or different from each other, and may be each independently selected from the group consisting of a substituted or unsubstituted C1 to C60 alkyl group; a substituted or unsubstituted C6 to C60 aryl group; a substituted or unsubstituted C2 to C60 heteroaryl group; and an amine group unsubstituted or substituted with a substituted or unsubstituted C1 to C60 alkyl group, a substituted or unsubstituted C6 to C60 aryl group, or a substituted or unsubstituted C2 to C60 heteroaryl group.

In another embodiment, R₅ and R₆ are the same as or different from each other, and may be each independently selected from the group consisting of a substituted or unsubstituted C1 to C40 alkyl group; a substituted or unsubstituted C6 to C40 aryl group; a substituted or unsubstituted C2 to C40 heteroaryl group; and an amine group unsubstituted or substituted with a substituted or unsubstituted C1 to C40 alkyl group, a substituted or unsubstituted C6 to C40 aryl group, or a substituted or unsubstituted C2 to C40 heteroaryl group.

In another embodiment, R₅ and R₆ are the same as or different from each other, and may be each independently a substituted or unsubstituted C6 to C40 aryl group.

In another embodiment, R₅ and R₆ are the same as or different from each other, and may be each independently a monocyclic or polycyclic C6 to C40 aryl group.

In another embodiment, R₅ and R₆ are the same as or different from each other, and may be each independently a phenyl group; or a naphthyl group.

Particularly, when R₅ and R₆ have a substituted or unsubstituted aryl group in the present application, the molecular weight increases compared to the compound having a heteroaryl group disubstituted or trisubstituted, and thermal stability is enhanced increasing a lifetime. In addition, the overall compound structure is planar, and an electron transfer ability is improved particularly increasing efficiency. The high T1 value blocks excitons, and holes in a light emitting layer, and as a result, a lifetime is improved.

In one embodiment of the present application, L may be a direct bond; a substituted or unsubstituted arylene group; or a substituted or unsubstituted heteroarylene group.

In another embodiment, L may be a direct bond; a substituted or unsubstituted C6 to C60 arylene group; or a substituted or unsubstituted C2 to C60 heteroarylene group.

In another embodiment, L may be a direct bond; a substituted or unsubstituted C6 to C40 arylene group; or a substituted or unsubstituted C2 to C40 heteroarylene group.

In another embodiment, L may be a direct bond; a C6 to C40 arylene group; or a C2 to C40 heteroarylene group unsubstituted or substituted with a C6 to C40 aryl group.

In another embodiment, L may be a direct bond; a phenylene group; a biphenylene group; a naphthylene group; an anthracenylene group; a divalent pyrimidine group unsubstituted or substituted with a phenyl group; or a divalent triazine group unsubstituted or substituted with a phenyl group.

In one embodiment of the present application, Z may be selected from the group consisting of deuterium; —CN; a substituted or unsubstituted alkyl group; a substituted or unsubstituted aryl group; a substituted or unsubstituted heteroaryl group; —SiRR′R″; —P(═O)RR′; and an amine group unsubstituted or substituted with a substituted or unsubstituted alkyl group, a substituted or unsubstituted aryl group, or a substituted or unsubstituted heteroaryl group.

In another embodiment, Z may be selected from the group consisting of deuterium; —CN; a substituted or unsubstituted C1 to C60 alkyl group; a substituted or unsubstituted C6 to C60 aryl group; a substituted or unsubstituted C2 to C60 heteroaryl group; —SiRR′R″; —P(═O)RR′; and an amine group unsubstituted or substituted with a substituted or unsubstituted C1 to C60 alkyl group, a substituted or unsubstituted C6 to C60 aryl group, or a substituted or unsubstituted C2 to C60 heteroaryl group.

In another embodiment, Z may be selected from the group consisting of deuterium; —CN; a substituted or unsubstituted C1 to C40 alkyl group; a substituted or unsubstituted C6 to C40 aryl group; a substituted or unsubstituted C2 to C40 heteroaryl group; —SiRR′R″; —P(═O)RR′; and an amine group unsubstituted or substituted with a substituted or unsubstituted C1 to C40 alkyl group, a substituted or unsubstituted C6 to C40 aryl group, or a substituted or unsubstituted C2 to C40 heteroaryl group.

In another embodiment, Z may be selected from the group consisting of —CN; a C6 to C40 aryl group unsubstituted or substituted with one or more substituents selected from the group consisting of a C1 to C40 alkyl group, a C6 to C40 aryl group, a C2 to C40 heteroaryl group and —P(═O)RR′; a C2 to C40 heteroaryl group unsubstituted or substituted with one or more substituents selected from the group consisting of a C1 to C40 alkyl group, a C6 to C40 aryl group and a C2 to C40 heteroaryl group; —P(═O)RR′; and an amine group unsubstituted or substituted with a C6 to C40 aryl group.

In another embodiment, Z may be selected from the group consisting of a C6 to C40 aryl group unsubstituted or substituted with one or more substituents selected from the group consisting of a C6 to C40 aryl group and —P(═O)RR′; a C2 to C40 heteroaryl group unsubstituted or substituted with one or more substituents selected from the group consisting of a C6 to C40 aryl group and a C2 to C40 heteroaryl group; —P(═O)RR′; and an amine group unsubstituted or substituted with a C6 to C40 aryl group.

In another embodiment, Z may be selected from the group consisting of a substituted or unsubstituted phenyl group; a substituted or unsubstituted naphthyl group; a substituted or unsubstituted pyridine group; a substituted or unsubstituted pyrimidine group; a substituted or unsubstituted triazine group; a substituted or unsubstituted phenanthroline group; a substituted or unsubstituted carbazole group; a substituted or unsubstituted dibenzofuran group; a substituted or unsubstituted dibenzothiophene group; —P(═O)RR′; and a substituted or unsubstituted amine group.

In another embodiment, Z may be selected from the group consisting of a phenyl group unsubstituted or substituted with —P(═O)RR′; a naphthyl group; a pyridine group unsubstituted or substituted with a pyridine group; a pyrimidine group unsubstituted or substituted with one or more substituents selected from the group consisting of a phenyl group and a biphenyl group; a triazine group unsubstituted or substituted with one or more substituents selected from the group consisting of a phenyl group and a biphenyl group; a phenanthroline group unsubstituted or substituted with a phenyl group; a carbazole group; a dibenzofuran group; a dibenzothiophene group; —P(═O)RR′; and an amine group unsubstituted or substituted with a phenyl group.

In one embodiment of the present application, Z may be substituted again with one or more substituents selected from the group consisting of a C6 to C40 aryl group; a C2 to C40 heteroaryl group; and an amine group unsubstituted or substituted with a substituted or unsubstituted C1 to C40 alkyl group, a substituted or unsubstituted C6 to C40 aryl group, or a substituted or unsubstituted C2 to C40 heteroaryl group.

In another embodiment, Z may be substituted again with one or more substituents selected from the group consisting of a carbazole group; a dibenzofuran group; a dibenzothiophene group; and a diphenylamine group.

Particularly, the compound represented by Chemical Formula 1 has a substituent of -(L)m-(Z)n on one side benzene ring of the carbazole group, and the π-conjugation structure of the compound of Chemical Formula 1 does not continue from the carbazole group to the fused quinoline group. As a result, the π-conjugation structure of the compound represented by Chemical Formula 1 is disconnected widening a bandgap of a HOMO level and a LUMO level, and the T1 value further increases increasing an effect of locking excitons in a light emitting layer. In addition, by decreasing the HOMO level, holes of the light emitting layer are blocked, and the compound may be used as a compound of a hole blocking layer.

In addition, thermal stability that the compound has may increase by changing substituents in the core structure of Chemical Formula 1. In addition, the structure of the compound is planar, which increases an electron transfer ability.

In one embodiment of the present application, R, R′ and R″ are the same as or different from each other, and may be each independently hydrogen; a substituted or unsubstituted alkyl group; a substituted or unsubstituted aryl group; or a substituted or unsubstituted heteroaryl group.

In another embodiment, R, R′ and R″ are the same as or different from each other, and may be each independently a substituted or unsubstituted C1 to C60 alkyl group; or a substituted or unsubstituted C6 to C60 aryl group.

In another embodiment, R, R′ and R″ are the same as or different from each other, and may be each independently a substituted or unsubstituted C1 to C40 alkyl group; or a substituted or unsubstituted C6 to C40 aryl group.

In another embodiment, R, R′ and R″ are the same as or different from each other, and may be each independently a C1 to C40 alkyl group; or a C6 to C40 aryl group.

In another embodiment, R, R′ and R″ are the same as or different from each other, and may be each independently a methyl group; or a phenyl group.

In another embodiment, R, R′ and R″ are the same as or different from each other, and may be each independently a C6 to C40 aryl group.

In another embodiment, R, R′ and R″ are the same as or different from each other, and may be each independently a phenyl group.

In one embodiment of the present application, -(L)m-(Z)n of the substituent

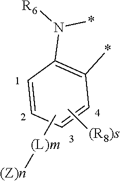

of Chemical Formula 3 may bond to the number 1 position.

In one embodiment of the present application, -(L)m-(Z)n of the substituent

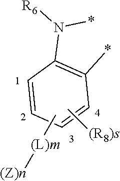

of Chemical Formula 3 may bond to the number 2 position.

In one embodiment of the present application, -(L)m-(Z)n of the substituent

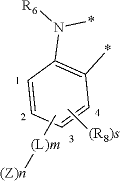

of Chemical Formula 3 may bond to the number 3 position.

In one embodiment of the present application, -(L)m-(Z)n of the substituent

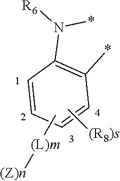

of Chemical Formula 3 may bond to the number 4 position.

In one embodiment of the present application, -(L)m-(Z)n of the substituent

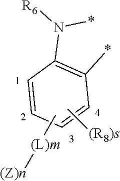

of Chemical Formula 4 may bond to the number 1 position.

In one embodiment of the present application, -(L)m-(Z)n of the substituent

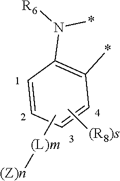

of Chemical Formula 4 may bond to the number 2 position.

In one embodiment of the present application, -(L)m-(Z)n of the substituent

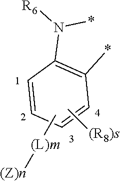

of Chemical Formula 4 may bond to the number 3 position.

In one embodiment of the present application, -(L)m-(Z)n of the substituent

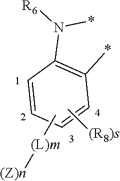

of Chemical Formula 5 may bond to the number 1 position.

In one embodiment of the present application, -(L)m-(Z)n of the substituent

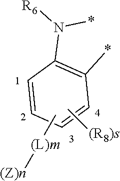

of Chemical Formula 5 may bond to the number 2 position.

In one embodiment of the present application, -(L)m-(Z)n of the substituent

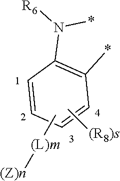

of Chemical Formula 5 may bond to the number 3 position.

In one embodiment of the present application, -(L)m-(Z)n of the substituent

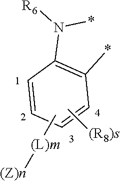

of Chemical Formula 5 may bond to the number 4 position.

In one embodiment of the present application, -(L)m-(Z)n of the substituent

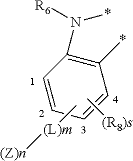

of Chemical Formula 6 may bond to the number 1 position.

In one embodiment of the present application, -(L)m-(Z)n of the substituent

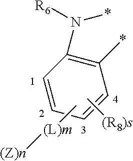

of Chemical Formula 6 may bond to the number 2 position.

In one embodiment of the present application, -(L)m-(Z)n of the substituent

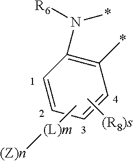

of Chemical Formula 6 may bond to the number 3 position.

In one embodiment of the present application, -(L)m-(Z)n of the substituent

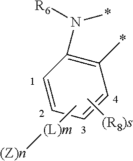

of Chemical Formula 6 may bond to the number 4 position.

In one embodiment of the present application, -(L)m-(Z)n of the substituent

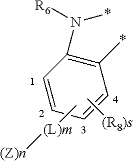

of Chemical Formula 7 may bond to the number 1 position.

In one embodiment of the present application, -(L)m-(Z)n of the substituent

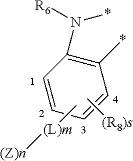

of Chemical Formula 7 may bond to the number 2 position. [symbol] means a site bonding to the chemical formula.

In the heterocyclic compound provided in one embodiment of the present application, Chemical Formula 1 is represented by any one of the following compounds.

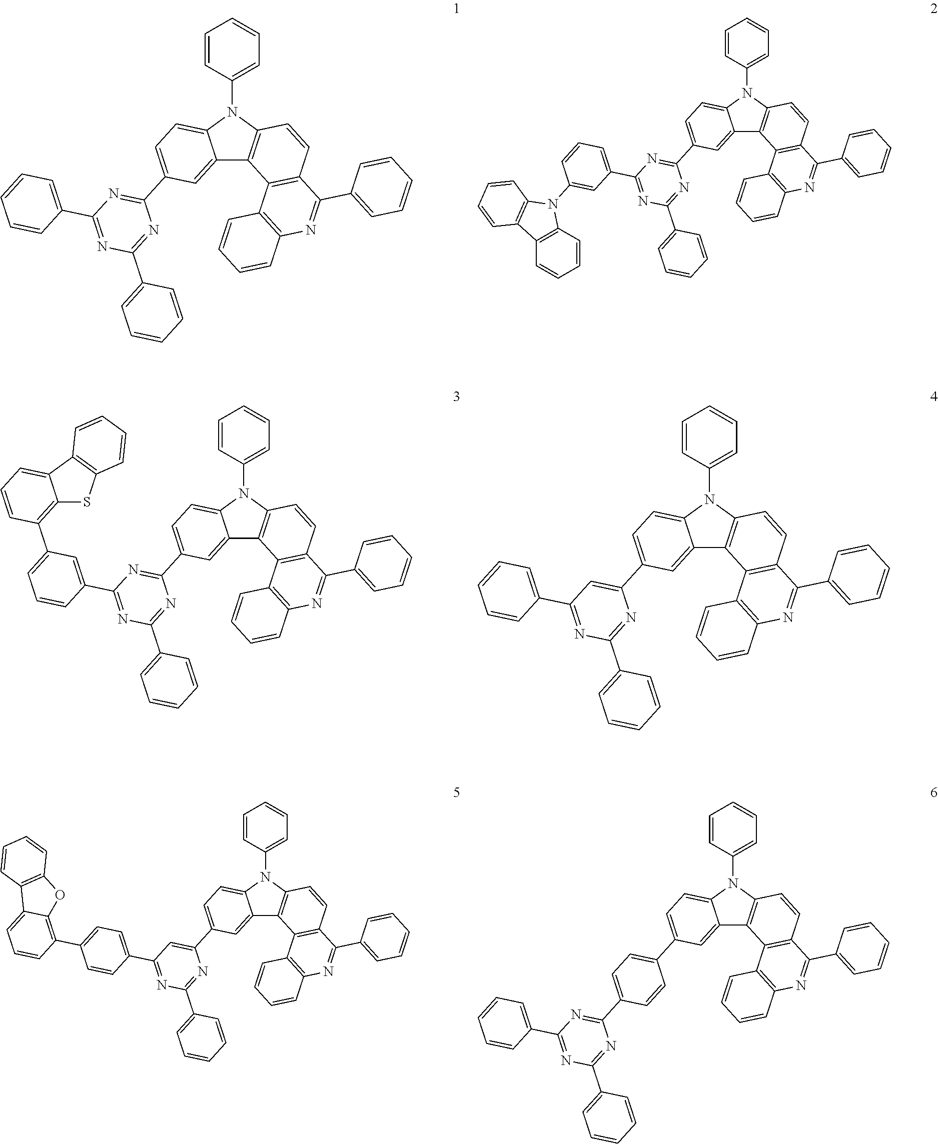

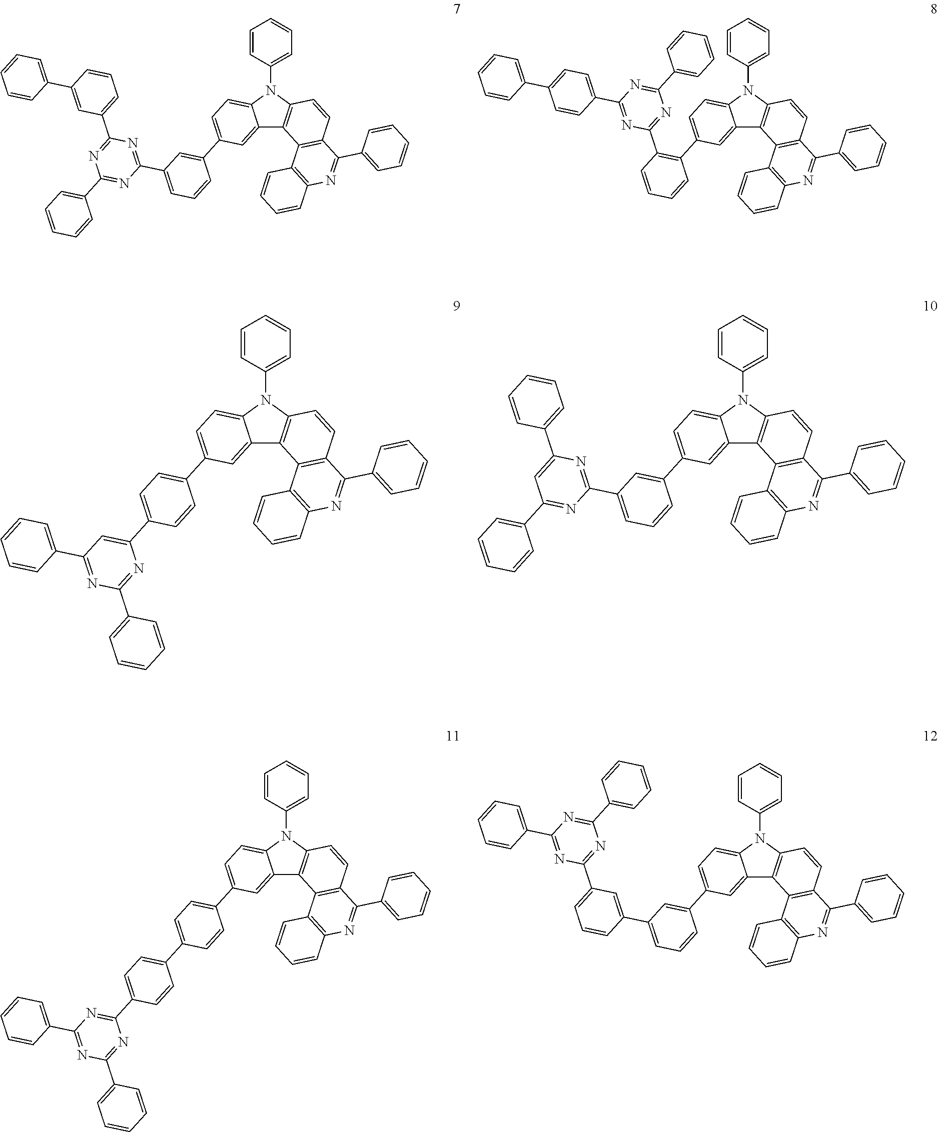

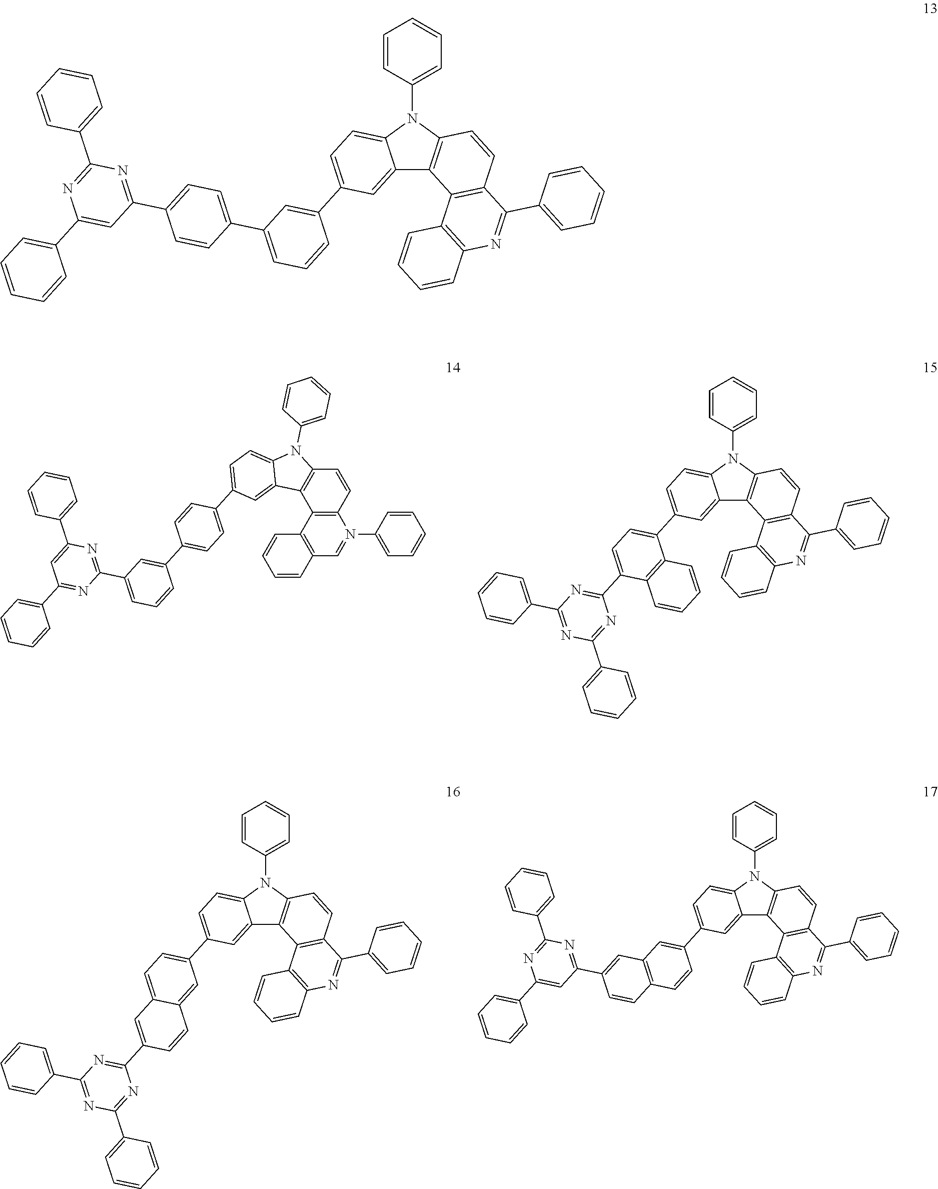

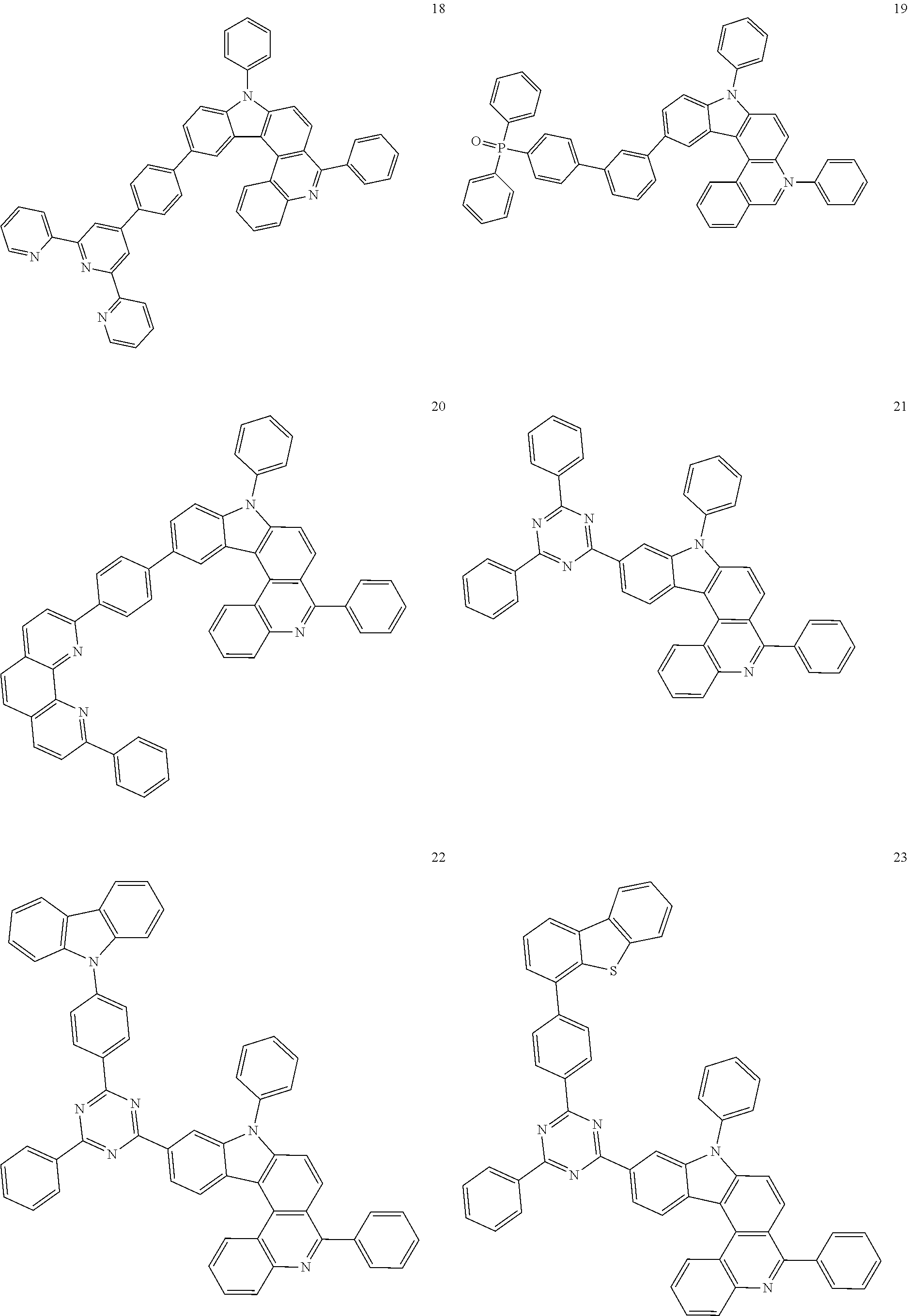

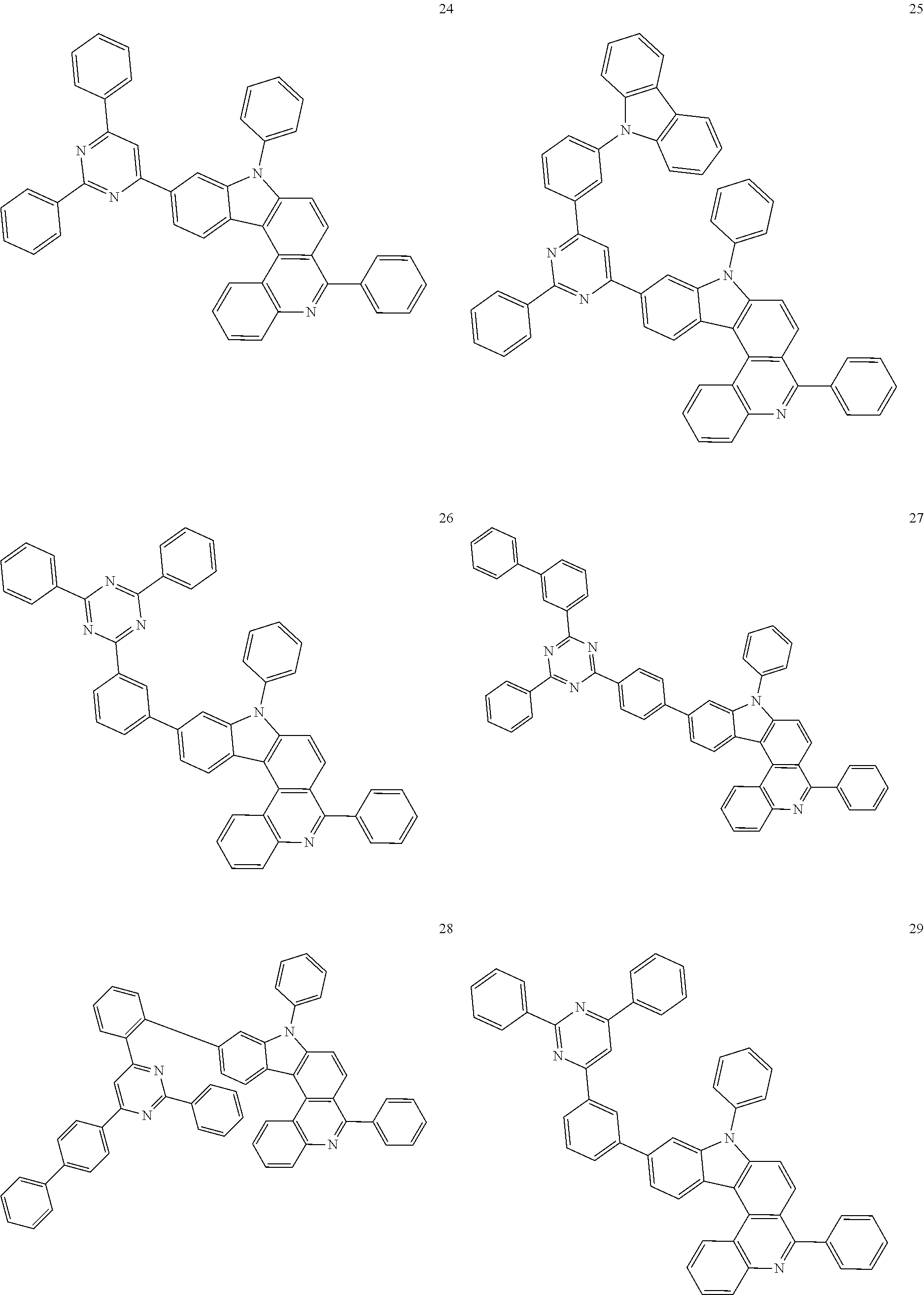

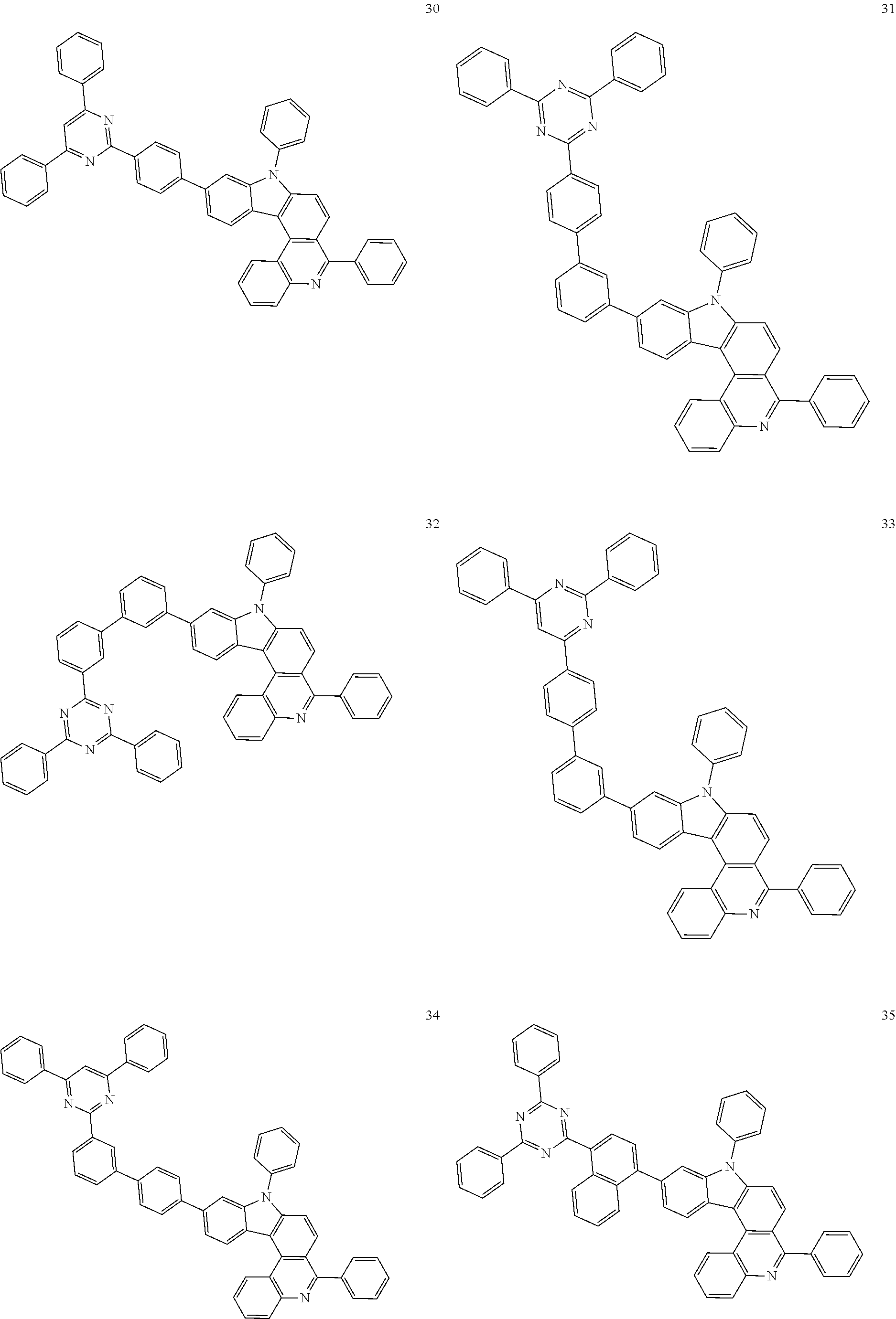

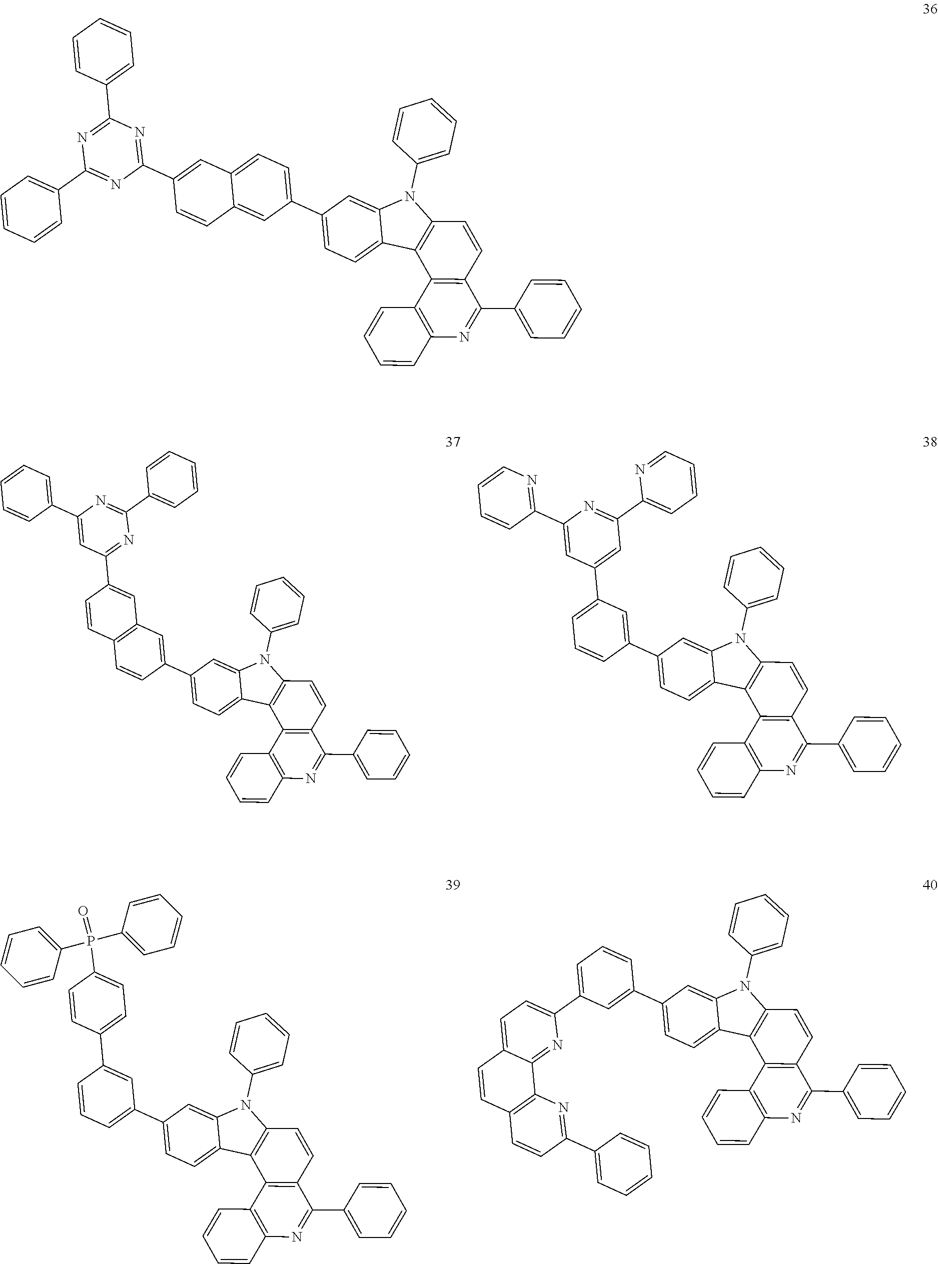

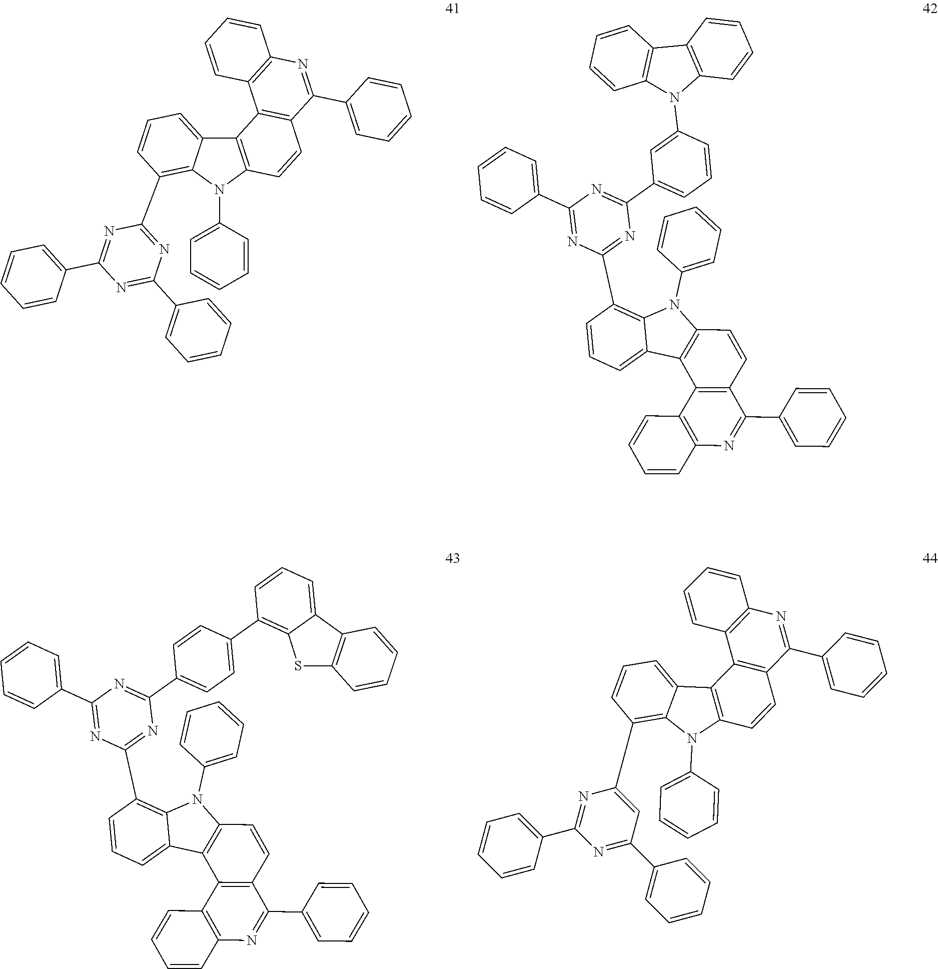 [SMILES: c1ccc(-c2nc(-c3ccccc3)nc(-c3cccc4c5c6c(ccc5n(-c5ccccc5)c34)c(-c3ccccc3)nc3ccccc36)n2)cc1 ; c1ccc(-c2nc(-c3cccc(-n4c5ccccc5c5ccccc54)c3)nc(-c3cccc4c5c6c(ccc5n(-c5ccccc5)c34)c(-c3ccccc3)nc3ccccc36)n2)cc1 ; c1ccc(-c2nc(-c3ccc(-c4cccc5c4sc4ccccc45)cc3)nc(-c3cccc4c5c6c(ccc5n(-c5ccccc5)c34)c(-c3ccccc3)nc3ccccc36)n2)cc1 ; c1ccc(-c2cc(-c3cccc4c5c6c(ccc5n(-c5ccccc5)c34)c(-c3ccccc3)nc3ccccc36)nc(-c3ccccc3)n2)cc1]

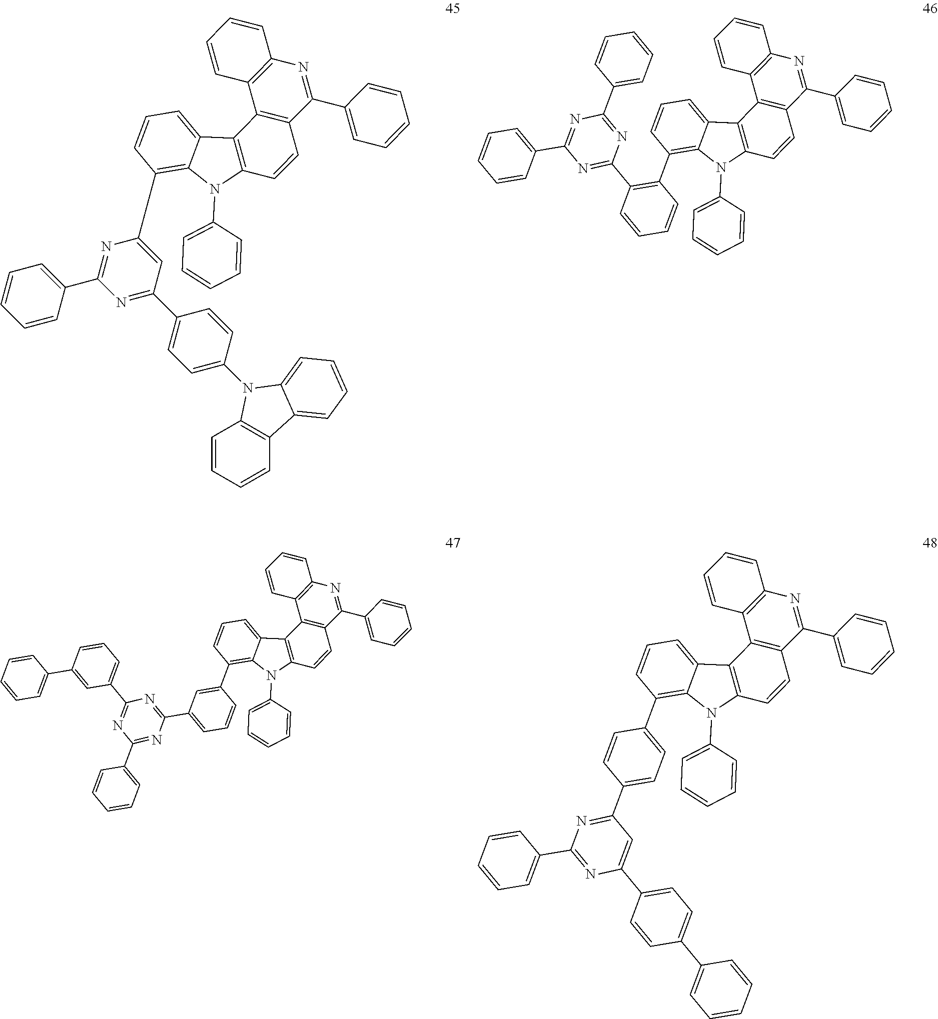 [SMILES: c1ccc(-c2nc(-c3ccc(-n4c5ccccc5c5ccccc54)cc3)cc(-c3cccc4c5c6c(ccc5n(-c5ccccc5)c34)c(-c3ccccc3)nc3ccccc36)n2)cc1 ; c1ccc(-c2nc(-c3ccccc3)nc(-c3ccccc3-c3cccc4c5c6c(ccc5n(-c5ccccc5)c34)c(-c3ccccc3)nc3ccccc36)n2)cc1 ; c1ccc(-c2cccc(-c3nc(-c4ccccc4)nc(-c4cccc(-c5cccc6c7c8c(ccc7n(-c7ccccc7)c56)c(-c5ccccc5)nc5ccccc58)c4)n3)c2)cc1 ; c1ccc(-c2ccc(-c3cc(-c4ccc(-c5cccc6c7c8c(ccc7n(-c7ccccc7)c56)c(-c5ccccc5)nc5ccccc58)cc4)nc(-c4ccccc4)n3)cc2)cc1]

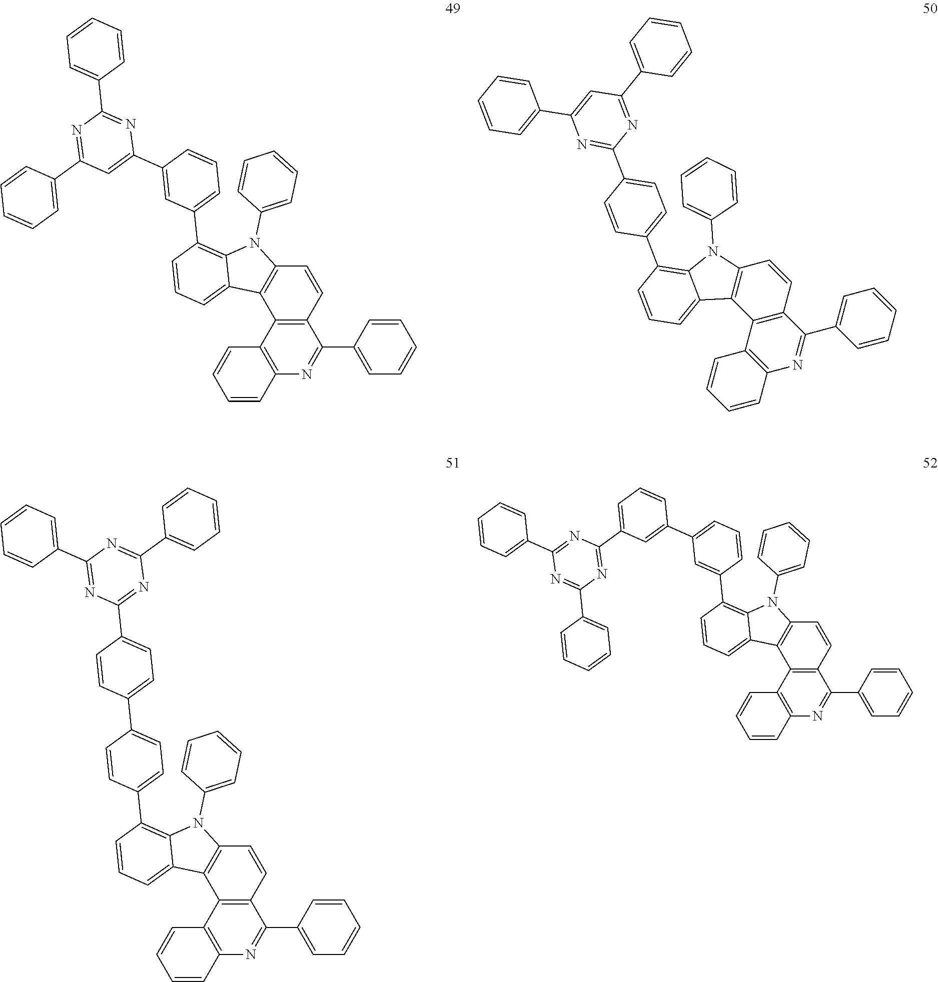 [SMILES: c1ccc(-c2cc(-c3cccc(-c4cccc5c6c7c(ccc6n(-c6ccccc6)c45)c(-c4ccccc4)nc4ccccc47)c3)nc(-c3ccccc3)n2)cc1 ; c1ccc(-c2cc(-c3ccccc3)nc(-c3ccc(-c4cccc5c6c7c(ccc6n(-c6ccccc6)c45)c(-c4ccccc4)nc4ccccc47)cc3)n2)cc1 ; c1ccc(-c2nc(-c3ccccc3)nc(-c3ccc(-c4ccc(-c5cccc6c7c8c(ccc7n(-c7ccccc7)c56)c(-c5ccccc5)nc5ccccc58)cc4)cc3)n2)cc1 ; c1ccc(-c2nc(-c3ccccc3)nc(-c3cccc(-c4cccc(-c5cccc6c7c8c(ccc7n(-c7ccccc7)c56)c(-c5ccccc5)nc5ccccc58)c4)c3)n2)cc1]

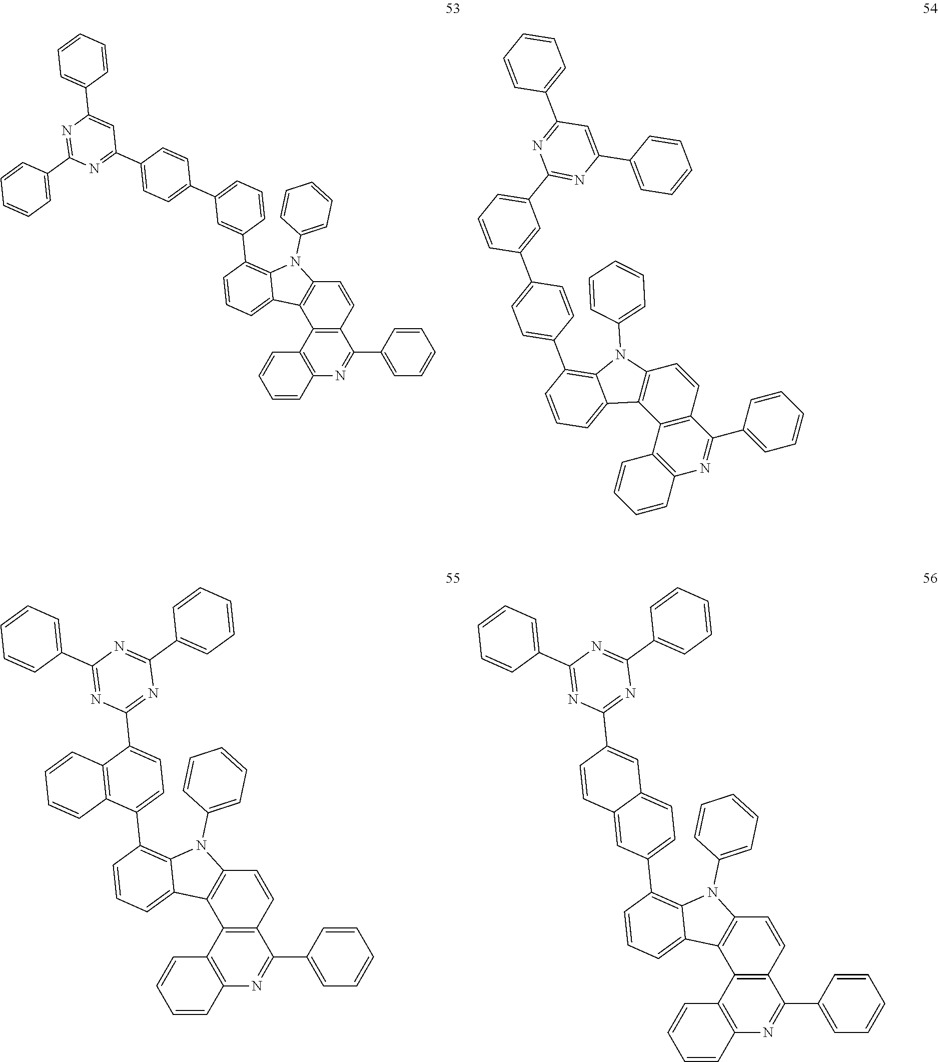 [SMILES: c1ccc(-c2cc(-c3ccc(-c4cccc(-c5cccc6c7c8c(ccc7n(-c7ccccc7)c56)c(-c5ccccc5)nc5ccccc58)c4)cc3)nc(-c3ccccc3)n2)cc1 ; c1ccc(-c2cc(-c3ccccc3)nc(-c3cccc(-c4ccc(-c5cccc6c7c8c(ccc7n(-c7ccccc7)c56)c(-c5ccccc5)nc5ccccc58)cc4)c3)n2)cc1 ; c1ccc(-c2nc(-c3ccccc3)nc(-c3ccc(-c4cccc5c6c7c(ccc6n(-c6ccccc6)c45)c(-c4ccccc4)nc4ccccc47)c4ccccc34)n2)cc1 ; c1ccc(-c2nc(-c3ccccc3)nc(-c3ccc4cc(-c5cccc6c7c8c(ccc7n(-c7ccccc7)c56)c(-c5ccccc5)nc5ccccc58)ccc4c3)n2)cc1]

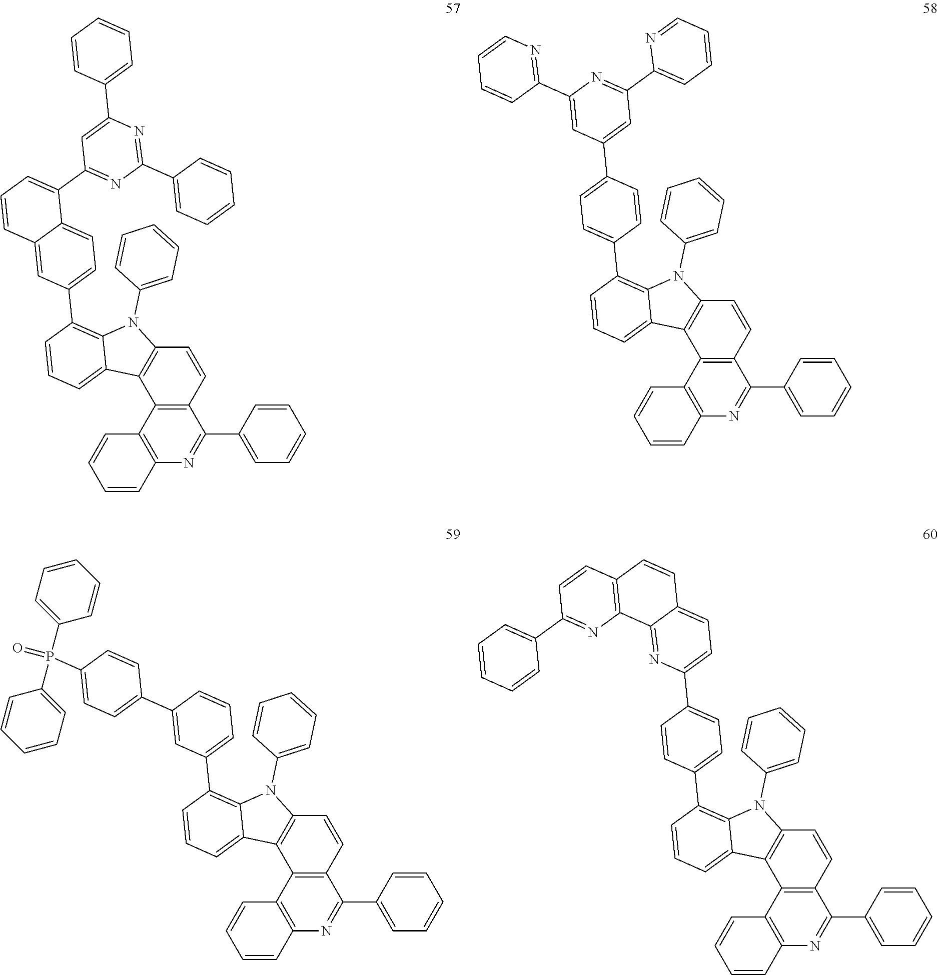 [SMILES: c1ccc(-c2cc(-c3cccc4cc(-c5cccc6c7c8c(ccc7n(-c7ccccc7)c56)c(-c5ccccc5)nc5ccccc58)ccc34)nc(-c3ccccc3)n2)cc1 ; c1ccc(-c2nc3ccccc3c3c2ccc2c3c3cccc(-c4ccc(-c5cc(-c6ccccn6)nc(-c6ccccn6)c5)cc4)c3n2-c2ccccc2)cc1 ; O=P(c1ccccc1)(c1ccccc1)c1ccc(-c2cccc(-c3cccc4c5c6c(ccc5n(-c5ccccc5)c34)c(-c3ccccc3)nc3ccccc36)c2)cc1 ; c1ccc(-c2ccc3ccc4ccc(-c5ccc(-c6cccc7c8c9c(ccc8n(-c8ccccc8)c67)c(-c6ccccc6)nc6ccccc69)cc5)nc4c3n2)cc1]

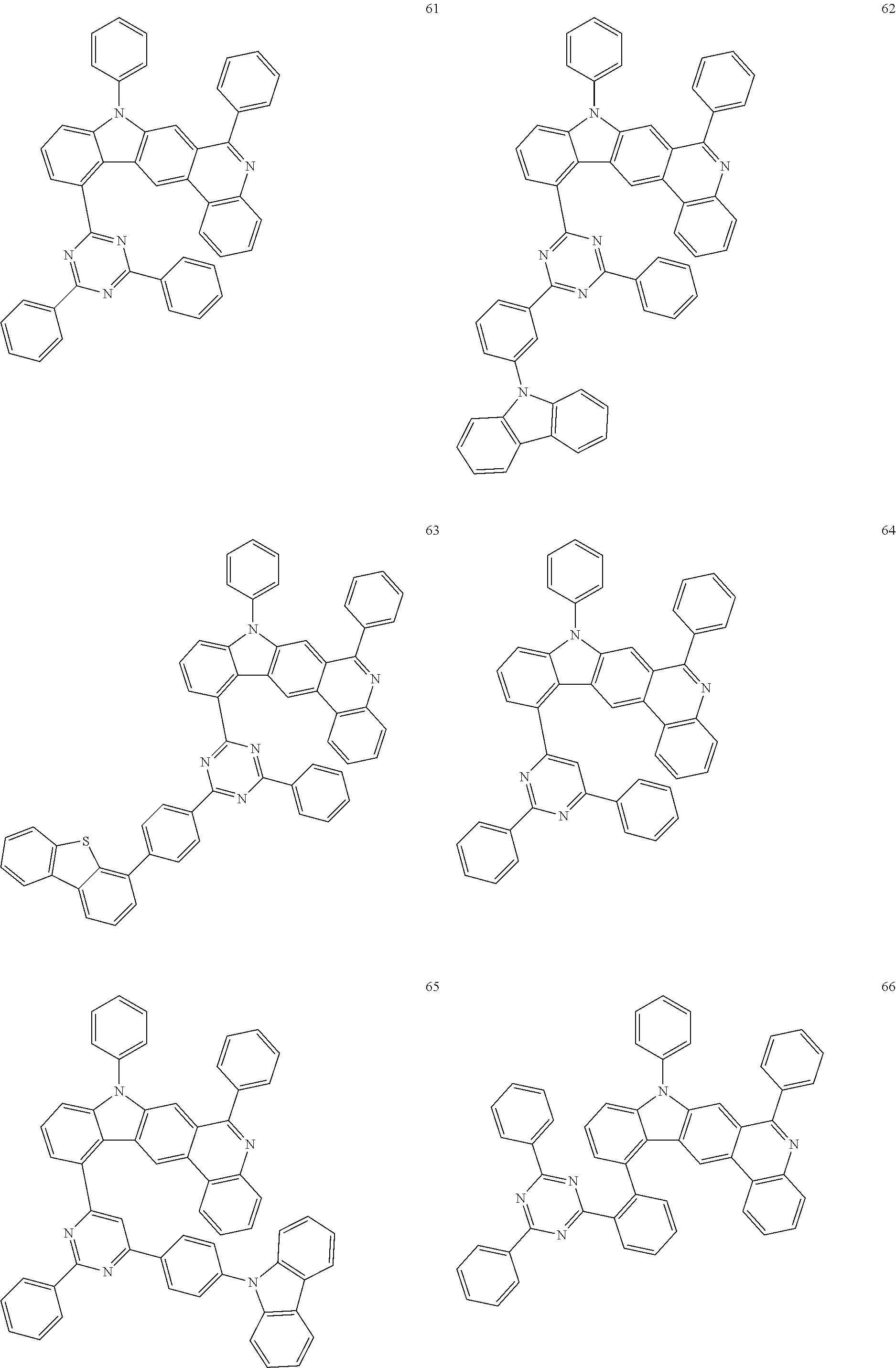

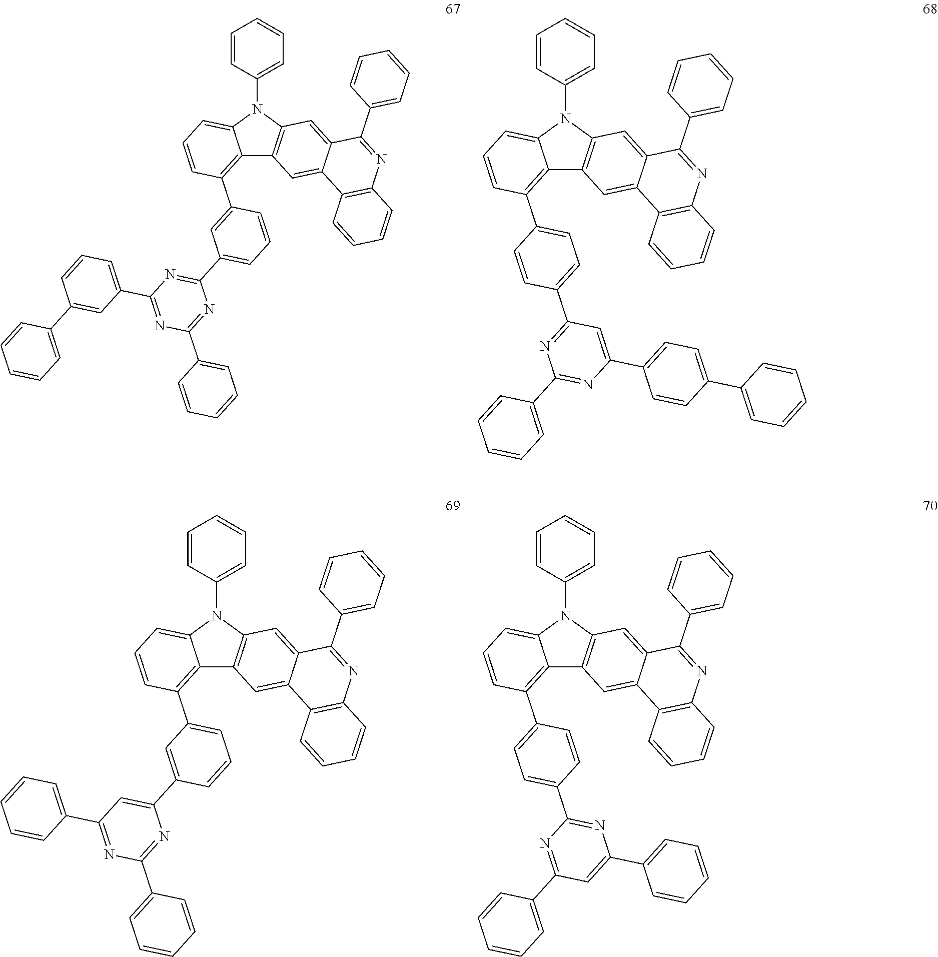 [SMILES: c1ccc(-c2cccc(-c3nc(-c4ccccc4)nc(-c4cccc(-c5cccc6c5c5cc7c(cc5n6-c5ccccc5)c(-c5ccccc5)nc5ccccc57)c4)n3)c2)cc1 ; c1ccc(-c2ccc(-c3cc(-c4ccc(-c5cccc6c5c5cc7c(cc5n6-c5ccccc5)c(-c5ccccc5)nc5ccccc57)cc4)nc(-c4ccccc4)n3)cc2)cc1 ; c1ccc(-c2cc(-c3cccc(-c4cccc5c4c4cc6c(cc4n5-c4ccccc4)c(-c4ccccc4)nc4ccccc46)c3)nc(-c3ccccc3)n2)cc1 ; c1ccc(-c2cc(-c3ccccc3)nc(-c3ccc(-c4cccc5c4c4cc6c(cc4n5-c4ccccc4)c(-c4ccccc4)nc4ccccc46)cc3)n2)cc1]

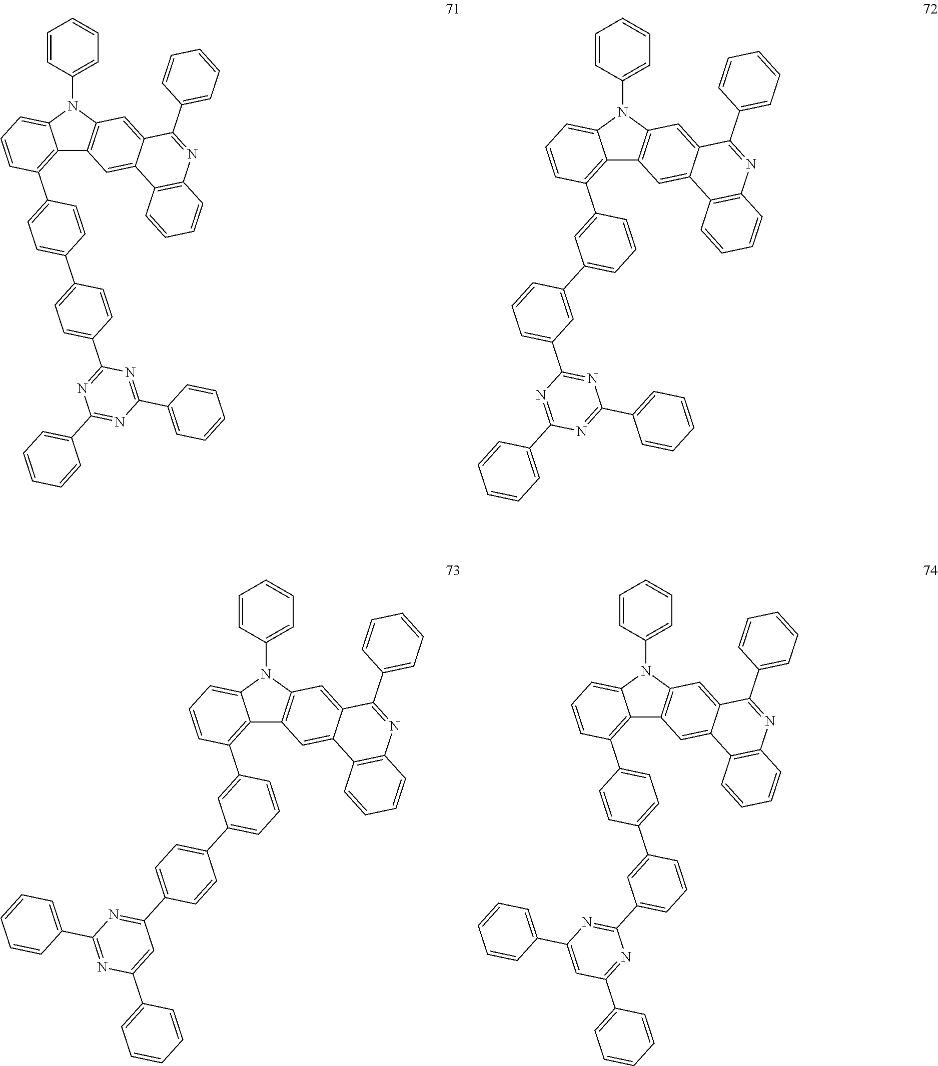 [SMILES: c1ccc(-c2nc(-c3ccccc3)nc(-c3ccc(-c4ccc(-c5cccc6c5c5cc7c(cc5n6-c5ccccc5)c(-c5ccccc5)nc5ccccc57)cc4)cc3)n2)cc1 ; c1ccc(-c2nc(-c3ccccc3)nc(-c3cccc(-c4cccc(-c5cccc6c5c5cc7c(cc5n6-c5ccccc5)c(-c5ccccc5)nc5ccccc57)c4)c3)n2)cc1 ; c1ccc(-c2cc(-c3ccc(-c4cccc(-c5cccc6c5c5cc7c(cc5n6-c5ccccc5)c(-c5ccccc5)nc5ccccc57)c4)cc3)nc(-c3ccccc3)n2)cc1 ; c1ccc(-c2cc(-c3ccccc3)nc(-c3cccc(-c4ccc(-c5cccc6c5c5cc7c(cc5n6-c5ccccc5)c(-c5ccccc5)nc5ccccc57)cc4)c3)n2)cc1]

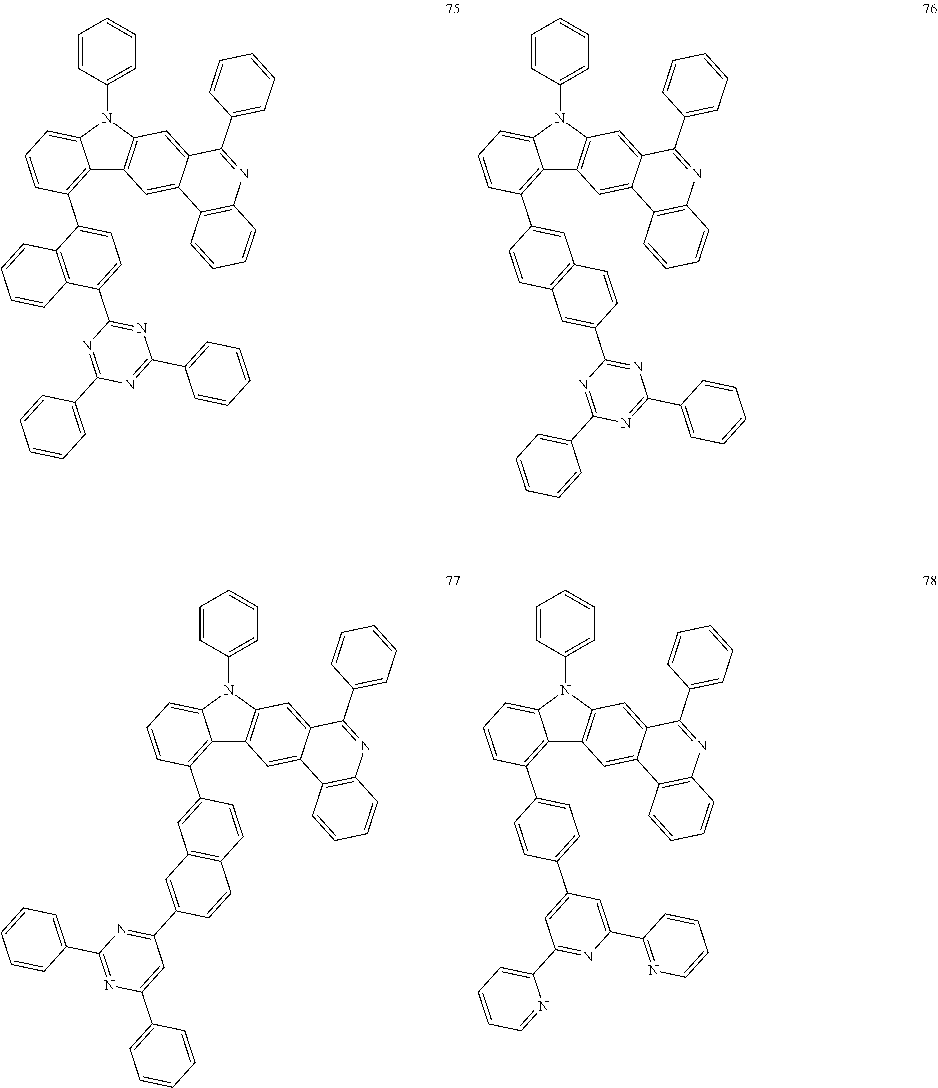 [SMILES: c1ccc(-c2nc(-c3ccccc3)nc(-c3ccc(-c4cccc5c4c4cc6c(cc4n5-c4ccccc4)c(-c4ccccc4)nc4ccccc46)c4ccccc34)n2)cc1 ; c1ccc(-c2nc(-c3ccccc3)nc(-c3ccc4cc(-c5cccc6c5c5cc7c(cc5n6-c5ccccc5)c(-c5ccccc5)nc5ccccc57)ccc4c3)n2)cc1 ; c1ccc(-c2cc(-c3ccc4ccc(-c5cccc6c5c5cc7c(cc5n6-c5ccccc5)c(-c5ccccc5)nc5ccccc57)cc4c3)nc(-c3ccccc3)n2)cc1 ; c1ccc(-c2nc3ccccc3c3cc4c5c(-c6ccc(-c7cc(-c8ccccn8)nc(-c8ccccn8)c7)cc6)cccc5n(-c5ccccc5)c4cc23)cc1]

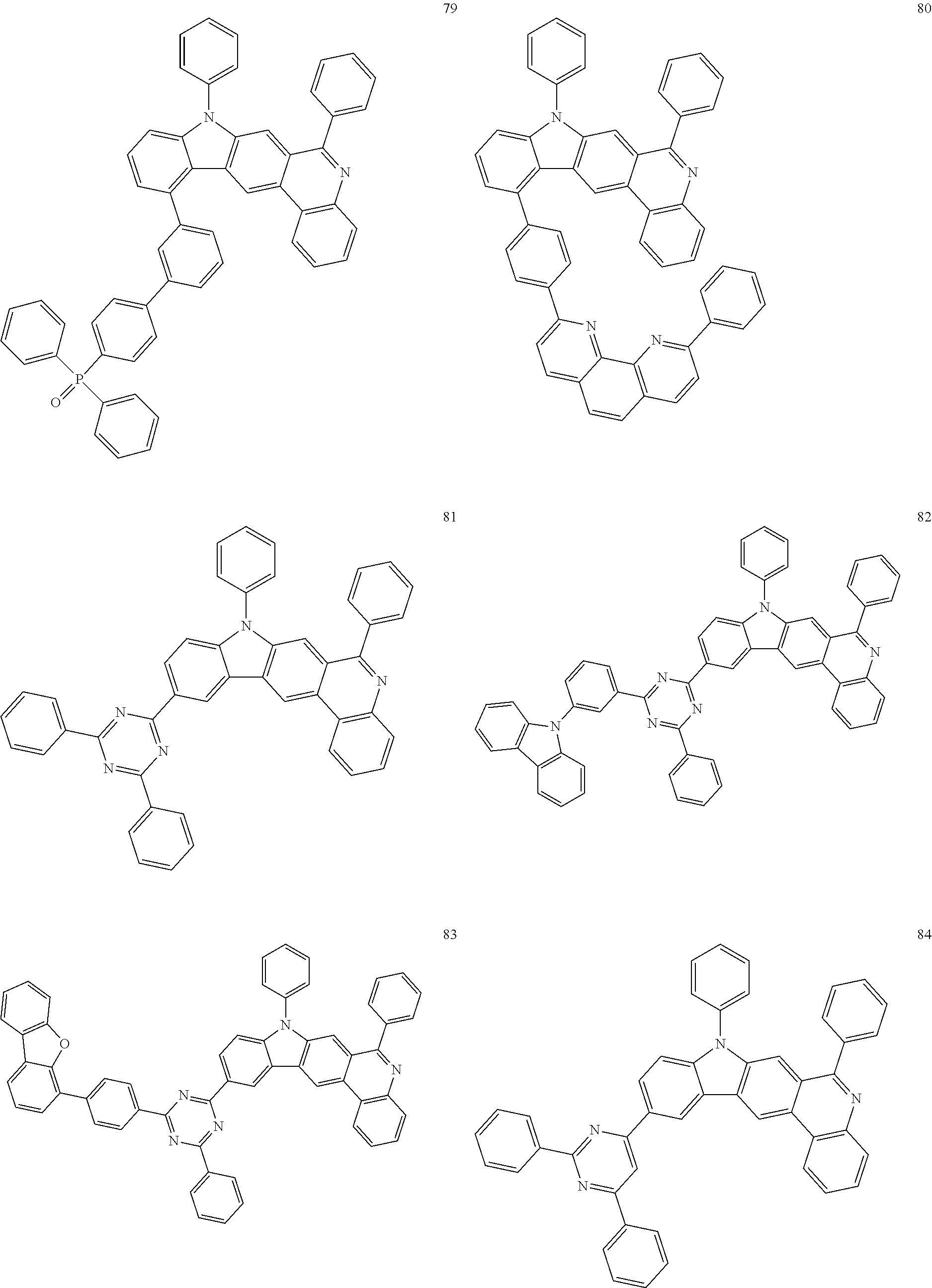

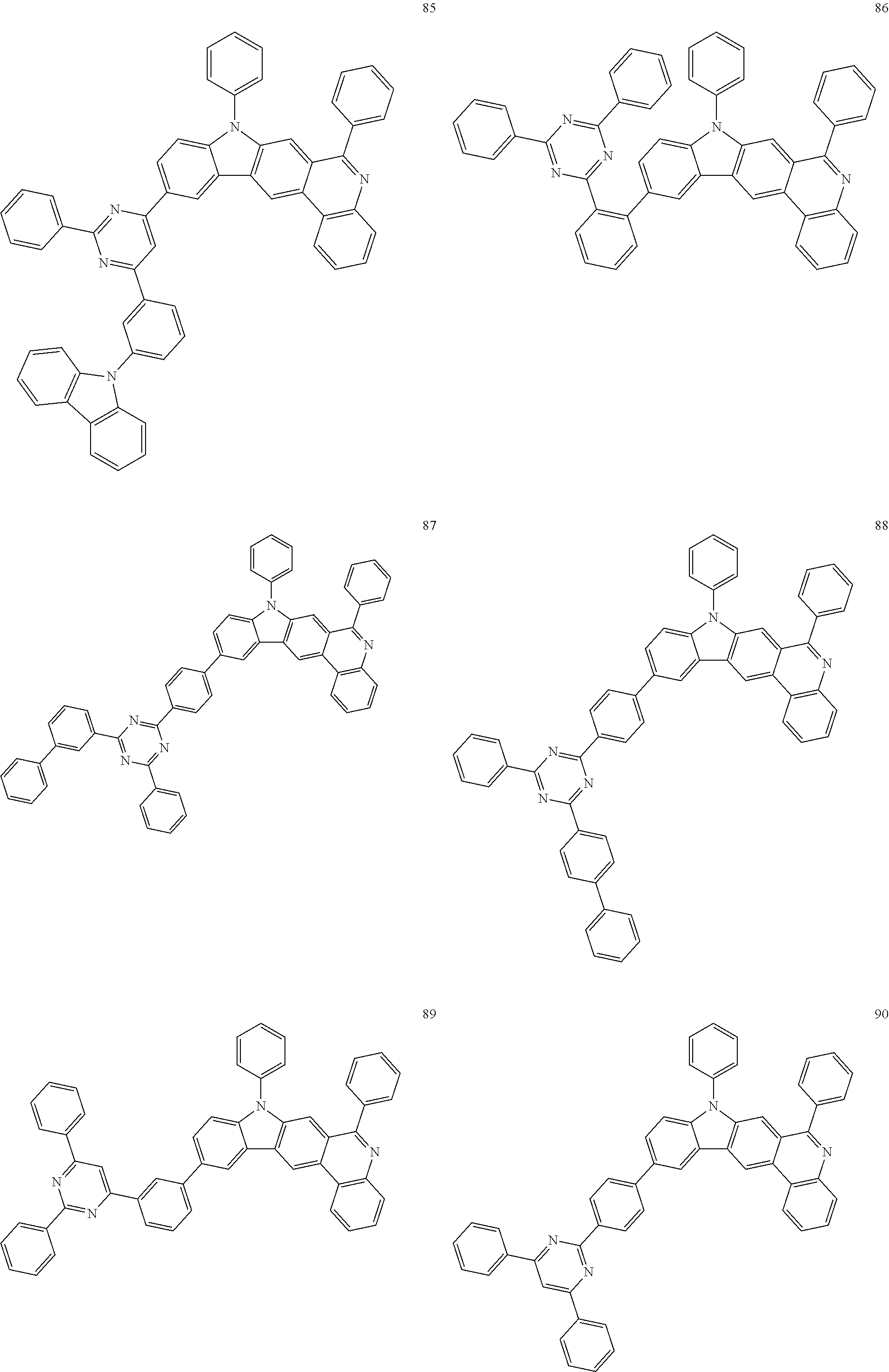

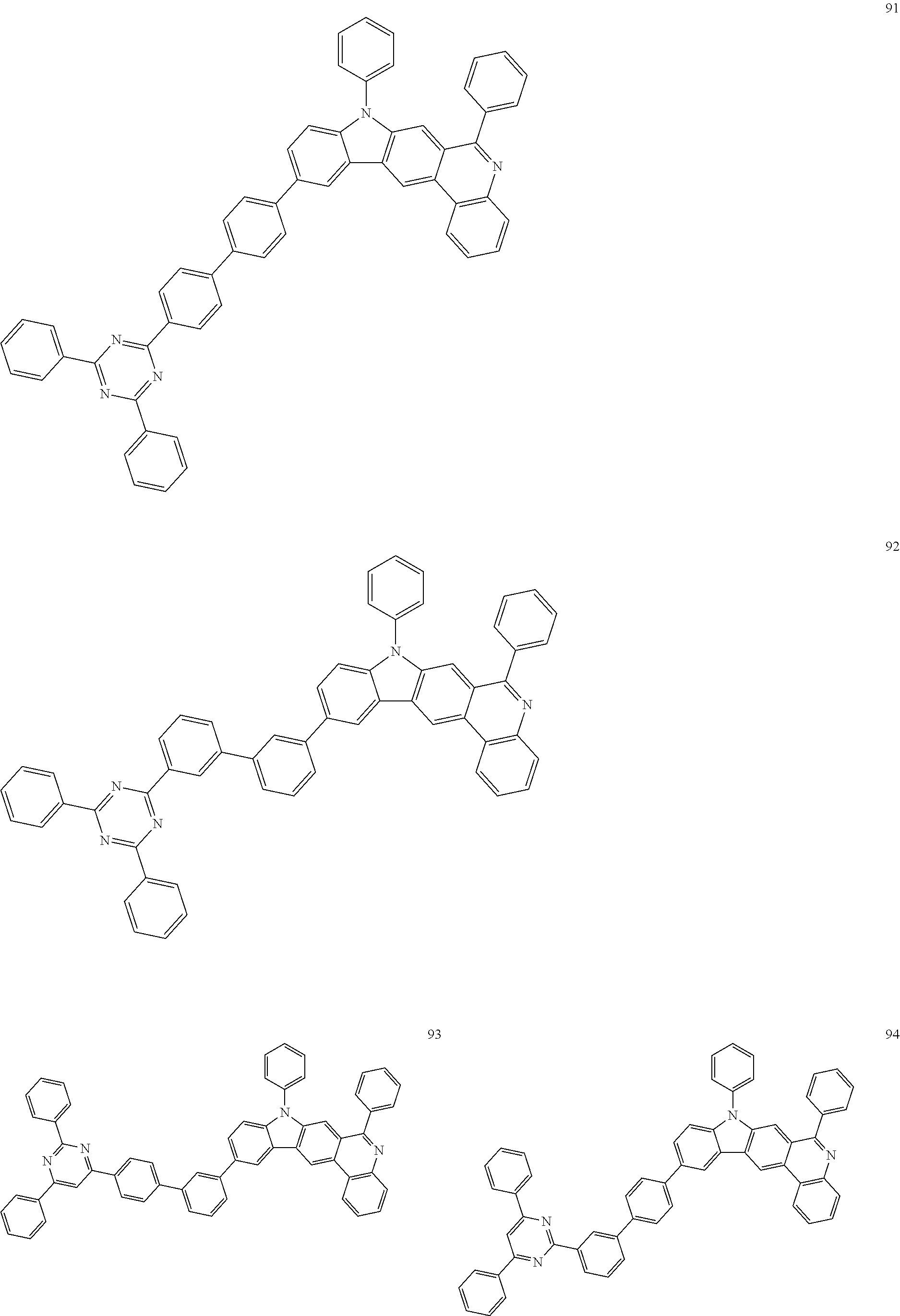 [SMILES: c1ccc(-c2nc(-c3ccccc3)nc(-c3ccc(-c4ccc(-c5ccc6c(c5)c5cc7c(cc5n6-c5ccccc5)c(-c5ccccc5)nc5ccccc57)cc4)cc3)n2)cc1 ; c1ccc(-c2nc(-c3ccccc3)nc(-c3cccc(-c4cccc(-c5ccc6c(c5)c5cc7c(cc5n6-c5ccccc5)c(-c5ccccc5)nc5ccccc57)c4)c3)n2)cc1 ; c1ccc(-c2cc(-c3ccc(-c4cccc(-c5ccc6c(c5)c5cc7c(cc5n6-c5ccccc5)c(-c5ccccc5)nc5ccccc57)c4)cc3)nc(-c3ccccc3)n2)cc1 ; c1ccc(-c2cc(-c3ccccc3)nc(-c3cccc(-c4ccc(-c5ccc6c(c5)c5cc7c(cc5n6-c5ccccc5)c(-c5ccccc5)nc5ccccc57)cc4)c3)n2)cc1]

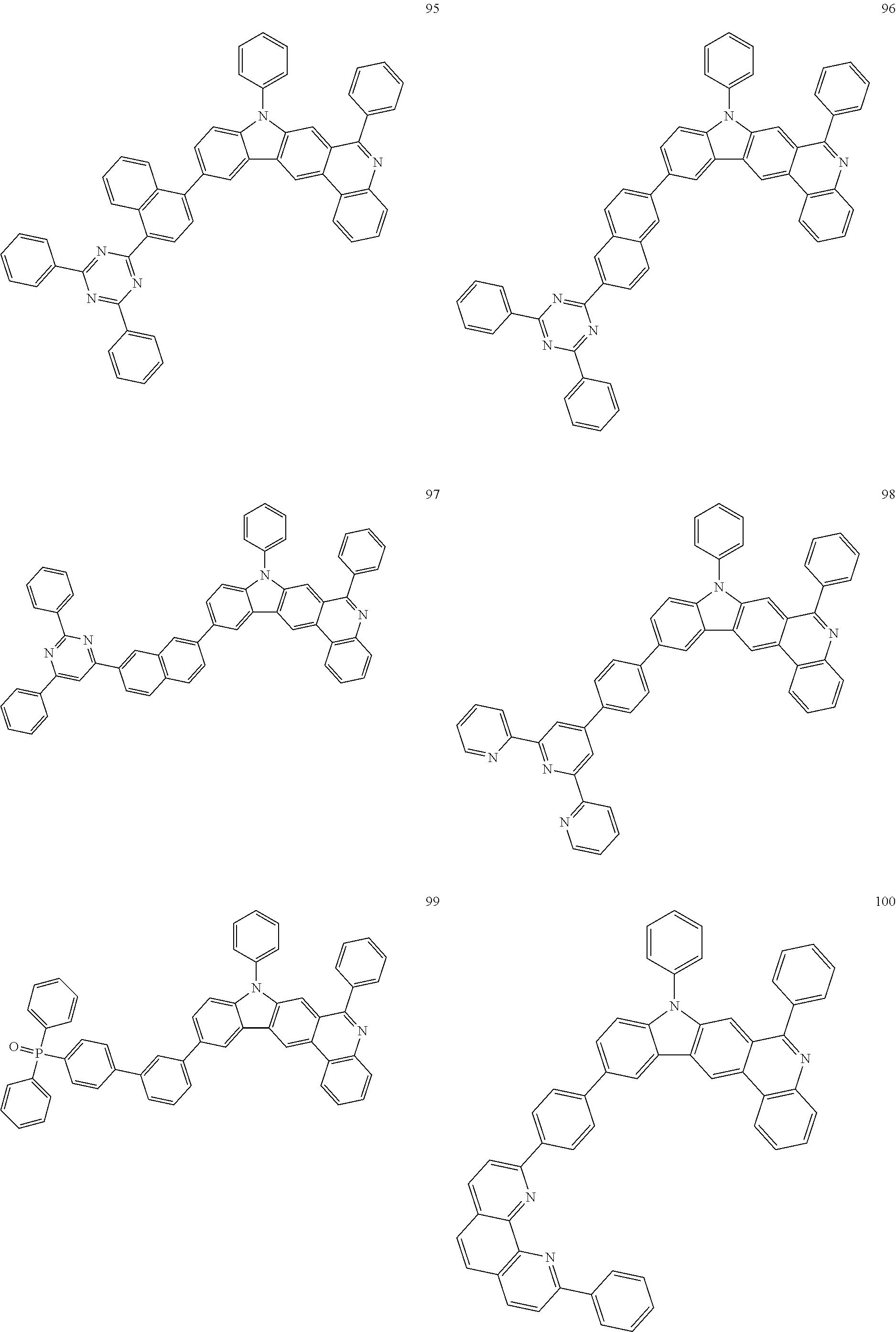

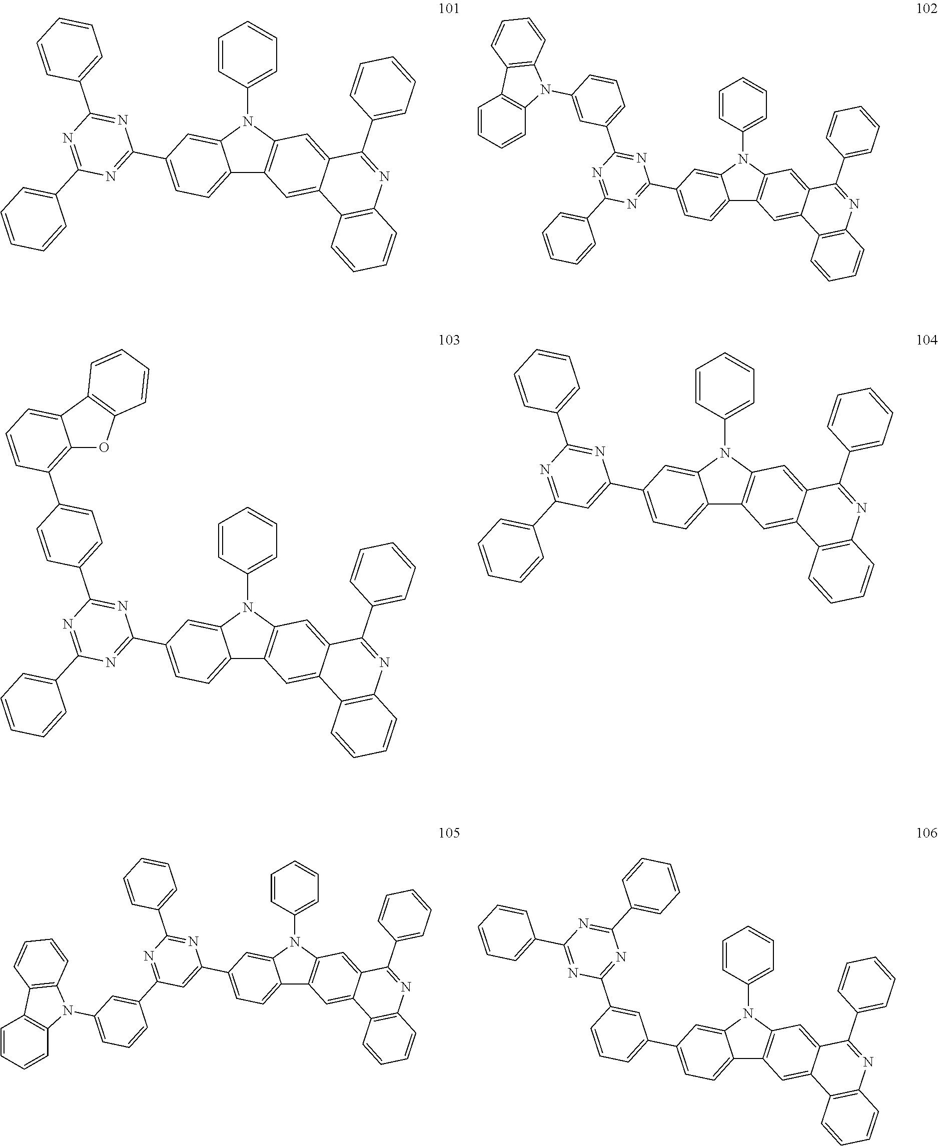

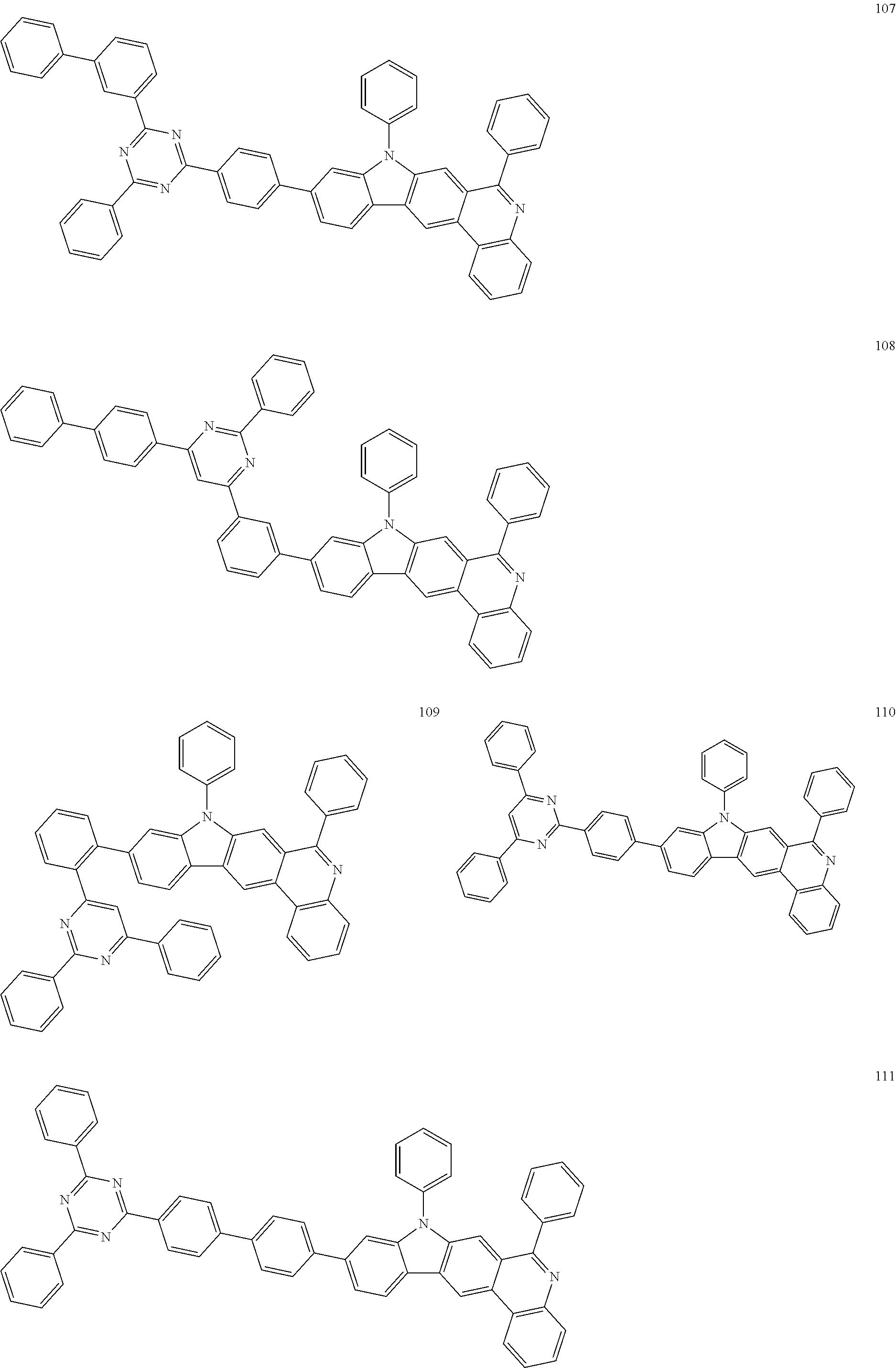

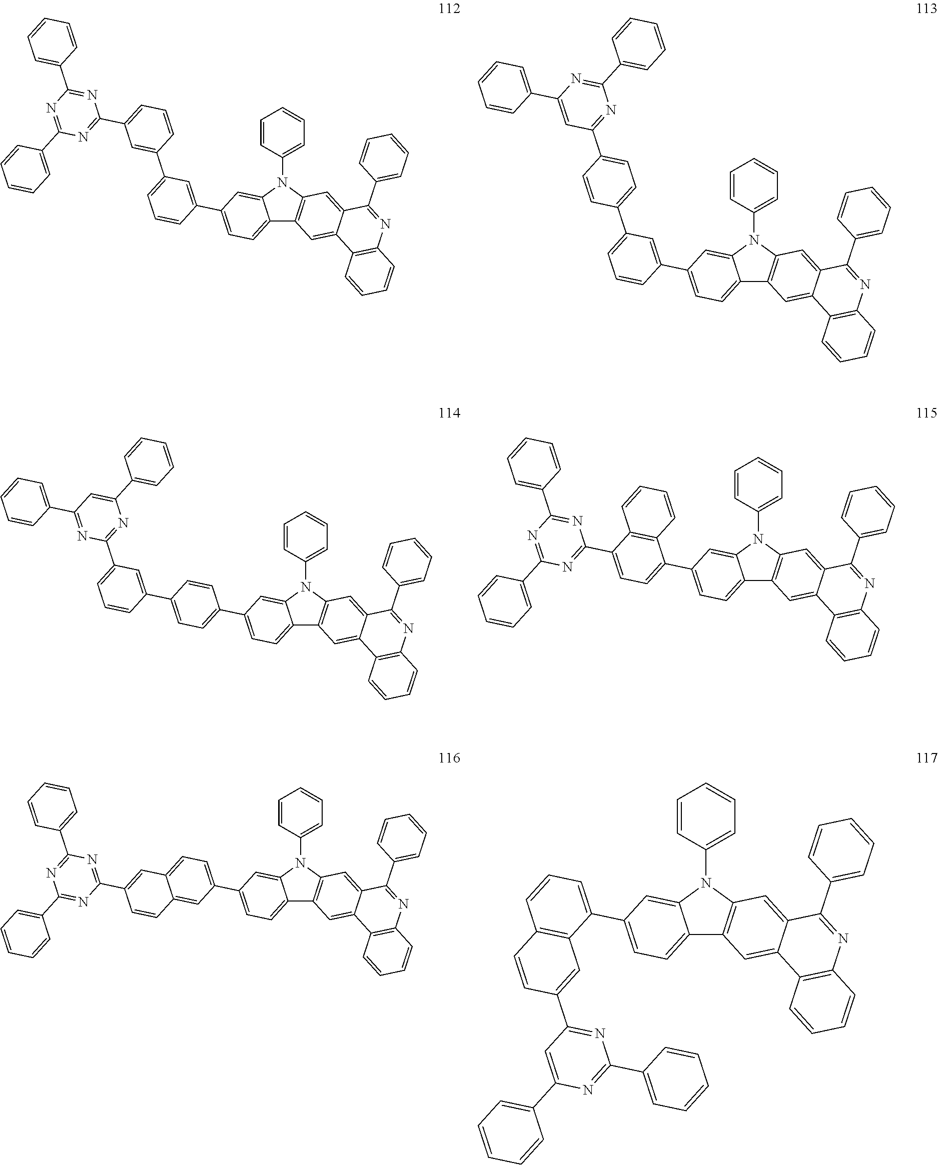

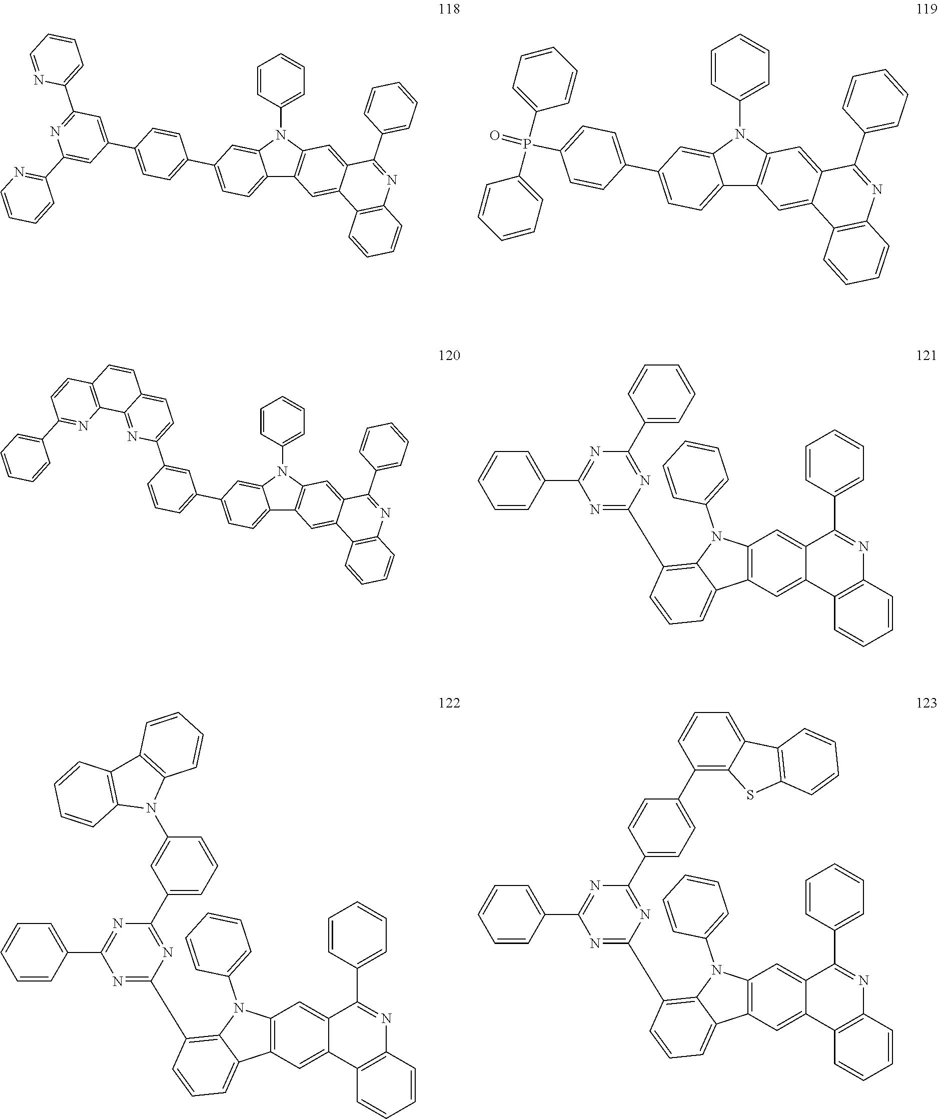

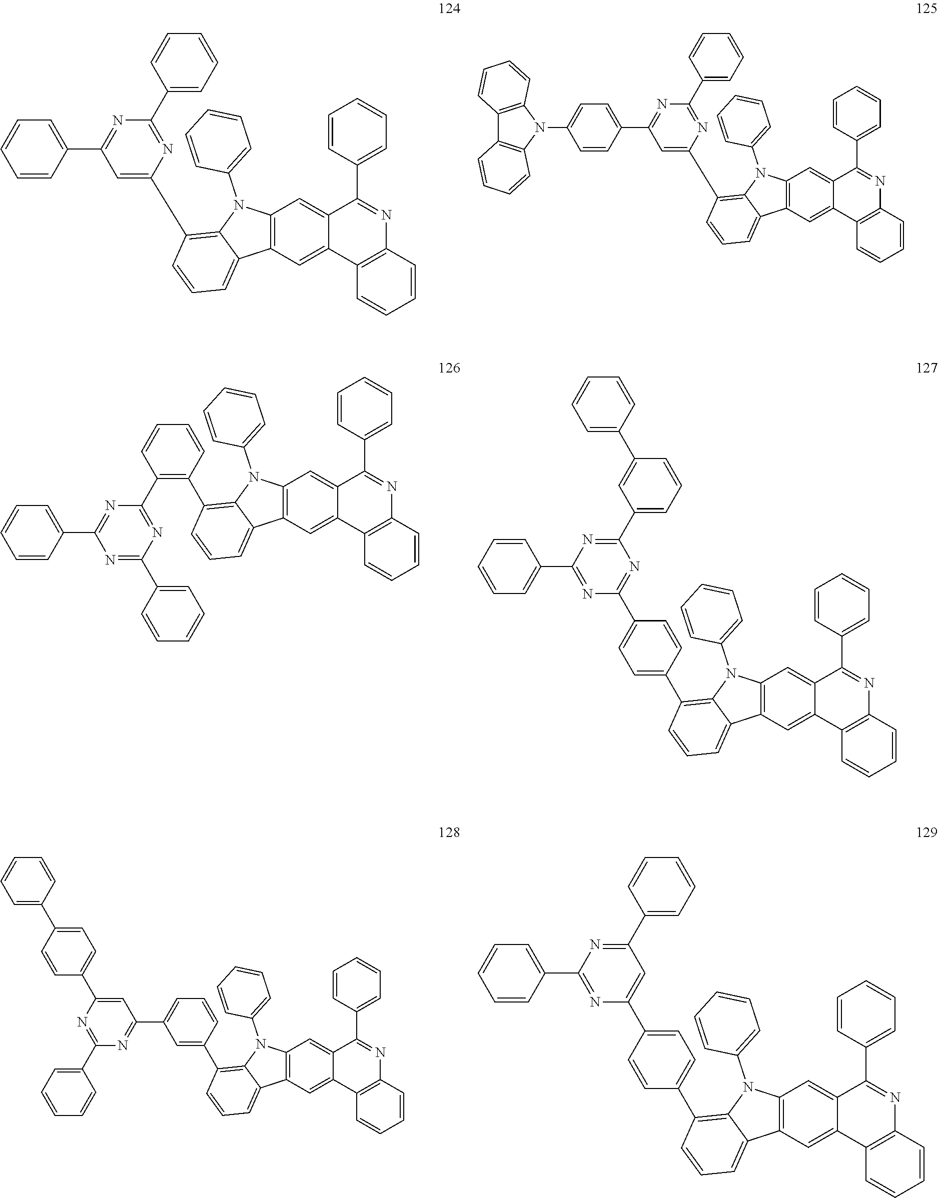

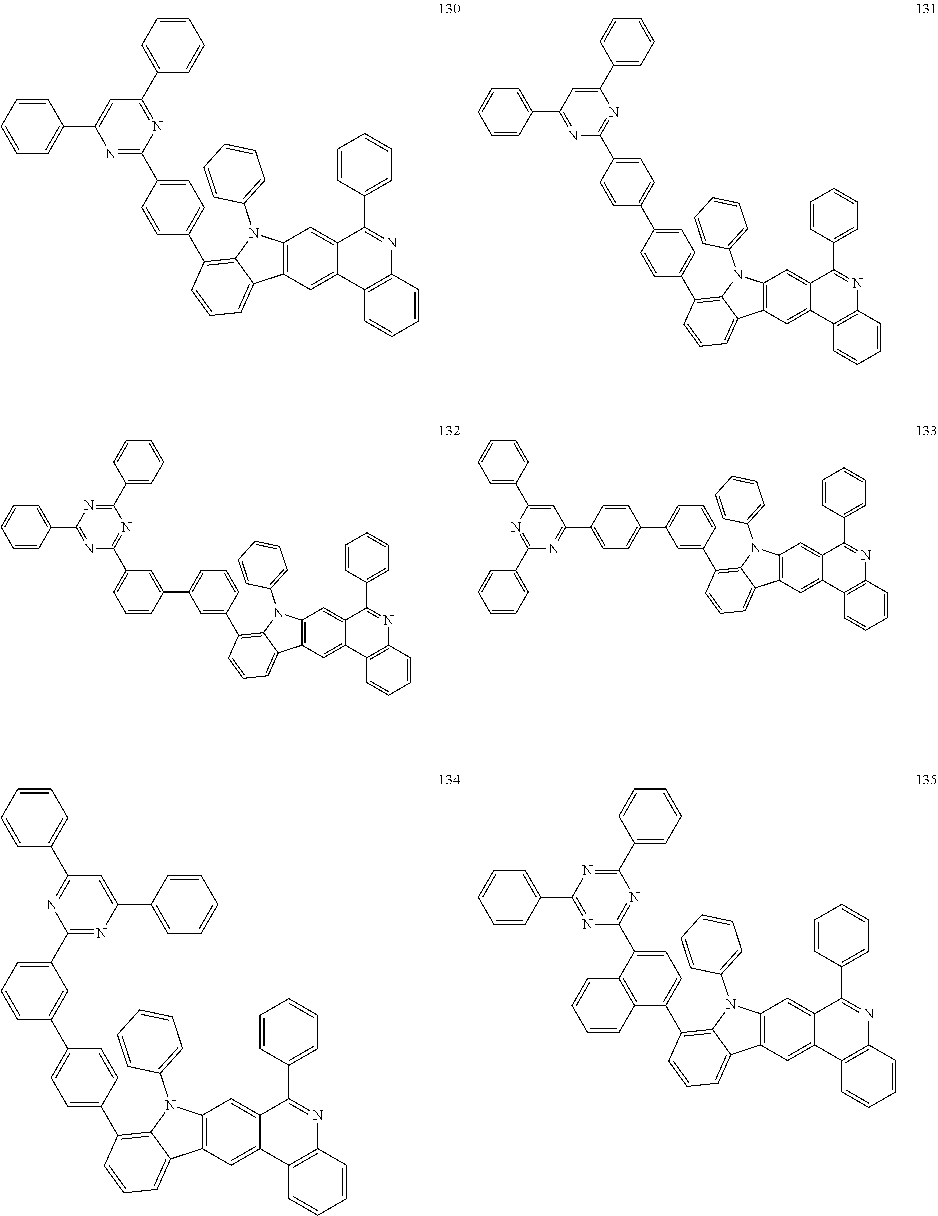

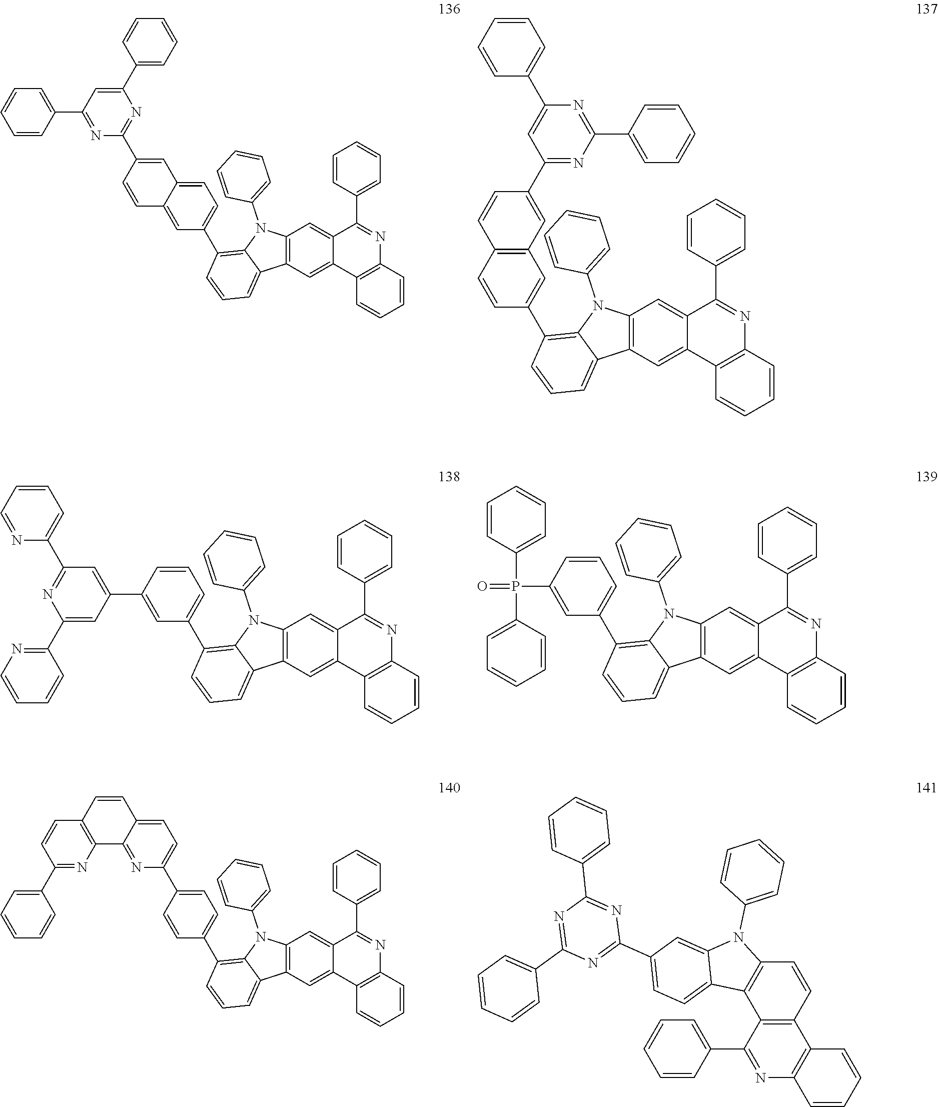

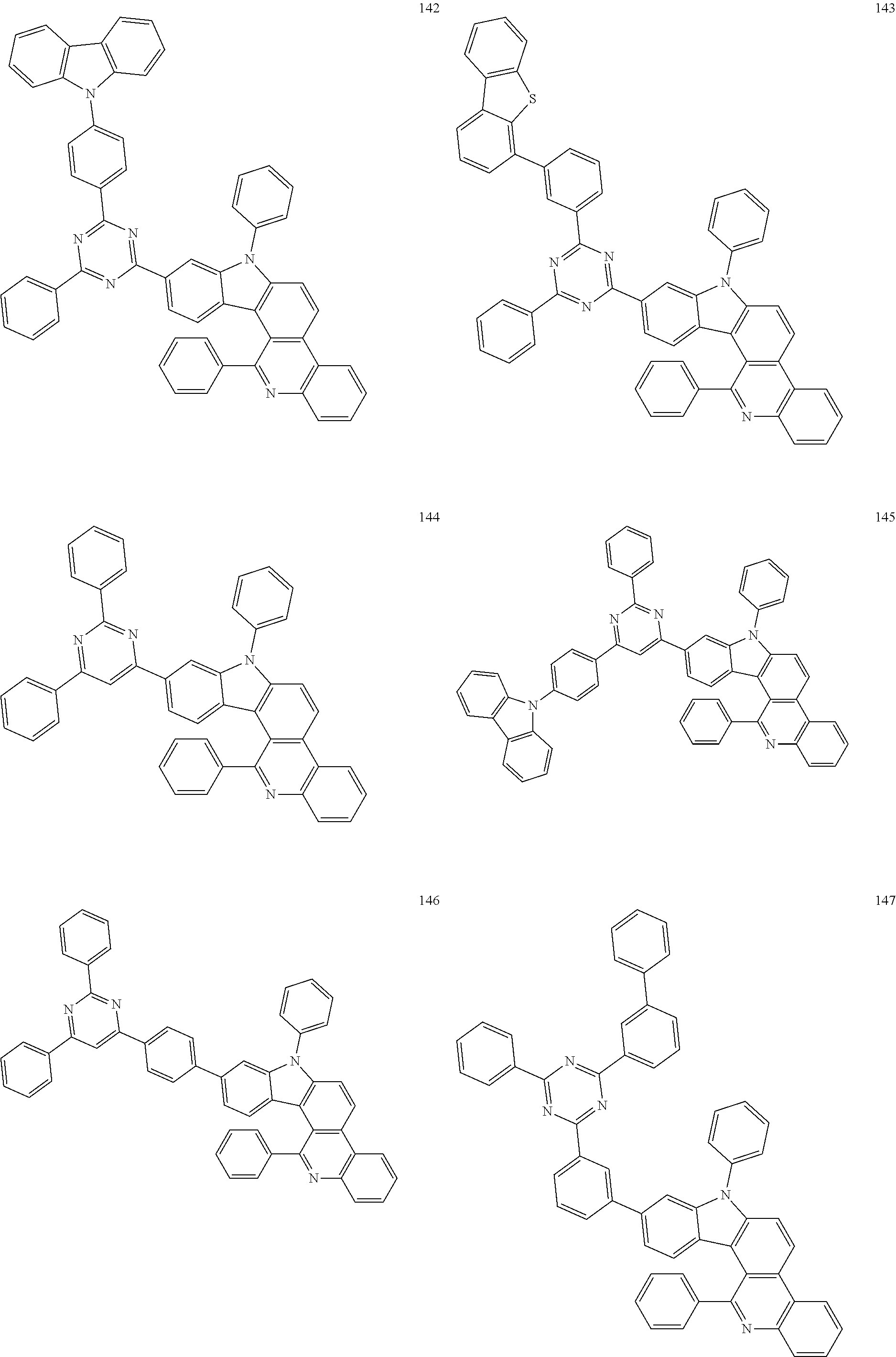

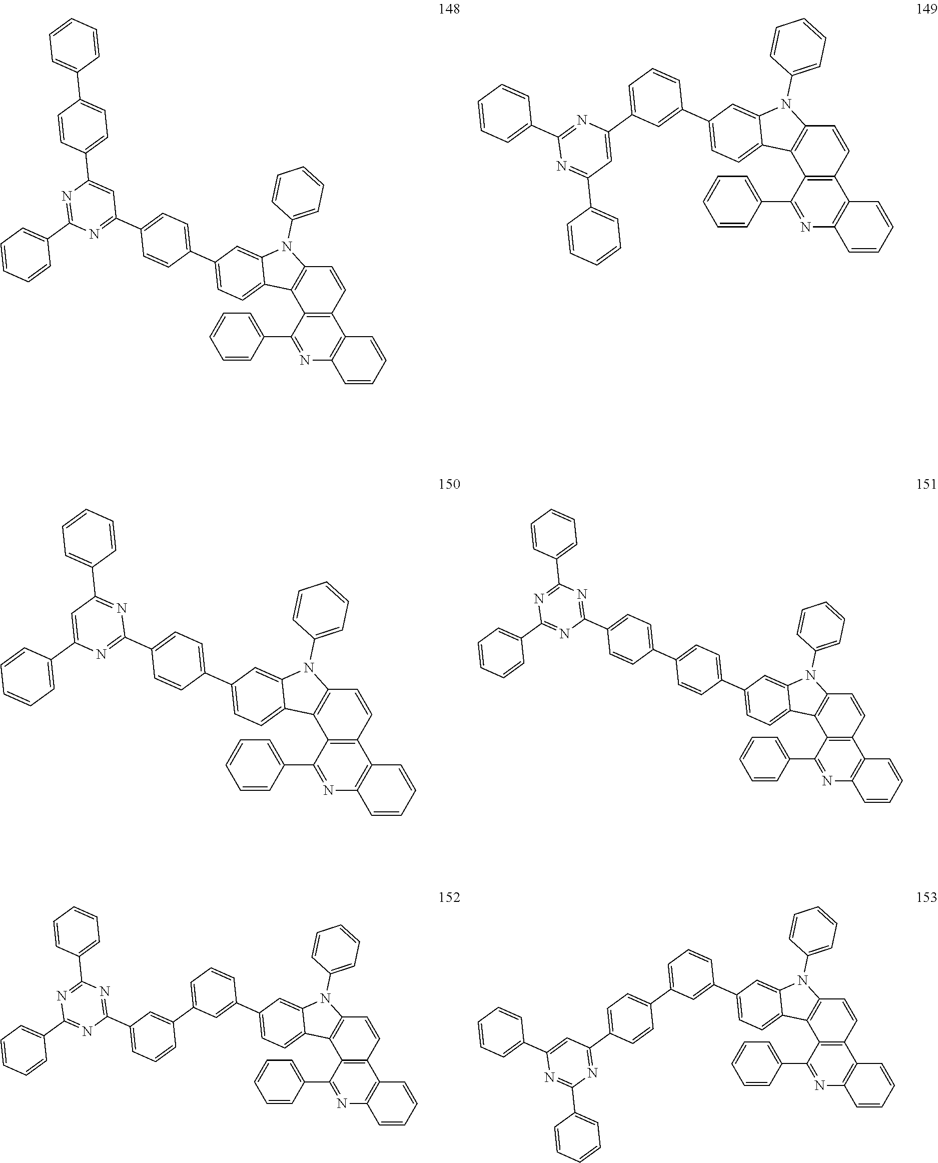

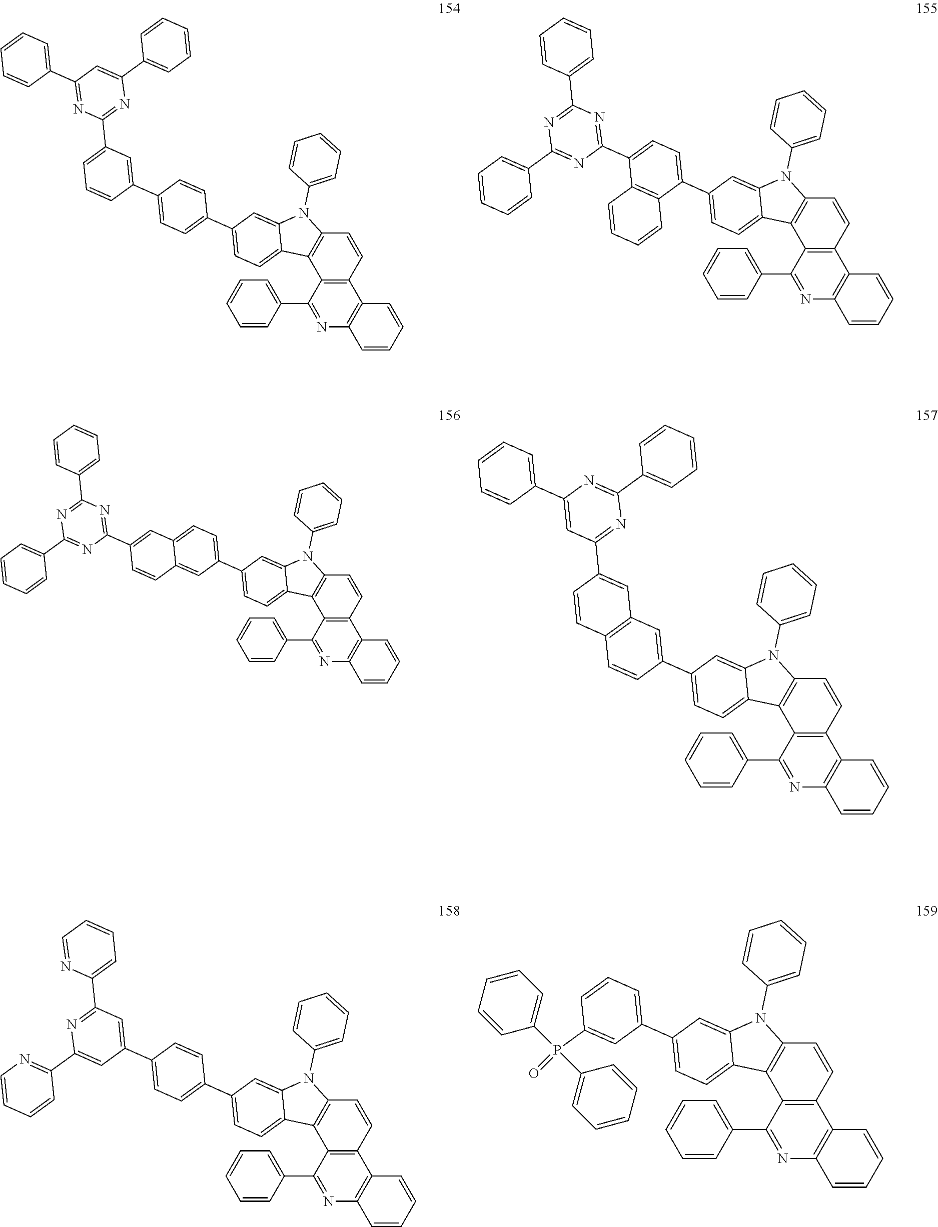

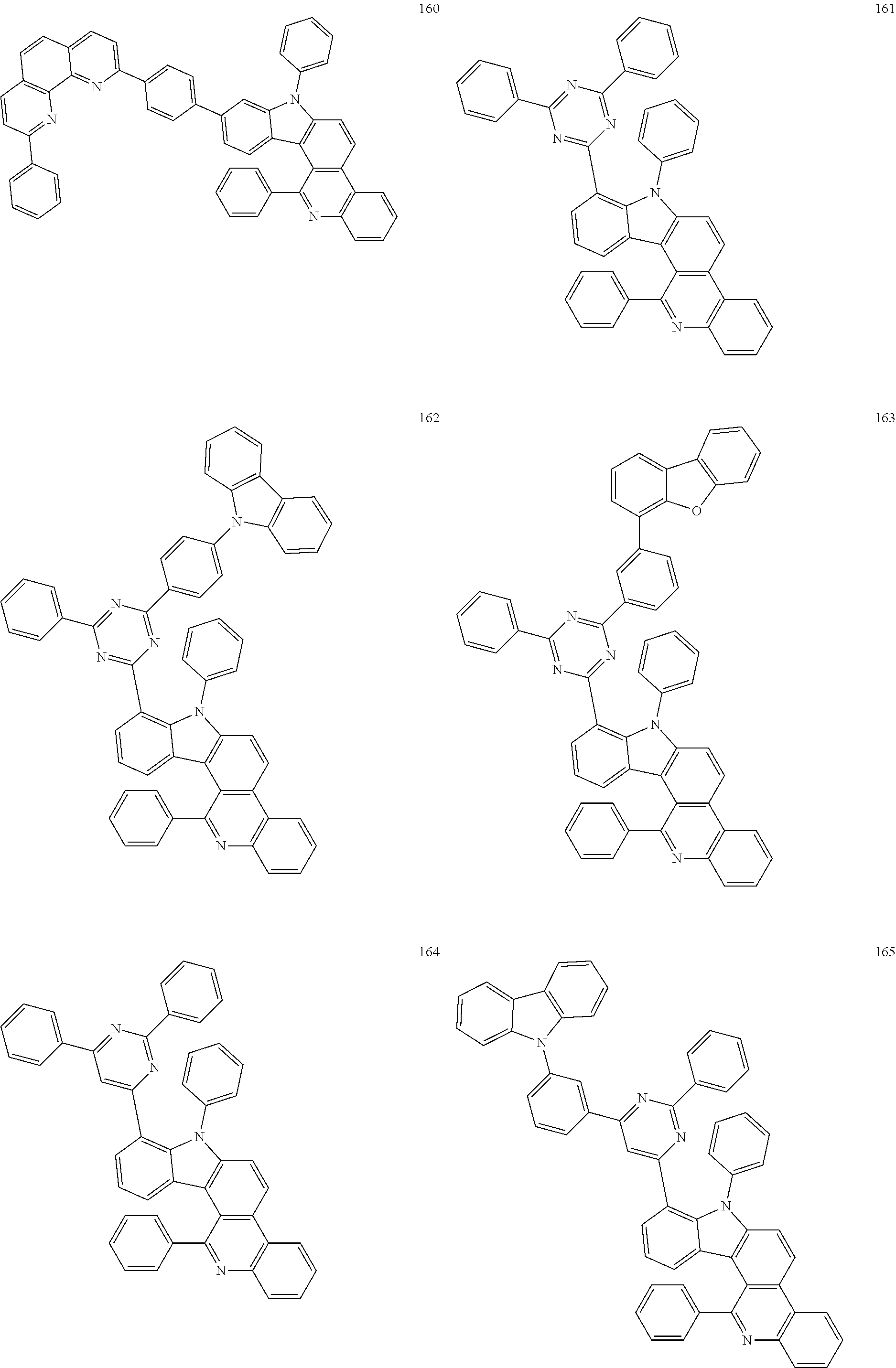

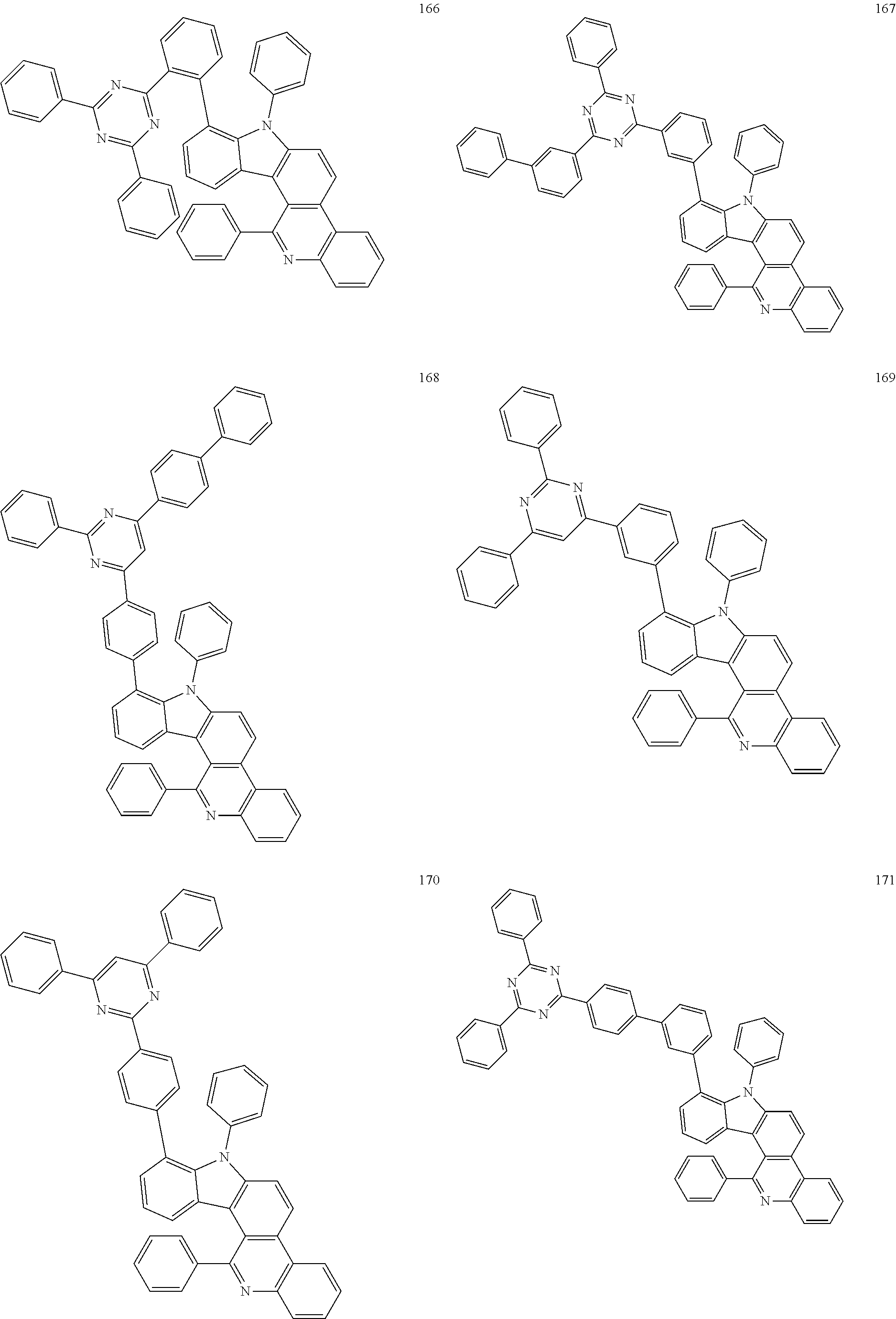

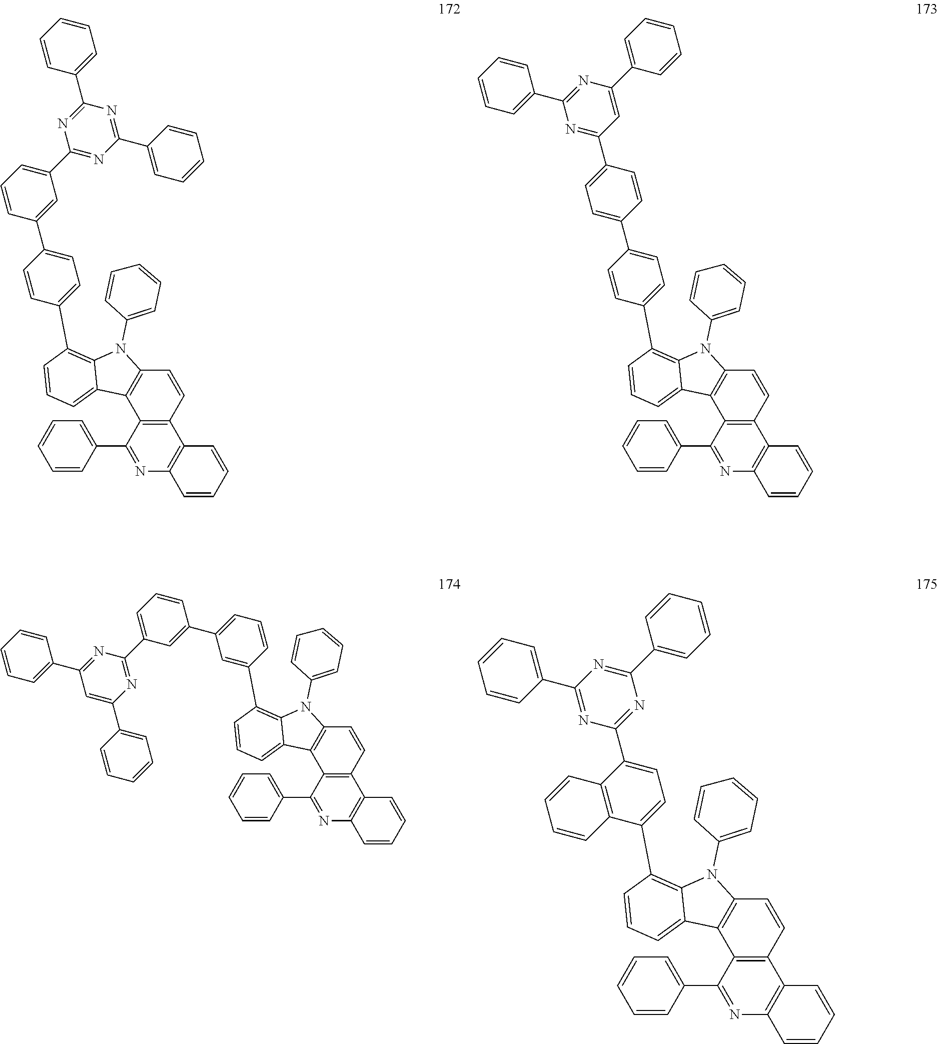 [SMILES: c1ccc(-c2nc(-c3ccccc3)nc(-c3cccc(-c4ccc(-c5cccc6c7c8c(-c9ccccc9)nc9ccccc9c8ccc7n(-c7ccccc7)c56)cc4)c3)n2)cc1 ; c1ccc(-c2cc(-c3ccc(-c4ccc(-c5cccc6c7c8c(-c9ccccc9)nc9ccccc9c8ccc7n(-c7ccccc7)c56)cc4)cc3)nc(-c3ccccc3)n2)cc1 ; c1ccc(-c2cc(-c3ccccc3)nc(-c3cccc(-c4cccc(-c5cccc6c7c8c(-c9ccccc9)nc9ccccc9c8ccc7n(-c7ccccc7)c56)c4)c3)n2)cc1 ; c1ccc(-c2nc(-c3ccccc3)nc(-c3ccc(-c4cccc5c6c7c(-c8ccccc8)nc8ccccc8c7ccc6n(-c6ccccc6)c45)c4ccccc34)n2)cc1]

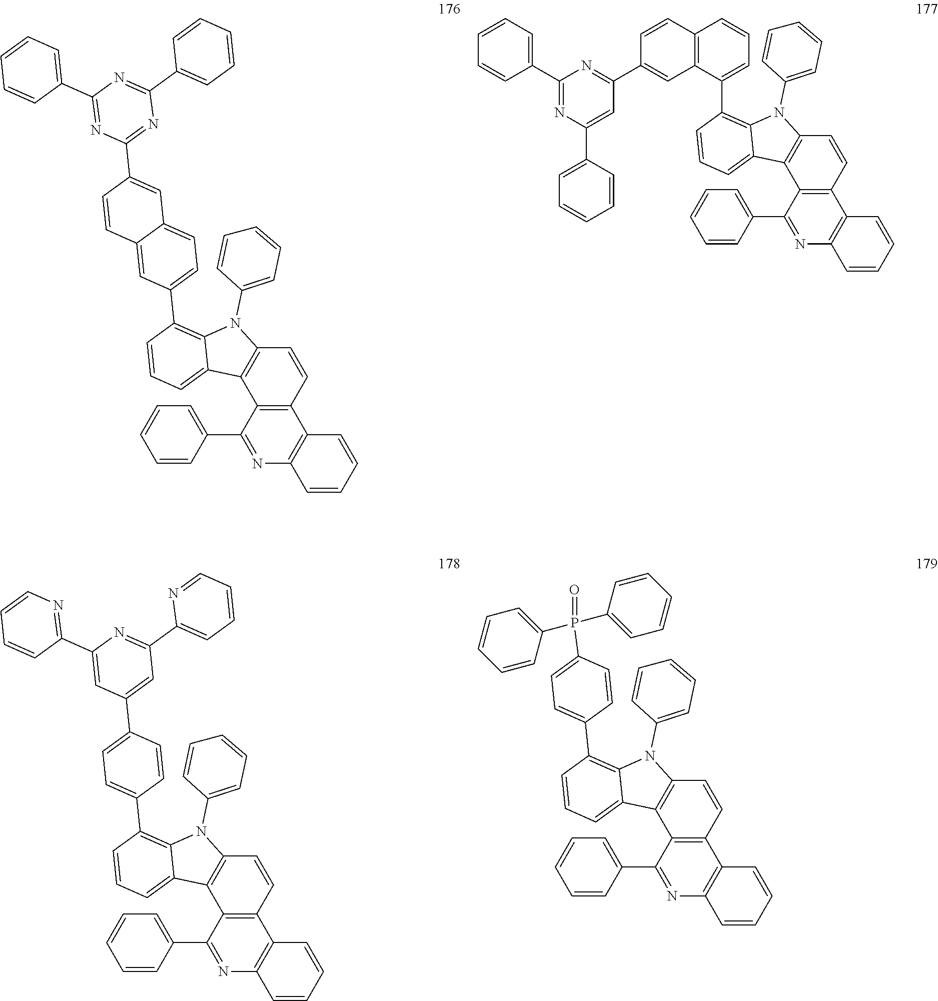 [SMILES: c1ccc(-c2nc(-c3ccccc3)nc(-c3ccc4cc(-c5cccc6c7c8c(-c9ccccc9)nc9ccccc9c8ccc7n(-c7ccccc7)c56)ccc4c3)n2)cc1 ; c1ccc(-c2cc(-c3ccc4cccc(-c5cccc6c7c8c(-c9ccccc9)nc9ccccc9c8ccc7n(-c7ccccc7)c56)c4c3)nc(-c3ccccc3)n2)cc1 ; c1ccc(-c2nc3ccccc3c3ccc4c(c5cccc(-c6ccc(-c7cc(-c8ccccn8)nc(-c8ccccn8)c7)cc6)c5n4-c4ccccc4)c23)cc1 ; O=P(c1ccccc1)(c1ccccc1)c1ccc(-c2cccc3c4c5c(-c6ccccc6)nc6ccccc6c5ccc4n(-c4ccccc4)c23)cc1]

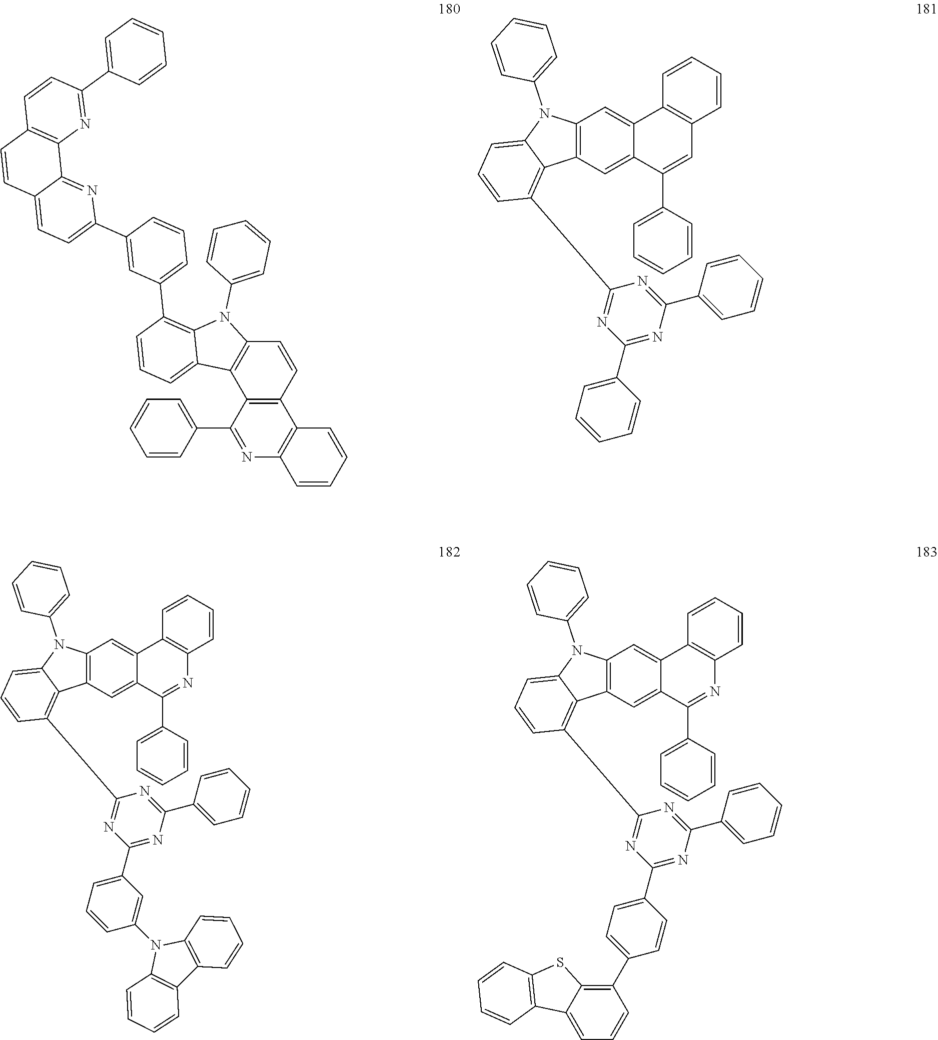 [SMILES: c1ccc(-c2ccc3ccc4ccc(-c5cccc(-c6cccc7c8c9c(-c%10ccccc%10)nc%10ccccc%10c9ccc8n(-c8ccccc8)c67)c5)nc4c3n2)cc1 ; c1ccc(-c2nc(-c3ccccc3)nc(-c3cccc4c3c3cc5c(-c6ccccc6)cc6ccccc6c5cc3n4-c3ccccc3)n2)cc1 ; c1ccc(-c2nc(-c3cccc(-n4c5ccccc5c5ccccc54)c3)nc(-c3cccc4c3c3cc5c(-c6ccccc6)nc6ccccc6c5cc3n4-c3ccccc3)n2)cc1 ; c1ccc(-c2nc(-c3ccc(-c4cccc5c4sc4ccccc45)cc3)nc(-c3cccc4c3c3cc5c(-c6ccccc6)nc6ccccc6c5cc3n4-c3ccccc3)n2)cc1]

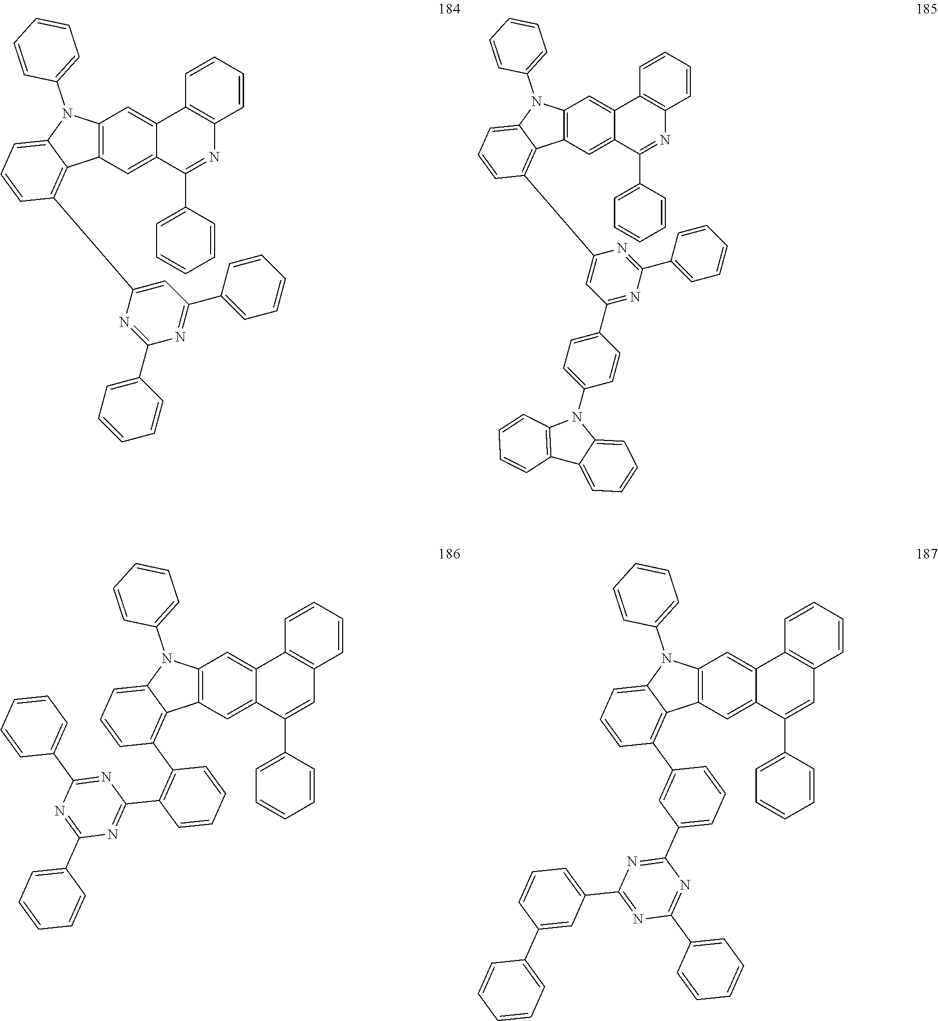 [SMILES: c1ccc(-c2cc(-c3cccc4c3c3cc5c(-c6ccccc6)nc6ccccc6c5cc3n4-c3ccccc3)nc(-c3ccccc3)n2)cc1 ; c1ccc(-c2nc(-c3ccc(-n4c5ccccc5c5ccccc54)cc3)cc(-c3cccc4c3c3cc5c(-c6ccccc6)nc6ccccc6c5cc3n4-c3ccccc3)n2)cc1 ; c1ccc(-c2nc(-c3ccccc3)nc(-c3ccccc3-c3cccc4c3c3cc5c(-c6ccccc6)cc6ccccc6c5cc3n4-c3ccccc3)n2)cc1 ; c1ccc(-c2cccc(-c3nc(-c4ccccc4)nc(-c4cccc(-c5cccc6c5c5cc7c(-c8ccccc8)cc8ccccc8c7cc5n6-c5ccccc5)c4)n3)c2)cc1]

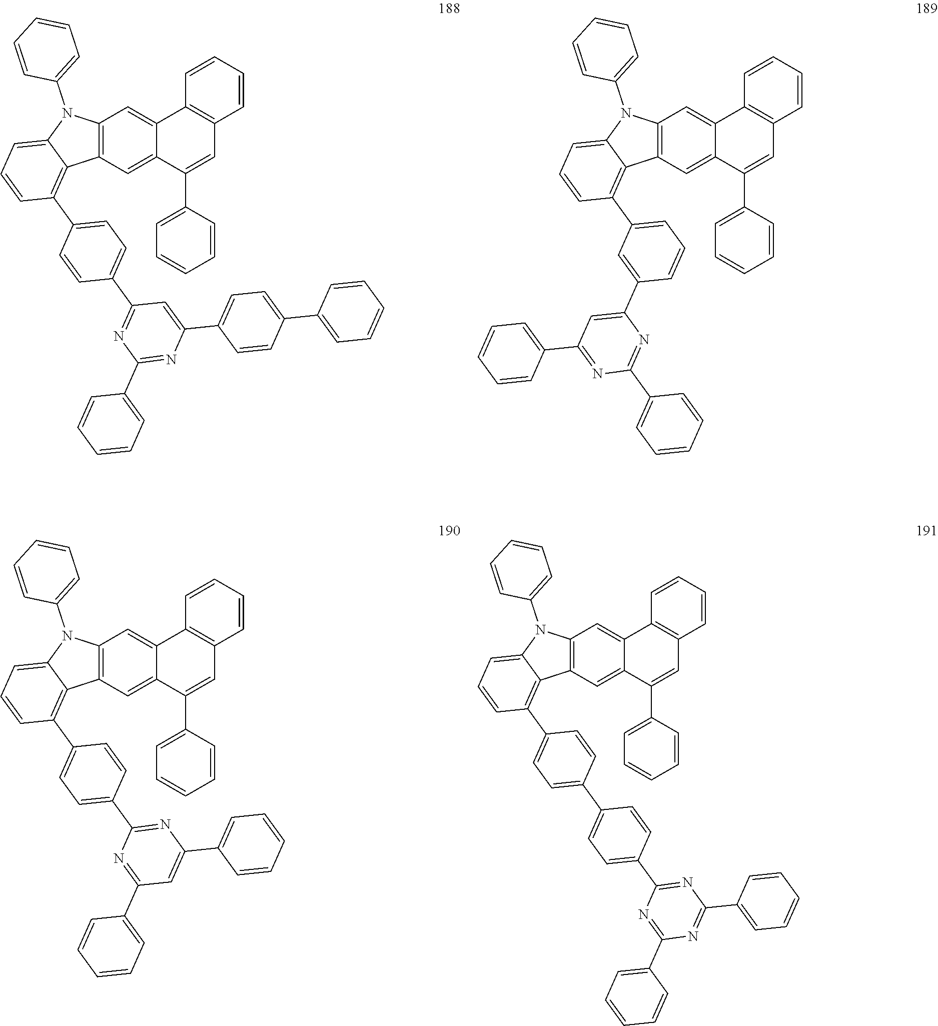 [SMILES: c1ccc(-c2ccc(-c3cc(-c4ccc(-c5cccc6c5c5cc7c(-c8ccccc8)cc8ccccc8c7cc5n6-c5ccccc5)cc4)nc(-c4ccccc4)n3)cc2)cc1 ; c1ccc(-c2cc(-c3cccc(-c4cccc5c4c4cc6c(-c7ccccc7)cc7ccccc7c6cc4n5-c4ccccc4)c3)nc(-c3ccccc3)n2)cc1 ; c1ccc(-c2cc(-c3ccccc3)nc(-c3ccc(-c4cccc5c4c4cc6c(-c7ccccc7)cc7ccccc7c6cc4n5-c4ccccc4)cc3)n2)cc1 ; c1ccc(-c2nc(-c3ccccc3)nc(-c3ccc(-c4ccc(-c5cccc6c5c5cc7c(-c8ccccc8)cc8ccccc8c7cc5n6-c5ccccc5)cc4)cc3)n2)cc1]

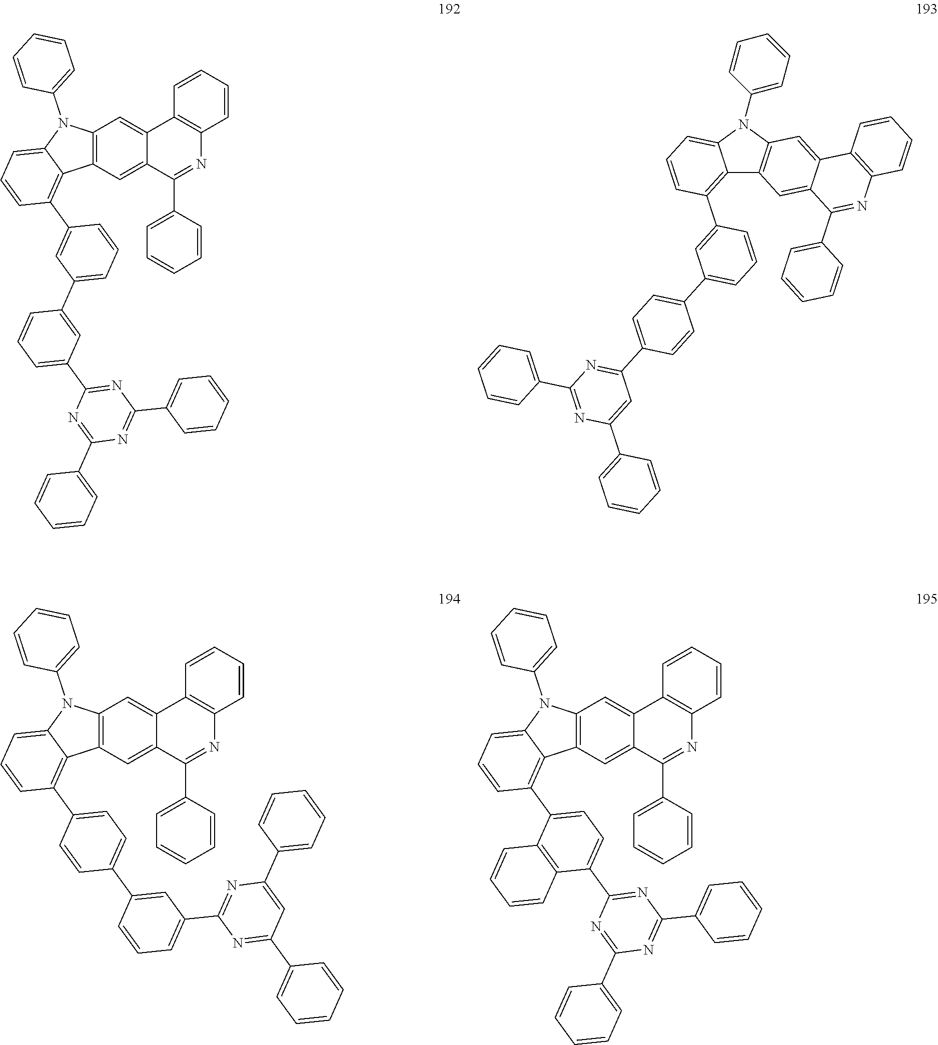 [SMILES: c1ccc(-c2nc(-c3ccccc3)nc(-c3cccc(-c4cccc(-c5cccc6c5c5cc7c(-c8ccccc8)nc8ccccc8c7cc5n6-c5ccccc5)c4)c3)n2)cc1 ; c1ccc(-c2cc(-c3ccc(-c4cccc(-c5cccc6c5c5cc7c(-c8ccccc8)nc8ccccc8c7cc5n6-c5ccccc5)c4)cc3)nc(-c3ccccc3)n2)cc1 ; c1ccc(-c2cc(-c3ccccc3)nc(-c3cccc(-c4ccc(-c5cccc6c5c5cc7c(-c8ccccc8)nc8ccccc8c7cc5n6-c5ccccc5)cc4)c3)n2)cc1 ; c1ccc(-c2nc(-c3ccccc3)nc(-c3ccc(-c4cccc5c4c4cc6c(-c7ccccc7)nc7ccccc7c6cc4n5-c4ccccc4)c4ccccc34)n2)cc1]

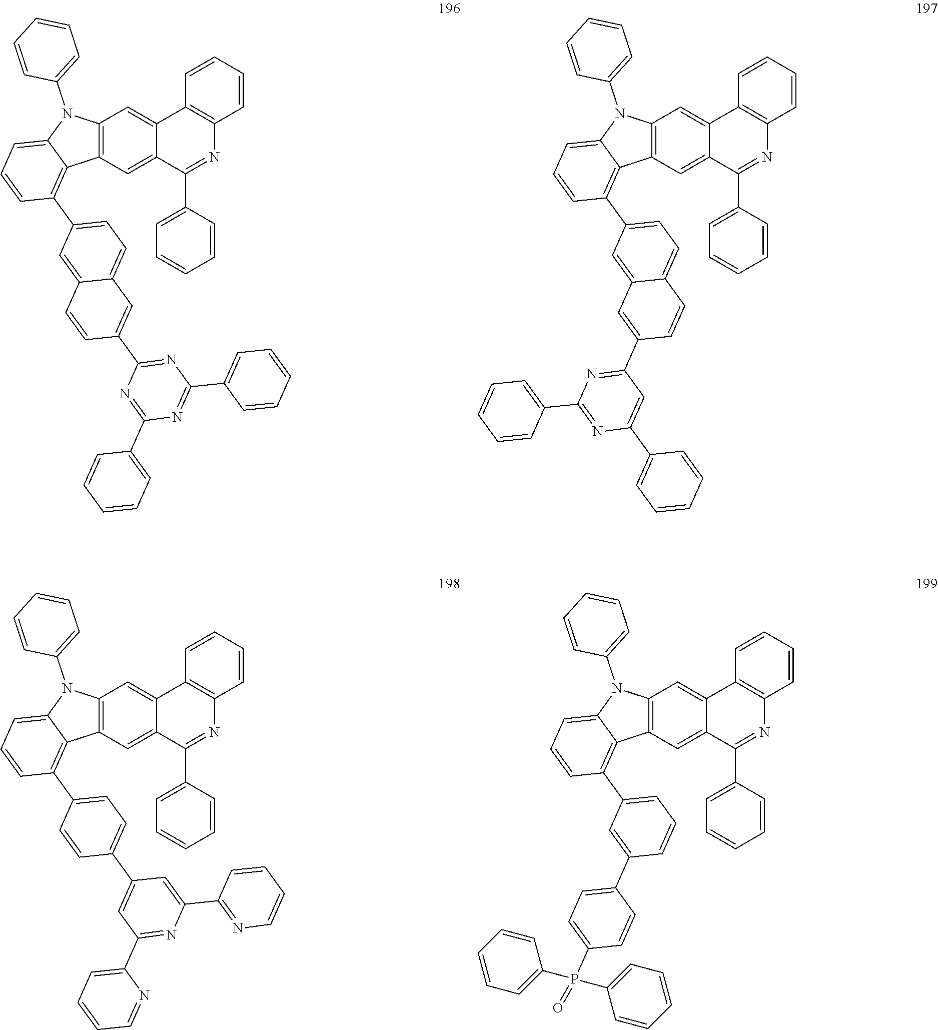 [SMILES: c1ccc(-c2nc(-c3ccccc3)nc(-c3ccc4cc(-c5cccc6c5c5cc7c(-c8ccccc8)nc8ccccc8c7cc5n6-c5ccccc5)ccc4c3)n2)cc1 ; c1ccc(-c2cc(-c3ccc4ccc(-c5cccc6c5c5cc7c(-c8ccccc8)nc8ccccc8c7cc5n6-c5ccccc5)cc4c3)nc(-c3ccccc3)n2)cc1 ; c1ccc(-c2nc3ccccc3c3cc4c(cc23)c2c(-c3ccc(-c5cc(-c6ccccn6)nc(-c6ccccn6)c5)cc3)cccc2n4-c2ccccc2)cc1 ; O=P(c1ccccc1)(c1ccccc1)c1ccc(-c2cccc(-c3cccc4c3c3cc5c(-c6ccccc6)nc6ccccc6c5cc3n4-c3ccccc3)c2)cc1]

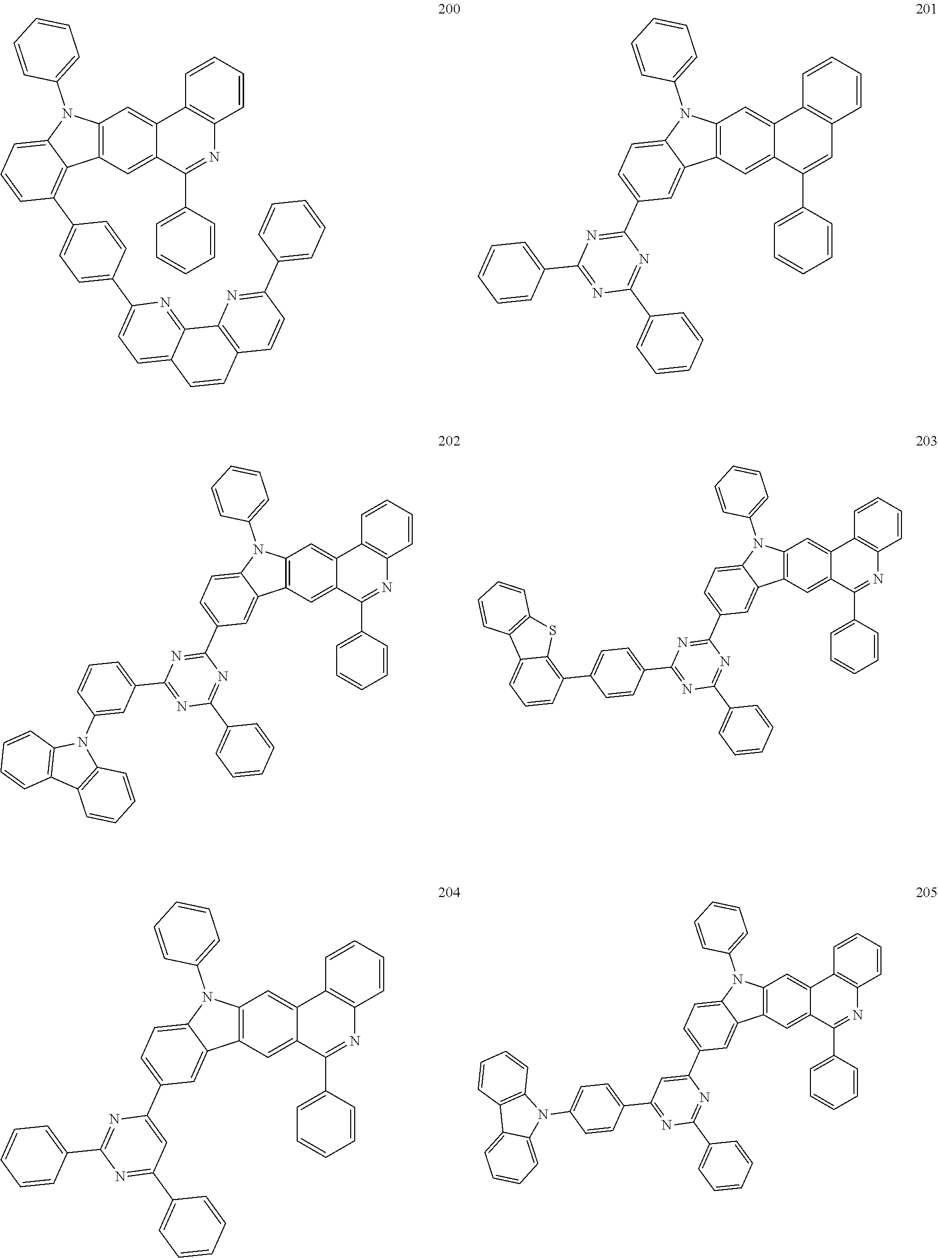

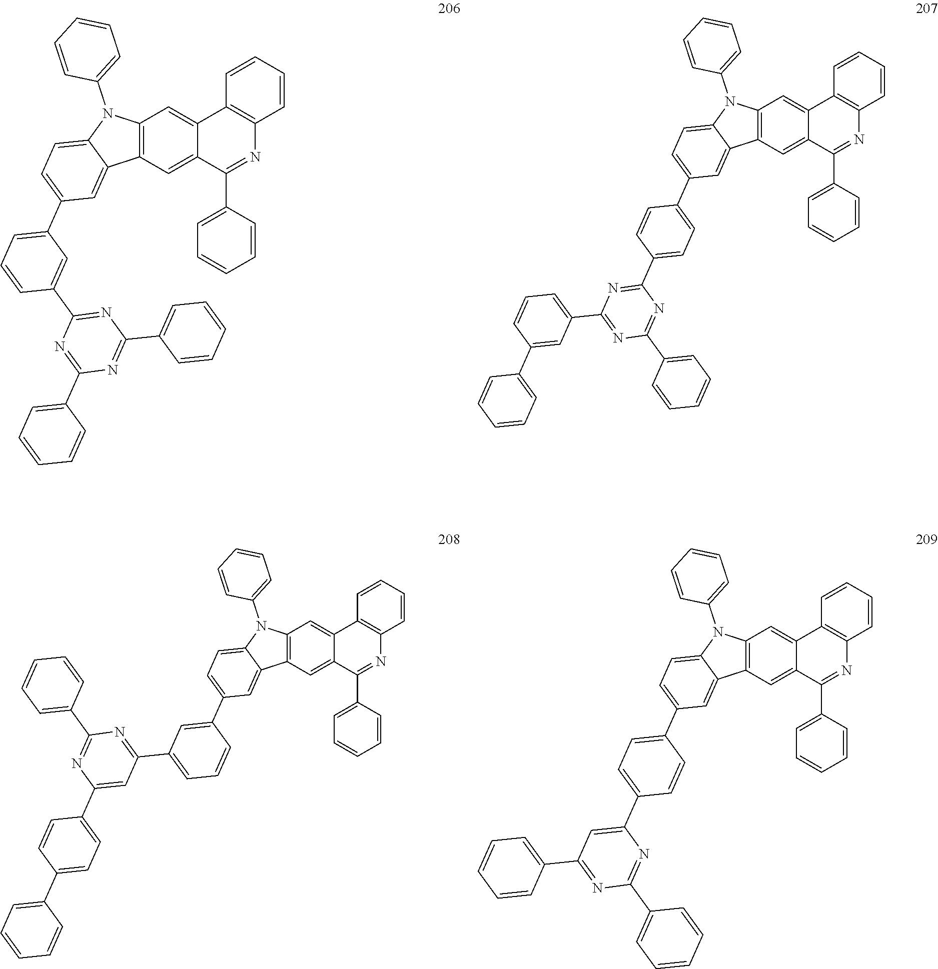 [SMILES: c1ccc(-c2nc(-c3ccccc3)nc(-c3cccc(-c4ccc5c(c4)c4cc6c(-c7ccccc7)nc7ccccc7c6cc4n5-c4ccccc4)c3)n2)cc1 ; c1ccc(-c2cccc(-c3nc(-c4ccccc4)nc(-c4ccc(-c5ccc6c(c5)c5cc7c(-c8ccccc8)nc8ccccc8c7cc5n6-c5ccccc5)cc4)n3)c2)cc1 ; c1ccc(-c2ccc(-c3cc(-c4cccc(-c5ccc6c(c5)c5cc7c(-c8ccccc8)nc8ccccc8c7cc5n6-c5ccccc5)c4)nc(-c4ccccc4)n3)cc2)cc1 ; c1ccc(-c2cc(-c3ccc(-c4ccc5c(c4)c4cc6c(-c7ccccc7)nc7ccccc7c6cc4n5-c4ccccc4)cc3)nc(-c3ccccc3)n2)cc1]

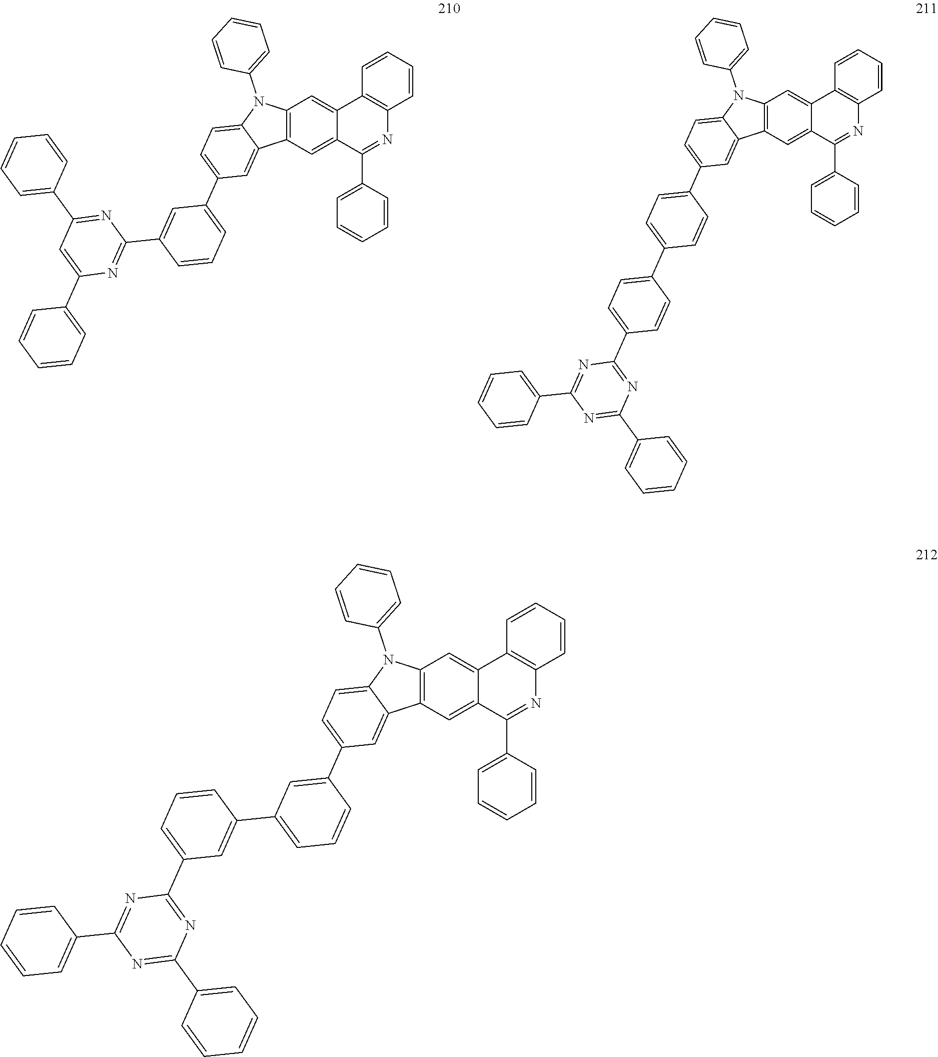 [SMILES: c1ccc(-c2cc(-c3ccccc3)nc(-c3cccc(-c4ccc5c(c4)c4cc6c(-c7ccccc7)nc7ccccc7c6cc4n5-c4ccccc4)c3)n2)cc1 ; c1ccc(-c2nc(-c3ccccc3)nc(-c3ccc(-c4ccc(-c5ccc6c(c5)c5cc7c(-c8ccccc8)nc8ccccc8c7cc5n6-c5ccccc5)cc4)cc3)n2)cc1 ; c1ccc(-c2nc(-c3ccccc3)nc(-c3cccc(-c4cccc(-c5ccc6c(c5)c5cc7c(-c8ccccc8)nc8ccccc8c7cc5n6-c5ccccc5)c4)c3)n2)cc1]

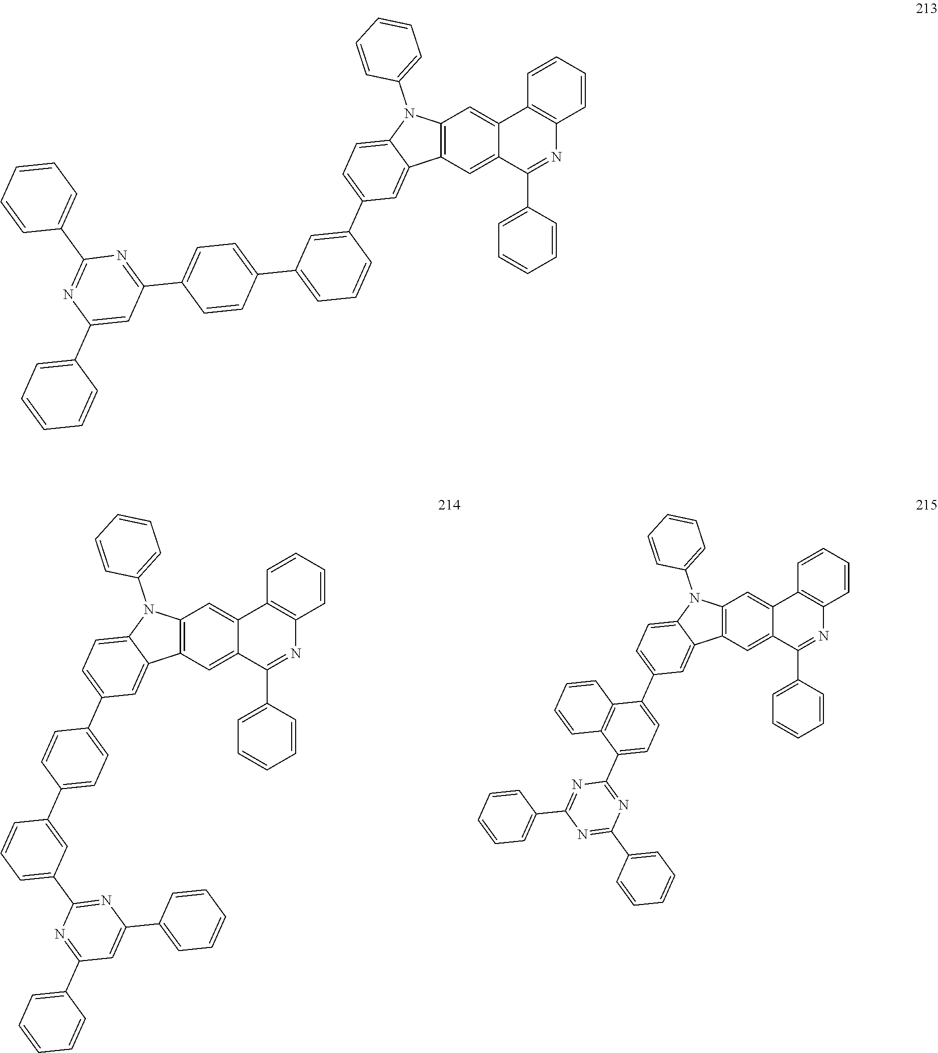 [SMILES: c1ccc(-c2cc(-c3ccc(-c4cccc(-c5ccc6c(c5)c5cc7c(-c8ccccc8)nc8ccccc8c7cc5n6-c5ccccc5)c4)cc3)nc(-c3ccccc3)n2)cc1 ; c1ccc(-c2cc(-c3ccccc3)nc(-c3cccc(-c4ccc(-c5ccc6c(c5)c5cc7c(-c8ccccc8)nc8ccccc8c7cc5n6-c5ccccc5)cc4)c3)n2)cc1 ; c1ccc(-c2nc(-c3ccccc3)nc(-c3ccc(-c4ccc5c(c4)c4cc6c(-c7ccccc7)nc7ccccc7c6cc4n5-c4ccccc4)c4ccccc34)n2)cc1]

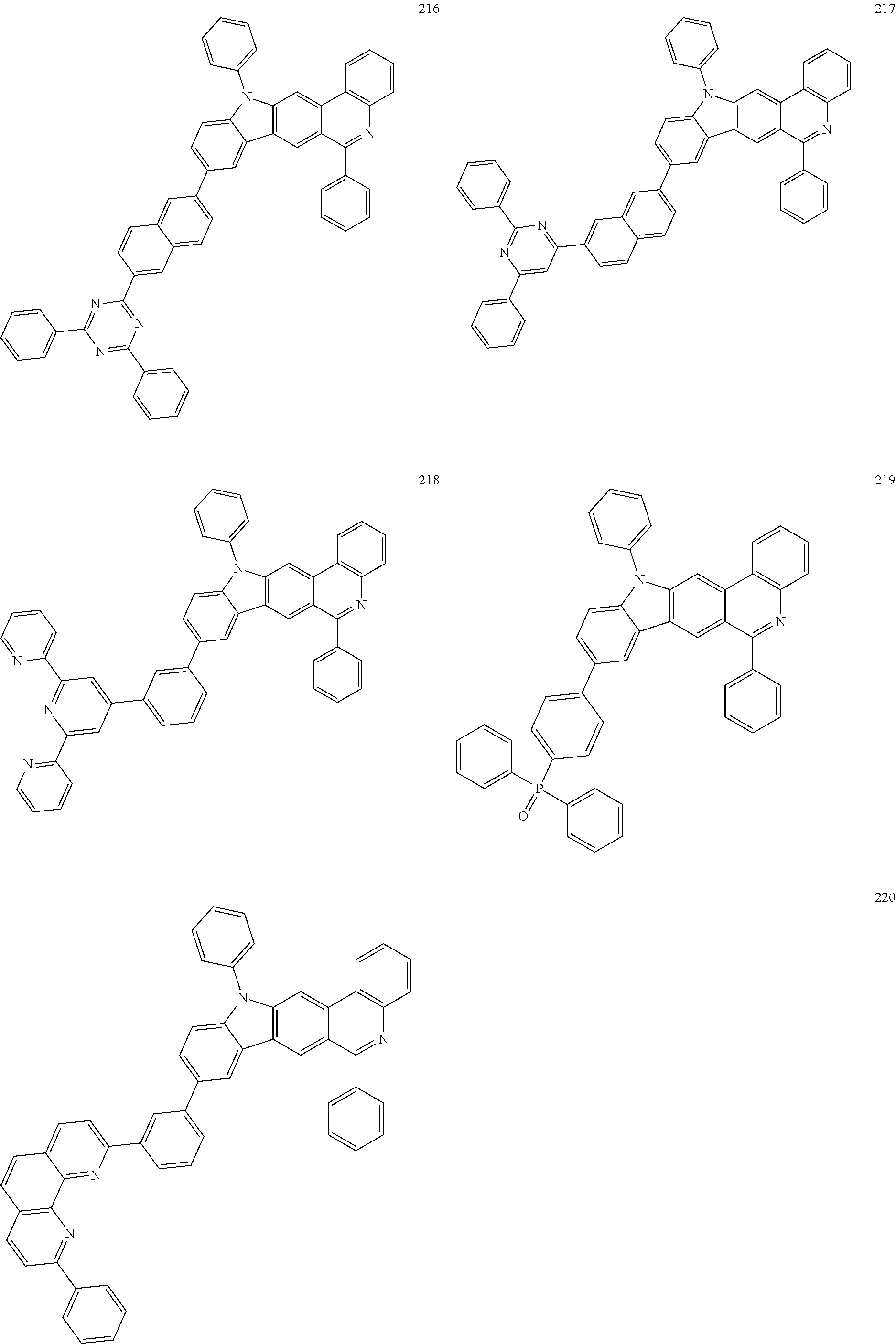 [SMILES: c1ccc(-c2nc(-c3ccccc3)nc(-c3ccc4cc(-c5ccc6c(c5)c5cc7c(-c8ccccc8)nc8ccccc8c7cc5n6-c5ccccc5)ccc4c3)n2)cc1 ; c1ccc(-c2cc(-c3ccc4ccc(-c5ccc6c(c5)c5cc7c(-c8ccccc8)nc8ccccc8c7cc5n6-c5ccccc5)cc4c3)nc(-c3ccccc3)n2)cc1 ; c1ccc(-c2nc3ccccc3c3cc4c(cc23)c2cc(-c3cccc(-c5cc(-c6ccccn6)nc(-c6ccccn6)c5)c3)ccc2n4-c2ccccc2)cc1 ; O=P(c1ccccc1)(c1ccccc1)c1ccc(-c2ccc3c(c2)c2cc4c(-c5ccccc5)nc5ccccc5c4cc2n3-c2ccccc2)cc1 ; c1ccc(-c2ccc3ccc4ccc(-c5cccc(-c6ccc7c(c6)c6cc8c(-c9ccccc9)nc9ccccc9c8cc6n7-c6ccccc6)c5)nc4c3n2)cc1]

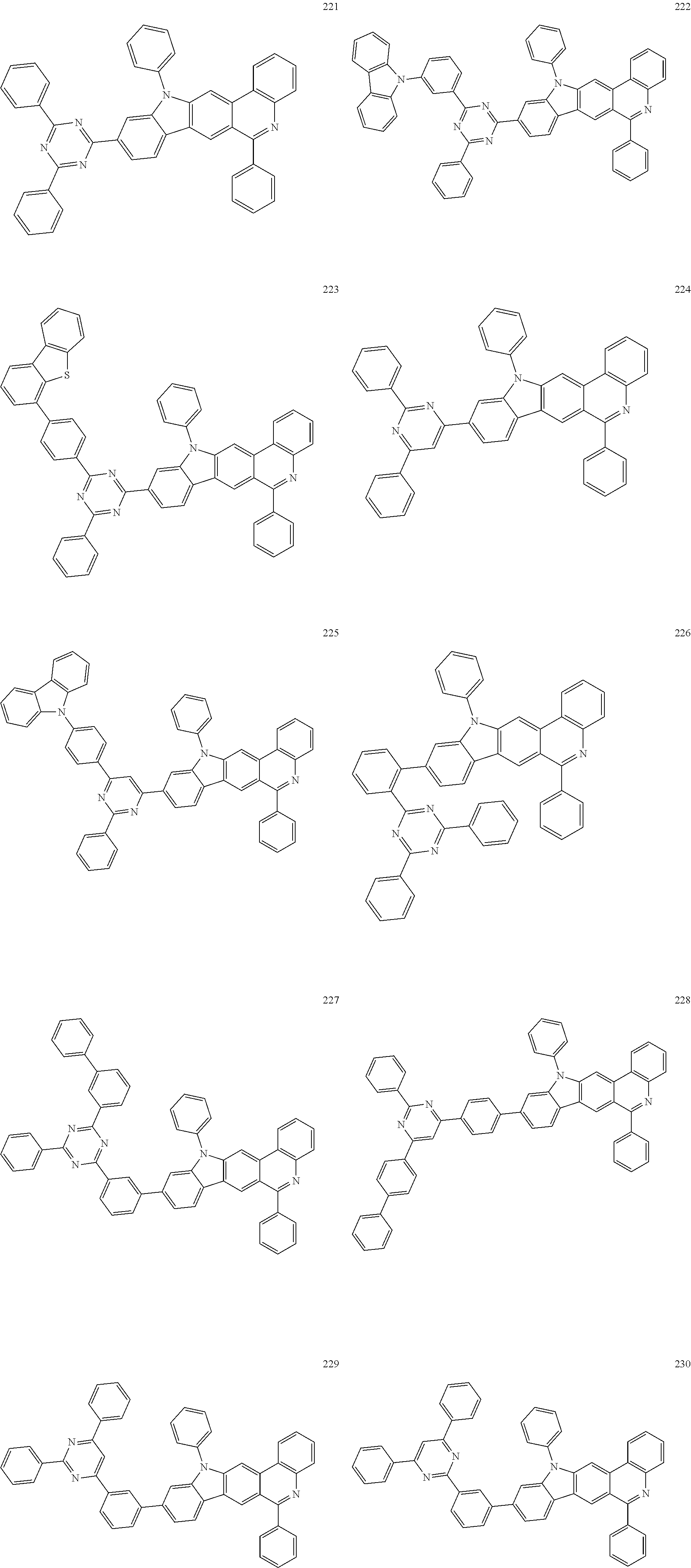

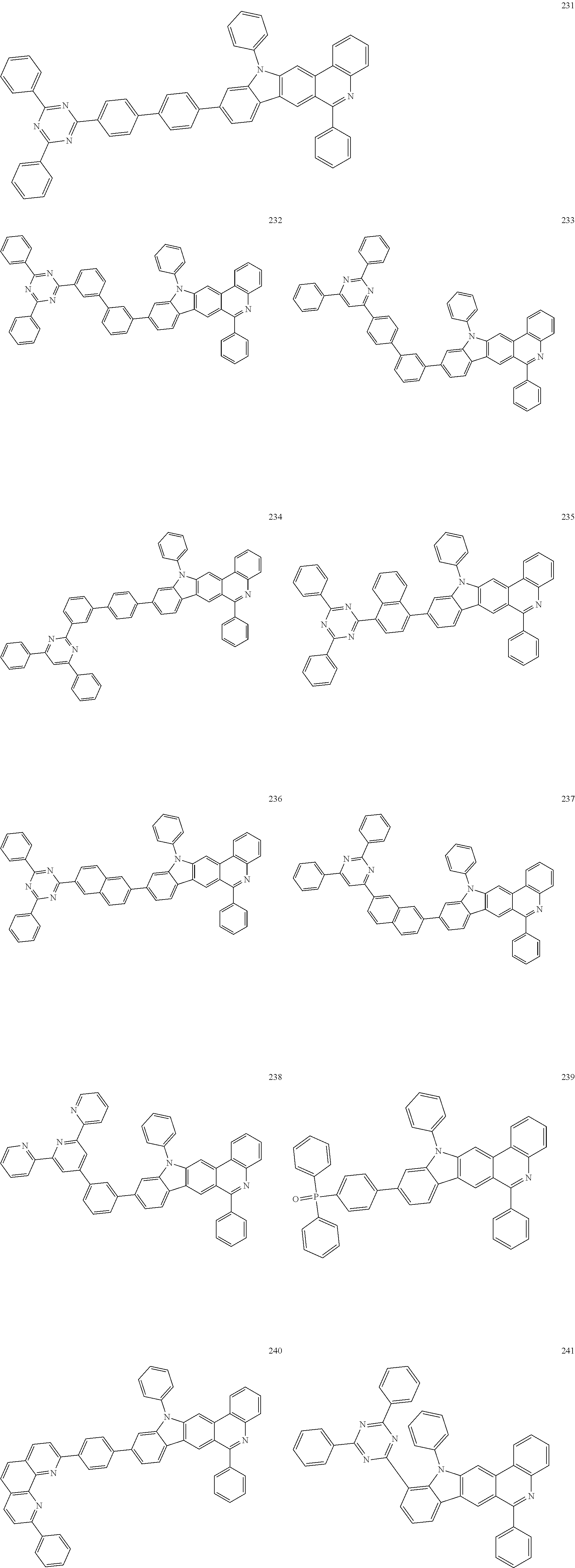

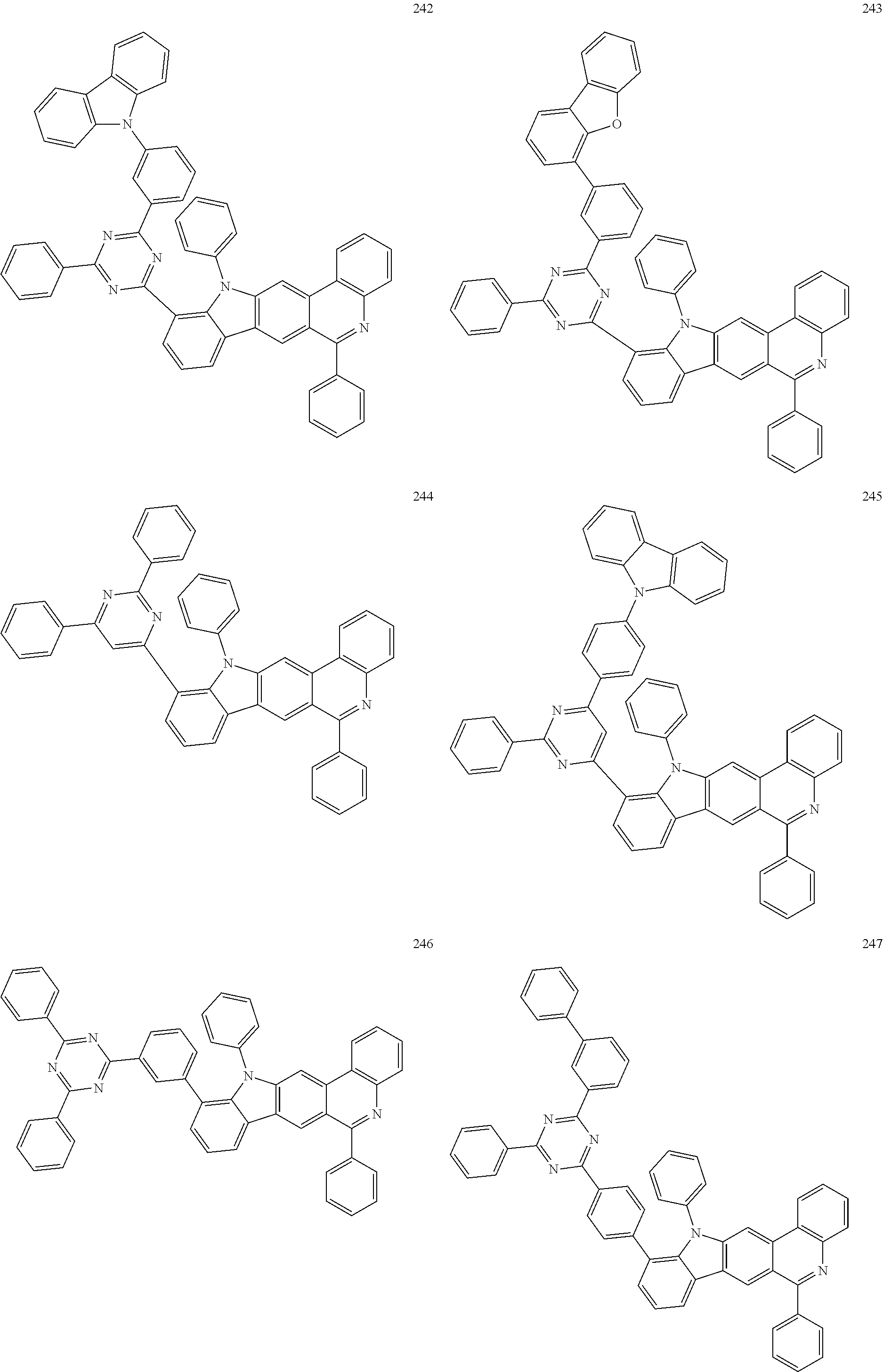

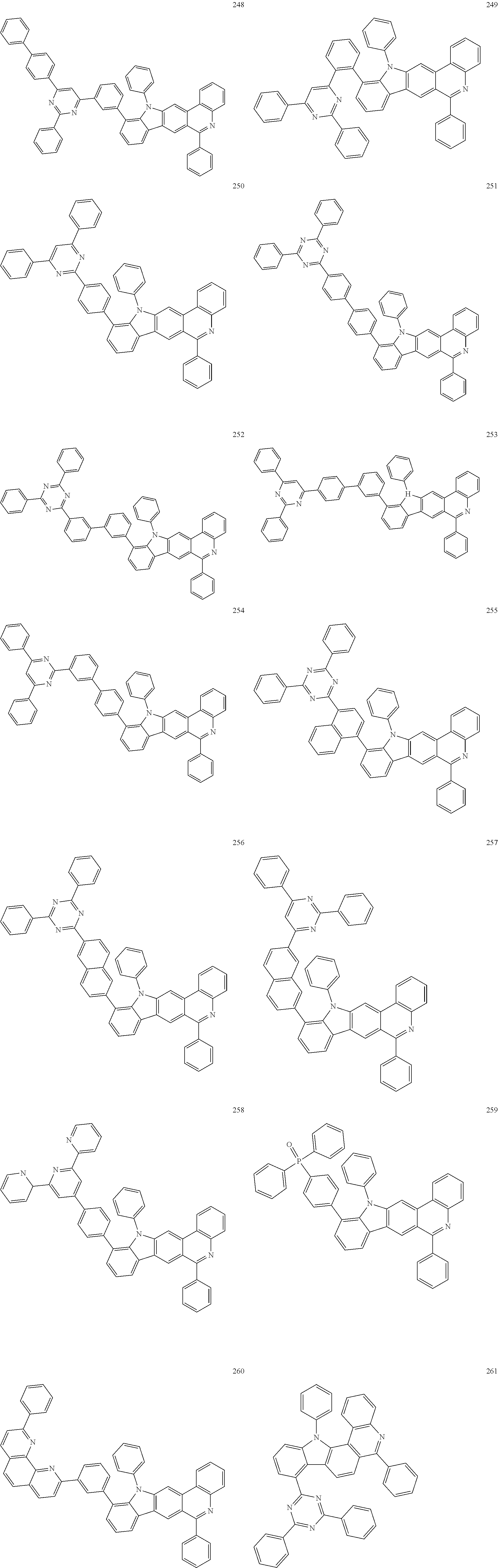

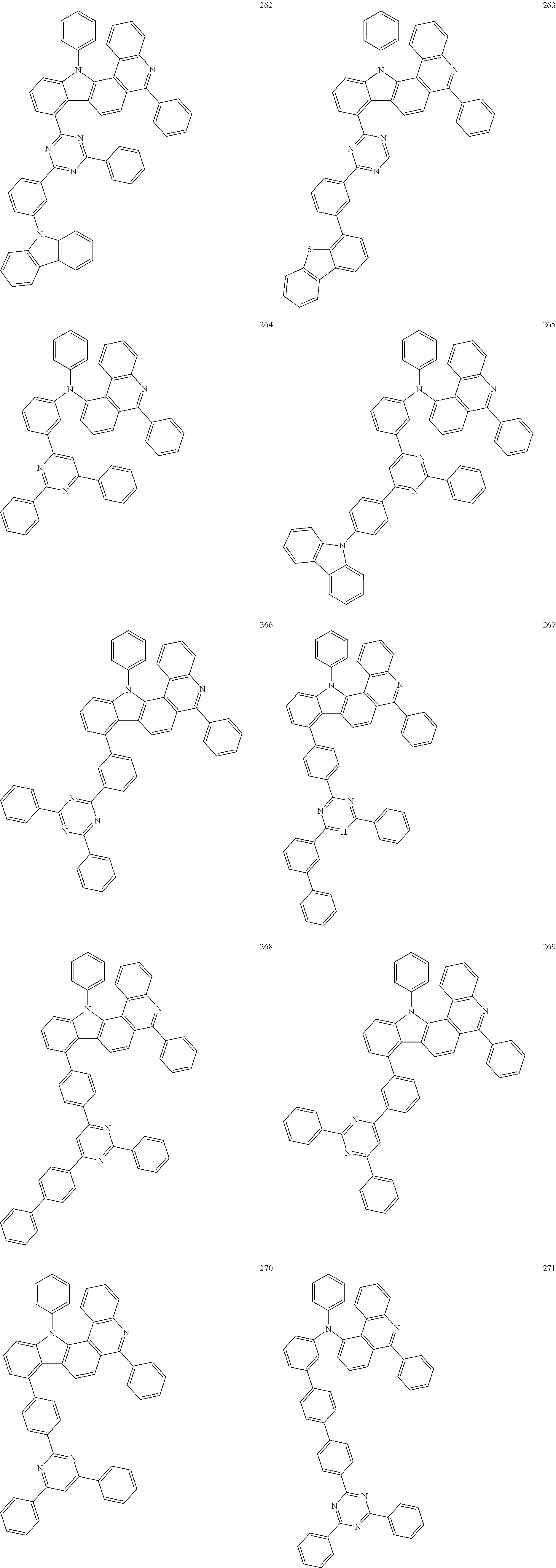

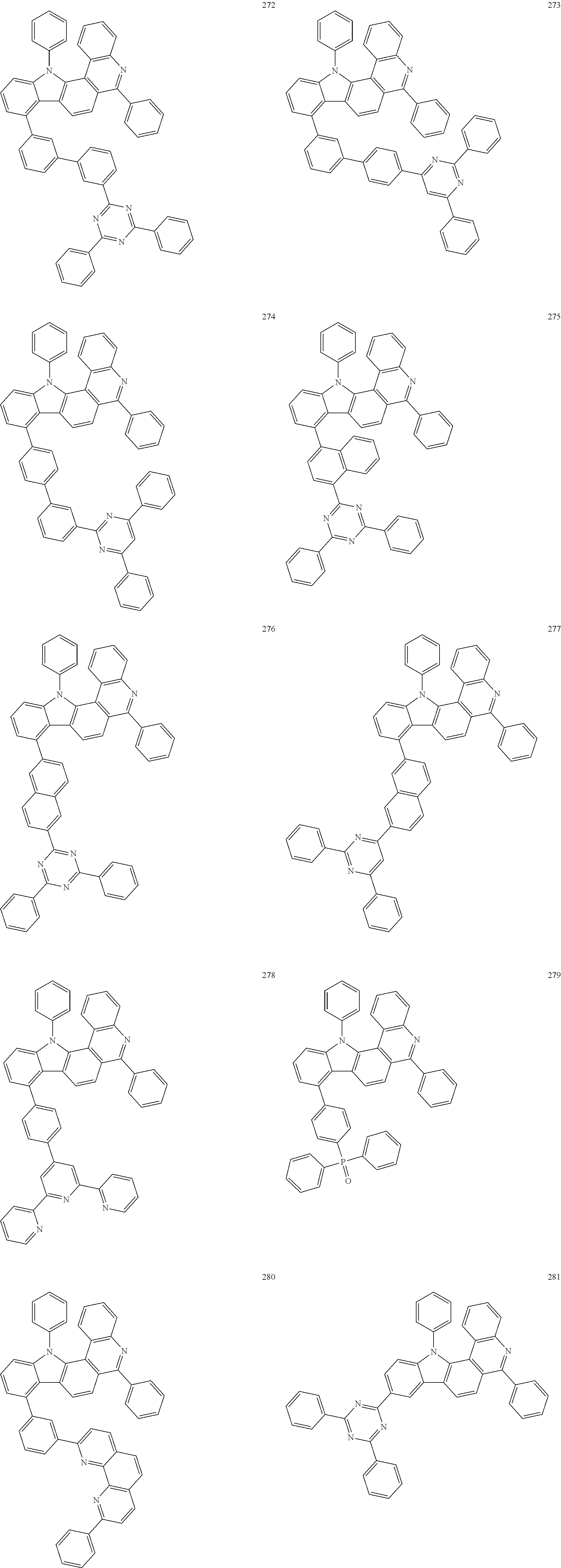

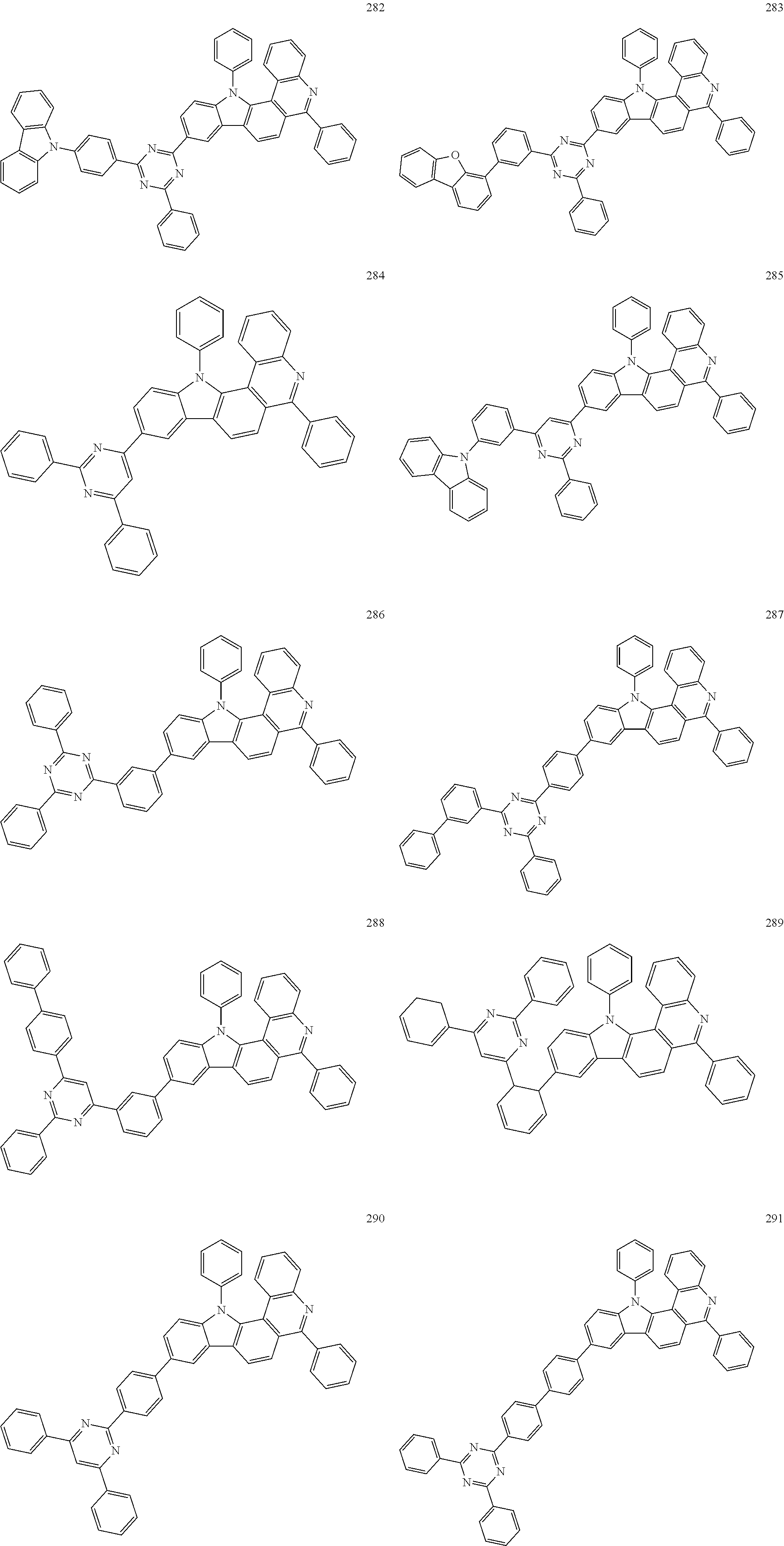

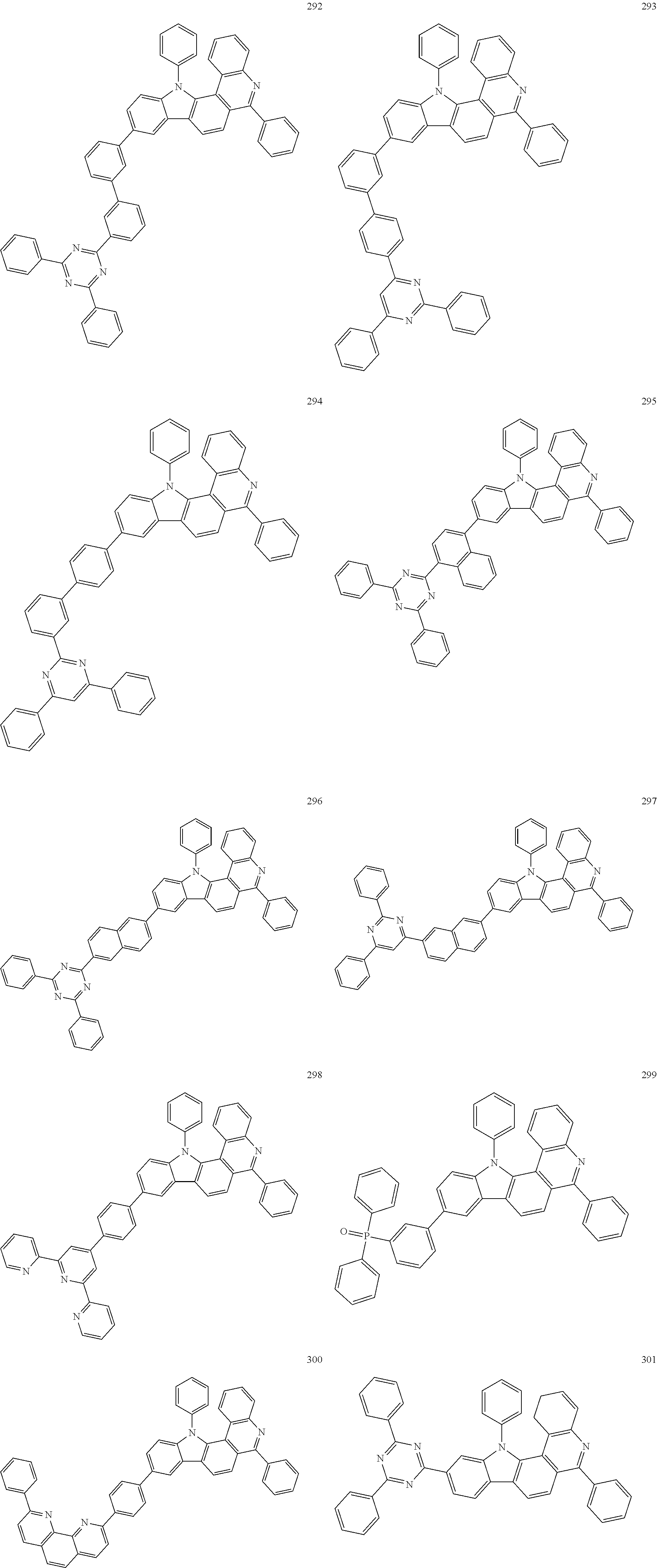

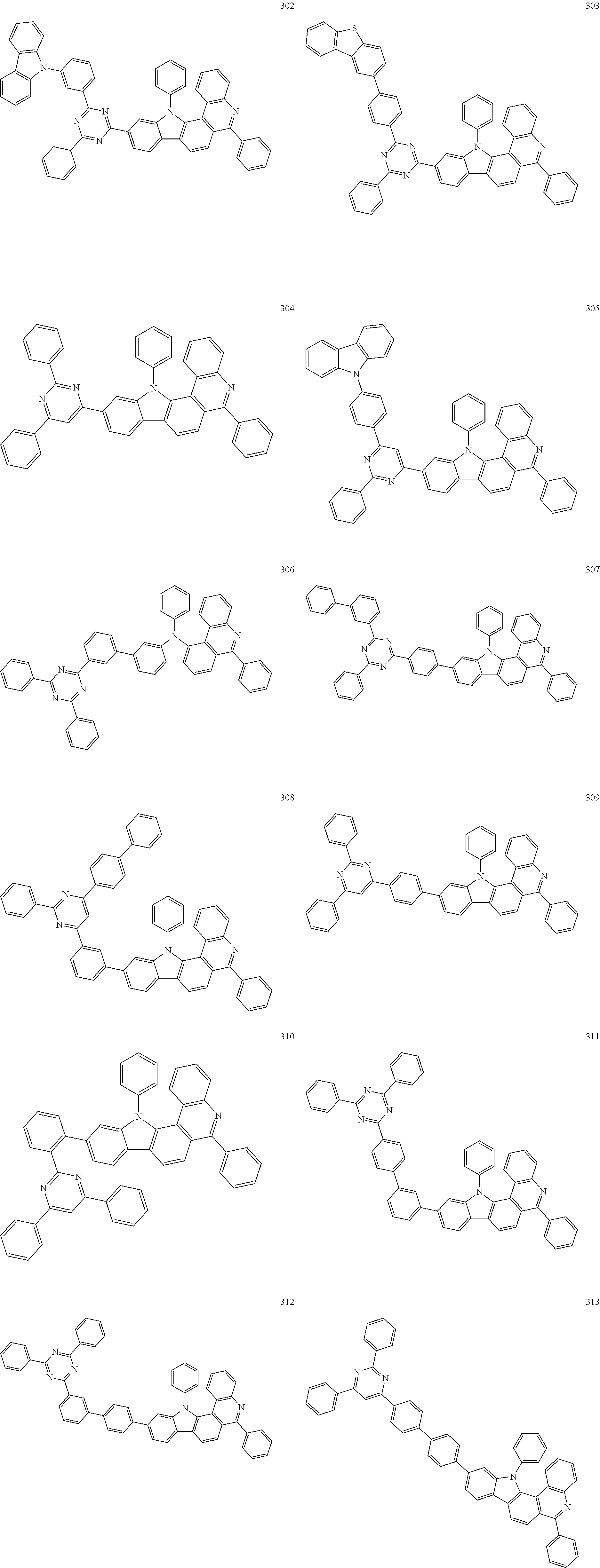

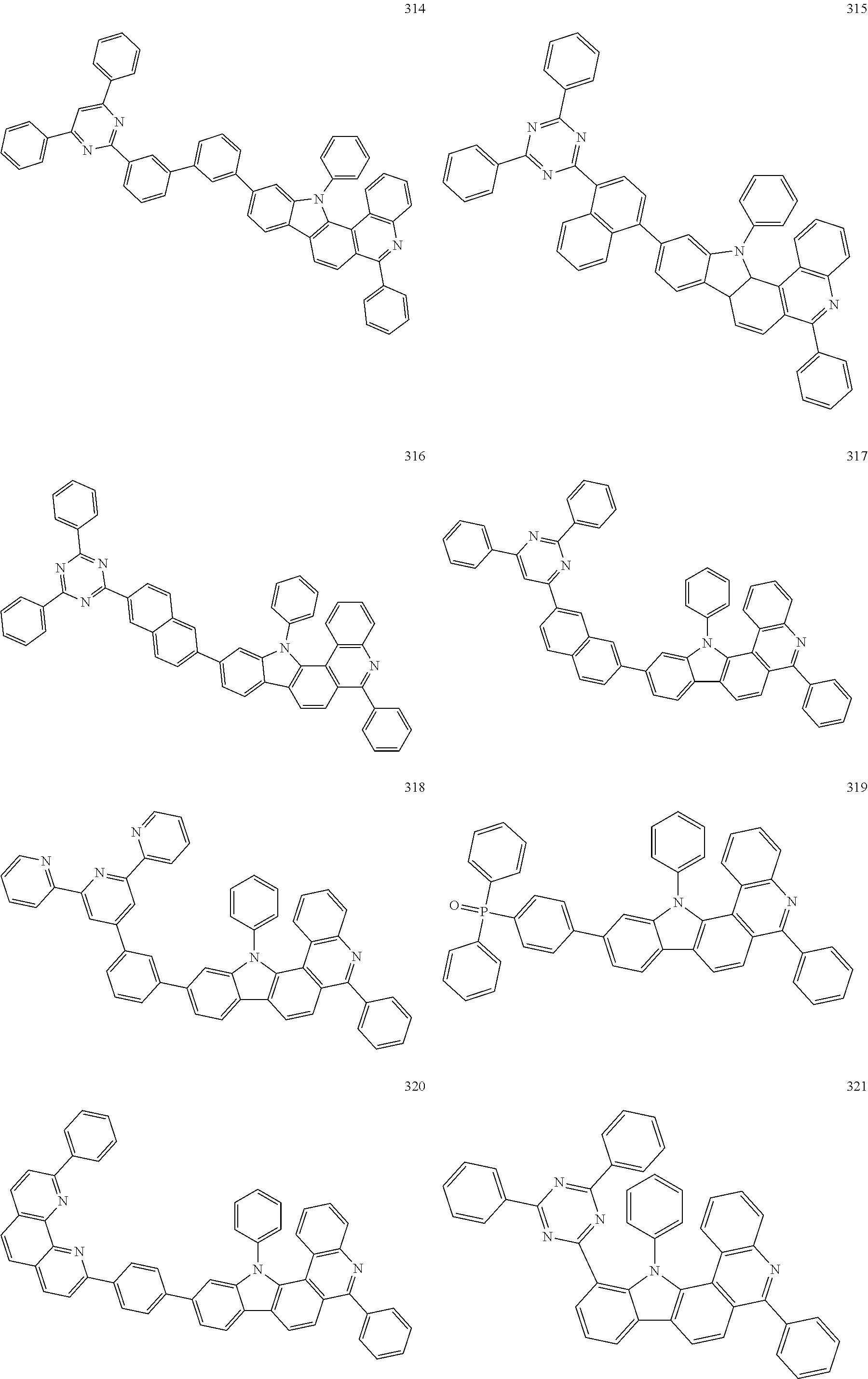

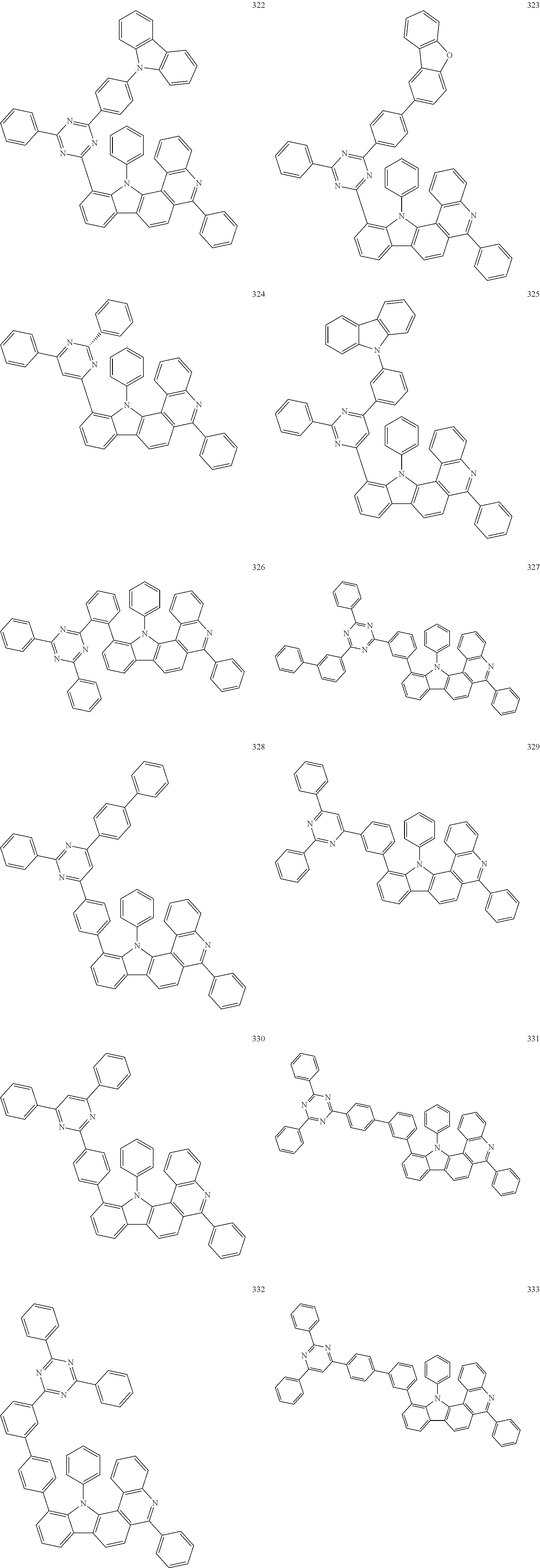

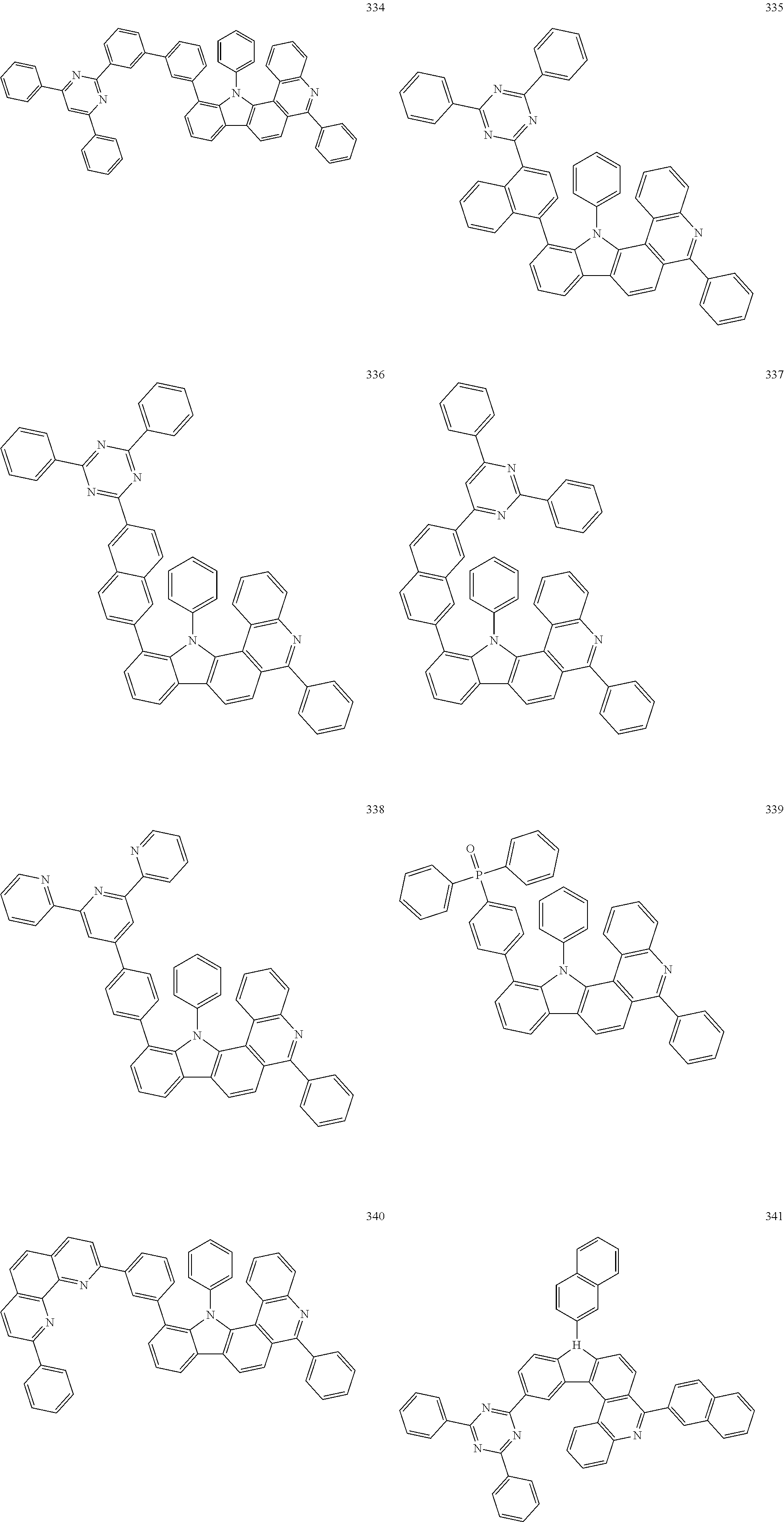

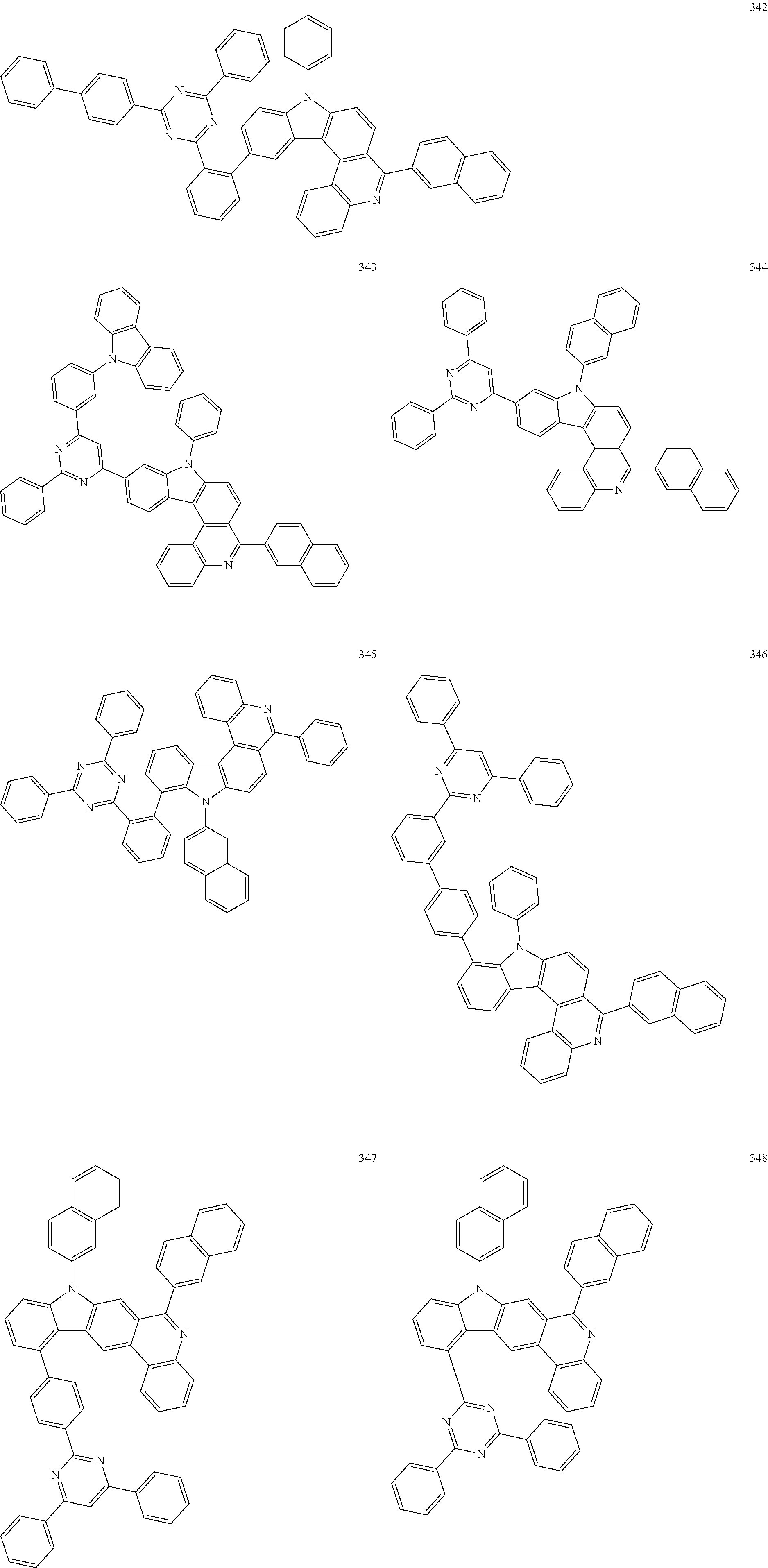

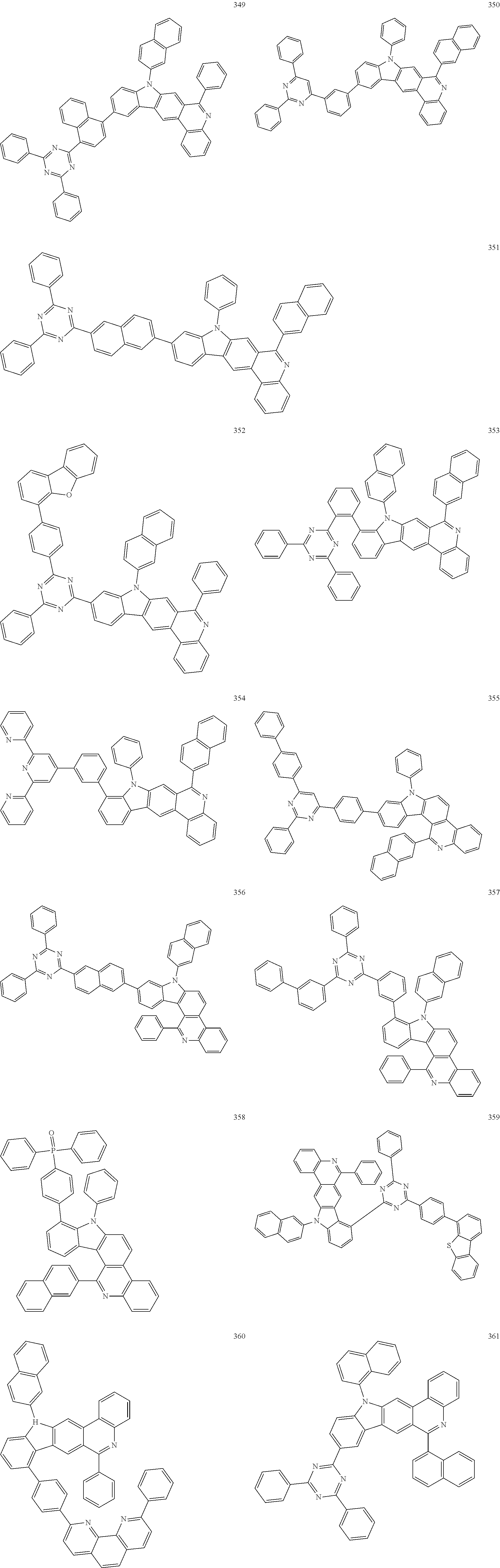

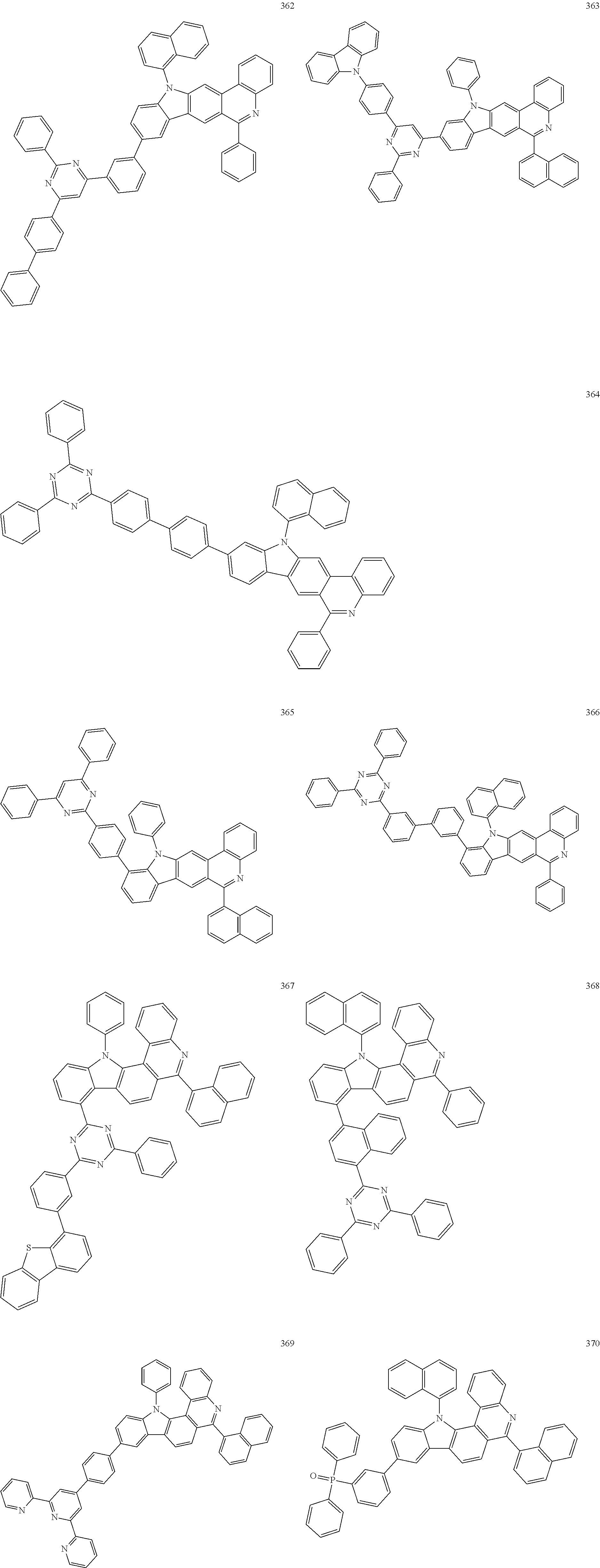

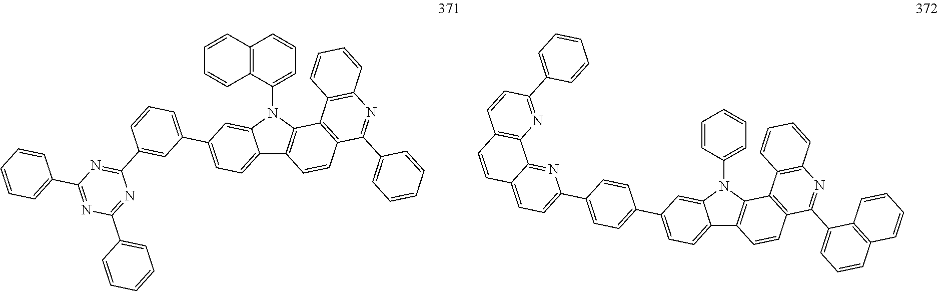 [SMILES: c1ccc(-c2nc(-c3ccccc3)nc(-c3cccc(-c4ccc5c6ccc7c(-c8ccccc8)nc8ccccc8c7c6n(-c6cccc7ccccc67)c5c4)c3)n2)cc1 ; c1ccc(-c2ccc3ccc4ccc(-c5ccc(-c6ccc7c8ccc9c(-c%10cccc%11ccccc%10%11)nc%10ccccc%10c9c8n(-c8ccccc8)c7c6)cc5)nc4c3n2)cc1]

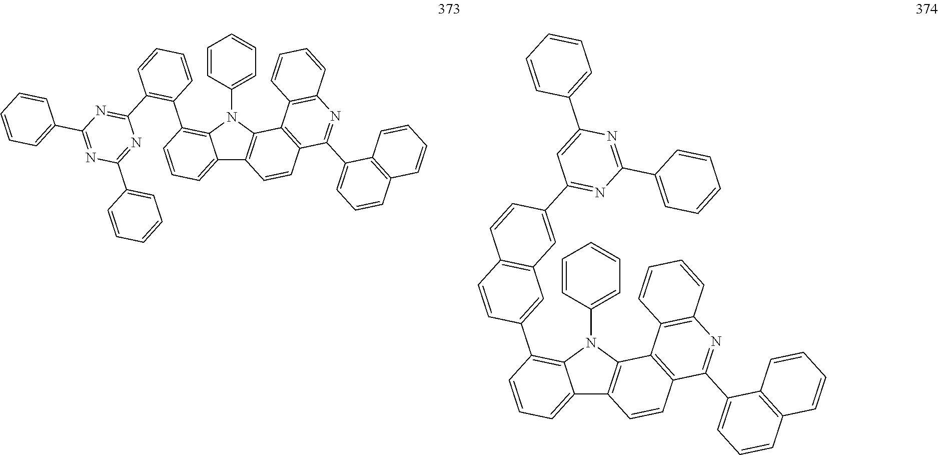 [SMILES: c1ccc(-c2nc(-c3ccccc3)nc(-c3ccccc3-c3cccc4c5ccc6c(-c7cccc8ccccc78)nc7ccccc7c6c5n(-c5ccccc5)c34)n2)cc1 ; c1ccc(-c2cc(-c3ccc4ccc(-c5cccc6c7ccc8c(-c9cccc%10ccccc9%10)nc9ccccc9c8c7n(-c7ccccc7)c56)cc4c3)nc(-c3ccccc3)n2)cc1]

In addition, by introducing various substituents to the structure of Chemical Formula 1, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

Meanwhile, the compound has a high glass transition temperature (Tg), and has excellent thermal stability. Such an increase in the thermal stability becomes an important factor providing driving stability to a device.

Another embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound represented by Chemical Formula 1.

In one embodiment of the present application, the first electrode may be an anode, and the second electrode may be a cathode.

In another embodiment, the first electrode may be a cathode, and the second electrode may be an anode.

Specific details on the heterocyclic compound represented by Chemical Formula 1 are the same as the descriptions provided above.

In one embodiment of the present application, the organic light emitting device may be a blue organic light emitting device, and the heterocyclic compound according to Chemical Formula 1 may be used as a material of the blue organic light emitting device.

In one embodiment of the present application, the organic light emitting device may be a green organic light emitting device, and the heterocyclic compound according to Chemical Formula 1 may be used as a material of the green organic light emitting device.

In one embodiment of the present application, the organic light emitting device may be a red organic light emitting device, and the heterocyclic compound according to Chemical Formula 1 may be used as a material of the red organic light emitting device.

The organic light emitting device of the present disclosure may be manufactured using common organic light emitting device manufacturing methods and materials except that one or more organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, or may also be formed in a multilayer structure in which two or more organic material layers are laminated. For example, the organic light emitting device according to one embodiment of the present disclosure may have a structure comprising a hole injection layer, a hole transfer layer, a light emitting layer, an electron transfer layer, an electron injection layer and the like as the organic material layer. However, the structure of the organic light emitting device is not limited thereto, and may comprise a smaller number of organic material layers.

In the organic light emitting device of the present disclosure, the organic material layer comprises an electron injection layer or an electron transfer layer, and the electron injection layer or the electron transfer layer may comprise the heterocyclic compound.

In the organic light emitting device of the present disclosure, the organic material layer comprises an electron transfer layer, and the electron transfer layer may comprise the heterocyclic compound.

In another organic light emitting device, the organic material layer comprises an electron blocking layer or a hole blocking layer, and the electron blocking layer or the hole blocking layer may comprise the heterocyclic compound.

In another organic light emitting device, the organic material layer comprises a hole blocking layer, and the hole blocking layer may comprise the heterocyclic compound.

In another organic light emitting device, the organic material layer comprises an electron transfer layer, a light emitting layer or a hole blocking layer, and the electron transfer layer, the light emitting layer or the hole blocking layer may comprise the heterocyclic compound.

The organic light emitting device of the present disclosure may further comprise one, two or more layers selected from the group consisting of a light emitting layer, a hole injection layer, a hole transfer layer, an electron injection layer, an electron transfer layer, an electron blocking layer and a hole blocking layer.

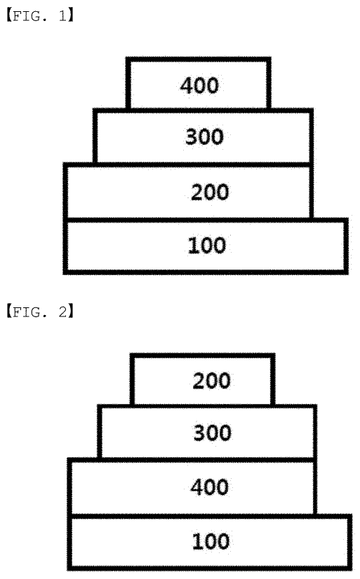

FIGS. 1 to 4 illustrate a lamination order of electrodes and organic material layers of an organic light emitting device according to one embodiment of the present application. However, the scope of the present application is not limited to these diagrams, and structures of organic light emitting devices known in the art may also be used in the present application.

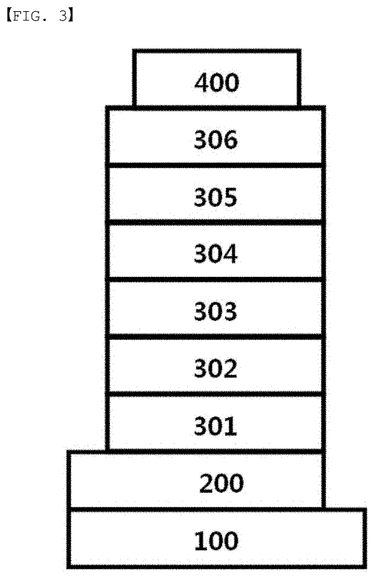

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

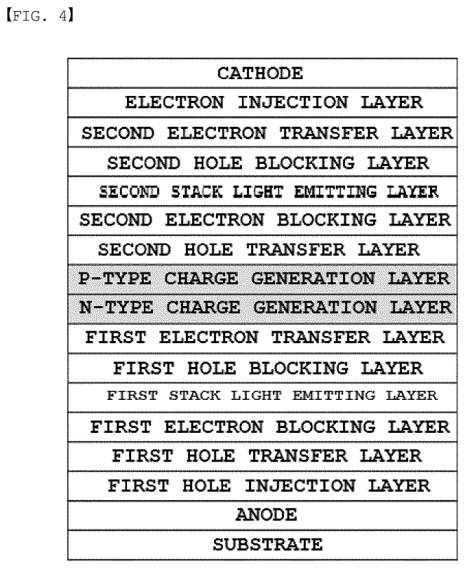

FIG. 3 illustrates a case of the organic material layer being a multilayer. The organic light emitting device according to FIG. 3 comprises a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, other layers except the light emitting layer may not be included, and other necessary functional layers may be further included.

The organic material layer comprising the compound of Chemical Formula 1 may further comprise other materials as necessary.

In addition, the organic light emitting device according to one embodiment of the present application comprises an anode, a cathode, and two or more stacks provided between the anode and the cathode, and the two or more stacks each independently comprise a light emitting layer, a charge generation layer is included between the two or more stacks, and the charge generation layer comprises the heterocyclic compound represented by Chemical Formula 1.

In addition, the organic light emitting device according to one embodiment of the present application comprises an anode, a first stack provided on the anode and comprising a first light emitting layer, a charge generation layer provided on the first stack, a second stack provided on the charge generation layer and comprising a second light emitting layer, and a cathode provided on the second stack. Herein, the charge generation layer may comprise the heterocyclic compound represented by Chemical Formula 1. In addition, the first stack and the second stack may each independently further comprise one or more types of the hole injection layer, the hole transfer layer, the hole blocking layer, the electron transfer layer, the electron injection layer and the like described above.

In the organic light emitting device provided in one embodiment of the present application, the charge generation layer is an N-type charge generation layer, and the charge generation layer comprises the heterocyclic compound.

The charge generation layer may be an N-type charge generation layer, and the charge generation layer may further comprise a dopant known in the art in addition to the heterocyclic compound represented by Chemical Formula 1.

As the organic light emitting device according to one embodiment of the present application, an organic light emitting device having a 2-stack tandem structure is schematically illustrated in the following FIG. 4.

Herein, the first electron blocking layer, the first hole blocking layer, the second hole blocking layer and the like described in FIG. 4 may not be included in some cases.

In the organic light emitting device according to one embodiment of the present application, materials other than the compound of Chemical Formula 1 are illustrated below, however, these are for illustrative purposes only and not for limiting the scope of the present application, and may be replaced by materials known in the art.

As the anode material, materials having relatively large work function may be used, and transparent conductive oxides, metals, conductive polymers or the like may be used. Specific examples of the anode material comprise metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the cathode material, materials having relatively small work function may be used, and metals, metal oxides, conductive polymers or the like may be used. Specific examples of the cathode material comprise metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrene-sulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection material, LiF is typically used in the art, however, the present application is not limited thereto.

As the light emitting material, red, green or blue light emitting materials may be used, and as necessary, two or more light emitting materials may be mixed and used. Herein, two or more light emitting materials may be used by being deposited as individual sources of supply or by being premixed and deposited as one source of supply. In addition, fluorescent materials may also be used as the light emitting material, however, phosphorescent materials may also be used. As the light emitting material, materials emitting light by bonding electrons and holes injected from an anode and a cathode, respectively, may be used alone, however, materials having a host material and a dopant material involving in light emission together may also be used.

When mixing light emitting material hosts, same series hosts may be mixed, or different series hosts may be mixed. For example, any two or more types of materials among n-type host materials or p-type host materials may be selected, and used as a host material of a light emitting layer.

The organic light emitting device according to one embodiment of the present application may be a top-emission type, a bottom-emission type or a dual-emission type depending on the materials used.

The heterocyclic compound according to one embodiment of the present application may also be used in an organic electronic device comprising an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### PREPARATION EXAMPLE

### [Preparation Example 1] Preparation of Compound 1

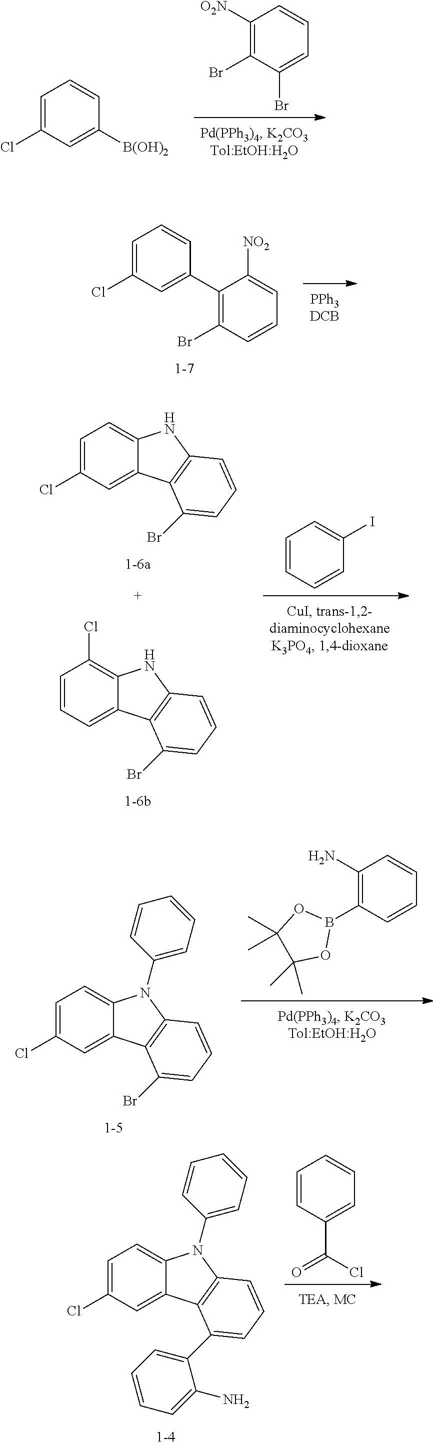

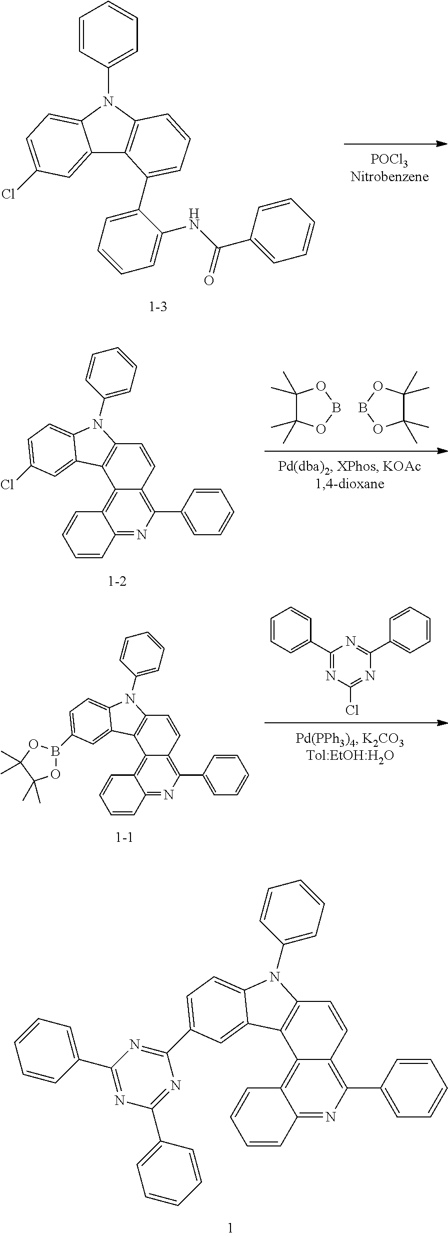

### Preparation of Compound 1-7

After dissolving (3-chlorophenyl)boronic acid (82 g, 525 mmol) and 1,2-dibromo-3-nitrobenzene (140 g, 500 mmol) in toluene, EtOH and H₂O (2000 mL:400 mL:400 mL), Pd(PPh₃)₄ (29 g, 25 mmol) and K₂CO₃ (126 g, 1500 mmol) were introduced thereto, and the result was refluxed for 4 hours. After the reaction was completed, the result was cooled to room temperature, and extracted with methylene chloride (MC). The result was dried with anhydrous MgSO₄, and the solvent was removed using a rotary evaporator. Target Compound 1-7 (125 g, 80%, green solid) was obtained using column chromatography (MC:Hx=1:3).

### Preparation of Compound 1-6

After dissolving Compound 1-7 (162 g, 400 mmol) and triphenylphosphine (314 g, 1200 mmol) in 1,2-dichlorobenzene (1000 mL), the result was refluxed for 12 hours. After the reaction was completed, the result was cooled to room temperature, and extracted with MC. The result was dried with anhydrous MgSO₄, and the solvent was removed using a rotary evaporator. Target Compounds 1-6a and 1-6b (100 g, 89%, green solid) were obtained in a ratio of 1:1 using column chromatography (MC:Hx=1:1).

### Preparation of Compound 1-5

After dissolving Compound 1-6a (50 g, 178 mmol) and iodobenzene (55 g, 267 mmol) in 1,4-dioxane (800 mL), CuI (15 g, 80 mmol), trans-1,2-diaminocyclohexane (9 g, 80 mmol) and K₃PO₄ (113 g, 530 mmol) were introduced thereto, and the result was refluxed for 12 hours. After the reaction was completed, the result was cooled to room temperature, and extracted with MC. The result was dried with anhydrous MgSO₄, and the solvent was removed using a rotary evaporator. Target Compound 1-5 (54 g, 85%, green solid) was obtained using column chromatography (MC:Hx=1:3).

### Preparation of Compound 1-4

After dissolving Compound 1-5 (54 g, 151 mmol) and 2-(4,4,5,5-tetramethyl-1,3,2-dioxaborolan-2-yl)aniline (37 g, 166 mmol) in toluene, EtOH and H₂O (800 mL:160 mL:160 mL), Pd(PPh₃)₄ (9 g, 8 mmol), K₂CO₃ (63 g, 453 mmol) were introduced thereto, and the result was refluxed for 12 hours. After the reaction was completed, the result was cooled to room temperature, and extracted with MC. The result was dried with anhydrous MgSO₄, and the solvent was removed using a rotary evaporator. Target Compound 1-4 (45 g, 82%, brown solid) was obtained using column chromatography (MC:Hx=1:3).

### Preparation of Compound 1-3

Compound 1-4 (45 g, 124 mmol) and triethylamine (51 mL, 370 mmol) were introduced to MC (900 mL) and dissolved therein. Benzoyl chloride (21 g, 149 mmol) was dissolved in MC (100 mL), and then slowly added dropwise to the mixture at 0° C. After the reaction was completed, MC and distilled water were introduced to the reaction solution, and the result was extracted. After that, the result was dried with anhydrous MgSO₄, and, after removing the solvent using a rotary evaporator, recrystallized with ethyl acetate (EA)/hexane (Hx) to obtain Compound 1-3 (53 g, 90%, white solid).

### Preparation of Compound 1-2

After dissolving Compound 1-3 (53 g, 112 mmol) in nitrobenzene (500 mL), POCl₃ (10 mL, 112 mmol) was slowly added dropwise thereto. After that, the result was stirred for 12 hours at 150° C. After the reaction was completed, the reaction solution was neutralized with an aqueous NaHCO₃ solution. Solids produced during the neutralization were filtered. The solids were recrystallized with MC/MeOH to obtain target Compound 1-2 (45 g, 88%, white solid).

### Preparation of Compound 1-1

After dissolving Compound 1-2 (45 g, 99 mmol), bis(pinacolato)diboron (33 g, 128 mmol), Pd(dba)₂ (1.6 g, 5 mmol), XPhos (1.7 g, 10 mmol) and KOAc (48 g, 297 mmol) in 1,4-dioxane (600 mL), the result was refluxed for 12 hours. After the reaction was completed, MC and distilled water were introduced to the reaction solution, and the result was extracted. After that, the result was dried with anhydrous MgSO₄, and the solvent was removed using a rotary evaporator. The result was silica passed and then MeOH slurried to obtain Compound 1-1 (51 g, 95%, pale pink solid).

### Preparation of Compound 1

After dissolving Compound 1-1 (10 g, 18 mmol) and 2-chloro-4,6-diphenyl-1,3,5-triazine (4.9 g, 18 mmol) in toluene, EtOH and H₂O (100 mL:20 mL:20 mL), Pd(PPh₃)₄ (1.2 g, 1 mmol) and K₂CO₃ (7.5 g, 54 mol) were introduced thereto, and the result was refluxed for 12 hours. After the reaction was completed, produced solids were filtered to obtain Compound 1 (11.4 g, 88%, white solid).

Target Compounds were synthesized in the same manner as in Preparation Example 1 except that Intermediate A of the following Table 1 was used instead of 2-chloro-4,6-diphenyl-1,3,5-triazine.

TABLE 1

| Compound | | | |
|---|---|---|---|
| No. | Intermediate A | Target Compound | Yield |
| 5 | 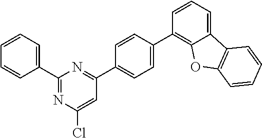 [SMILES: Clc1cc(-c2ccc(-c3cccc4c3oc3ccccc34)cc2)nc(-c2ccccc2)n1] | 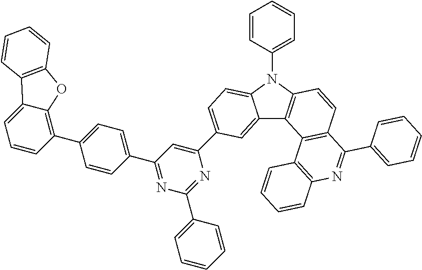 [SMILES: c1ccc(-c2nc(-c3ccc(-c4cccc5c4oc4ccccc45)cc3)cc(-c3ccc4c(c3)c3c5c(ccc3n4-c3ccccc3)c(-c3ccccc3)nc3ccccc35)n2)cc1] | 82% |
| 12 | 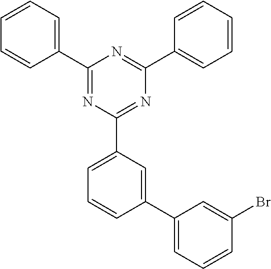 [SMILES: Brc1cccc(-c2cccc(-c3nc(-c4ccccc4)nc(-c4ccccc4)n3)c2)c1] | 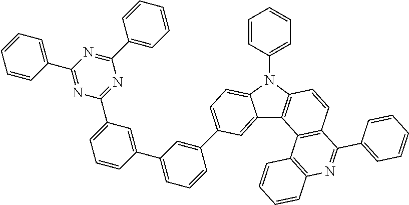 [SMILES: c1ccc(-c2nc(-c3ccccc3)nc(-c3cccc(-c4cccc(-c5ccc6c(c5)c5c7c(ccc5n6-c5ccccc5)c(-c5ccccc5)nc5ccccc57)c4)c3)n2)cc1] | 85% |
| 13 | 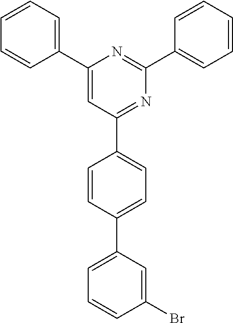 [SMILES: Brc1cccc(-c2ccc(-c3cc(-c4ccccc4)nc(-c4ccccc4)n3)cc2)c1] | 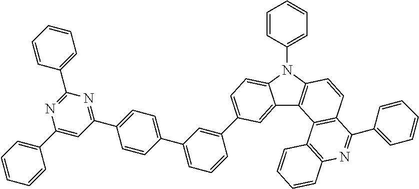 [SMILES: c1ccc(-c2cc(-c3ccc(-c4cccc(-c5ccc6c(c5)c5c7c(ccc5n6-c5ccccc5)c(-c5ccccc5)nc5ccccc57)c4)cc3)nc(-c3ccccc3)n2)cc1] | 79% |
| 19 | 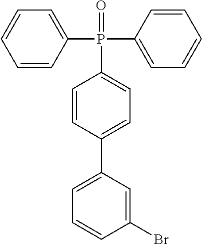 [SMILES: O=P(c1ccccc1)(c1ccccc1)c1ccc(-c2cccc(Br)c2)cc1] | 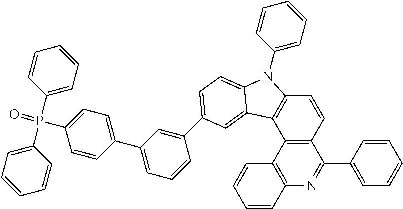 [SMILES: O=P(c1ccccc1)(c1ccccc1)c1ccc(-c2cccc(-c3ccc4c(c3)c3c5c(ccc3n4-c3ccccc3)c(-c3ccccc3)nc3ccccc35)c2)cc1] | 83% |

Target Compounds were synthesized in the same manner as in Preparation Example 1 except that Compound 1-6b was used instead of Compound 1-6a, and Intermediate B of the following Table 2 was used instead of 2-chloro-4,6-diphenyl-1,3,5-triazine.

TABLE 2

| Com- | | | |
|---|---|---|---|
| pound | | | |
| No. | Intermediate B | Target Compound | Yield |
| 43 | 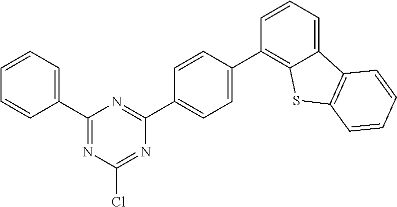 [SMILES: Clc1nc(-c2ccccc2)nc(-c2ccc(-c3cccc4c3sc3ccccc34)cc2)n1] | 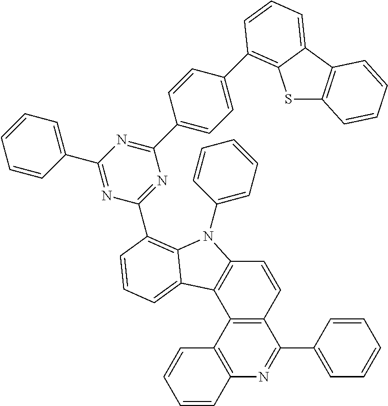 [SMILES: c1ccc(-c2nc(-c3ccc(-c4cccc5c4sc4ccccc45)cc3)nc(-c3cccc4c5c6c(ccc5n(-c5ccccc5)c34)c(-c3ccccc3)nc3ccccc36)n2)cc1] | 89% |
| 45 | 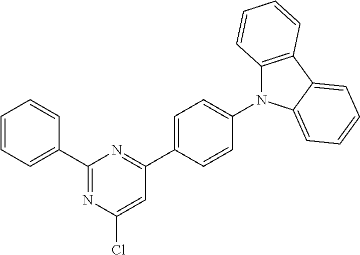 [SMILES: Clc1cc(-c2ccc(-n3c4ccccc4c4ccccc43)cc2)nc(-c2ccccc2)n1] | 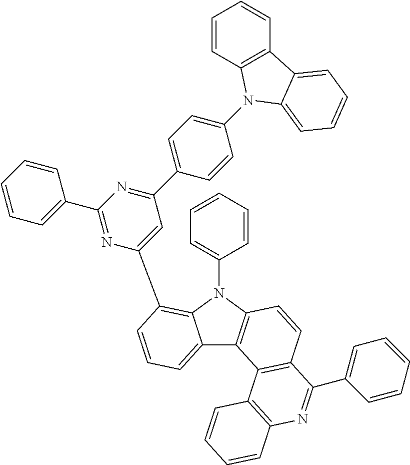 [SMILES: c1ccc(-c2nc(-c3ccc(-n4c5ccccc5c5ccccc54)cc3)cc(-c3cccc4c5c6c(ccc5n(-c5ccccc5)c34)c(-c3ccccc3)nc3ccccc36)n2)cc1] | 81% |
| 51 | 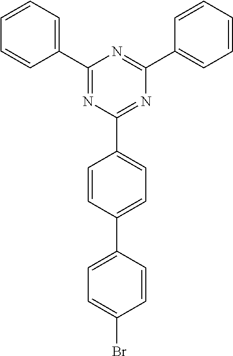 [SMILES: Brc1ccc(-c2ccc(-c3nc(-c4ccccc4)nc(-c4ccccc4)n3)cc2)cc1] | 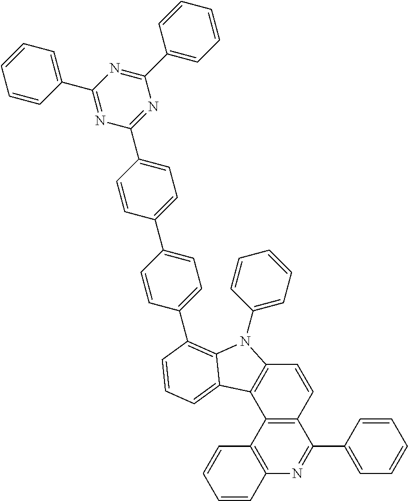 [SMILES: c1ccc(-c2nc(-c3ccccc3)nc(-c3ccc(-c4ccc(-c5cccc6c7c8c(ccc7n(-c7ccccc7)c56)c(-c5ccccc5)nc5ccccc58)cc4)cc3)n2)cc1] | 79% |
| 54 | 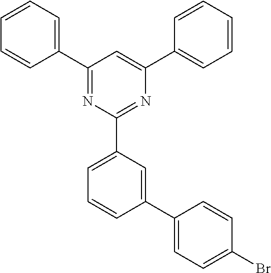 [SMILES: Brc1ccc(-c2cccc(-c3nc(-c4ccccc4)cc(-c4ccccc4)n3)c2)cc1] | 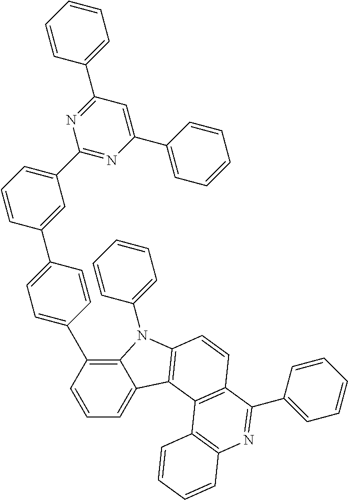 [SMILES: c1ccc(-c2cc(-c3ccccc3)nc(-c3cccc(-c4ccc(-c5cccc6c7c8c(ccc7n(-c7ccccc7)c56)c(-c5ccccc5)nc5ccccc58)cc4)c3)n2)cc1] | 86% |
| 57 | 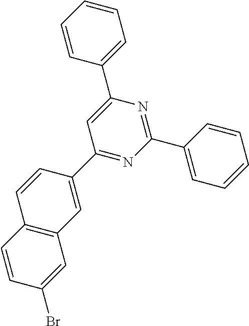 [SMILES: Brc1ccc2ccc(-c3cc(-c4ccccc4)nc(-c4ccccc4)n3)cc2c1] | 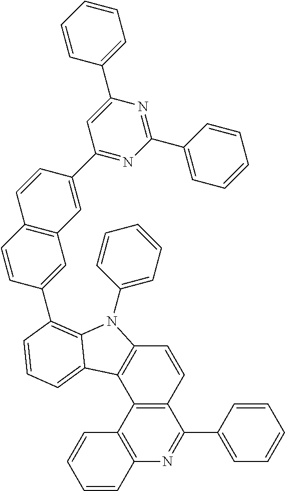 [SMILES: c1ccc(-c2cc(-c3ccc4ccc(-c5cccc6c7c8c(ccc7n(-c7ccccc7)c56)c(-c5ccccc5)nc5ccccc58)cc4c3)nc(-c3ccccc3)n2)cc1] | 81% |

### [Preparation Example 2] Preparation of Compound 64

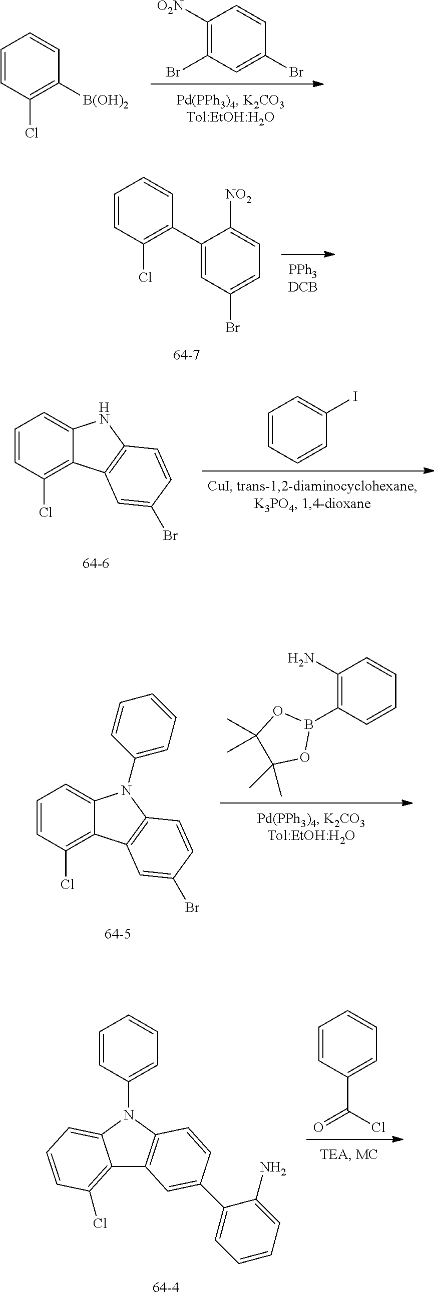

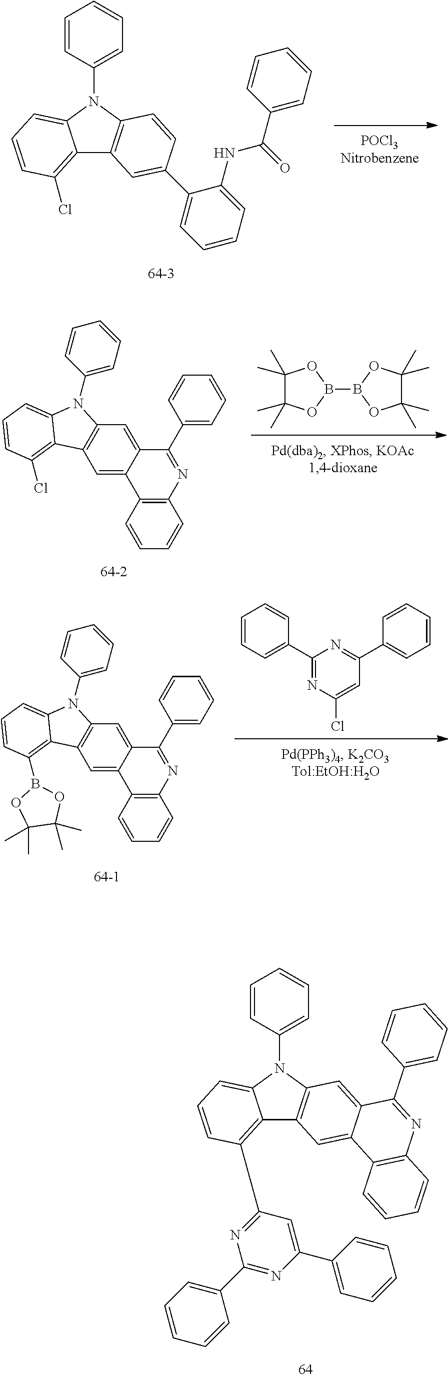

### Preparation of Compound 64-7

After dissolving (2-chlorophenyl)boronic acid (82 g, 525 mmol) and 2,4-dibromo-1-nitrobenzene (140 g, 500 mmol) in toluene, EtOH and H₂O (2000 mL:400 mL:400 mL), Pd(PPh₃)₄ (29 g, 25 mmol) and K₂CO₃ (126 g, 1500 mmol) were introduced thereto, and the result was refluxed for 4 hours. After the reaction was completed, the result was cooled to room temperature, and extracted with MC. The result was dried with anhydrous MgSO₄, and the solvent was removed using a rotary evaporator. Target Compound 64-7 (110 g, 70%, green solid) was obtained using column chromatography (MC:Hx=1:3).

### Preparation of Compound 64-6

After dissolving Compound 64-7 (110 g, 352 mmol) and triphenylphosphine (277 g, 1055 mmol) in 1,2-dichlorobenzene (1000 mL), the result was refluxed for 12 hours. After the reaction was completed, the result was cooled to room temperature, and extracted with MC. The result was dried with anhydrous MgSO₄, and the solvent was removed using a rotary evaporator. Target Compound 64-6 (78 g, 79%, green solid) was obtained using column chromatography (MC:Hx=1:1).

### Preparation of Compound 64-5

After dissolving Compound 64-6 (78 g, 278 mmol) and iodobenzene (85 g, 417 mmol) in 1,4-dioxane (800 mL), CuI (15 g, 80 mmol), trans-1,2-diaminocyclohexane (9 g, 80 mmol) and K₃PO₄ (177 g, 834 mmol) were introduced thereto, and the result was refluxed for 12 hours. After the reaction was completed, the result was cooled to room temperature, and extracted with MC. The result was dried with anhydrous MgSO₄, and the solvent was removed using a rotary evaporator. Target Compound 64-5 (84 g, 85%, green solid) was obtained using column chromatography (MC:Hx=1:3).

### Preparation of Compound 64-4

After dissolving Compound 64-5 (84 g, 235 mmol) and 2-(4,4,5,5-tetramethyl-1,3,2-dioxaborolan-2-yl)aniline (57 g, 260 mmol) in toluene, EtOH and H₂O (800 mL:160 mL:160 mL), Pd(PPh₃)₄ (14 g, 12 mmol) and K₂CO₃ (97 g, 705 mmol) were introduced thereto, and the result was refluxed for 12 hours. After the reaction was completed, the result was cooled to room temperature, and extracted with MC. The result was dried with anhydrous MgSO₄, and the solvent was removed using a rotary evaporator. Target Compound 64-4 (65 g, 75%, brown solid) was obtained using column chromatography (MC:Hx=1:3).

### Preparation of Compound 64-3

Compound 64-4 (65 g, 176 mmol) and triethylamine (74 mL, 528 mmol) were dissolved in MC (900 mL) and dissolved therein. Benzoyl chloride (25 g, 176 mmol) was dissolved in MC (100 mL), and then slowly added dropwise to the mixture at 0° C. After the reaction was completed, MC and distilled water were introduced to the reaction solution, and the result was extracted. After that, the result was dried with anhydrous MgSO₄, and, after removing the solvent using a rotary evaporator, recrystallized with EA/Hx to obtain Compound 64-3 (76 g, 92%, white solid).

### Preparation of Compound 64-2

After dissolving Compound 64-3 (76 g, 162 mmol) in nitrobenzene (500 mL), POCl₃ (15 mL, 162 mmol) was slowly added dropwise thereto. After that, the result was stirred for 12 hours at 150° C. After the reaction was completed, the reaction solution was neutralized with an aqueous NaHCO₂ solution. Solids produced during the neutralization were filtered. The solids were recrystallized with MC/MeOH to obtain target Compound 64-2 (65 g, 88%, white solid).

### Preparation of Compound 64-1

After dissolving Compound 64-2 (65 g, 143 mmol), bis(pinacolato)diboron (54 g, 215 mmol), Pd(dba)₂ (2 g, 7 mmol), XPhos (2.5 g, 14 mmol) and KOAc (42 g, 429 mmol) in 1,4-dioxane (600 mL), the result was refluxed for 12 hours. After the reaction was completed, MC and distilled water were introduced to the reaction solution, and the result was extracted. After that, the result was dried with anhydrous MgSO₄, and the solvent was removed using a rotary evaporator. The result was silica passed and then MeOH slurried to obtain Compound 64-1 (66 g, 85%, pale pink solid).

### Preparation of Compound 64

After dissolving Compound 64-1 (10 g, 18 mmol) and 4-chloro-2,6-diphenylpyrimidine (4.9 g, 18 mmol) in toluene, EtOH and H₂O (100 mL:20 mL:20 mL), Pd(PPh₃)₄ (1.2 g, 1 mmol) and K₂CO₃ (7.5 g, 54 mol) were introduced thereto, and the result was refluxed for 12 hours. After the reaction was completed, produced solids were filtered to obtain Compound 64 (11.4 g, 88%, white solid).

Target Compounds were synthesized in the same manner as in Preparation Example 2 except that Intermediate C of the following Table 3 was used instead of 4-chloro-2,6-diphenylpyrimidine.

TABLE 3

| Compound | | | |
|---|---|---|---|
| No. | Intermediate C | Target Compound | Yield |
| 66 | 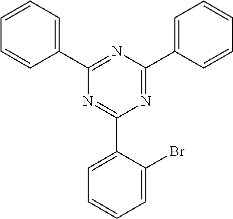 [SMILES: Brc1ccccc1-c1nc(-c2ccccc2)nc(-c2ccccc2)n1] | 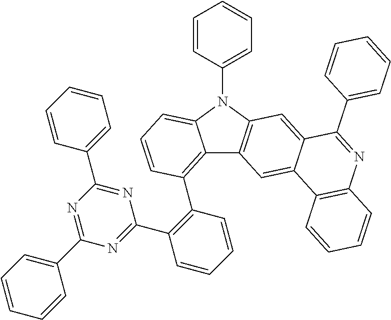 [SMILES: c1ccc(-c2nc(-c3ccccc3)nc(-c3ccccc3-c3cccc4c3c3cc5c(cc3n4-c3ccccc3)c(-c3ccccc3)nc3ccccc35)n2)cc1] | 85% |
| 70 | 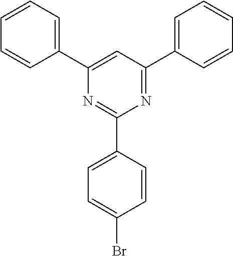 [SMILES: Brc1ccc(-c2nc(-c3ccccc3)cc(-c3ccccc3)n2)cc1] | 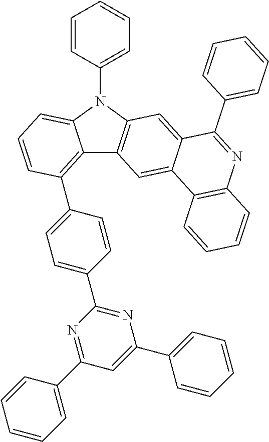 [SMILES: c1ccc(-c2cc(-c3ccccc3)nc(-c3ccc(-c4cccc5c4c4cc6c(cc4n5-c4ccccc4)c(-c4ccccc4)nc4ccccc46)cc3)n2)cc1] | 81% |
| 76 | 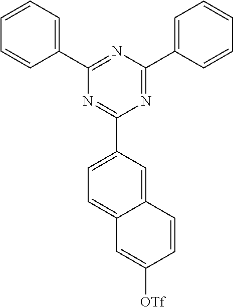 [SMILES: O=S(=O)(Oc1ccc2cc(-c3nc(-c4ccccc4)nc(-c4ccccc4)n3)ccc2c1)C(F)(F)F] | 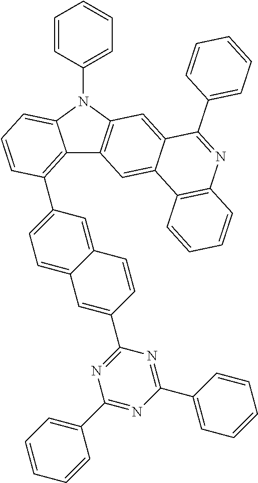 [SMILES: c1ccc(-c2nc(-c3ccccc3)nc(-c3ccc4cc(-c5cccc6c5c5cc7c(cc5n6-c5ccccc5)c(-c5ccccc5)nc5ccccc57)ccc4c3)n2)cc1] | 79% |
| 78 | 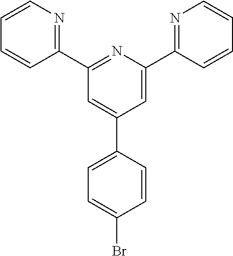 [SMILES: Brc1ccc(-c2cc(-c3ccccn3)nc(-c3ccccn3)c2)cc1] | 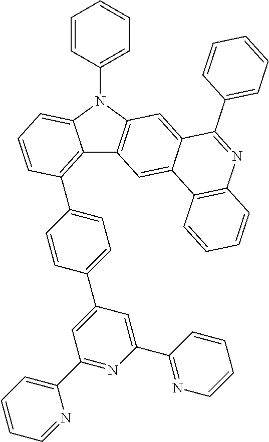 [SMILES: c1ccc(-c2nc3ccccc3c3cc4c5c(-c6ccc(-c7cc(-c8ccccn8)nc(-c8ccccn8)c7)cc6)cccc5n(-c5ccccc5)c4cc23)cc1] | 83% |

### [Preparation Example 3] Preparation of Compound 82

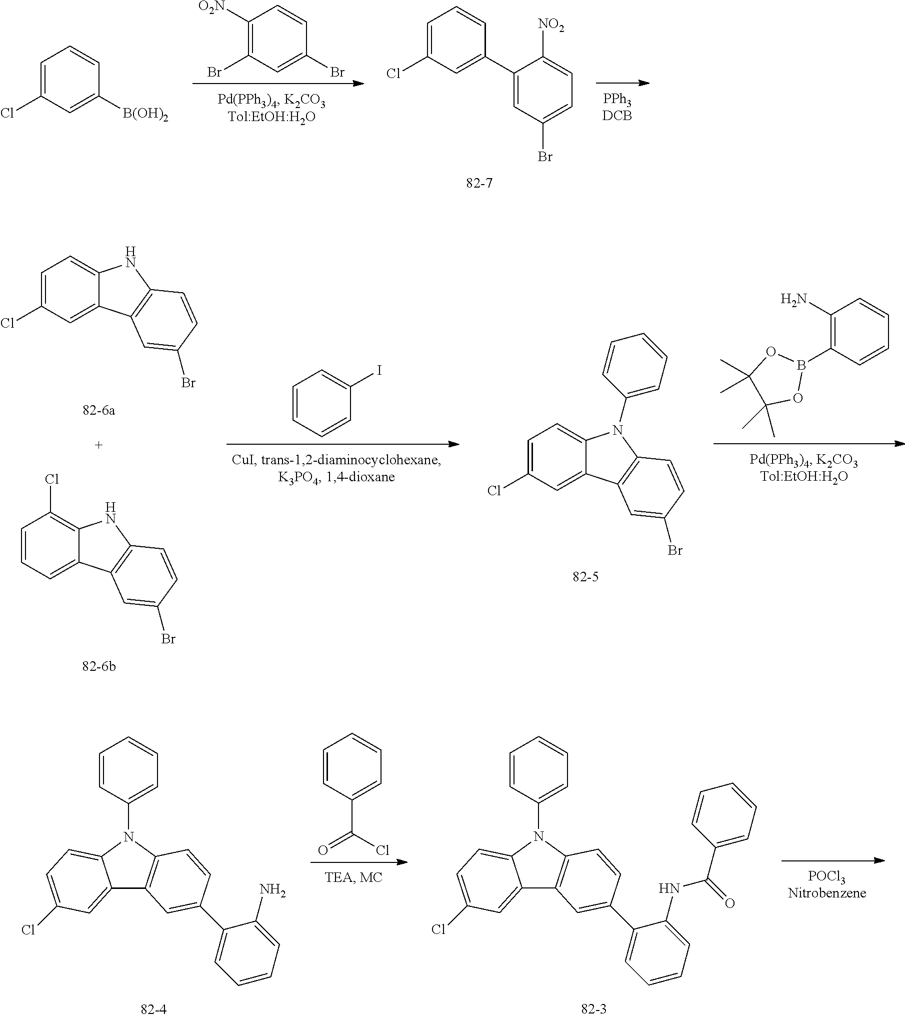

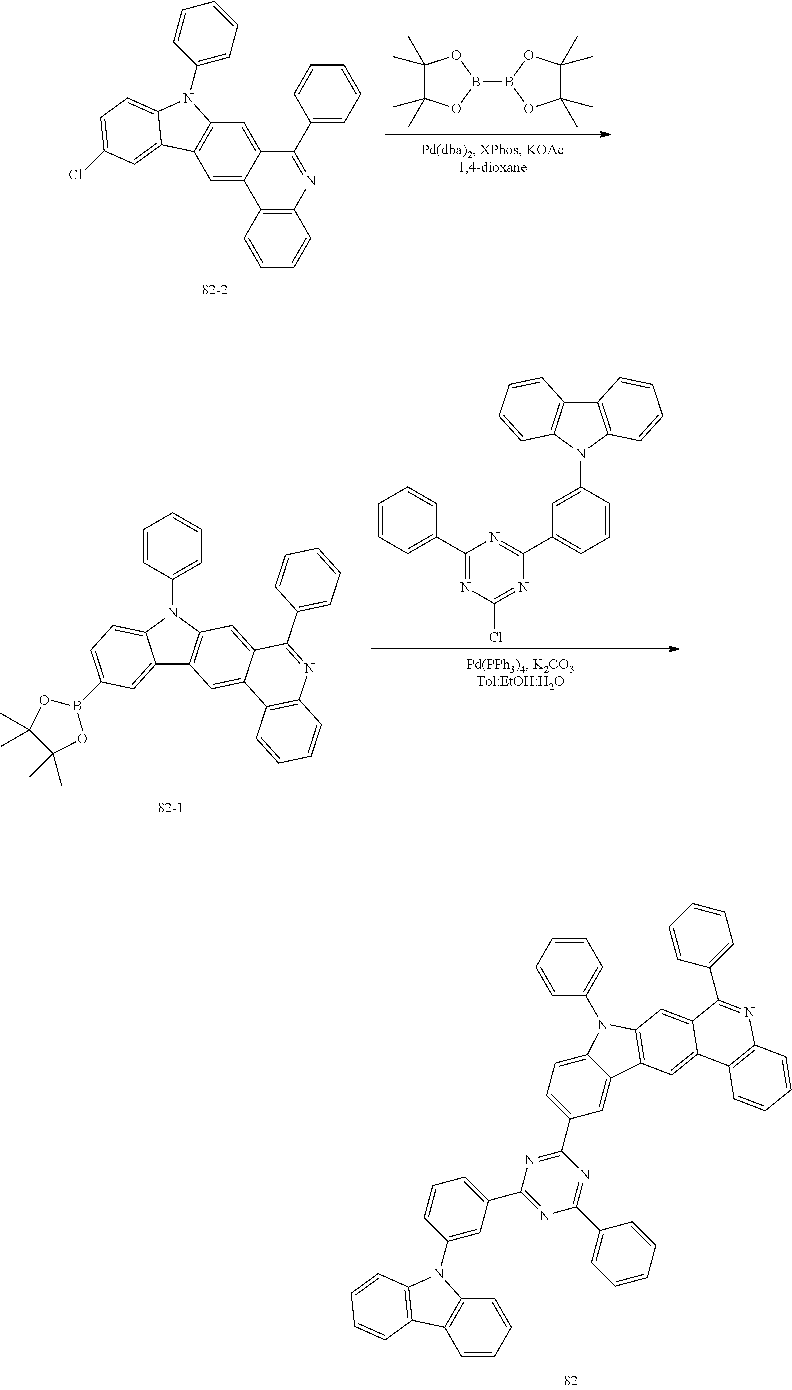

### Preparation of Compound 82-7

After dissolving (3-chlorophenyl)boronic acid (82 g, 525 mmol) and 2,4-dibromo-1-nitrobenzene (140 g, 500 mmol) in toluene, EtOH and H₂O (2000 mL:400 mL:400 mL), Pd(PPh₃)₄ (29 g, 25 mmol) and K₂CO₃ (126 g, 1500 mmol) were introduced thereto, and the result was refluxed for 4 hours. After the reaction was completed, the result was cooled to room temperature, and extracted with MC. The result was dried with anhydrous MgSO₄, and the solvent was removed using a rotary evaporator. Target Compound 82-7 (110 g, 70%, green solid) was obtained using column chromatography (MC:Hx=1:3).

### Preparation of Compound 82-6

After dissolving Compound 82-7 (110 g, 352 mmol) and triphenylphosphine (277 g, 1055 mmol) in 1,2-dichlorobenzene (1000 mL), the result was refluxed for 12 hours. After the reaction was completed, the result was cooled to room temperature, and extracted with MC. The result was dried with anhydrous MgSO₄, and the solvent was removed using a rotary evaporator. Target Compounds 82-6a and 82-6b (78 g, 79%, green solid) were obtained in a ratio of 1:1 using column chromatography (MC:Hx=1:1).

### Preparation of Compound 82-5

After dissolving Compound 82-6 (39 g, 184 mmol) and iodobenzene (42 g, 208 mmol) in 1,4-dioxane (800 mL), CuI (7.5 g, 40 mmol), trans-1,2-diaminocyclohexane (5 g, 40 mmol) and K₃PO₄ (90 g, 421 mmol) were introduced thereto, and the result was refluxed for 12 hours. After the reaction was completed, the result was cooled to room temperature, and extracted with MC. The result was dried with anhydrous MgSO₄, and the solvent was removed using a rotary evaporator. Target Compound 82-5 (42 g, 85%, green solid) was obtained using column chromatography (MC:Hx=1:3).

### Preparation of Compound 82-4

After dissolving Compound 82-5 (42 g, 167 mmol) and 2-(4,4,5,5-tetramethyl-1,3,2-dioxaborolan-2-yl)aniline (29 g, 130 mmol) in toluene, EtOH and H₂O (800 mL:160 mL:160 mL), Pd(PPh₃)₄ (7 g, 6 mmol) and K₂CO₃ (48 g, 350 mmol) were introduced thereto, and the result was refluxed for 12 hours. After the reaction was completed, the result was cooled to room temperature, and extracted with MC. The result was dried with anhydrous MgSO₄, and the solvent was removed using a rotary evaporator. Target Compound 82-4 (33 g, 80%, brown solid) was obtained using column chromatography (MC:Hx=1:3).

### Preparation of Compound 82-3

Compound 82-4 (33 g, 85 mmol) and triethylamine (37 mL, 260 mmol) were dissolved in MC (900 mL) and dissolved therein. Benzoyl chloride (13 g, 85 mmol) was dissolved in MC (100 mL), and then slowly added dropwise to the mixture at 0° C. After the reaction was completed, MC and distilled water were introduced to the reaction solution, and the result was extracted. After that, the result was dried with anhydrous MgSO₄, and, after removing the solvent using a rotary evaporator, recrystallized with EA/Hx to obtain Compound 82-3 (38 g, 88%, white solid).

### Preparation of Compound 82-2

After dissolving Compound 82-3 (38 g, 75 mmol) in nitrobenzene (500 mL), POCl₃ (8 mL, 75 mmol) was slowly added dropwise thereto. After that, the result was stirred for 12 hours at 150° C. After the reaction was completed, the reaction solution was neutralized with an aqueous NaHCO₃ solution. Solids produced during the neutralization were filtered. The solids were recrystallized with MC/MeOH to obtain target Compound 82-2 (33 g, 81%, white solid).

### Preparation of Compound 82-1

After dissolving Compound 82-2 (16 g, 38 mmol), bis(pinacolato)diboron (21 g, 100 mmol), Pd(dba)₂ (1 g, 4 mmol), XPhos (1.3 g, 8 mmol) and KOAc (42 g, 429 mmol) in 1,4-dioxane (600 mL), the result was refluxed for 12 hours. After the reaction was completed, MC and distilled water were introduced to the reaction solution, and the result was extracted. After that, the result was dried with anhydrous MgSO₄, and the solvent was removed using a rotary evaporator. The result was silica passed and then MeOH slurried to obtain Compound 82-1 (22 g, 89%, pale pink solid).

### Preparation of Compound 82

After dissolving Compound 82-1 (10 g, 18 mmol) and 4-chloro-2,6-diphenylpyrimidine (4.9 g, 18 mmol) in toluene, EtOH and H₂O (100 mL:20 mL:20 mL), Pd(PPh₃)₄ (1.2 g, 1 mmol) and K₂CO₃ (7.5 g, 54 mol) were introduced thereto, and the result was refluxed for 12 hours. After the reaction was completed, produced solids were filtered to obtain Compound 82 (11.4 g, 80%, white solid).

Target Compounds were synthesized in the same manner as in Preparation Example 3 except that Intermediate D of the following Table 4 was used instead of 9-(3-(4-chloro-6-phenyl-1,3,5-triazin-2-yl)phenyl)-9H-carbazole.

TABLE 4

| Com- | | | |
|---|---|---|---|
| pound | | | |
| No. | Intermediate D | Target Compound | Yield |
| 87 | 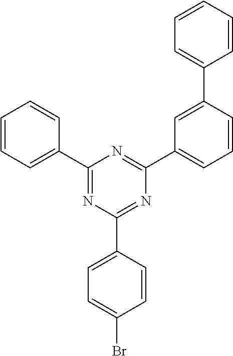 [SMILES: Brc1ccc(-c2nc(-c3ccccc3)nc(-c3cccc(-c4ccccc4)c3)n2)cc1] | 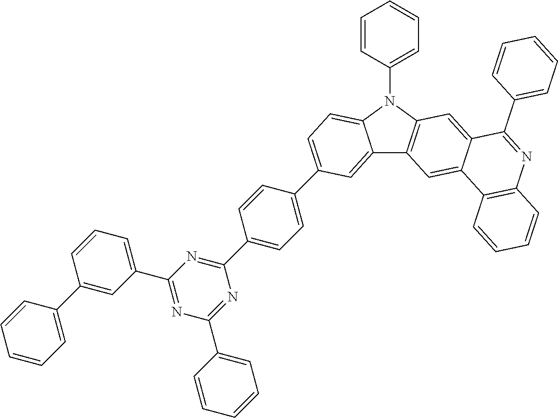 [SMILES: c1ccc(-c2cccc(-c3nc(-c4ccccc4)nc(-c4ccc(-c5ccc6c(c5)c5cc7c(cc5n6-c5ccccc5)c(-c5ccccc5)nc5ccccc57)cc4)n3)c2)cc1] | 85% |
| 89 | 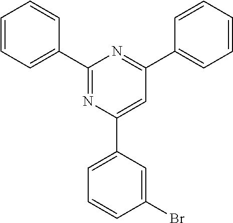 [SMILES: Brc1cccc(-c2cc(-c3ccccc3)nc(-c3ccccc3)n2)c1] | 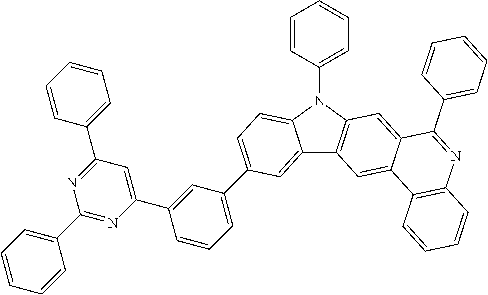 [SMILES: c1ccc(-c2cc(-c3cccc(-c4ccc5c(c4)c4cc6c(cc4n5-c4ccccc4)c(-c4ccccc4)nc4ccccc46)c3)nc(-c3ccccc3)n2)cc1] | 81% |
| 93 | 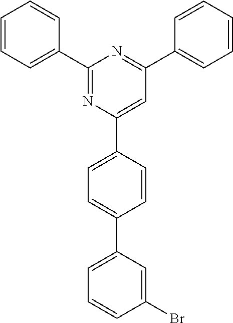 [SMILES: Brc1cccc(-c2ccc(-c3cc(-c4ccccc4)nc(-c4ccccc4)n3)cc2)c1] | 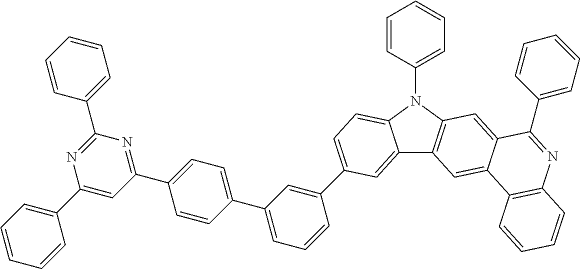 [SMILES: c1ccc(-c2cc(-c3ccc(-c4cccc(-c5ccc6c(c5)c5cc7c(cc5n6-c5ccccc5)c(-c5ccccc5)nc5ccccc57)c4)cc3)nc(-c3ccccc3)n2)cc1] | 79% |
| 99 | 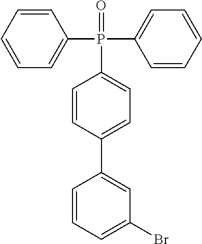 [SMILES: O=P(c1ccccc1)(c1ccccc1)c1ccc(-c2cccc(Br)c2)cc1] | 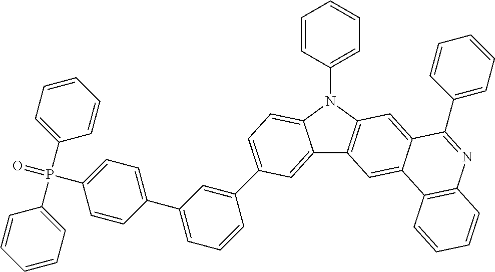 [SMILES: O=P(c1ccccc1)(c1ccccc1)c1ccc(-c2cccc(-c3ccc4c(c3)c3cc5c(cc3n4-c3ccccc3)c(-c3ccccc3)nc3ccccc35)c2)cc1] | 83% |

Target Compounds were synthesized in the same manner as in Preparation Example 3 except that (4-chlorophenyl)boronic acid was used instead of (3-chlorophenyl)boronic acid, and Intermediate E of the following Table 5 was used instead of 9-(3-(4-chloro-6-phenyl-1,3,5-triazin-2-yl)phenyl)-9H-carbazole.

TABLE 5

| Com- | | | |
|---|---|---|---|
| pound | | | |
| No. | Intermediate E | Target Compound | Yield |
| 103 | 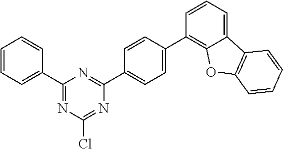 [SMILES: Clc1nc(-c2ccccc2)nc(-c2ccc(-c3cccc4c3oc3ccccc34)cc2)n1] | 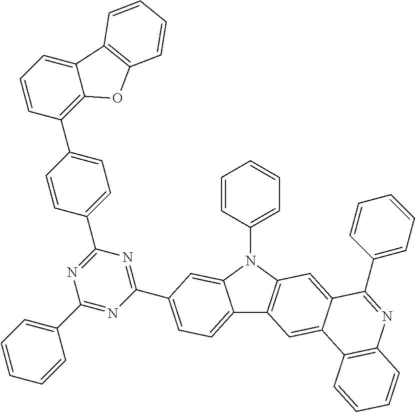 [SMILES: c1ccc(-c2nc(-c3ccc(-c4cccc5c4oc4ccccc45)cc3)nc(-c3ccc4c5cc6c(cc5n(-c5ccccc5)c4c3)c(-c3ccccc3)nc3ccccc36)n2)cc1] | 80% |
| 106 | 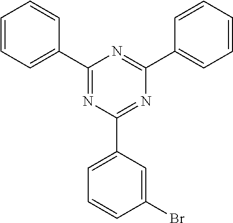 [SMILES: Brc1cccc(-c2nc(-c3ccccc3)nc(-c3ccccc3)n2)c1] | 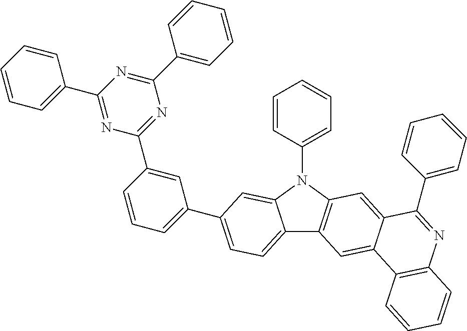 [SMILES: c1ccc(-c2nc(-c3ccccc3)nc(-c3cccc(-c4ccc5c6cc7c(cc6n(-c6ccccc6)c5c4)c(-c4ccccc4)nc4ccccc47)c3)n2)cc1] | 83% |
| 112 | 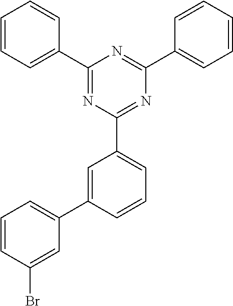 [SMILES: Brc1cccc(-c2cccc(-c3nc(-c4ccccc4)nc(-c4ccccc4)n3)c2)c1] | 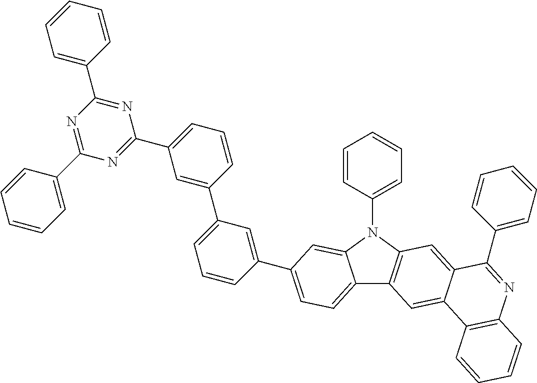 [SMILES: c1ccc(-c2nc(-c3ccccc3)nc(-c3cccc(-c4cccc(-c5ccc6c7cc8c(cc7n(-c7ccccc7)c6c5)c(-c5ccccc5)nc5ccccc58)c4)c3)n2)cc1] | 75% |
| 114 | 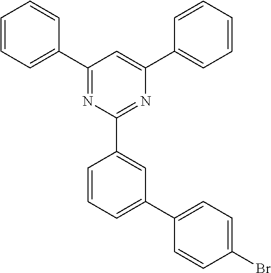 [SMILES: Brc1ccc(-c2cccc(-c3nc(-c4ccccc4)cc(-c4ccccc4)n3)c2)cc1] | 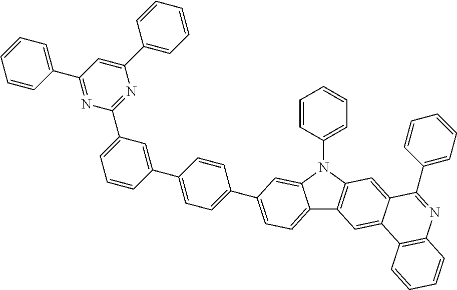 [SMILES: c1ccc(-c2cc(-c3ccccc3)nc(-c3cccc(-c4ccc(-c5ccc6c7cc8c(cc7n(-c7ccccc7)c6c5)c(-c5ccccc5)nc5ccccc58)cc4)c3)n2)cc1] | 77% |
| 119 | 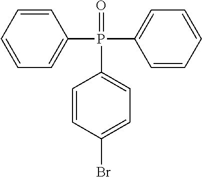 [SMILES: O=P(c1ccccc1)(c1ccccc1)c1ccc(Br)cc1] | 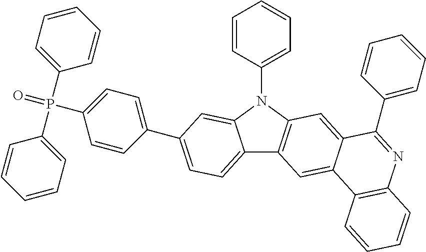 [SMILES: O=P(c1ccccc1)(c1ccccc1)c1ccc(-c2ccc3c4cc5c(cc4n(-c4ccccc4)c3c2)c(-c2ccccc2)nc2ccccc25)cc1] | 76% |

Target Compounds were synthesized in the same manner as in Preparation Example 3 except that Compound 82-6b was used instead of Compound 82-6a, and Intermediate F of the following Table 6 was used instead of 9-(3-(4-chloro-6-phenyl-1,3,5-triazin-2-yl)phenyl)-9H-carbazole.

TABLE 6

| Com- | | | |
|---|---|---|---|
| pound | | | |
| No. | Intermediate F | Target Compound | Yield |
| 122 | 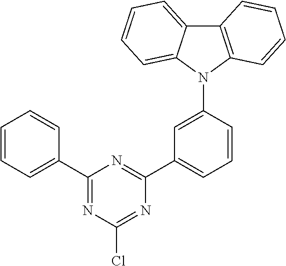 [SMILES: Clc1nc(-c2ccccc2)nc(-c2cccc(-n3c4ccccc4c4ccccc43)c2)n1] | 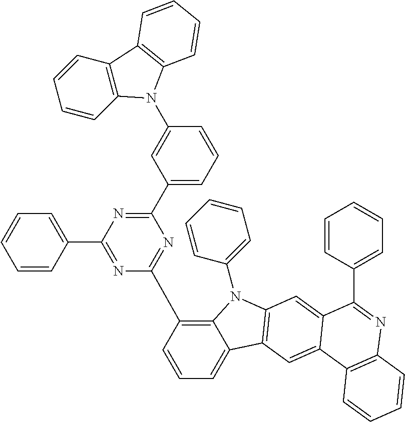 [SMILES: c1ccc(-c2nc(-c3cccc(-n4c5ccccc5c5ccccc54)c3)nc(-c3cccc4c5cc6c(cc5n(-c5ccccc5)c34)c(-c3ccccc3)nc3ccccc36)n2)cc1] | 75% |
| 128 | 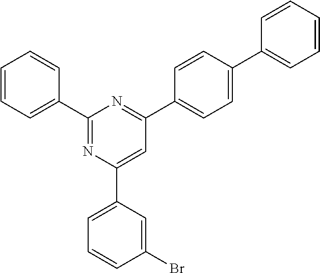 [SMILES: Brc1cccc(-c2cc(-c3ccc(-c4ccccc4)cc3)nc(-c3ccccc3)n2)c1] | 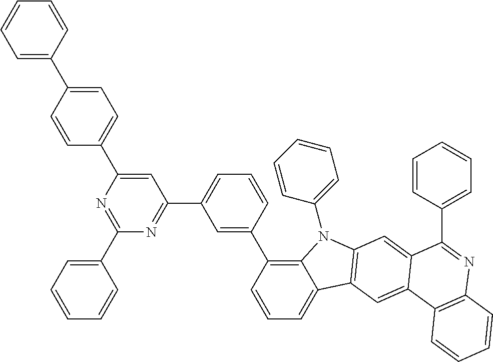 [SMILES: c1ccc(-c2ccc(-c3cc(-c4cccc(-c5cccc6c7cc8c(cc7n(-c7ccccc7)c56)c(-c5ccccc5)nc5ccccc58)c4)nc(-c4ccccc4)n3)cc2)cc1] | 86% |
| 130 | 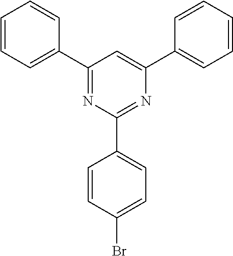 [SMILES: Brc1ccc(-c2nc(-c3ccccc3)cc(-c3ccccc3)n2)cc1] | 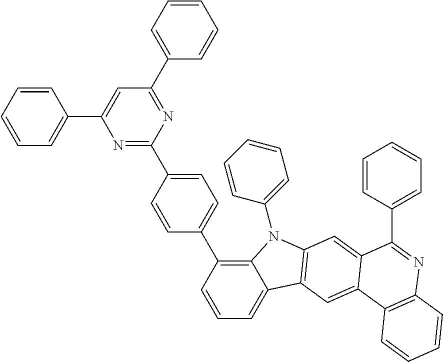 [SMILES: c1ccc(-c2cc(-c3ccccc3)nc(-c3ccc(-c4cccc5c6cc7c(cc6n(-c6ccccc6)c45)c(-c4ccccc4)nc4ccccc47)cc3)n2)cc1] | 90% |
| 135 | 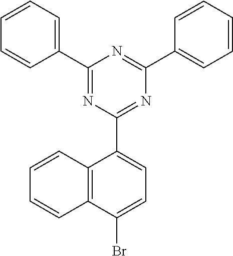 [SMILES: Brc1ccc(-c2nc(-c3ccccc3)nc(-c3ccccc3)n2)c2ccccc12] | 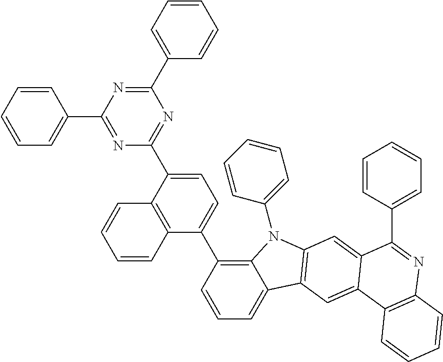 [SMILES: c1ccc(-c2nc(-c3ccccc3)nc(-c3ccc(-c4cccc5c6cc7c(cc6n(-c6ccccc6)c45)c(-c4ccccc4)nc4ccccc47)c4ccccc34)n2)cc1] | 82% |
| 137 | 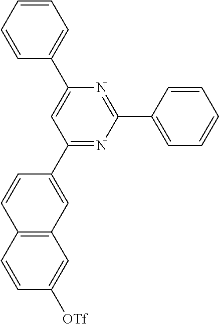 [SMILES: O=S(=O)(Oc1ccc2ccc(-c3cc(-c4ccccc4)nc(-c4ccccc4)n3)cc2c1)C(F)(F)F] | 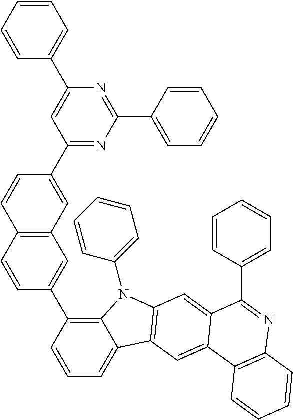 [SMILES: c1ccc(-c2cc(-c3ccc4ccc(-c5cccc6c7cc8c(cc7n(-c7ccccc7)c56)c(-c5ccccc5)nc5ccccc58)cc4c3)nc(-c3ccccc3)n2)cc1] | 79% |

### [Preparation Example 4] Preparation of Compound 144

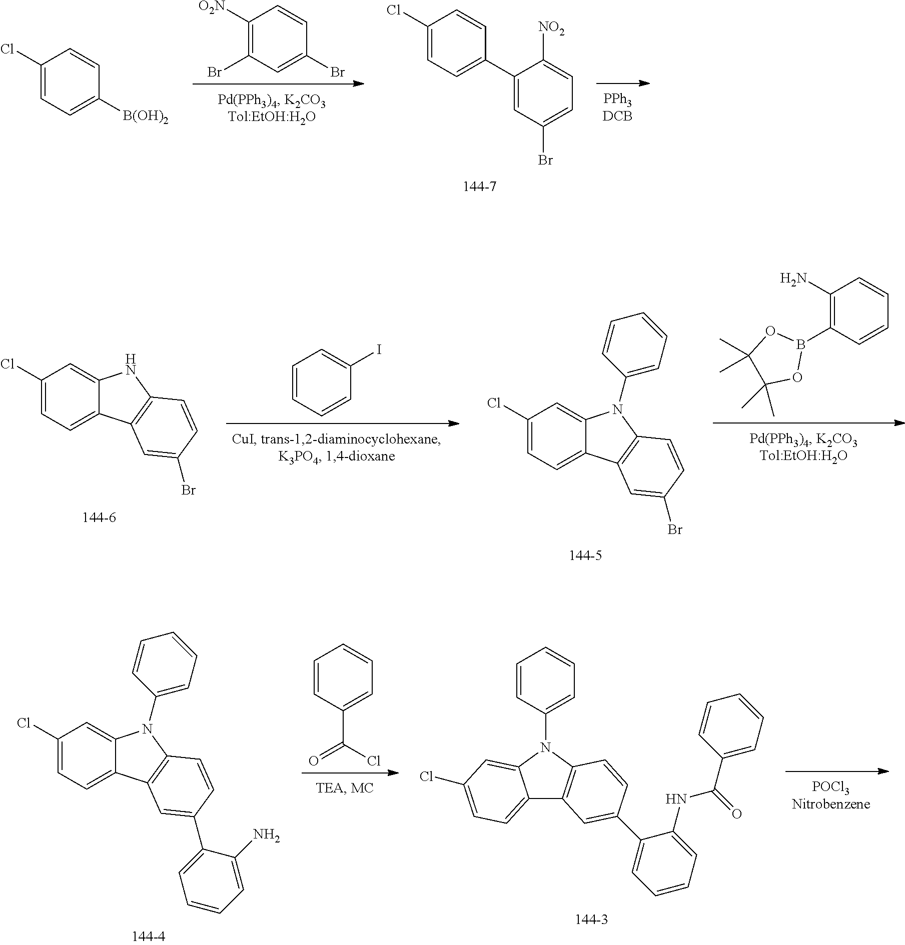

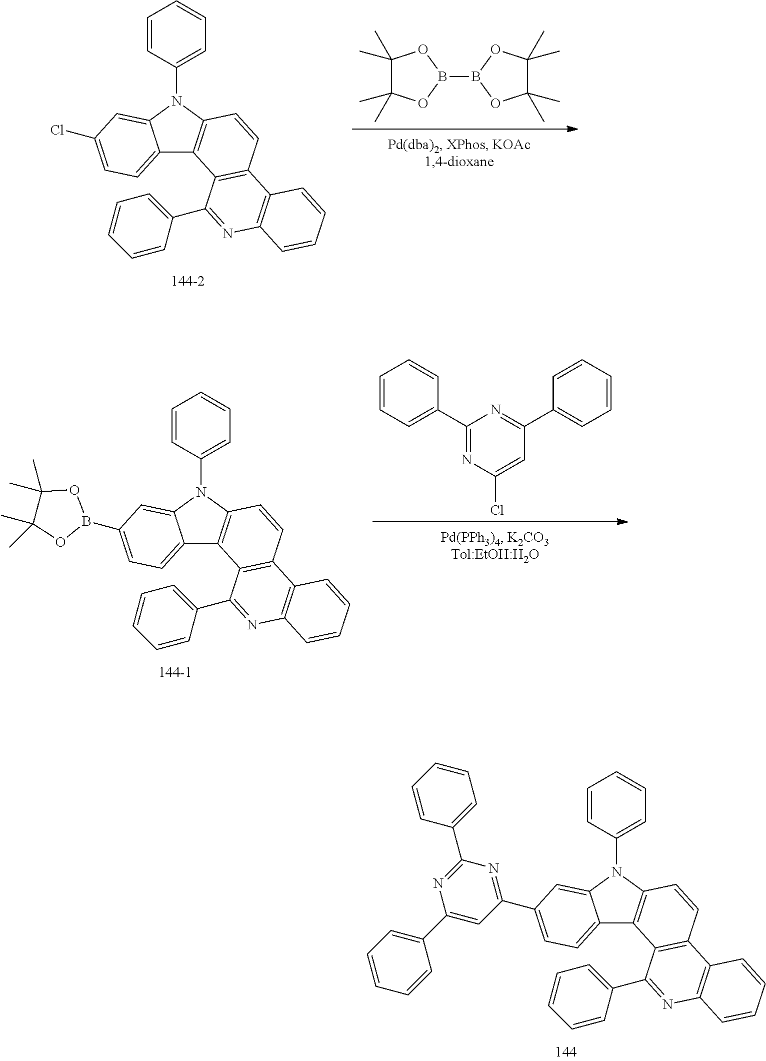

### Preparation of Compound 144-7

After dissolving (4-chlorophenyl)boronic acid (82 g, 525 mmol) and 2,4-dibromo-1-nitrobenzene (140 g, 500 mmol) in toluene, EtOH and H₂O (2000 mL:400 mL:400 mL), Pd(PPh₃)₄ (29 g, 25 mmol) and K₂CO₃ (126 g, 1500 mmol) were introduced thereto, and the result was refluxed for 4 hours. After the reaction was completed, the result was cooled to room temperature, and extracted with MC. The result was dried with anhydrous MgSO₄, and the solvent was removed using a rotary evaporator. Target Compound 144-7 (110 g, 70%, green solid) was obtained using column chromatography (MC:Hx=1:3).

### Preparation of Compound 114-6

After dissolving Compound 144-7 (110 g, 352 mmol) and triphenylphosphine (277 g, 1055 mmol) in 1,2-dichlorobenzene (1000 mL), the result was refluxed for 12 hours. After the reaction was completed, the result was cooled to room temperature, and extracted with MC. The result was dried with anhydrous MgSO₄, and the solvent was removed using a rotary evaporator. Target Compound 144-6 (78 g, 79%, green solid) was obtained using column chromatography (MC:Hx=1:1).

### Preparation of Compound 114-5

After dissolving Compound 144-6 (39 g, 184 mmol) and iodobenzene (42 g, 208 mmol) in 1,4-dioxane (800 mL), CuI (7.5 g, 40 mmol), trans-1,2-diaminocyclohexane (5 g, 40 mmol) and K₃PO₄ (90 g, 421 mmol) were introduced thereto, and the result was refluxed for 12 hours. After the reaction was completed, the result was cooled to room temperature, and extracted with MC. The result was dried with anhydrous MgSO₄, and the solvent was removed using a rotary evaporator. Target Compound 144-5 (42 g, 85%, green solid) was obtained using column chromatography (MC:Hx=1:3).

### Preparation of Compound 114-4

After dissolving Compound 144-5 (42 g, 167 mmol) and 2-(4,4,5,5-tetramethyl-1,3,2-dioxaborolan-2-yl)aniline (29 g, 130 mmol) in toluene, EtOH and H₂O (800 mL:160 mL:160 mL), Pd(PPh₃)₄ (7 g, 6 mmol) and K₂CO₃ (48 g, 350 mmol) were introduced thereto, and the result was refluxed for 12 hours. After the reaction was completed, the result was cooled to room temperature, and extracted with MC. The result was dried with anhydrous MgSO₄, and the solvent was removed using a rotary evaporator. Target Compound 144-4 (33 g, 80%, brown solid) was obtained using column chromatography (MC:Hx=1:3).

### Preparation of Compound 114-3

Compound 144-4 (33 g, 85 mmol) and triethylamine (37 mL, 260 mmol) were dissolved in MC (900 mL) and dissolved therein. Benzoyl chloride (13 g, 85 mmol) was dissolved in MC (100 mL), and then slowly added dropwise to the mixture at 0° C. After the reaction was completed, MC and distilled water were introduced to the reaction solution, and the result was extracted. After that, the result was dried with anhydrous MgSO₄, and, after removing the solvent using a rotary evaporator, recrystallized with EA/Hx to obtain Compound 144-3 (38 g, 88%, white solid).

### Preparation of Compound 114-2

After dissolving Compound 144-3 (38 g, 75 mmol) in nitrobenzene (500 mL), POCl₃ (8 mL, 75 mmol) was slowly added dropwise thereto. After that, the result was stirred for 12 hours at 150° C. After the reaction was completed, the reaction solution was neutralized with an aqueous NaHCO₃ solution. Solids produced during the neutralization were filtered. The solids were recrystallized with MC/MeOH to obtain target Compound 144-2 (33 g, 81%, white solid).

### Preparation of Compound 144-1

After dissolving Compound 144-2 (38 g, 80 mmol), bis(pinacolato)diboron (21 g, 100 mmol), Pd(dba)₂ (1 g, 4 mmol), XPhos (1.3 g, 8 mmol) and KOAc (42 g, 429 mmol) in 1,4-dioxane (600 mL), the result was refluxed for 12 hours. After the reaction was completed, MC and distilled water were introduced to the reaction solution, and the result was extracted. After that, the result was dried with anhydrous MgSO₄, and the solvent was removed using a rotary evaporator. The result was silica passed and then MeOH slurried to obtain Compound 144-1 (22 g, 89%, pale pink solid).

### Preparation of Compound 144

After dissolving Compound 144-1 (10 g, 18 mmol), 4-chloro-2,6-diphenylpyrimidine (4.9 g, 18 mmol) in toluene, EtOH and H₂O (100 mL:20 mL:20 mL), Pd(PPh₃)₄ (1.2 g, 1 mmol) and K₂CO₃ (7.5 g, 54 mol) were introduced thereto, and the result was refluxed for 12 hours. After the reaction was completed, produced solids were filtered to obtain Compound 144 (11.4 g, 80%, white solid).

Target Compounds were synthesized in the same manner as in Preparation Example 4 except that Intermediate G of the following Table 7 was used instead of 4-chloro-2,6-diphenylpyrimidine.

TABLE 7

| Compound | | | |
|---|---|---|---|
| No. | Intermediate G | Target Compound | Yield |
| 147 | 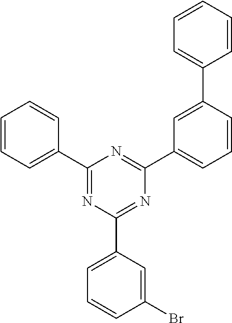 [SMILES: Brc1cccc(-c2nc(-c3ccccc3)nc(-c3cccc(-c4ccccc4)c3)n2)c1] | 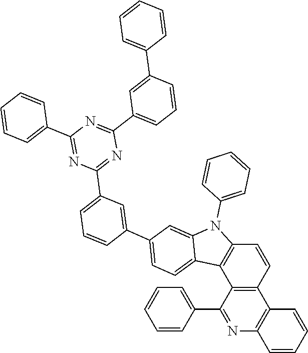 [SMILES: c1ccc(-c2cccc(-c3nc(-c4ccccc4)nc(-c4cccc(-c5ccc6c7c8c(-c9ccccc9)nc9ccccc9c8ccc7n(-c7ccccc7)c6c5)c4)n3)c2)cc1] | 75% |
| 151 | 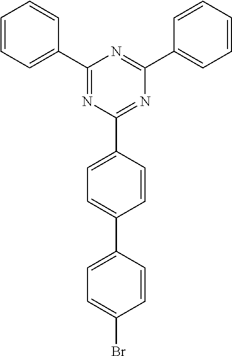 [SMILES: Brc1ccc(-c2ccc(-c3nc(-c4ccccc4)nc(-c4ccccc4)n3)cc2)cc1] | 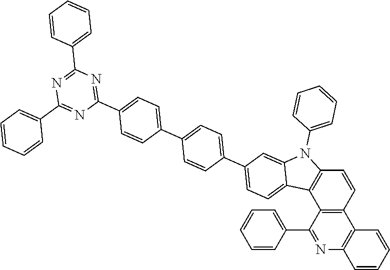 [SMILES: c1ccc(-c2nc(-c3ccccc3)nc(-c3ccc(-c4ccc(-c5ccc6c7c8c(-c9ccccc9)nc9ccccc9c8ccc7n(-c7ccccc7)c6c5)cc4)cc3)n2)cc1] | 91% |
| 154 | 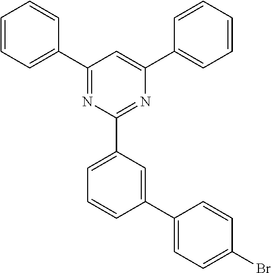 [SMILES: Brc1ccc(-c2cccc(-c3nc(-c4ccccc4)cc(-c4ccccc4)n3)c2)cc1] | 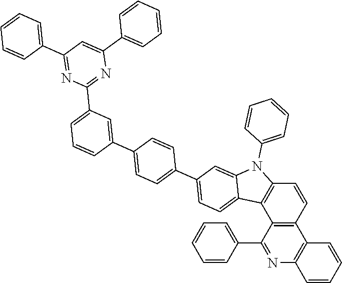 [SMILES: c1ccc(-c2cc(-c3ccccc3)nc(-c3cccc(-c4ccc(-c5ccc6c7c8c(-c9ccccc9)nc9ccccc9c8ccc7n(-c7ccccc7)c6c5)cc4)c3)n2)cc1] | 86% |
| 157 | 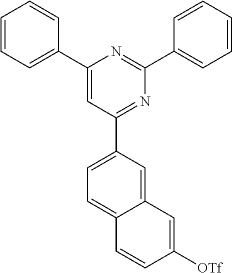 [SMILES: O=S(=O)(Oc1ccc2ccc(-c3cc(-c4ccccc4)nc(-c4ccccc4)n3)cc2c1)C(F)(F)F] | 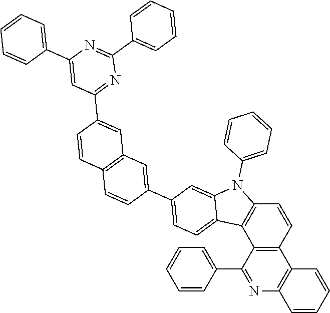 [SMILES: c1ccc(-c2cc(-c3ccc4ccc(-c5ccc6c7c8c(-c9ccccc9)nc9ccccc9c8ccc7n(-c7ccccc7)c6c5)cc4c3)nc(-c3ccccc3)n2)cc1] | 81% |

Target Compounds were synthesized in the same manner as in Preparation Example 4 except that (3-chlorophenyl)boronic acid was used instead of (4-chlorophenyl)boronic acid, and Intermediate H of the following Table 8 was used instead of 4-chloro-2,6-diphenylpyrimidine.

TABLE 8

| Compound | | | |
|---|---|---|---|
| No. | Intermediate H | Target Compound | Yield |
| 161 | 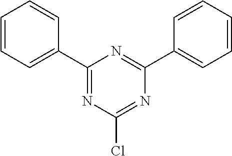 [SMILES: Clc1nc(-c2ccccc2)nc(-c2ccccc2)n1] | 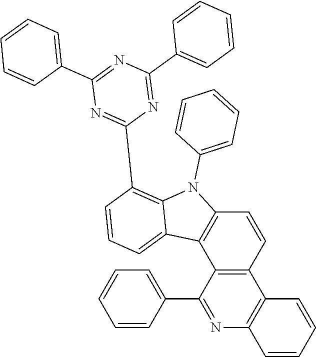 [SMILES: c1ccc(-c2nc(-c3ccccc3)nc(-c3cccc4c5c6c(-c7ccccc7)nc7ccccc7c6ccc5n(-c5ccccc5)c34)n2)cc1] | 75% |
| 166 | 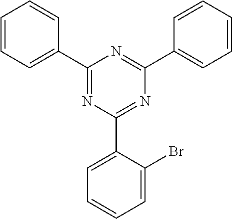 [SMILES: Brc1ccccc1-c1nc(-c2ccccc2)nc(-c2ccccc2)n1] | 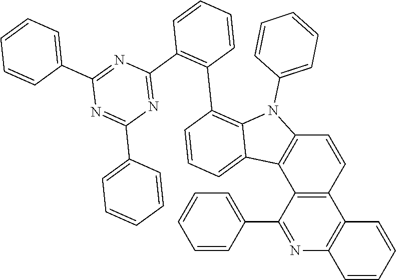 [SMILES: c1ccc(-c2nc(-c3ccccc3)nc(-c3ccccc3-c3cccc4c5c6c(-c7ccccc7)nc7ccccc7c6ccc5n(-c5ccccc5)c34)n2)cc1] | 91% |
| 171 | 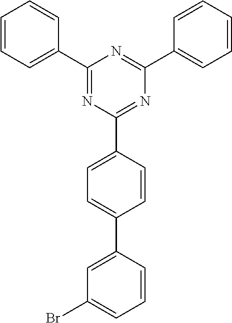 [SMILES: Brc1cccc(-c2ccc(-c3nc(-c4ccccc4)nc(-c4ccccc4)n3)cc2)c1] | 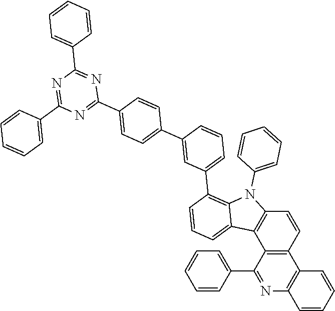 [SMILES: c1ccc(-c2nc(-c3ccccc3)nc(-c3ccc(-c4cccc(-c5cccc6c7c8c(-c9ccccc9)nc9ccccc9c8ccc7n(-c7ccccc7)c56)c4)cc3)n2)cc1] | 86% |
| 176 | 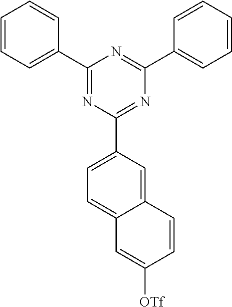 [SMILES: O=S(=O)(Oc1ccc2cc(-c3nc(-c4ccccc4)nc(-c4ccccc4)n3)ccc2c1)C(F)(F)F] | 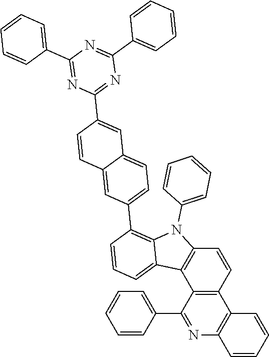 [SMILES: c1ccc(-c2nc(-c3ccccc3)nc(-c3ccc4cc(-c5cccc6c7c8c(-c9ccccc9)nc9ccccc9c8ccc7n(-c7ccccc7)c56)ccc4c3)n2)cc1] | 81% |
| 180 | 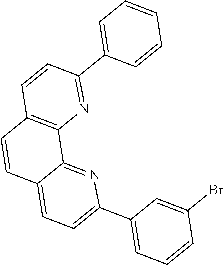 [SMILES: Brc1cccc(-c2ccc3ccc4ccc(-c5ccccc5)nc4c3n2)c1] | 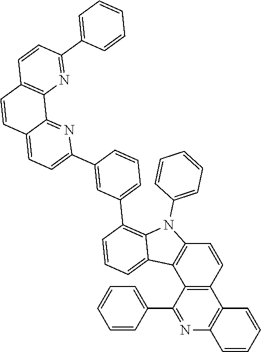 [SMILES: c1ccc(-c2ccc3ccc4ccc(-c5cccc(-c6cccc7c8c9c(-c%10ccccc%10)nc%10ccccc%10c9ccc8n(-c8ccccc8)c67)c5)nc4c3n2)cc1] | 75% |

### [Preparation Example 5] Preparation of Compound 183

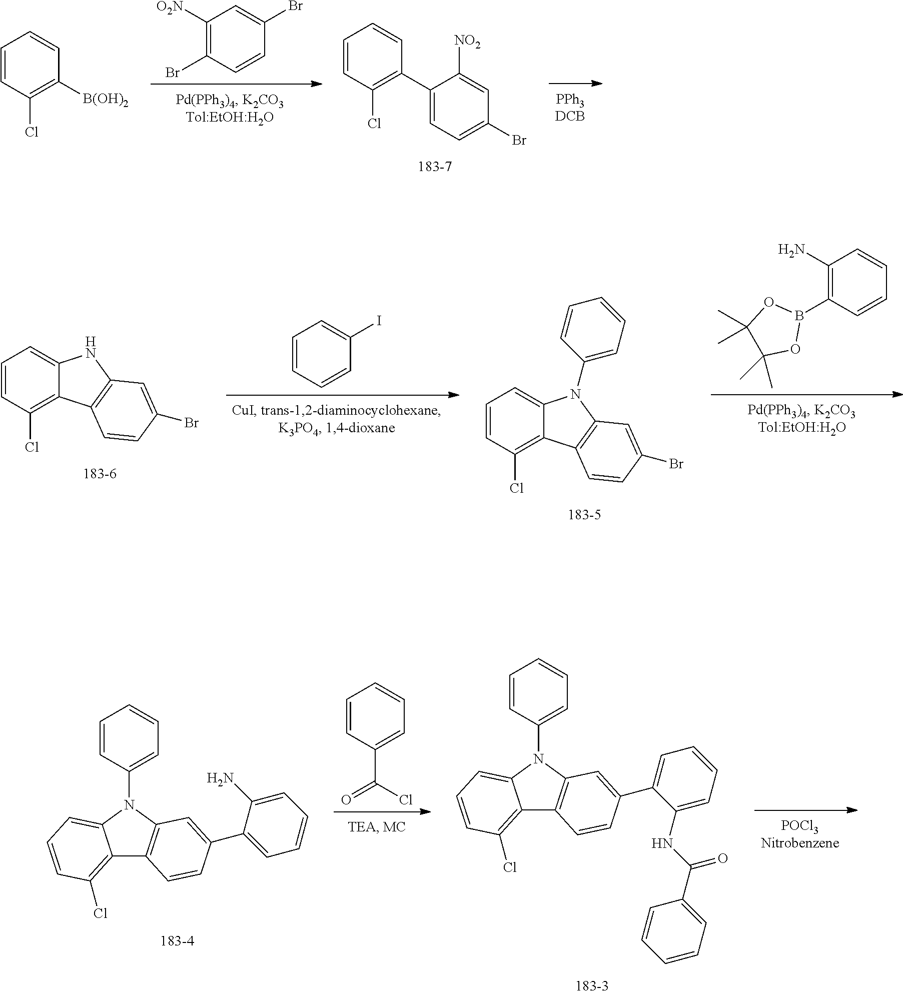

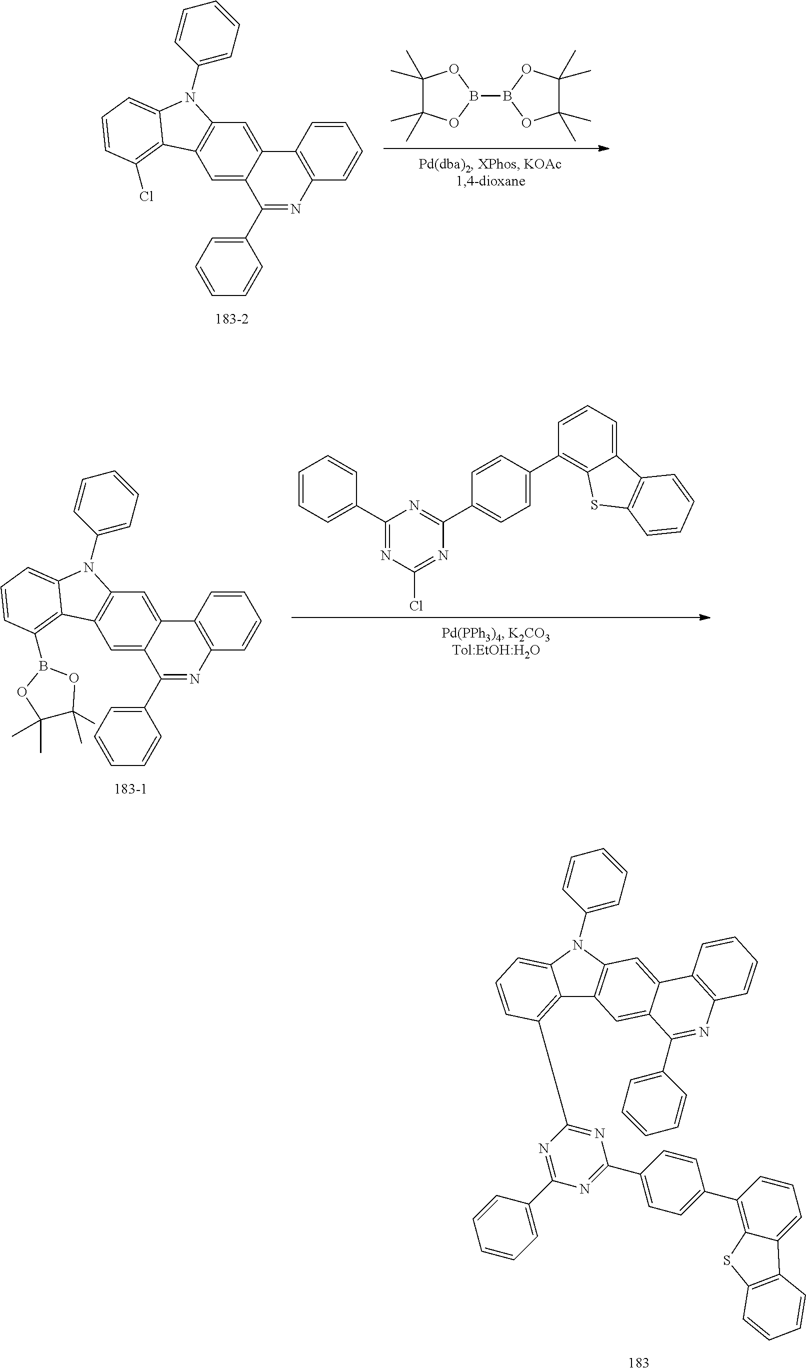

### Preparation of Compound 183-7

After dissolving (2-chlorophenyl)boronic acid (82 g, 525 mmol) and 2,4-dibromo-1-nitrobenzene (140 g, 500 mmol) in toluene, EtOH and H₂O (2000 mL:400 mL:400 mL), Pd(PPh₃)₄ (29 g, 25 mmol) and K₂CO₃ (126 g, 1500 mmol) were introduced thereto, and the result was refluxed for 4 hours. After the reaction was completed, the result was cooled to room temperature, and extracted with MC. The result was dried with anhydrous MgSO₄, and the solvent was removed using a rotary evaporator. Target Compound 183-7 (110 g, 70%, green solid) was obtained using column chromatography (MC:Hx=1:3).

### Preparation of Compound 183-6

After dissolving Compound 183-7 (110 g, 352 mmol) and triphenylphosphine (277 g, 1055 mmol) in 1,2-dichlorobenzene (1000 mL), the result was refluxed for 12 hours. After the reaction was completed, the result was cooled to room temperature, and extracted with MC. The result was dried with anhydrous MgSO₄, and the solvent was removed using a rotary evaporator. Target Compound 183-6 (78 g, 79%, green solid) was obtained using column chromatography (MC:Hx=1:1).

### Preparation of Compound 183-5

After dissolving Compound 183-6 (78 g, 278 mmol) and iodobenzene (85 g, 417 mmol) in 1,4-dioxane (800 mL), CuI (15 g, 80 mmol), trans-1,2-diaminocyclohexane (9 g, 80 mmol) and K₃PO₄ (177 g, 834 mmol) were introduced thereto, and the result was refluxed for 12 hours. After the reaction was completed, the result was cooled to room temperature, and extracted with MC. The result was dried with anhydrous MgSO₄, and the solvent was removed using a rotary evaporator. Target Compound 183-5 (84 g, 85%, green solid) was obtained using column chromatography (MC:Hx=1:3).

### Preparation of Compound 183-4

After dissolving Compound 183-5 (84 g, 235 mmol) and 2-(4,4,5,5-tetramethyl-1,3,2-dioxaborolan-2-yl)aniline (57 g, 260 mmol) in toluene, EtOH and H₂O (800 mL:160 mL:160 mL), Pd(PPh₃)₄ (14 g, 12 mmol) and K₂CO₃ (97 g, 705 mmol) were introduced thereto, and the result was refluxed for 12 hours. After the reaction was completed, the result was cooled to room temperature, and extracted with MC. The result was dried with anhydrous MgSO₄, and the solvent was removed using a rotary evaporator. Target Compound 183-4 (65 g, 75%, brown solid) was obtained using column chromatography (MC:Hx=1:3).

### Preparation of Compound 183-3

Compound 183-4 (65 g, 176 mmol) and triethylamine (74 mL, 528 mmol) were dissolved in MC (900 mL) and dissolved therein. Benzoyl chloride (25 g, 176 mmol) was dissolved in MC (100 mL), and then slowly added dropwise to the mixture at 0° C. After the reaction was completed, MC and distilled water were introduced to the reaction solution, and the result was extracted. After that, the result was dried with anhydrous MgSO₄, and, after removing the solvent using a rotary evaporator, recrystallized with EA/Hx to obtain Compound 183-3 (76 g, 92%, white solid).

### Preparation of Compound 183-2

After dissolving Compound 183-3 (76 g, 162 mmol) in nitrobenzene (500 mL), POCl₃ (15 mL, 162 mmol) was slowly added dropwise thereto. After that, the result was stirred for 12 hours at 150° C. After the reaction was completed, the reaction solution was neutralized with an aqueous NaHCO₃ solution. Solids produced during the neutralization were filtered. The solids were recrystallized with MC/MeOH to obtain target Compound 183-2 (65 g, 88%, white solid).

### Preparation of Compound 183-1

After dissolving Compound 183-2 (65 g, 143 mmol), bis(pinacolato)diboron (54 g, 215 mmol), Pd(dba)₂ (2 g, 7 mmol), XPhos (2.5 g, 14 mmol) and KOAc (42 g, 429 mmol) in 1,4-dioxane (600 mL), the result was refluxed for 12 hours. After the reaction was completed, MC and distilled water were introduced to the reaction solution, and the result was extracted. After that, the result was dried with anhydrous MgSO₄, and the solvent was removed using a rotary evaporator. The result was silica passed and then MeOH slurried to obtain Compound 183-1 (66 g, 85%, pale pink solid).

### Preparation of Compound 183

After dissolving Compound 183-1 (10 g, 18 mmol) and 4-chloro-2,6-diphenylpyrimidine (4.9 g, 18 mmol) in toluene, EtOH and H₂O (100 mL:20 mL:20 mL), Pd(PPh₃)₄ (1.2 g, 1 mmol) and K₂CO₃ (7.5 g, 54 mol) were introduced thereto, and the result was refluxed for 12 hours. After the reaction was completed, produced solids were filtered to obtain Compound 183 (11.4 g, 88%, white solid).

Target Compounds were synthesized in the same manner as in Preparation Example 5 except that Intermediate I of the following Table 9 was used instead of 2-chloro-4-(4-(dibenzo[b,d]thiophen-4-yl)phenyl)-6-phenyl-1,3,5-triazine.

TABLE 9

| Compound | | | |
|---|---|---|---|
| No. | Intermediate I | Target Compound | Yield |
| 187 | 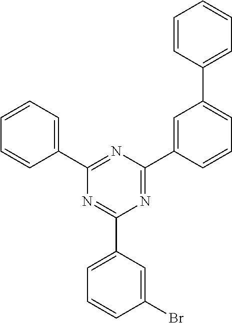 [SMILES: Brc1cccc(-c2nc(-c3ccccc3)nc(-c3cccc(-c4ccccc4)c3)n2)c1] | 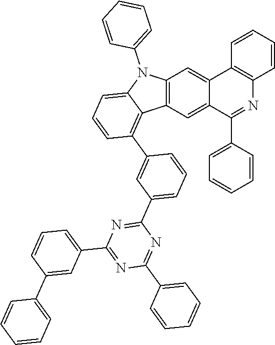 [SMILES: c1ccc(-c2cccc(-c3nc(-c4ccccc4)nc(-c4cccc(-c5cccc6c5c5cc7c(-c8ccccc8)nc8ccccc8c7cc5n6-c5ccccc5)c4)n3)c2)cc1] | 78% |
| 192 | 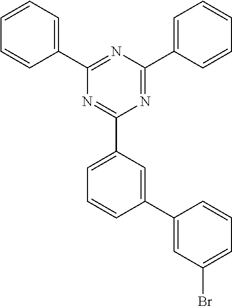 [SMILES: Brc1cccc(-c2cccc(-c3nc(-c4ccccc4)nc(-c4ccccc4)n3)c2)c1] | 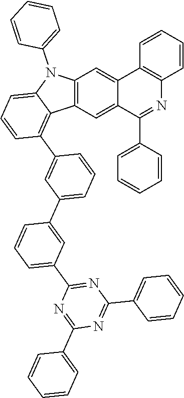 [SMILES: c1ccc(-c2nc(-c3ccccc3)nc(-c3cccc(-c4cccc(-c5cccc6c5c5cc7c(-c8ccccc8)nc8ccccc8c7cc5n6-c5ccccc5)c4)c3)n2)cc1] | 85% |
| 196 | 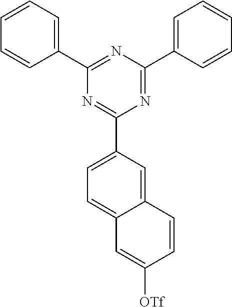 [SMILES: O=S(=O)(Oc1ccc2cc(-c3nc(-c4ccccc4)nc(-c4ccccc4)n3)ccc2c1)C(F)(F)F] | 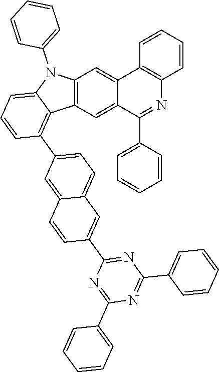 [SMILES: c1ccc(-c2nc(-c3ccccc3)nc(-c3ccc4cc(-c5cccc6c5c5cc7c(-c8ccccc8)nc8ccccc8c7cc5n6-c5ccccc5)ccc4c3)n2)cc1] | 79% |
| 199 | 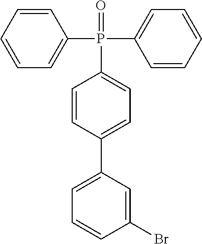 [SMILES: O=P(c1ccccc1)(c1ccccc1)c1ccc(-c2cccc(Br)c2)cc1] | 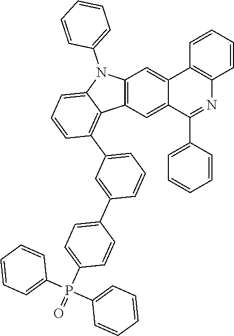 [SMILES: O=P(c1ccccc1)(c1ccccc1)c1ccc(-c2cccc(-c3cccc4c3c3cc5c(-c6ccccc6)nc6ccccc6c5cc3n4-c3ccccc3)c2)cc1] | 83% |

Target Compounds were synthesized in the same manner as in Preparation Example 5 except that (3-chlorophenyl)boronic acid was used instead of (2-chlorophenyl)boronic acid, and Intermediate J of the following Table 10 was used instead of 2-chloro-4-(4-(dibenzo[b,d]thiophen-4-yl)phenyl)-6-phenyl-1,3,5-triazine.

TABLE 10

| Com- | | | |
|---|---|---|---|
| pound | | | |
| No. | Intermediate J | Target Compound | Yield |
| 204 | 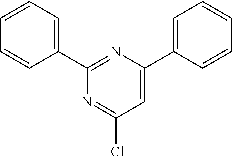 [SMILES: Clc1cc(-c2ccccc2)nc(-c2ccccc2)n1] | 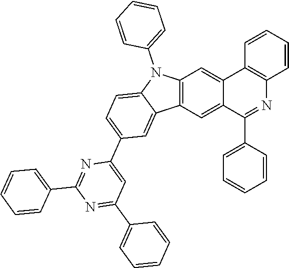 [SMILES: c1ccc(-c2cc(-c3ccc4c(c3)c3cc5c(-c6ccccc6)nc6ccccc6c5cc3n4-c3ccccc3)nc(-c3ccccc3)n2)cc1] | 81% |
| 207 | 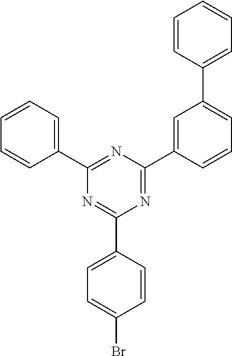 [SMILES: Brc1ccc(-c2nc(-c3ccccc3)nc(-c3cccc(-c4ccccc4)c3)n2)cc1] | 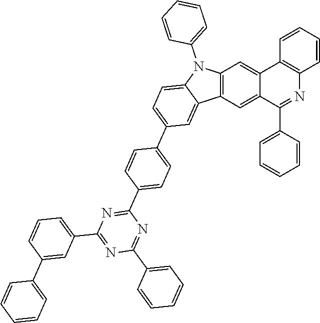 [SMILES: c1ccc(-c2cccc(-c3nc(-c4ccccc4)nc(-c4ccc(-c5ccc6c(c5)c5cc7c(-c8ccccc8)nc8ccccc8c7cc5n6-c5ccccc5)cc4)n3)c2)cc1] | 86% |
| 210 | 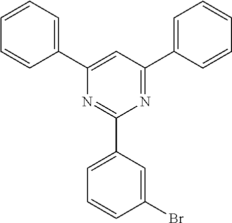 [SMILES: Brc1cccc(-c2nc(-c3ccccc3)cc(-c3ccccc3)n2)c1] | 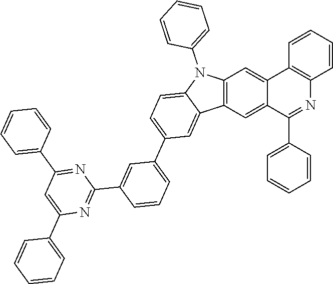 [SMILES: c1ccc(-c2cc(-c3ccccc3)nc(-c3cccc(-c4ccc5c(c4)c4cc6c(-c7ccccc7)nc7ccccc7c6cc4n5-c4ccccc4)c3)n2)cc1] | 79% |
| 215 | 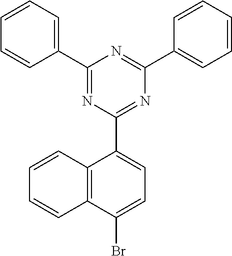 [SMILES: Brc1ccc(-c2nc(-c3ccccc3)nc(-c3ccccc3)n2)c2ccccc12] | 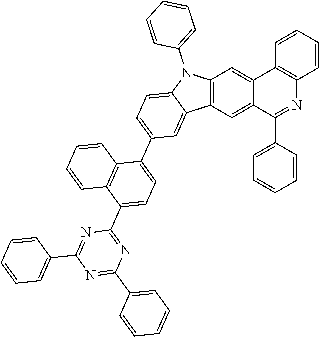 [SMILES: c1ccc(-c2nc(-c3ccccc3)nc(-c3ccc(-c4ccc5c(c4)c4cc6c(-c7ccccc7)nc7ccccc7c6cc4n5-c4ccccc4)c4ccccc34)n2)cc1] | 83% |
| 218 | 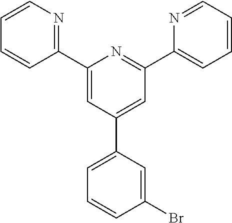 [SMILES: Brc1cccc(-c2cc(-c3ccccn3)nc(-c3ccccn3)c2)c1] | 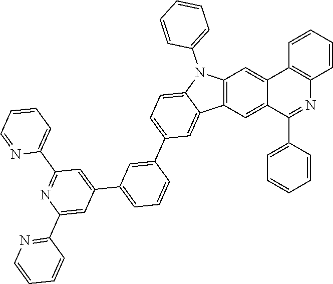 [SMILES: c1ccc(-c2nc3ccccc3c3cc4c(cc23)c2cc(-c3cccc(-c5cc(-c6ccccn6)nc(-c6ccccn6)c5)c3)ccc2n4-c2ccccc2)cc1] | 86% |
| 244 | 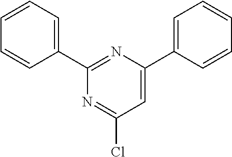 [SMILES: Clc1cc(-c2ccccc2)nc(-c2ccccc2)n1] | 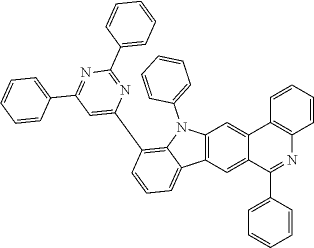 [SMILES: c1ccc(-c2cc(-c3cccc4c5cc6c(-c7ccccc7)nc7ccccc7c6cc5n(-c5ccccc5)c34)nc(-c3ccccc3)n2)cc1] | 71% |
| 248 | 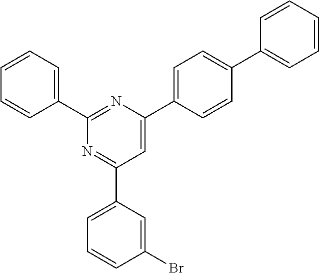 [SMILES: Brc1cccc(-c2cc(-c3ccc(-c4ccccc4)cc3)nc(-c3ccccc3)n2)c1] | 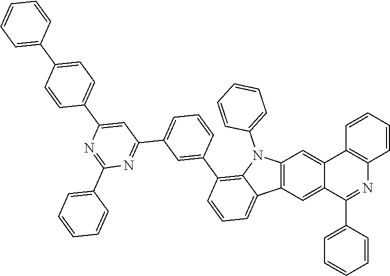 [SMILES: c1ccc(-c2ccc(-c3cc(-c4cccc(-c5cccc6c7cc8c(-c9ccccc9)nc9ccccc9c8cc7n(-c7ccccc7)c56)c4)nc(-c4ccccc4)n3)cc2)cc1] | 80% |
| 252 | 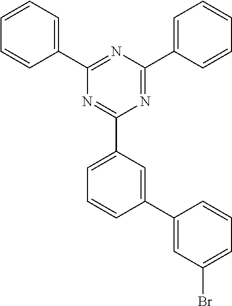 [SMILES: Brc1cccc(-c2cccc(-c3nc(-c4ccccc4)nc(-c4ccccc4)n3)c2)c1] | 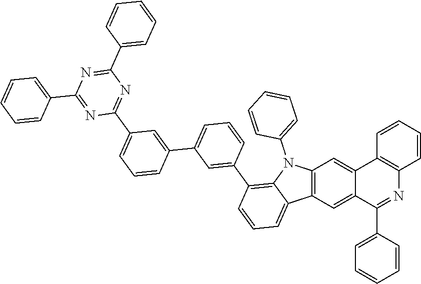 [SMILES: c1ccc(-c2nc(-c3ccccc3)nc(-c3cccc(-c4cccc(-c5cccc6c7cc8c(-c9ccccc9)nc9ccccc9c8cc7n(-c7ccccc7)c56)c4)c3)n2)cc1] | 83% |
| 256 | 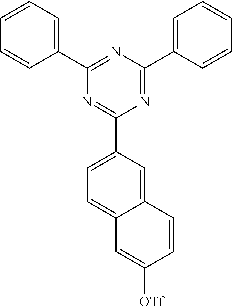 [SMILES: O=S(=O)(Oc1ccc2cc(-c3nc(-c4ccccc4)nc(-c4ccccc4)n3)ccc2c1)C(F)(F)F] | 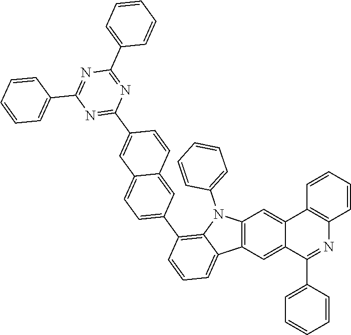 [SMILES: c1ccc(-c2nc(-c3ccccc3)nc(-c3ccc4cc(-c5cccc6c7cc8c(-c9ccccc9)nc9ccccc9c8cc7n(-c7ccccc7)c56)ccc4c3)n2)cc1] | 77% |
| 260 | 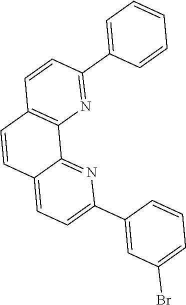 [SMILES: Brc1cccc(-c2ccc3ccc4ccc(-c5ccccc5)nc4c3n2)c1] | 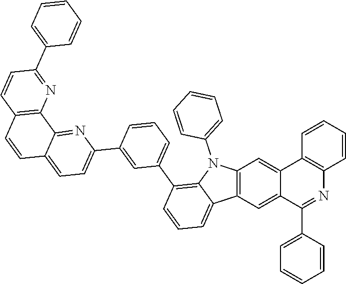 [SMILES: c1ccc(-c2ccc3ccc4ccc(-c5cccc(-c6cccc7c8cc9c(-c%10ccccc%10)nc%10ccccc%10c9cc8n(-c8ccccc8)c67)c5)nc4c3n2)cc1] | 87% |

Target Compounds were synthesized in the same manner as in Preparation Example 5 except that (4-chlorophenyl)boronic acid was used instead of (2-chlorophenyl)boronic acid, and Intermediate K of the following Table 11 was used instead of 2-chloro-4-(4-(dibenzo[b,d]thiophen-4-yl)phenyl)-6-phenyl-1,3,5-triazine.

TABLE 11

| Compound | | | |
|---|---|---|---|
| No. | Intermediate K | Target Compound | Yield |
| 222 | 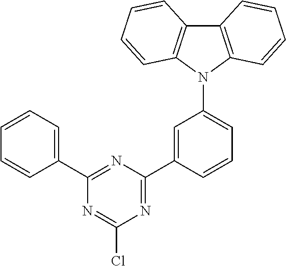 [SMILES: Clc1nc(-c2ccccc2)nc(-c2cccc(-n3c4ccccc4c4ccccc43)c2)n1] | 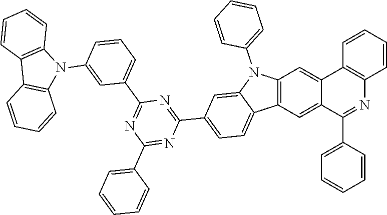 [SMILES: c1ccc(-c2nc(-c3cccc(-n4c5ccccc5c5ccccc54)c3)nc(-c3ccc4c5cc6c(-c7ccccc7)nc7ccccc7c6cc5n(-c5ccccc5)c4c3)n2)cc1] | 78% |
| 228 | 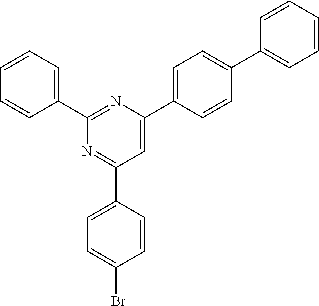 [SMILES: Brc1ccc(-c2cc(-c3ccc(-c4ccccc4)cc3)nc(-c3ccccc3)n2)cc1] | 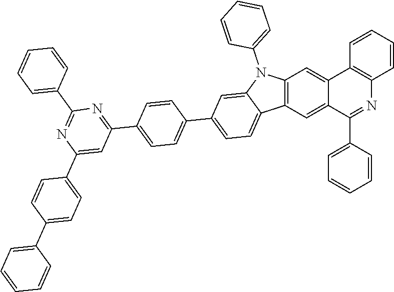 [SMILES: c1ccc(-c2ccc(-c3cc(-c4ccc(-c5ccc6c7cc8c(-c9ccccc9)nc9ccccc9c8cc7n(-c7ccccc7)c6c5)cc4)nc(-c4ccccc4)n3)cc2)cc1] | 85% |
| 230 | 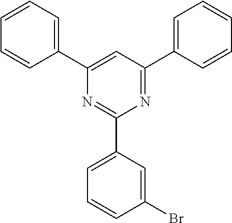 [SMILES: Brc1cccc(-c2nc(-c3ccccc3)cc(-c3ccccc3)n2)c1] | 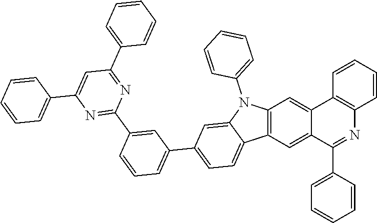 [SMILES: c1ccc(-c2cc(-c3ccccc3)nc(-c3cccc(-c4ccc5c6cc7c(-c8ccccc8)nc8ccccc8c7cc6n(-c6ccccc6)c5c4)c3)n2)cc1] | 79% |
| 235 | 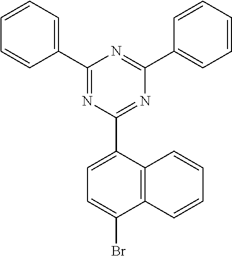 [SMILES: Brc1ccc(-c2nc(-c3ccccc3)nc(-c3ccccc3)n2)c2ccccc12] | 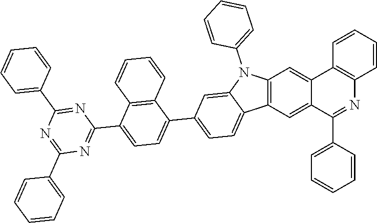 [SMILES: c1ccc(-c2nc(-c3ccccc3)nc(-c3ccc(-c4ccc5c6cc7c(-c8ccccc8)nc8ccccc8c7cc6n(-c6ccccc6)c5c4)c4ccccc34)n2)cc1] | 83% |
| 239 | 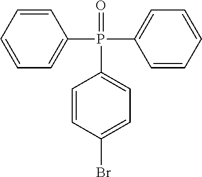 [SMILES: O=P(c1ccccc1)(c1ccccc1)c1ccc(Br)cc1] | 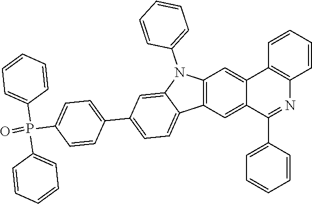 [SMILES: O=P(c1ccccc1)(c1ccccc1)c1ccc(-c2ccc3c4cc5c(-c6ccccc6)nc6ccccc6c5cc4n(-c4ccccc4)c3c2)cc1] | 82% |

### [Preparation Example 6] Preparation of Compound 262

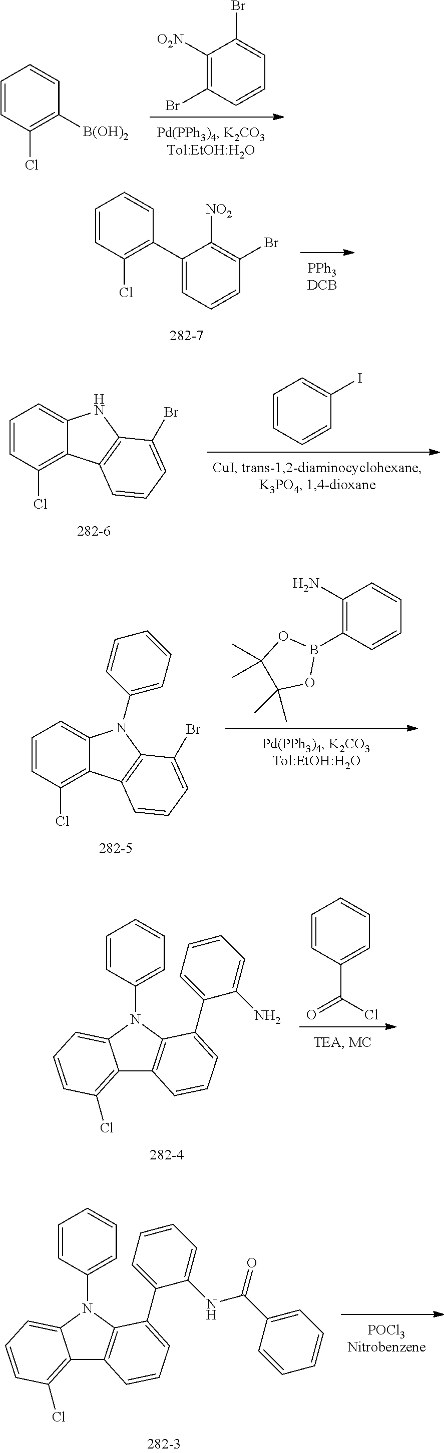

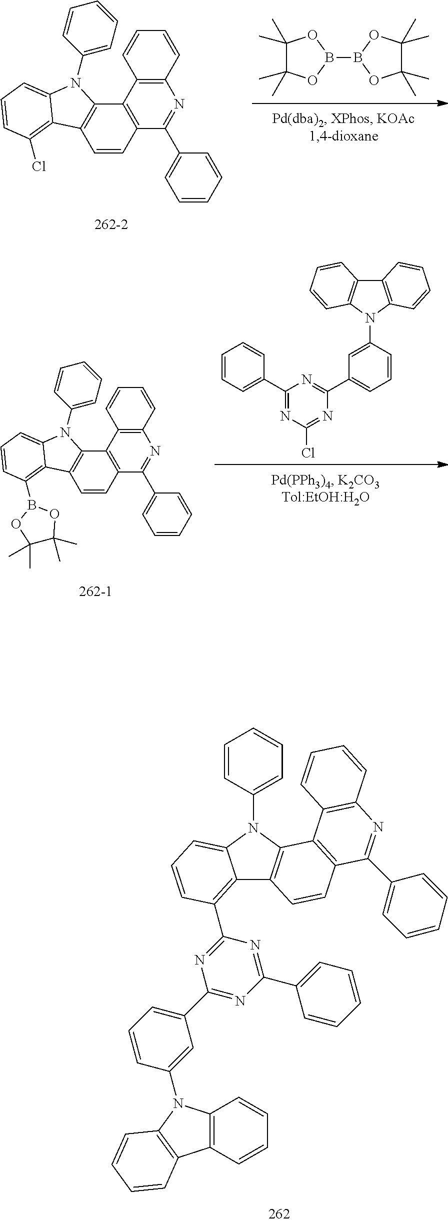

### Preparation of Compound 262-7

After dissolving (2-chlorophenyl)boronic acid (82 g, 525 mmol) and 2,4-dibromo-1-nitrobenzene (140 g, 500 mmol) in toluene, EtOH and H₂O (2000 mL:400 mL:400 mL), Pd(PPh₃)₄ (29 g, 25 mmol) and K₂CO₃ (126 g, 1500 mmol) were introduced thereto, and the result was refluxed for 4 hours. After the reaction was completed, the result was cooled to room temperature, and extracted with MC. The result was dried with anhydrous MgSO₄, and the solvent was removed using a rotary evaporator. Target Compound 262-7 (110 g, 70%, green solid) was obtained using column chromatography (MC:Hx=1:3).

### Preparation of Compound 262-6

After dissolving Compound 262-7 (110 g, 352 mmol) and triphenylphosphine (277 g, 1055 mmol) in 1,2-dichlorobenzene (1000 mL), the result was refluxed for 12 hours. After the reaction was completed, the result was cooled to room temperature, and extracted with MC. The result was dried with anhydrous MgSO₄, and the solvent was removed using a rotary evaporator. Target Compound 262-6 (78 g, 79%, green solid) was obtained using column chromatography (MC:Hx=1:1).

### Preparation of Compound 262-5

After dissolving Compound 262-6 (78 g, 278 mmol) and iodobenzene (85 g, 417 mmol) in 1,4-dioxane (800 mL), CuI (15 g, 80 mmol), trans-1,2-diaminocyclohexane (9 g, 80 mmol) and K₃PO₄ (177 g, 834 mmol) were introduced thereto, and the result was refluxed for 12 hours. After the reaction was completed, the result was cooled to room temperature, and extracted with MC. The result was dried with anhydrous MgSO₄, and the solvent was removed using a rotary evaporator. Target Compound 262-5 (84 g, 85%, green solid) was obtained using column chromatography (MC:Hx=1:3).

### Preparation of Compound 262-4

After dissolving Compound 262-5 (84 g, 235 mmol) and 2-(4,4,5,5-tetramethyl-1,3,2-dioxaborolan-2-yl)aniline (57 g, 260 mmol) in toluene, EtOH and H₂O (800 mL:160 mL:160 mL), Pd(PPh₃)₄ (14 g, 12 mmol) and K₂CO₃ (97 g, 705 mmol) were introduced thereto, and the result was refluxed for 12 hours. After the reaction was completed, the result was cooled to room temperature, and extracted with MC. The result was dried with anhydrous MgSO₄, and the solvent was removed using a rotary evaporator. Target Compound 262-4 (65 g, 75%, brown solid) was obtained using column chromatography (MC:Hx=1:3).

### Preparation of Compound 262-3

Compound 262-4 (65 g, 176 mmol) and triethylamine (74 mL, 528 mmol) were dissolved in MC (900 mL) and dissolved therein. Benzoyl chloride (25 g, 176 mmol) was dissolved in MC (100 mL), and then slowly added dropwise to the mixture at 0° C. After the reaction was completed, MC and distilled water were introduced to the reaction solution, and the result was extracted. After that, the result was dried with anhydrous MgSO₄, and, after removing the solvent using a rotary evaporator, recrystallized with EA/Hx to obtain Compound 262-3 (76 g, 92%, white solid).

### Preparation of Compound 262-2

After dissolving Compound 262-3 (76 g, 162 mmol) in nitrobenzene (500 mL), POCl₃ (15 mL, 162 mmol) was slowly added dropwise thereto. After that, the result was stirred for 12 hours at 150° C. After the reaction was completed, the reaction solution was neutralized with an aqueous NaHCO₃ solution. Solids produced during the neutralization were filtered. The solids were recrystallized with MC/MeOH to obtain target Compound 262-2 (65 g, 88%, white solid).

### Preparation of Compound 262-1

After dissolving Compound 262-2 (65 g, 143 mmol), bis(pinacolato)diboron (54 g, 215 mmol), Pd(dba)₂ (2 g, 7 mmol), XPhos (2.5 g, 14 mmol) and KOAc (42 g, 429 mmol) in 1,4-dioxane (600 mL), the result was refluxed for 12 hours. After the reaction was completed, MC and distilled water were introduced to the reaction solution, and the result was extracted. After that, the result was dried with anhydrous MgSO₄, and the solvent was removed using a rotary evaporator. The result was silica passed and then MeOH slurried to obtain Compound 262-1 (66 g, 85%, pale pink solid).

### Preparation of Compound 262

After dissolving Compound 262-1 (10 g, 18 mmol) and 4-chloro-2,6-diphenylpyrimidine (4.9 g, 18 mmol) in toluene, EtOH and H₂O (100 mL:20 mL:20 mL), Pd(PPh₃)₄ (1.2 g, 1 mmol) and K₂CO₃ (7.5 g, 54 mol) were introduced thereto, and the result was refluxed for 12 hours. After the reaction was completed, produced solids were filtered to obtain Compound 262 (11.4 g, 81%, white solid).

Target Compounds were synthesized in the same manner as in Preparation Example 6 except that Intermediate L of the following Table 12 was used instead of 9-(3-(4-chloro-6-phenyl-1,3,5-triazin-2-yl)phenyl)-9H-carbazole.

TABLE 12

| Compound | | | |
|---|---|---|---|
| No. | Intermediate L | Target Compound | Yield |
| 265 | 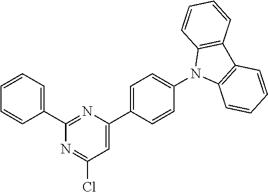 [SMILES: Clc1cc(-c2ccc(-n3c4ccccc4c4ccccc43)cc2)nc(-c2ccccc2)n1] | 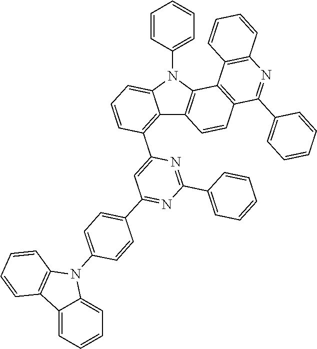 [SMILES: c1ccc(-c2nc(-c3ccc(-n4c5ccccc5c5ccccc54)cc3)cc(-c3cccc4c3c3ccc5c(-c6ccccc6)nc6ccccc6c5c3n4-c3ccccc3)n2)cc1] | 78% |
| 271 | 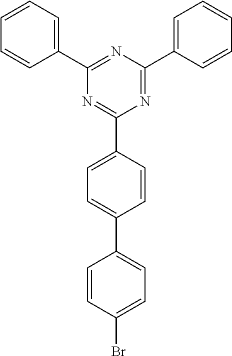 [SMILES: Brc1ccc(-c2ccc(-c3nc(-c4ccccc4)nc(-c4ccccc4)n3)cc2)cc1] | 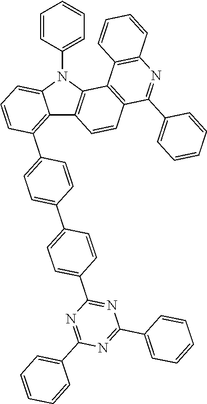 [SMILES: c1ccc(-c2nc(-c3ccccc3)nc(-c3ccc(-c4ccc(-c5cccc6c5c5ccc7c(-c8ccccc8)nc8ccccc8c7c5n6-c5ccccc5)cc4)cc3)n2)cc1] | 81% |
| 274 | 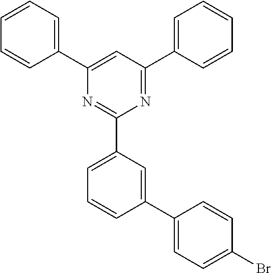 [SMILES: Brc1ccc(-c2cccc(-c3nc(-c4ccccc4)cc(-c4ccccc4)n3)c2)cc1] | 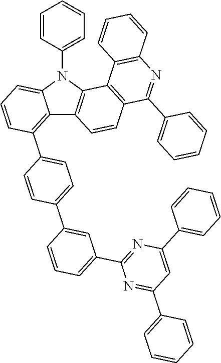 [SMILES: c1ccc(-c2cc(-c3ccccc3)nc(-c3cccc(-c4ccc(-c5cccc6c5c5ccc7c(-c8ccccc8)nc8ccccc8c7c5n6-c5ccccc5)cc4)c3)n2)cc1] | 89% |
| 279 | 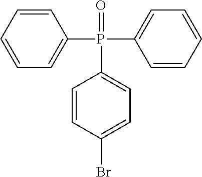 [SMILES: O=P(c1ccccc1)(c1ccccc1)c1ccc(Br)cc1] | 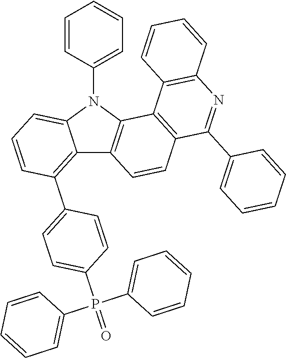 [SMILES: O=P(c1ccccc1)(c1ccccc1)c1ccc(-c2cccc3c2c2ccc4c(-c5ccccc5)nc5ccccc5c4c2n3-c2ccccc2)cc1] | 73% |

Target Compounds were synthesized in the same manner as in Preparation Example 6 except that (3-chlorophenyl)boronic acid was used instead of (2-chlorophenyl)boronic acid, and Intermediate M of the following Table 13 was used instead of 9-(3-(4-chloro-6-phenyl-1,3,5-triazin-2-yl)phenyl)-9H-carbazole.

TABLE 13

| Com- | | | |
|---|---|---|---|
| pound | | | |
| No. | Intermediate M | Target Compound | Yield |
| 283 | 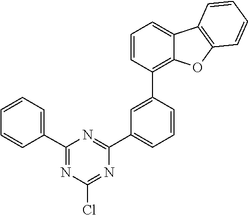 [SMILES: Clc1nc(-c2ccccc2)nc(-c2cccc(-c3cccc4c3oc3ccccc34)c2)n1] | 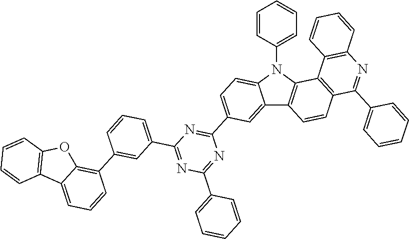 [SMILES: c1ccc(-c2nc(-c3cccc(-c4cccc5c4oc4ccccc45)c3)nc(-c3ccc4c(c3)c3ccc5c(-c6ccccc6)nc6ccccc6c5c3n4-c3ccccc3)n2)cc1] | 79% |
| 286 | 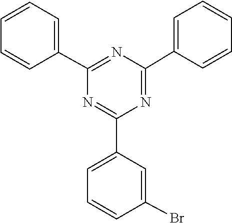 [SMILES: Brc1cccc(-c2nc(-c3ccccc3)nc(-c3ccccc3)n2)c1] | 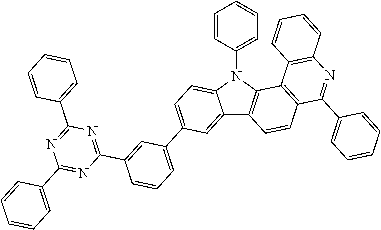 [SMILES: c1ccc(-c2nc(-c3ccccc3)nc(-c3cccc(-c4ccc5c(c4)c4ccc6c(-c7ccccc7)nc7ccccc7c6c4n5-c4ccccc4)c3)n2)cc1] | 86% |
| 289 | 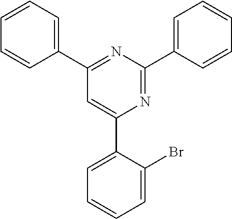 [SMILES: Brc1ccccc1-c1cc(-c2ccccc2)nc(-c2ccccc2)n1] | 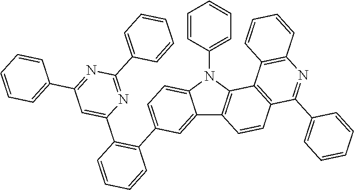 [SMILES: c1ccc(-c2cc(-c3ccccc3-c3ccc4c(c3)c3ccc5c(-c6ccccc6)nc6ccccc6c5c3n4-c3ccccc3)nc(-c3ccccc3)n2)cc1] | 79% |
| 294 | 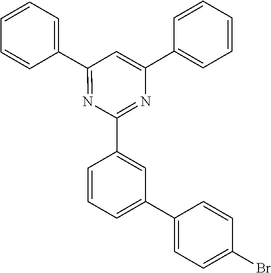 [SMILES: Brc1ccc(-c2cccc(-c3nc(-c4ccccc4)cc(-c4ccccc4)n3)c2)cc1] | 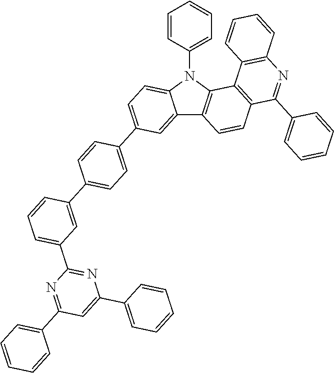 [SMILES: c1ccc(-c2cc(-c3ccccc3)nc(-c3cccc(-c4ccc(-c5ccc6c(c5)c5ccc7c(-c8ccccc8)nc8ccccc8c7c5n6-c5ccccc5)cc4)c3)n2)cc1] | 83% |
| 297 | 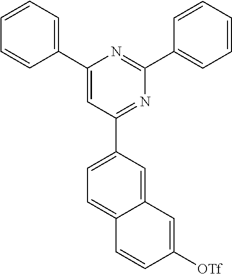 [SMILES: O=S(=O)(Oc1ccc2ccc(-c3cc(-c4ccccc4)nc(-c4ccccc4)n3)cc2c1)C(F)(F)F] | 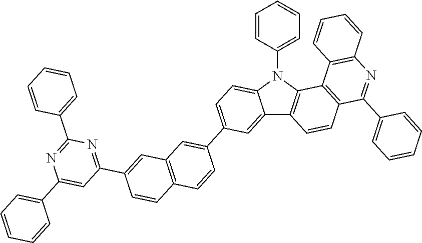 [SMILES: c1ccc(-c2cc(-c3ccc4ccc(-c5ccc6c(c5)c5ccc7c(-c8ccccc8)nc8ccccc8c7c5n6-c5ccccc5)cc4c3)nc(-c3ccccc3)n2)cc1] | 86% |
| 323 | 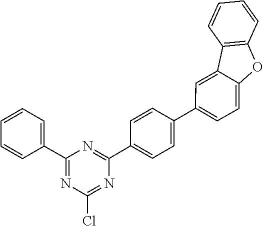 [SMILES: Clc1nc(-c2ccccc2)nc(-c2ccc(-c3ccc4oc5ccccc5c4c3)cc2)n1] | 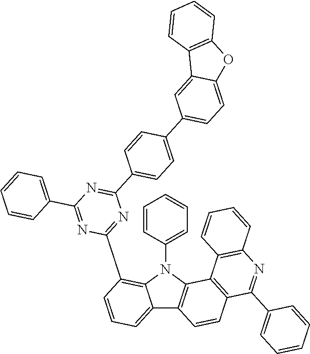 [SMILES: c1ccc(-c2nc(-c3ccc(-c4ccc5oc6ccccc6c5c4)cc3)nc(-c3cccc4c5ccc6c(-c7ccccc7)nc7ccccc7c6c5n(-c5ccccc5)c34)n2)cc1] | 71% |
| 326 | 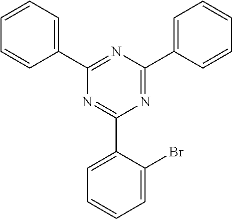 [SMILES: Brc1ccccc1-c1nc(-c2ccccc2)nc(-c2ccccc2)n1] | 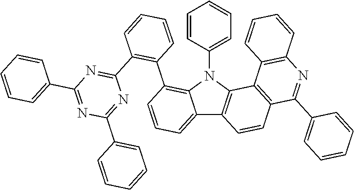 [SMILES: c1ccc(-c2nc(-c3ccccc3)nc(-c3ccccc3-c3cccc4c5ccc6c(-c7ccccc7)nc7ccccc7c6c5n(-c5ccccc5)c34)n2)cc1] | 80% |
| 330 | 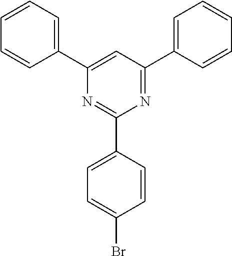 [SMILES: Brc1ccc(-c2nc(-c3ccccc3)cc(-c3ccccc3)n2)cc1] | 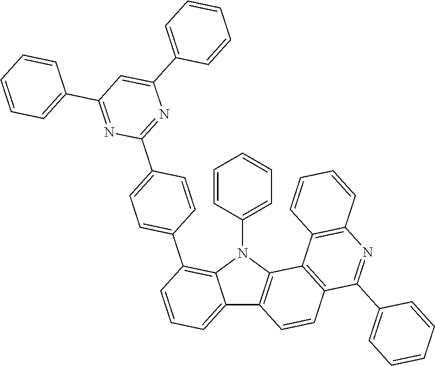 [SMILES: c1ccc(-c2cc(-c3ccccc3)nc(-c3ccc(-c4cccc5c6ccc7c(-c8ccccc8)nc8ccccc8c7c6n(-c6ccccc6)c45)cc3)n2)cc1] | 83% |
| 335 | 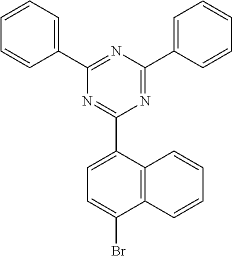 [SMILES: Brc1ccc(-c2nc(-c3ccccc3)nc(-c3ccccc3)n2)c2ccccc12] | 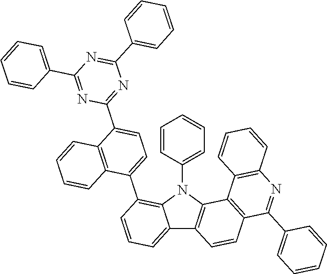 [SMILES: c1ccc(-c2nc(-c3ccccc3)nc(-c3ccc(-c4cccc5c6ccc7c(-c8ccccc8)nc8ccccc8c7c6n(-c6ccccc6)c45)c4ccccc34)n2)cc1] | 77% |
| 339 | 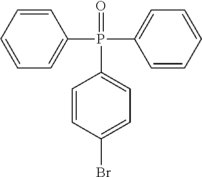 [SMILES: O=P(c1ccccc1)(c1ccccc1)c1ccc(Br)cc1] | 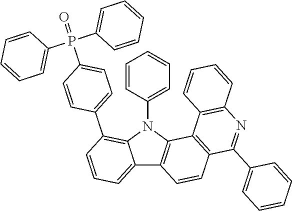 [SMILES: O=P(c1ccccc1)(c1ccccc1)c1ccc(-c2cccc3c4ccc5c(-c6ccccc6)nc6ccccc6c5c4n(-c4ccccc4)c23)cc1] | 87% |

Target Compounds were synthesized in the same manner as in Preparation Example 6 except that (4-chlorophenyl)boronic acid was used instead of (2-chlorophenyl)boronic acid, and Intermediate N of the following Table 14 was used instead of 9-(3-(4-chloro-6-phenyl-1,3,5-triazin-2-yl)phenyl)-9H-carbazole.

TABLE 14

| Compound | | | |
|---|---|---|---|
| No. | Intermediate N | Target Compound | Yield |
| 283 | 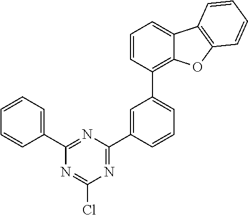 [SMILES: Clc1nc(-c2ccccc2)nc(-c2cccc(-c3cccc4c3oc3ccccc34)c2)n1] | 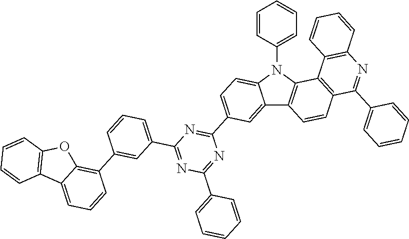 [SMILES: c1ccc(-c2nc(-c3cccc(-c4cccc5c4oc4ccccc45)c3)nc(-c3ccc4c(c3)c3ccc5c(-c6ccccc6)nc6ccccc6c5c3n4-c3ccccc3)n2)cc1] | 78% |
| 286 | 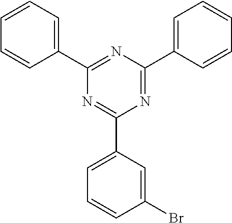 [SMILES: Brc1cccc(-c2nc(-c3ccccc3)nc(-c3ccccc3)n2)c1] | 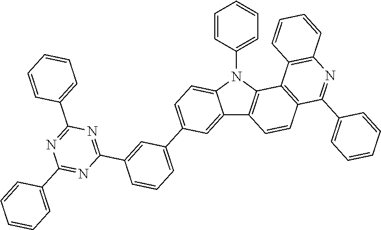 [SMILES: c1ccc(-c2nc(-c3ccccc3)nc(-c3cccc(-c4ccc5c(c4)c4ccc6c(-c7ccccc7)nc7ccccc7c6c4n5-c4ccccc4)c3)n2)cc1] | 85% |
| 289 | 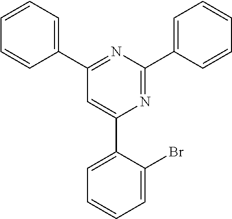 [SMILES: Brc1ccccc1-c1cc(-c2ccccc2)nc(-c2ccccc2)n1] | 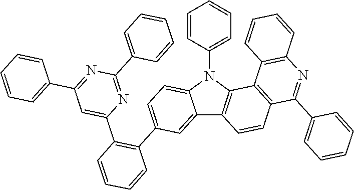 [SMILES: c1ccc(-c2cc(-c3ccccc3-c3ccc4c(c3)c3ccc5c(-c6ccccc6)nc6ccccc6c5c3n4-c3ccccc3)nc(-c3ccccc3)n2)cc1] | 79% |
| 294 | 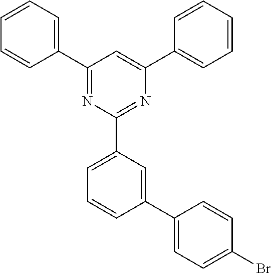 [SMILES: Brc1ccc(-c2cccc(-c3nc(-c4ccccc4)cc(-c4ccccc4)n3)c2)cc1] | 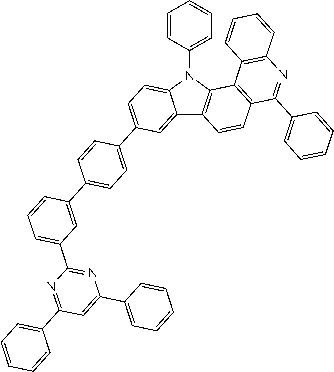 [SMILES: c1ccc(-c2cc(-c3ccccc3)nc(-c3cccc(-c4ccc(-c5ccc6c(c5)c5ccc7c(-c8ccccc8)nc8ccccc8c7c5n6-c5ccccc5)cc4)c3)n2)cc1] | 83% |
| 297 | 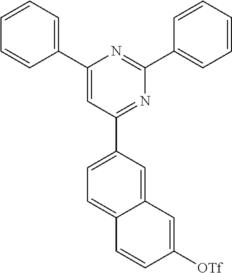 [SMILES: O=S(=O)(Oc1ccc2ccc(-c3cc(-c4ccccc4)nc(-c4ccccc4)n3)cc2c1)C(F)(F)F] | 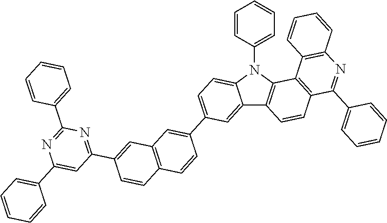 [SMILES: c1ccc(-c2cc(-c3ccc4ccc(-c5ccc6c(c5)c5ccc7c(-c8ccccc8)nc8ccccc8c7c5n6-c5ccccc5)cc4c3)nc(-c3ccccc3)n2)cc1] | 82% |

Compounds other than the compounds described in Preparation Examples 1 to 6 and Tables 1 to 14 were also prepared in the same manner as the compounds described in Preparation Examples 1 to 6 and Tables 1 to 14, and the synthesis identification results are shown in the following Table 15 and Table 16.

TABLE 15

| Compound | ¹H NMR (CDCl₃, 400 Mz) |
|---|---|
| 1 | δ = 8.30-8.28(m, 6H), 8.18(d, 1H), 8.06-7.92(m, 4H), |
| | 7.78(t, 2H), 7.60-7.41(m, 16H) |
| 5 | δ = 8.30-8.28(m, 6H), 8.23(s, 1H), 8.18(d, 1H), 7.99(d, |
| | 2H), 7.92-7.77(m, 7H), 7.66-7.32(m, 17H), 7.25(d, 2H) |
| 12 | δ = 8.30-8.28(m, 6H), 8.24(d, 1H), 8.18(d, 1H), 8.06- |
| | 7.92(m, 4H), 7.77(t, 2H), 7.70(s, 2H), 7.60-7.41(m, |
| | 21H) |
| 13 | δ = 8.30-8.23(m, 7H), 8.18(d, 1H), 8.06-7.92(m, 4H), |
| | 7.85-7.77(m, 6H), 7.70(s, 1H), 7.60-7.41(m, 19H) |
| 19 | δ = 8.30(d, 2H), 8.18(d, 1H), 8.06-7.98(m, 3H), 7.92(d, |
| | 1H), 7.83-7.77(m, 10H), 7.70(s, 1H), 7.60-7.45(m, 19H) |
| 24 | δ = 8.49(d, 1H), 8.30-8.23(m, 5H), 8.08(d, 2H), 7.98(d, |
| | 1H), 7.92(d, 1H), 7.78(t, 3H), 7.60-7.41(m, 17H) |
| 26 | δ = 8.49(d, 1H), 8.30-8.24(m, 7H), 8.08(d, 2H), 7.98(d, |
| | 1H), 7.92(d, 1H), 7.78(t, 1H), 7.70(s, 1H), 7.60- |
| | 7.41(m, 19H) |
| 29 | δ = 8.49(d, 1H), 8.30-8.23(m, 5H), 8.08(d, 2H), 7.79- |
| | 7.70(m, 5H), 7.60-7.41(m, 19H) |
| 35 | δ = 8.55(d, 2H), 8.49(d, 1H), 8.30-8.24(m, 6H), 8.06- |
| | 7.92(m, 6H), 7.78(t, 1H), 7.60-7.41(m, 19H) |
| 40 | δ = 8.49(d, 1H), 8.30-8.21(m, 6H), 8.10-8.06(m, 5H), |
| | 7.98(d, 1H), 7.92(d, 1H), 7.80(t, 2H), 7.62-7.47(m, |
| | 16H), 7.35(d, 2H) |
| 43 | δ = 8.51-8.41(m, 3H), 8.29(d, 4H), 8.19(d, 2H), 8.06(d, |
| | 1H), 7.98-7.85(m, 5H), 7.78(d, 1H), 7.60-7.41(m, 16H), |
| | 7.31-7.25(m, 3H) |
| 45 | δ = 8.55-8.49(m, 3H), 8.30-8.23(m, 5H), 8.12(d, 1H), |
| | 8.06(d, 1H), 7.98-7.92(m, 3H), 7.78(t, 3H), 7.68- |
| | 7.41(m, 17H), 7.33-7.25(m, 4H) |
| 51 | δ = 8.51(d, 1H), 8.30-8.28(m, 7H), 8.06(d, 1H), 7.98- |
| | 7.85(m, 4H), 7.78(t, 1H), 7.60-7.45(m, 10H), 7.31- |
| | 7.25(m, 7H) |
| 54 | δ = 8.51(d, 1H), 8.30-8.23(m, 5H), 8.06(d, 1H), 7.96(d, |
| | 2H), 7.79(d, 5H), 7.70(s, 1H), 7.60-7.41(m, 18H), |
| | 7.31-7.25(m, 5H) |
| 57 | δ = 8.51(d, 1H), 8.34-8.23(m, 7H), 8.06(d, 1H), 7.98- |
| | 7.87(m, 5H), 7.79-7.73(m, 4H), 7.60-7.41(m, 17H), |
| | 7.31(t, 1H) |
| 64 | δ = 8.30-8.22(m, 6H), 8.06(d, 1H), 7.98(d, 1H), 7.89(d, |
| | 2H), 7.79(td, 3H), 7.71(s, 1H), 7.60-7.41(m, 16H) |
| 66 | δ = 8.30-8.28(m, 6H), 8.08(d, 2H), 7.98(d, 1H), 7.90- |
| | 7.78(m, 5H), 7.71(s, 1H), 7.60-7.41(m, 18H) |
| 70 | δ = 8.30(d, 2H), 8.23(s, 1H), 8.08(d, 2H), 7.98(d, 1H), |
| | 7.90--7.71(m, 10H), 7.60-7.41(m, 16H), 7.25(d, 2H) |
| 76 | δ = 9.09(s, 1H), 8.49(d, 1H), 8.30-8.28(m, 6H), 8.08(d, |
| | 2H), 7.98-7.87(m, 5H), 7.78-7.71(m, 3H), 7.60-7.41(m, |
| | 18H) |
| 78 | δ = 9.30(d, 2H), 9.15(s, 2H), 8.53(d, 2H), 8.30(d, 2H), |
| | 8.08(d, 2H), 7.98(d, 1H), 7.88(d, 2H), 7.78-7.71(m, |
| | 4H), 7.60-7.39(m, 10H), 7.25(d, 4H), 7.14(t, 2H) |
| 82 | δ = 8.55(d, 1H), 8.30-8.28(m, 5H), 8.18-7.94(m, 7H), |
| | 7.87(s, 1H), 7.78(t, 2H), 7.71(s, 1H), 7.63-7.41(m, |
| | 16H), 7.33-7.25(m, 3H) |
| 87 | δ = 8.30-8.18(m, 6H), 8.06-7.98(m, 3H), 7.87-7.77(m, |
| | 5H), 7.71(s, 2H), 7.60-7.41(m, 19H), 7.25(d, 2H) |
| 89 | δ = 8.30-8.18(m, 6H), 8.06-7.98(m, 3H), 7.87(s, 1H), |
| | 7.79-7.70(m, 7H), 7.60-7.41(m, 17H) |
| 93 | δ = 8.30-8.18(m, 8H), 8.06-7.98(m, 3H), 7.87-7.77(m, |
| | 7H), 7.70(s, 2H), 7.60-7.41(m, 18H) |
| 99 | δ = 8.30(d, 2H), 8.18(d, 1H), 8.08-7.98(m, 3H), 7.87- |
| | 7.70(m, 13H), 7.60-7.41(m, 18H) |
| 103 | δ = 8.49(d, 1H), 8.30-8.28(m, 4H), 8.08(d, 2H), 7.98(d, |
| | 1H), 7.87-7.71(m, 7H), 7.66-7.41(m, 14H), 7.38-7.25(m, |
| | 5H) |
| 106 | δ = 8.49(d, 1H), 8.30-8.24(m, 5H), 8.08(d, 2H), 7.98(d, |
| | 1H), 7.87(s, 1H), 7.78-7.70(m, 3H), 7.60-7.41(m, 18H) |
| 112 | δ = 8.49(d, 1H), 8.30-8.24(m, 7H), 8.08(d, 2H), 7.98(d, |
| | 1H), 7.87(s, 1H), 7.78-7.70(m, 4H), 7.60-7.41(m, 20H) |
| 114 | δ = 8.49(d, 1H), 8.30-8.23(m, 4H), 8.08(d, 2H), 7.98(d, |
| | 1H), 7.87(s, 1H), 7.79-7.70(m, 7H), 7.60-7.41(m, 18H), |
| | 7.25(d, 4H) |
| 119 | δ = 8.49(d, 1H), 8.30(d, 2H), 8.08(d, 2H), 7.98(d, 1H), |
| | 7.87-7.71(m, 11H), 7.62-7.41(m, 16H) |
| 122 | δ = 8.53(d, 2H), 8.30-8.28(m, 5H), 8.18-8.06(m, 4H), |
| | 7.96(d, 2H), 7.87(s, 1H), 7.78(t, 1H), 7.71(s, 1H), |
| | 7.60-7.41(m, 16H), 7.33-7.25(m, 4H) |
| 128 | δ = 8.51(d, 1H), 8.30-8.23(m, 8H), 8.06(d, 1H), 7.98(d, |
| | 1H), 7.87(s, 1H), 7.78-7.70(m, 6H), 7.60-7.41(m, 14H), |
| | 7.31(t, 1H) |
| 130 | δ = 8.51(d, 1H), 8.30(d, 3H), 8.23(s, 1H), 8.06(d, 1H), |
| | 7.98(d, 1H), 7.87(s, 1H), 7.78-7.71(m, 8H), 7.60- |
| | 7.41(m, 15H), 7.31-7.25(m, 3H) |
| 135 | δ = 8.55-8.51(m, 3H), 8.29(d, 7H), 8.06-7.98(m, 4H), |
| | 7.87(s, 1H), 7.78(t, 1H), 7.71(s, 1H), 7.60-7.41(m, |
| | 17H), 7.31(t, 1H) |
| 137 | δ = 8.51(d, 1H), 8.31-8.23(m, 7H), 8.06(d, 1H), 7.98- |
| | 7.87(m, 5H), 7.79-7.71(m, 5H), 7.60-7.41(m, 15H), |
| | 7.31(t, 1H) |
| 144 | δ = 8.49(d, 1H), 8.30-8.23(m, 5H), 8.08(d, 2H), 7.98(d, |
| | 1H), 7.79-7.76(m, 5H), 7.62-7.41(m, 17H) |
| 147 | δ = 8.49(d, 1H), 8.30-8.24(m, 6H), 8.08(d, 2H), 7.98(d, |
| | 1H), 7.78-7.70(m, 4H), 7.62-7.41(m, 23H) |
| 151 | δ = 8.49(d, 1H), 8.30-8.24(m, 6H), 8.08(d, 2H), 7.98(d, |
| | 1H), 7.85-7.76(m, 4H), 7.62-7.41(m, 17H), 7.25(d, 6H) |
| 154 | δ = 8.49(d, 1H), 8.30-8.24(m, 4H), 8.08(d, 2H), 7.98(d, |
| | 1H), 7.79-7.70(m, 7H), 7.62-7.41(m, 19H), 7.25(d, 4H) |
| 157 | δ = 8.49(d, 1H), 8.34-8.23(m, 6H), 8.08(d, 2H), 7.98- |
| | 7.87(m, 4H), 7.79-7.73(m, 5H), 7.62-7.41(m, 18H) |
| 161 | δ = 8.49(d, 1H), 8.30-8.24(m, 6H), 8.08(d, 2H), 7.98(d, |
| | 1H), 7.78-7.70(m, 4H), 7.62-7.41(m, 23H) |
| 166 | δ = 8.51(d, 1H), 8.30-8.28(m, 7H), 8.06(d, 1H), 7.98(d, |
| | 1H), 7.85-7.76(m, 4H), 7.60-7.41(m, 18H), 7.31(t, 1H) |
| 171 | δ = 8.51(d, 1H), 8.30-8.28(m, 7H), 8.06(d, 1H), 7.98(d, |
| | 1H), 7.85-7.70(m, 5H), 7.60-7.41(m, 19H), 7.31-7.25(m, |
| | 3H) |
| 176 | δ = 9.09(s, 1H), 8.50(d, 2H), 8.30-8.28(m, 7H), 8.06(d, |
| | 1H), 7.98-7.92(m, 3H), 7.78-7.73(m, 3H), 7.60-7.41(m, |
| | 17H), 7.31(t, 1H) |
| 180 | δ = 8.51(d, 1H), 8.30-8.21(m, 7H), 8.08(d, 4H), 7.98(d, |
| | 1H), 7.81-7.76(m, 3H), 7.60-7.41(m, 15H), 7.35-7.31(m, |
| | 3H) |
| 183 | δ = 8.43(d, 2H), 8.29(dd, 4H), 8.20(d, 1H), 8.08(d, 2H), |
| | 7.98(d, 1H), 7.90-7.78(m, 5H), 7.71(s, 1H), 7.60- |
| | 7.39(m, 16H), 7.25(d, 2H) |
| 187 | δ = 8.30-8.24(m, 6H), 8.08(d, 2H), 7.98(d, 1H), 7.90(d, |
| | 1H), 7.87(s, 1H), 7.78(t, 1H), 7.71(s, 3H), 7.60- |
| | 7.39(m, 22H) |
| 192 | δ = 8.30-8.24(m, 7H), 8.08(d, 2H), 7.98(d, 1H), 7.90(d, |
| | 1H), 7.87(s, 1H), 7.78(t, 1H), 7.71(s, 3H), 7.60- |
| | 7.39(m, 21H) |
| 196 | δ = 9.09(s, 1H), 8.49(d, 1H), 8.30-8.28(m, 6H), 8.08(d, |
| | 2H), 7.98-7.87(m, 5H), 7.78-7.71(m, 3H), 7.60-7.41(m, |
| | 17H) |
| 199 | δ = 8.30(d, 2H), 8.08(d, 2H), 7.98(d, 1H), 7.90-7.70(m, |
| | 13H), 7.60-7.39(m, 19H) |
| 204 | δ = 8.30-8.23(m, 5H), 8.18(d, 1H), 8.00-7.98(m, 3H), |
| | 7.87(s, 1H), 7.78(m, 4H), 7.71(s, 1H), 7.60-7.41(m, |
| | 15H) |
| 207 | δ = 8.30-8.18(m, 6H), 8.00-7.98(m, 3H), 7.85-7.78(m, |
| | 5H), 7.71(s, 2H), 7.60-7.41(m, 19H), 7.25(d, 2H) |
| 210 | δ = 8.30-8.18(m, 5H), 8.00-7.98(m, 3H), 7.87(s, 1H), |
| | 7.77(m, 6H), 7.71(s, 2H), 7.60-7.41(m, 17H) |
| 215 | δ = 8.55(d, 2H), 8.30-8.28(m, 6H), 8.18(d, 1H), 8.06- |
| | 7.98(m, 5H), 7.87(s, 1H), 7.77(t, 2H), 7.71(s, 1H), |
| | 7.60-7.41(m, 17H) |
| 218 | δ = 9.30(d, 2H), 9.15(s, 2H), 8.53(d, 2H), 8.30 (d, 2H), |
| | 8.18(d, 1H), 8.06-7.98(m, 3H), 7.87(s, 1H), 7.78- |
| | 7.70(m, 5H), 7.60-7.45(m, 12H), 7.14(t, 2H) |
| 222 | δ = 8.55(d, 1H), 8.49(d, 1H), 8.30-8.28(m, 5H), 8.12- |
| | 8.06(m, 4H), 7.96(d, 2H), 7.87(s, 1H), 7.78(t, 1H), |
| | 7.71(s, 1H), 7.63-7.41(m, 17H), 7.33-7.25(m, 3H) |
| 228 | δ = 8.49(d, 1H), 8.30-8.23(m, 9H), 8.08(d, 2H), 7.98(d, |
| | 1H), 7.87-7.78(m, 6H), 7.71(s, 1H), 7.62-7.41(m, 18H) |
| 230 | δ = 8.49(d, 1H), 8.30-8.23(m, 4H), 8.08(d, 2H), 7.98(d, |
| | 1H), 7.87(s, 1H), 7.79(td, 5H), 7.71(s, 2H), 7.62- |
| | 7.41(m, 18H) |
| 235 | δ = 8.55(d, 2H), 8.49(d, 1H), 8.30(d, 6H), 8.10-7.98(m, |
| | 5H), 7.87(s, 1H), 7.78(t, 1H), 7.71(s, 1H), 7.62- |
| | 7.41(m, 18H) |
| 239 | δ = 8.49(d, 1H), 8.30(d, 2H), 8.08(d, 2H), 7.98(d, 1H), |
| | 7.87-7.71(m, 11H), 7.62-7.41(m, 16H) |
| 244 | δ = 8.50(d, 2H), 8.30-8.23(m, 5H), 8.06(d, 1H), 7.98(d, |
| | 1H), 7.87(s, 1H), 7.79(td, 3H), 7.71(s, 1H), 7.62- |
| | 7.41(m, 15H), 7.31(t, 1H) |
| 248 | δ = 8.51(d, 1H), 8.30-8.23(m, 8H), 8.06(d, 1H), 7.98(d, |
| | 1H), 7.87-7.71(m, 7H), 7.60-7.41(m, 19H), 7.31(t, 1H) |
| 252 | δ = 8.51(d, 1H), 8.30-8.24(m, 8H), 8.06(d, 1H), 7.98(d, |
| | 1H), 7.87(s, 1H), 7.78(t, 1H), 7.70(s, 3H), 7.60- |
| | 7.41(m, 20H), 7.31(t, 1H) |
| 256 | δ = 9.09(s, 1H), 8.50(d, 2H), 8.29(m, 7H), 8.06(d, 1H), |
| | 7.98-7.87(m, 4H), 7.78-7.71(m, 3H), 7.60-7.41(m, 16H), |
| | 7.31(t, 1H) |
| 260 | δ = 8.51(d, 1H), 8.30-8.21(m, 7H),8.08(dd, 4H), 7.98(d, |
| | 1H), 7.87(s, 1H), 7.79(t, 2H), 7.71(s, 1H), 7.60- |
| | 7.41(m, 14H), 7.35-7.31(m, 3H) |
| 262 | δ = 8.55(d, 1H), 8.30-8.28(m, 5H), 8.08(d, 3H), 7.98- |
| | 7.92(m, 4H), 7.78(t, 1H), 7.63-7.39(m, 18H), 7.33- |
| | 7.25(m, 3H) |
| 265 | δ = 8.55(d, 1H), 8.30-8.22(m, 6H), 8.08(d, 2H), 7.98- |
| | 7.90(m, 3H), 7.79(td, 3H), 7.68-7.39(m, 18H), 7.33- |
| | 7.25(m, 3H) |
| 271 | δ = 8.30-8.28(m, 6H), 8.08(d, 2H), 7.98-7.85(m, 4H), |
| | 7.78(t, 1H), 7.68-7.39(m, 17H), 7.25(d, 6H) |
| 274 | δ = 8.30(d, 2H), 8.24(d, 2H), 8.08(d, 2H), 7.98-7.90(m, |
| | 3H), 7.78(td, 4H), 7.70(s, 1H), 7.58-7.39(m, 19H), |
| | 7.25(d, 4H) |
| 279 | δ = 8.30(d, 2H), 8.08(d, 2H), 7.98-7.78(m, 12H), 7.58- |
| | 7.39(m, 17H) |
| 283 | δ = 8.30-8.18(m, 6H), 8.06-7.77(m, 9H), 7.70-7.41(m, |
| | 20H) |
| 286 | δ = 8.30-8.18(m, 8H), 8.06-7.92(m, 4H), 7.78(t, 2H), |
| | 7.70(s, 1H), 7.58-7.41(m, 18H) |
| 289 | δ = 8.30-8.18(m, 5H), 8.06-7.78(m, 10H), 7.58-7.41(m, |
| | 18H) |
| 294 | δ = 8.30-8.18(m, 5H), 8.06-7.92(m, 4H), 7.78(t, 6H), |
| | 7.70(s, 1H), 7.58-7.41(m, 18H), 7.25(d, 4H) |
| 297 | δ = 8.34-8.18(m, 7H), 8.06-7.79(m, 12H), 7.58-7.41(m, |
| | 17H) |
| 304 | δ = 8.49(d, 1H), 8.30-8.23(m, 5H), 8.08(d, 2H), 7.95(d, |
| | 2H), 7.79(td, 3H), 7.62-7.39(m, 17H) |
| 307 | δ = 8.49(d, 1H), 8.30-8.24(m, 5H), 8.08(d, 2H), 7.98- |
| | 7.78(m, 5H), 7.70(s, 1H), 7.62-7.41(m, 21H), 7.25(d, |
| | 2H) |
| 310 | δ = 8.49(d, 1H), 8.30(d, 2H), 8.23(s, 1H), 8.08(d, 2H), |
| | 7.98-7.78(m, 9H), 7.62-7.41(m, 19H) |
| 315 | δ = 8.55(d, 2H), 8.49(d, 1H), 8.29(d, 6H), 8.10-7.98(m, |
| | 6H), 7.78(t, 1H), 7.62-7.41(m, 19H) |
| 318 | δ = 9.30(d, 2H), 9.15(s, 2H), 8.53(d, 2H), 8.49(d, 1H), |
| | 8.30 (d, 2H), 8.08(d, 2H), 7.95(d, 2H), 7.78-7.70(m, |
| | 4H), 7.62-7.47(m, 16H), 7.14(t, 2H) |
| 323 | δ = 8.51(d, 1H), 8.29(d, 4H), 8.18(d, 1H), 8.06(d, 1H), |
| | 7.98-7.78(m, 7H), 7.60-7.41(m, 18H), 7.31-7.25(m, 3H) |
| 326 | δ = 8.51(d, 1H), 8.30-8.28(m, 7H), 8.06(d, 1H), 7.98- |
| | 7.78(m, 5H), 7.60-7.41(m, 18H), 7.31(t, 1H) |
| 330 | δ = 8.51(d, 1H), 8.30(d, 3H), 8.23(s, 1H), 8.06(d, 1H), |
| | 7.98-7.78(m, 9H), 7.60-7.41(m, 16H), 7.31-7.25(m, 3H) |
| 335 | δ = 8.55(d, 2H), 8.51(d, 1H), 8.30-8.28(m, 7H), 8.06- |
| | 7.78(m, 5H), 7.78(t, 1H), 7.60-7.41(m, 18H), 7.31(t, |
| | 1H) |
| 339 | δ = 8.51(d, 1H), 8.30(d, 3H), 8.06(d, 1H), 7.95(d, 2H), |
| | 7.83-7.78(m, 9H), 7.60-7.41(m, 16H), 7.31(t, 1H) |

TABLE 16

| Compound | FD-Mass | Compound | FD-Mass |
|---|---|---|---|
| 1 | m/z = 651.76 | 2 | m/z = 816.95 |
| | (C46H29N5 = 651.24) | | (C58H36N6 = 816.30) |
| 3 | m/z = 834.00 | 4 | m/z = 650.77 |
| | (C58H35N5S = 833.26) | | (C47H30N4 = 650.25) |
| 5 | m/z = 816.94 | 6 | m/z = 727.85 |
| | (C59H36N4O = 816.29) | | (C52H33N5 = 727.27) |
| 7 | m/z = 803.95 | 8 | m/z = 803.95 |
| | (C58H37N5 = 803.30) | | (C58H37N5 = 803.30) |
| 9 | m/z = 726.86 | 10 | m/z = 726.86 |
| | (C53H34N4 = 726.28) | | (C53H34N4 = 726.28) |
| 11 | m/z = 803.95 | 12 | m/z = 803.95 |
| | (C58H37N5 = 803.30) | | (C58H37N5 = 803.30) |
| 13 | m/z = 802.96 | 14 | m/z = 802.96 |
| | (C59H38N4 = 802.31) | | (C59H38N4 = 802.31) |
| 15 | m/z = 777.91 | 16 | m/z = 777.91 |
| | (C56H35N5 = 777.29) | | (C56H35N5 = 777.29) |
| 17 | m/z = 776.92 | 18 | m/z = 727.85 |
| | (C57H36N4 = 776.29) | | (C52H33N5 = 727.27) |
| 19 | m/z = 772.87 | 20 | m/z = 750.89 |
| | (C55H37N2OP = 772.26) | | (C55H34N4 = 750.28) |
| 21 | m/z = 651.76 | 22 | m/z = 816.95 |
| | (C46H29N5 = 651.24) | | (C58H36N6 = 816.30) |
| 23 | m/z = 834.00 | 24 | m/z = 650.77 |
| | (C58H35N5S = 833.26) | | (C47H30N4 = 650.25) |
| 25 | m/z = 815.96 | 26 | m/z = 727.85 |
| | (C59H37N5 = 815.30) | | (C52H33N5 = 727.27) |
| 27 | m/z = 803.95 | 28 | m/z = 802.96 |
| | (C58H37N5 = 803.30) | | (C59H37N4 = 802.31) |
| 29 | m/z = 726.86 | 30 | m/z = 726.86 |
| | (C53H34N4 = 726.28) | | (C53H34N4 = 726.28) |
| 31 | m/z = 803.95 | 32 | m/z = 803.95 |
| | (C58H37N5 = 803.30) | | (C58H37N5 = 803.30) |
| 33 | m/z = 802.96 | 34 | m/z = 802.96 |
| | (C59H37N4 = 802.31) | | (C59H37N4 = 802.31) |
| 35 | m/z = 777.91 | 36 | m/z = 777.91 |
| | (C56H35N5 = 777.29) | | (C56H35N5 = 777.29) |
| 37 | m/z = 776.92 | 38 | m/z = 727.85 |
| | (C57H36N4 = 776.29) | | (C52H33N5 = 727.27) |
| 39 | m/z = 772.87 | 40 | m/z = 750.89 |
| | (C55H37N2OP = 772.26) | | (C55H34N4 = 750.28) |
| 41 | m/z = 651.76 | 42 | m/z = 816.95 |
| | (C46H29N5 = 651.24) | | (C58H36N6 = 816.30) |
| 43 | m/z = 834.00 | 44 | m/z = 650.77 |
| | (C58H35N5S = 833.26) | | (C47H30N4 = 650.25) |
| 45 | m/z = 815.96 | 46 | m/z = 727.85 |
| | (C59H37N5 = 815.30) | | (C52H33N5 = 727.27) |
| 47 | m/z = 803.95 | 48 | m/z = 802.96 |
| | (C58H37N5 = 803.30) | | (C59H37N4 = 802.31) |
| 49 | m/z = 726.86 | 50 | m/z = 726.86 |
| | (C53H34N4 = 726.28) | | (C53H34N4 = 726.28) |
| 51 | m/z = 803.95 | 52 | m/z = 803.95 |
| | (C58H37N5 = 803.30) | | (C58H37N5 = 803.30) |
| 53 | m/z = 802.96 | 54 | m/z = 802.96 |
| | (C59H37N4 = 802.31) | | (C59H37N4 = 802.31) |
| 55 | m/z = 777.91 | 56 | m/z = 777.91 |
| | (C56H35N5 = 777.29) | | (C56H35N5 = 777.29) |
| 57 | m/z = 776.92 | 58 | m/z = 727.85 |
| | (C57H36N4 = 776.29) | | (C52H33N5 = 727.27) |
| 59 | m/z = 772.87 | 60 | m/z = 750.89 |
| | (C55H37N2OP = 772.26) | | (C55H34N4 = 750.28) |
| 61 | m/z = 651.76 | 62 | m/z = 816.95 |
| | (C46H29N5 = 651.24) | | (C58H36N6 = 816.30) |
| 63 | m/z = 834.00 | 64 | m/z = 650.77 |
| | (C58H35N5S = 833.26) | | (C47H30N4 = 650.25) |
| 65 | m/z = 815.96 | 66 | m/z = 727.85 |
| | (C59H37N5 = 815.30) | | (C52H33N5 = 727.27) |
| 67 | m/z = 803.95 | 68 | m/z = 802.96 |
| | (C58H37N5 = 803.30) | | (C59H37N4 = 802.31) |
| 69 | m/z = 726.86 | 70 | m/z = 726.86 |
| | (C53H34N4 = 726.28) | | (C53H34N4 = 726.28) |
| 71 | m/z = 803.95 | 72 | m/z = 803.95 |
| | (C58H37N5 = 803.30) | | (C58H37N5 = 803.30) |
| 73 | m/z = 802.96 | 74 | m/z = 802.96 |
| | (C59H37N4 = 802.31) | | (C59H37N4 = 802.31) |
| 75 | m/z = 777.91 | 76 | m/z = 777.91 |
| | (C56H35N5 = 777.29) | | (C56H35N5 = 777.29) |
| 77 | m/z = 776.92 | 78 | m/z = 727.85 |
| | (C57H36N4 = 776.29) | | (C52H33N5 = 727.27) |
| 79 | m/z = 772.87 | 80 | m/z = 750.89 |
| | (C55H37N2OP = 772.26) | | (C55H34N4 = 750.28) |
| 81 | m/z = 651.76 | 82 | m/z = 816.95 |
| | (C46H29N5 = 651.24) | | (C58H36N6 = 816.30) |
| 83 | m/z = 817.93 | 84 | m/z = 650.77 |
| | (C58H35N5O = 817.28) | | (C47H30N4 = 650.25) |
| 85 | m/z = 815.96 | 86 | m/z = 727.85 |
| | (C59H37N5 = 815.30) | | (C52H33N5 = 727.27) |
| 87 | m/z = 803.95 | 88 | m/z = 802.96 |
| | (C58H37N5 = 803.30) | | (C59H37N4 = 802.31) |
| 89 | m/z = 726.86 | 90 | m/z = 726.86 |
| | (C53H34N4 = 726.28) | | (C53H34N4 = 726.28) |
| 91 | m/z = 803.95 | 92 | m/z = 803.95 |
| | (C58H37N5 = 803.30) | | (C58H37N5 = 803.30) |
| 93 | m/z = 802.96 | 94 | m/z = 802.96 |
| | (C59H37N4 = 802.31) | | (C59H37N4 = 802.31) |
| 95 | m/z = 777.91 | 96 | m/z = 777.91 |
| | (C56H35N5 = 777.29) | | (C56H35N5 = 777.29) |
| 97 | m/z = 776.92 | 98 | m/z = 727.85 |
| | (C57H36N4 = 776.29) | | (C52H33N5 = 727.27) |
| 99 | m/z = 772.87 | 100 | m/z = 750.89 |
| | (C55H37N2OP = 772.26) | | (C55H34N4 = 750.28) |
| 101 | m/z = 651.76 | 102 | m/z = 816.95 |
| | (C46H29N5 = 651.24) | | (C58H36N6 = 816.30) |
| 103 | m/z = 817.93 | 104 | m/z = 650.77 |
| | (C58H35N5O = 817.28) | | (C47H30N4 = 650.25) |
| 105 | m/z = 815.96 | 106 | m/z = 727.85 |
| | (C59H37N5 = 815.30) | | (C52H33N5 = 727.27) |
| 107 | m/z = 803.95 | 108 | m/z = 802.96 |
| | (C58H37N5 = 803.30) | | (C59H37N4 = 802.31) |
| 109 | m/z = 726.86 | 110 | m/z = 726.86 |
| | (C53H34N4 = 726.28) | | (C53H34N4 = 726.28) |
| 111 | m/z = 803.95 | 112 | m/z = 803.95 |
| | (C58H37N5 = 803.30) | | (C58H37N5 = 803.30) |
| 113 | m/z = 802.96 | 114 | m/z = 802.96 |
| | (C59H37N4 = 802.31) | | (C59H37N4 = 802.31) |
| 115 | m/z = 777.91 | 116 | m/z = 777.91 |
| | (C56H35N5 = 777.29) | | (C56H35N5 = 777.29) |
| 117 | m/z = 776.92 | 118 | m/z = 727.85 |
| | (C57H36N4 = 776.29) | | (C52H33N5 = 727.27) |
| 119 | m/z = 696.77 | 120 | m/z = 750.89 |
| | (C49H33N2OP = 696.23) | | (C55H34N4 = 750.28) |
| 121 | m/z = 651.76 | 122 | m/z = 816.95 |
| | (C46H29N5 = 651.24) | | (C58H36N6 = 816.30) |
| 123 | m/z = 834.00 | 124 | m/z = 650.77 |
| | (C58H35N5S = 833.26) | | (C47H30N4 = 650.25) |
| 125 | m/z = 815.96 | 126 | m/z = 727.85 |
| | (C59H37N5 = 815.30) | | (C52H33N5 = 727.27) |
| 127 | m/z = 803.95 | 128 | m/z = 802.96 |
| | (C58H37N5 = 803.30) | | (C59H37N4 = 802.31) |
| 129 | m/z = 726.86 | 130 | m/z = 726.86 |
| | (C53H34N4 = 726.28) | | (C53H34N4 = 726.28) |
| 131 | m/z = 803.95 | 132 | m/z = 803.95 |
| | (C58H37N5 = 803.30) | | (C58H37N5 = 803.30) |
| 133 | m/z = 802.96 | 134 | m/z = 802.96 |
| | (C59H37N4 = 802.31) | | (C59H37N4 = 802.31) |
| 135 | m/z = 777.91 | 136 | m/z = 777.91 |
| | (C56H35N5 = 777.29) | | (C56H35N5 = 777.29) |
| 137 | m/z = 776.92 | 138 | m/z = 727.85 |
| | (C57H36N4 = 776.29) | | (C52H33N5 = 727.27) |
| 139 | m/z = 696.77 | 140 | m/z = 750.89 |
| | (C49H33N2OP = 696.23) | | (C55H34N4 = 750.28) |
| 141 | m/z = 651.76 | 142 | m/z = 816.95 |
| | (C46H29N5 = 651.24) | | (C58H36N6 = 816.30) |
| 143 | m/z = 834.00 | 144 | m/z = 650.77 |
| | (C58H35N5S = 833.26) | | (C47H30N4 = 650.25) |
| 145 | m/z = 815.96 | 146 | m/z = 727.85 |
| | (C59H37N5 = 815.30) | | (C52H33N5 = 727.27) |
| 147 | m/z = 803.95 | 148 | m/z = 802.96 |
| | (C58H37N5 = 803.30) | | (C59H37N4 = 802.31) |
| 149 | m/z = 726.86 | 150 | m/z = 726.86 |
| | (C53H34N4 = 726.28) | | (C53H34N4 = 726.28) |
| 151 | m/z = 803.95 | 152 | m/z = 803.95 |
| | (C58H37N5 = 803.30) | | (C58H37N5 = 803.30) |
| 153 | m/z = 802.96 | 154 | m/z = 802.96 |
| | (C59H37N4 = 802.31) | | (C59H37N4 = 802.31) |
| 155 | m/z = 777.91 | 156 | m/z = 777.91 |
| | (C56H35N5 = 777.29) | | (C56H35N5 = 777.29) |
| 157 | m/z = 776.92 | 158 | m/z = 727.85 |
| | (C57H36N4 = 776.29) | | (C52H33N5 = 727.27) |
| 159 | m/z = 696.77 | 160 | m/z = 750.89 |
| | (C49H33N2OP = 696.23) | | (C55H34N4 = 750.28) |
| 161 | m/z = 651.76 | 162 | m/z = 816.95 |
| | (C46H29N5 = 651.24) | | (C58H36N6 = 816.30) |
| 163 | m/z = 817.93 | 164 | m/z = 650.77 |
| | (C58H35N5O = 816.28) | | (C47H30N4 = 650.25) |
| 165 | m/z = 815.96 | 166 | m/z = 727.85 |
| | (C59H37N5 = 815.30) | | (C52H33N5 = 727.27) |
| 167 | m/z = 803.95 | 168 | m/z = 802.96 |
| | (C58H37N5 = 803.30) | | (C59H37N4 = 802.31) |
| 169 | m/z = 726.86 | 170 | m/z = 726.86 |
| | (C53H34N4 = 726.28) | | (C53H34N4 = 726.28) |
| 171 | m/z = 803.95 | 172 | m/z = 803.95 |
| | (C58H37N5 = 803.30) | | (C58H37N5 = 803.30) |
| 173 | m/z = 802.96 | 174 | m/z = 802.96 |
| | (C59H37N4 = 802.31) | | (C59H37N4 = 802.31) |
| 175 | m/z = 777.91 | 176 | m/z = 777.91 |
| | (C56H35N5 = 777.29) | | (C56H35N5 = 777.29) |
| 177 | m/z = 776.92 | 178 | m/z = 727.85 |
| | (C57H36N4 = 776.29) | | (C52H33N5 = 727.27) |
| 179 | m/z = 696.77 | 180 | m/z = 750.89 |
| | (C49H33N2OP = 696.23) | | (C55H34N4 = 750.28) |
| 181 | m/z = 651.76 | 182 | m/z = 816.95 |
| | (C46H29N5 = 651.24) | | (C58H36N6 = 816.30) |
| 183 | m/z = 834.00 | 184 | m/z = 650.77 |
| | (C58H35N5S = 833.26) | | (C47H30N4 = 650.25) |
| 185 | m/z = 815.96 | 186 | m/z = 727.85 |
| | (C59H37N5 = 815.30) | | (C52H33N5 = 727.27) |
| 187 | m/z = 803.95 | 188 | m/z = 802.96 |
| | (C58H37N5 = 803.30) | | (C59H37N4 = 802.31) |
| 189 | m/z = 726.86 | 190 | m/z = 726.86 |
| | (C53H34N4 = 726.28) | | (C53H34N4 = 726.28) |
| 191 | m/z = 803.95 | 192 | m/z = 803.95 |
| | (C58H37N5 = 803.30) | | (C58H37N5 = 803.30) |
| 193 | m/z = 802.96 | 194 | m/z = 802.96 |
| | (C59H37N4 = 802.31) | | (C59H37N4 = 802.31) |
| 195 | m/z = 777.91 | 196 | m/z = 777.91 |
| | (C56H35N5 = 777.29) | | (C56H35N5 = 777.29) |
| 197 | m/z = 776.92 | 198 | m/z = 727.85 |
| | (C57H36N4 = 776.29) | | (C52H33N5 = 727.27) |
| 199 | m/z = 772.87 | 200 | m/z = 750.89 |
| | (C55H37N2OP = 772.26) | | (C55H34N4 = 750.28) |
| 201 | m/z = 651.76 | 202 | m/z = 816.95 |
| | (C46H29N5 = 651.24) | | (C58H36N6 = 816.30) |
| 203 | m/z = 834.00 | 204 | m/z = 650.77 |
| | (C58H35N5S = 833.26) | | (C47H30N4 = 650.25) |
| 205 | m/z = 815.96 | 206 | m/z = 727.85 |
| | (C59H37N5 = 815.30) | | (C52H33N5 = 727.27) |
| 207 | m/z = 803.95 | 208 | m/z = 802.96 |
| | (C58H37N5 = 803.30) | | (C59H37N4 = 802.31) |
| 209 | m/z = 726.86 | 210 | m/z = 726.86 |
| | (C53H34N4 = 726.28) | | (C53H34N4 = 726.28) |
| 211 | m/z = 803.95 | 212 | m/z = 803.95 |
| | (C58H37N5 = 803.30) | | (C58H37N5 = 803.30) |
| 213 | m/z = 802.96 | 214 | m/z = 802.96 |
| | (C59H37N4 = 802.31) | | (C59H37N4 = 802.31) |
| 215 | m/z = 777.91 | 216 | m/z = 777.91 |
| | (C56H35N5 = 777.29) | | (C56H35N5 = 777.29) |
| 217 | m/z = 776.92 | 218 | m/z = 727.85 |
| | (C57H36N4 = 776.29) | | (C52H33N5 = 727.27) |
| 219 | m/z = 696.77 | 220 | m/z = 750.89 |
| | (C49H33N2OP = 696.23) | | (C55H34N4 = 750.28) |
| 221 | m/z = 651.76 | 222 | m/z = 816.95 |
| | (C46H29N5 = 651.24) | | (C58H36N6 = 816.30) |
| 223 | m/z = 834.00 | 224 | m/z = 650.77 |
| | (C58H35N5S = 833.26) | | (C47H30N4 = 650.25) |
| 225 | m/z = 815.96 | 226 | m/z = 727.85 |
| | (C59H37N5 = 815.30) | | (C52H33N5 = 727.27) |
| 227 | m/z = 803.95 | 228 | m/z = 802.96 |
| | (C58H37N5 = 803.30) | | (C59H37N4 = 802.31) |
| 229 | m/z = 726.86 | 230 | m/z = 726.86 |
| | (C53H34N4 = 726.28) | | (C53H34N4 = 726.28) |
| 231 | m/z = 803.95 | 232 | m/z = 803.95 |
| | (C58H37N5 = 803.30) | | (C58H37N5 = 803.30) |
| 233 | m/z = 802.96 | 234 | m/z = 802.96 |
| | (C59H37N4 = 802.31) | | (C59H37N4 = 802.31) |
| 235 | m/z = 777.91 | 236 | m/z = 777.91 |
| | (C56H35N5 = 777.29) | | (C56H35N5 = 777.29) |
| 237 | m/z = 776.92 | 238 | m/z = 727.85 |
| | (C57H36N4 = 776.29) | | (C52H33N5 = 727.27) |
| 239 | m/z = 696.77 | 240 | m/z = 750.89 |
| | (C49H33N2OP = 696.23) | | (C55H34N4 = 750.28) |
| 241 | m/z = 651.76 | 242 | m/z = 816.95 |
| | (C46H29N5 = 651.24) | | (C58H36N6 = 816.30) |
| 243 | m/z = 817.93 | 244 | m/z = 650.77 |
| | (C58H35N5O = 816.28) | | (C47H30N4 = 650.25) |
| 245 | m/z = 815.96 | 246 | m/z = 727.85 |
| | (C59H37N5 = 815.30) | | (C52H33N5 = 727.27) |
| 247 | m/z = 803.95 | 248 | m/z = 802.96 |
| | (C58H37N5 = 803.30) | | (C59H37N4 = 802.31) |
| 249 | m/z = 726.86 | 250 | m/z = 726.86 |
| | (C53H34N4 = 726.28) | | (C53H34N4 = 726.28) |
| 251 | m/z = 803.95 | 252 | m/z = 803.95 |
| | (C58H37N5 = 803.30) | | (C58H37N5 = 803.30) |
| 253 | m/z = 802.96 | 254 | m/z = 802.96 |
| | (C59H37N4 = 802.31) | | (C59H37N4 = 802.31) |
| 255 | m/z = 777.91 | 256 | m/z = 777.91 |
| | (C56H35N5 = 777.29) | | (C56H35N5 = 777.29) |
| 257 | m/z = 776.92 | 258 | m/z = 727.85 |
| | (C57H36N4 = 776.29) | | (C52H33N5 = 727.27) |
| 259 | m/z = 696.77 | 260 | m/z = 750.89 |
| | (C49H33N2OP = 696.23) | | (C55H34N4 = 750.28) |
| 261 | m/z = 651.76 | 262 | m/z = 816.95 |
| | (C46H29N5 = 651.24) | | (C58H36N6 = 816.30) |
| 263 | m/z = 834.00 | 264 | m/z = 650.77 |
| | (C58H35N5O = 833.26) | | (C47H30N4 = 650.25) |
| 265 | m/z = 815.96 | 266 | m/z = 727.85 |
| | (C59H37N5 = 815.30) | | (C52H33N5 = 727.27) |
| 267 | m/z = 803.95 | 268 | m/z = 802.96 |
| | (C58H37N5 = 803.30) | | (C59H37N4 = 802.31) |
| 269 | m/z = 726.86 | 270 | m/z = 726.86 |
| | (C53H34N4 = 726.28) | | (C53H34N4 = 726.28) |
| 271 | m/z = 803.95 | 272 | m/z = 803.95 |
| | (C58H37N5 = 803.30) | | (C58H37N5 = 803.30) |
| 273 | m/z = 802.96 | 274 | m/z = 802.96 |
| | (C59H37N4 = 802.31) | | (C59H37N4 = 802.31) |
| 275 | m/z = 777.91 | 276 | m/z = 777.91 |
| | (C56H35N5 = 777.29) | | (C56H35N5 = 777.29) |
| 277 | m/z = 776.92 | 278 | m/z = 727.85 |
| | (C57H36N4 = 776.29) | | (C52H33N5 = 727.27) |
| 279 | m/z = 696.77 | 280 | m/z = 750.89 |
| | (C49H33N2OP = 696.23) | | (C55H34N4 = 750.28) |
| 281 | m/z = 651.76 | 282 | m/z = 816.95 |
| | (C46H29N5 = 651.24) | | (C58H36N6 = 816.30) |
| 283 | m/z = 817.93 | 284 | m/z = 650.77 |
| | (C58H35N5O = 816.28) | | (C47H30N4 = 650.25) |
| 285 | m/z = 815.96 | 286 | m/z = 727.85 |
| | (C59H37N5 = 815.30) | | (C52H33N5 = 727.27) |
| 287 | m/z = 803.95 | 288 | m/z = 802.96 |
| | (C58H37N5 = 803.30) | | (C59H37N4 = 802.31) |
| 289 | m/z = 726.86 | 290 | m/z = 726.86 |
| | (C53H34N4 = 726.28) | | (C53H34N4 = 726.28) |
| 291 | m/z = 803.95 | 292 | m/z = 803.95 |
| | (C58H37N5 = 803.30) | | (C58H37N5 = 803.30) |
| 293 | m/z = 802.96 | 294 | m/z = 802.96 |
| | (C59H37N4 = 802.31) | | (C59H37N4 = 802.31) |
| 295 | m/z = 777.91 | 296 | m/z = 777.91 |
| | (C56H35N5 = 777.29) | | (C56H35N5 = 777.29) |
| 297 | m/z = 776.92 | 298 | m/z = 727.85 |
| | (C57H36N4 = 776.29) | | (C52H33N5 = 727.27) |
| 299 | m/z = 696.77 | 300 | m/z = 750.89 |
| | (C49H33N2OP = 696.23) | | (C55H34N4 = 750.28) |
| 301 | m/z = 651.76 | 302 | m/z = 816.95 |
| | (C46H29N5 = 651.24) | | (C58H36N6 = 816.30) |
| 303 | m/z = 834.00 | 304 | m/z = 650.77 |
| | (C58H35N5O = 833.26) | | (C47H30N4 = 650.25) |
| 305 | m/z = 815.96 | 306 | m/z = 727.85 |
| | (C59H37N5 = 815.30) | | (C52H33N5 = 727.27) |
| 307 | m/z = 803.95 | 308 | m/z = 802.96 |
| | (C58H37N5 = 803.30) | | (C59H37N4 = 802.31) |
| 309 | m/z = 726.86 | 310 | m/z = 726.86 |
| | (C53H34N4 = 726.28) | | (C53H34N4 = 726.28) |
| 311 | m/z = 803.95 | 312 | m/z = 803.95 |
| | (C58H37N5 = 803.30) | | (C58H37N5 = 803.30) |
| 313 | m/z = 802.96 | 314 | m/z = 802.96 |
| | (C59H37N4 = 802.31) | | (C59H37N4 = 802.31) |
| 315 | m/z = 777.91 | 316 | m/z = 777.91 |
| | (C56H35N5 = 777.29) | | (C56H35N5 = 777.29) |
| 317 | m/z = 776.92 | 318 | m/z = 727.85 |
| | (C57H36N4 = 776.29) | | (C52H33N5 = 727.27) |
| 319 | m/z = 696.77 | 320 | m/z = 750.89 |
| | (C49H33N2OP = 696.23) | | (C55H34N4 = 750.28) |
| 321 | m/z = 651.76 | 322 | m/z = 816.95 |
| | (C46H29N5 = 651.24) | | (C58H36N6 = 816.30) |
| 323 | m/z = 817.93 | 324 | m/z = 650.77 |
| | (C58H35N5O = 816.28) | | (C47H30N4 = 650.25) |
| 325 | m/z = 815.96 | 326 | m/z = 727.85 |
| | (C59H37N5 = 815.30) | | (C52H33N5 = 727.27) |
| 327 | m/z = 803.95 | 328 | m/z = 802.96 |
| | (C58H37N5 = 803.30) | | (C59H37N4 = 802.31) |
| 329 | m/z = 726.86 | 330 | m/z = 726.86 |
| | (C53H34N4 = 726.28) | | (C53H34N4 = 726.28) |
| 331 | m/z = 803.95 | 332 | m/z = 803.95 |
| | (C58H37N5 = 803.30) | | (C58H37N5 = 803.30) |
| 333 | m/z = 802.96 | 334 | m/z = 802.96 |
| | (C59H37N4 = 802.31) | | (C59H37N4 = 802.31) |
| 335 | m/z = 777.91 | 336 | m/z = 777.91 |
| | (C56H35N5 = 777.29) | | (C56H35N5 = 777.29) |
| 337 | m/z = 776.92 | 338 | m/z = 727.85 |
| | (C57H36N4 = 776.29) | | (C52H33N5 = 727.27) |
| 339 | m/z = 696.77 | 340 | m/z = 750.89 |
| | (C49H33N2OP = 696.23) | | (C55H34N4 = 750.28) |
| 341 | m/z = 751.87 | 342 | m/z = 854.01 |
| | (C54H33N5 = 751.27) | | (C62H39N5 = 853.32) |
| 343 | m/z = 866.02 | 344 | m/z = 750.89 |
| | (C63H39N5 = 865.32) | | (C55H34N4 = 750.28) |
| 345 | m/z = 777.91 | 346 | m/z = 853.02 |
| | (C56H35N5 = 777.29) | | (C63H40N4 = 852.33) |
| 347 | m/z = 826.98 | 348 | m/z = 751.87 |
| | (C61H38N4 = 826.31) | | (C54H33N5 = 751.27) |
| 349 | m/z = 827.97 | 350 | m/z = 776.92 |
| | (C60H37N5 = 827.30) | | (C57H36N4 = 776.29) |
| 351 | m/z = 827.97 | 352 | m/z = 867.99 |
| | (C60H37N5 = 827.30) | | (C62H37N5O = 867.30) |
| 353 | m/z = 827.97 | 354 | m/z = 777.91 |
| | (C60H37N5 = 827.30) | | (C56H35N5 = 777.29) |
| 355 | m/z = 853.02 | 356 | m/z = 827.97 |
| | (C63H40N4 = 852.33) | | (C60H37N5 = 827.30) |
| 357 | m/z = 854.01 | 358 | m/z = 746.83 |
| | (C62H39N5 = 853.32) | | (C53H35N2OP = 746.25) |
| 359 | m/z = 884.06 | 360 | m/z = 800.94 |
| | (C62H37N5S = 883.28) | | (C59H36N4 = 800.29) |
| 361 | m/z = 751.87 | 362 | m/z = 853.02 |
| | (C54H33N5 = 751.27) | | (C63H40N4 = 852.33) |
| 363 | m/z = 866.02 | 364 | m/z = 854.01 |
| | (C63H39N5 = 865.32) | | (C62H39N5 = 853.32) |
| 365 | m/z = 776.92 | 366 | m/z = 854.01 |
| | (C57H36N4 = 776.29) | | (C62H39N5 = 853.32) |
| 367 | m/z = 884.06 | 368 | m/z = 827.97 |
| | (C62H37N5S = 883.28) | | (C60H37N5 = 827.30) |
| 369 | m/z = 777.91 | 370 | m/z = 796.89 |
| | (C56H35N5 = 777.29) | | (C57H37N2OP = 796.26) |
| 371 | m/z = 777.91 | 372 | m/z = 800.94 |
| | (C56H35N5 = 777.29) | | (C59H36N4 = 800.29) |
| 373 | m/z = 777.91 | 374 | m/z = 826.98 |
| | (C56H35N5 = 777.29) | | (C61H38N4 = 826.31) |

### EXPERIMENTAL EXAMPLE

### Experimental Example 1

Manufacture of Organic Light Emitting Device

A transparent indium tin oxide (ITO) electrode thin film obtained from glass for an OLED (manufactured by Samsung-Corning Co., Ltd.) was ultrasonic cleaned using trichloroethylene, acetone, ethanol and distilled water consecutively for 5 minutes each, stored in isopropanol, and used.

Next, an ITO substrate was installed in a substrate folder of a vacuum deposition apparatus, and the following 4,4′,4″-tris(N,N-(2-naphthyl)-phenylamino)triphenylamine (2-TNATA) was introduced to a cell in the vacuum deposition apparatus.

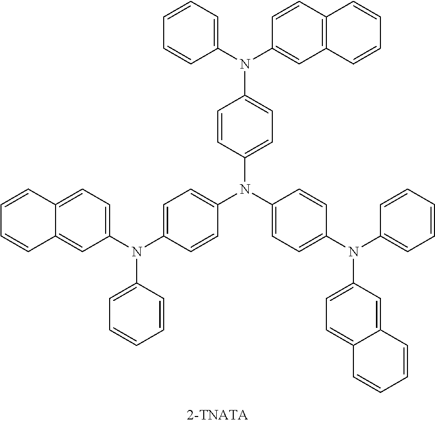 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then 2-TNATA was evaporated by applying a current to the cell to deposit a hole injection layer having a thickness of 600 Å on the ITO substrate.

To another cell of the vacuum deposition apparatus, the following N,N′-bis(α-naphthyl)-N,N′-diphenyl-4,4′-diamine (NPB) was introduced, and evaporated by applying a current to the cell to deposit a hole transfer layer having a thickness of 300 Å on the hole injection layer.

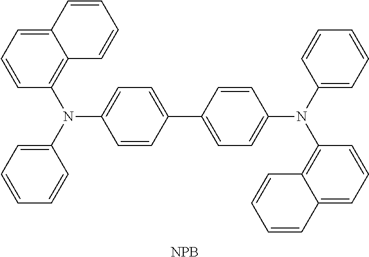 [SMILES: c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transfer layer as above, a blue light emitting material having a structure as below was deposited thereon as a light emitting layer. Specifically, in one side cell in the vacuum deposition apparatus, H1, a blue light emitting host material, was vacuum deposited to a thickness of 200 Å, and D1, a blue light emitting dopant material, was vacuum deposited thereon by 5% with respect to the host material.

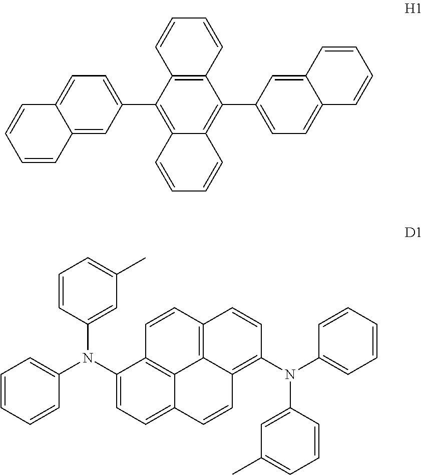

Subsequently, a compound of the following Table 17 was deposited to a thickness of 300 Å as an electron transfer layer.

As an electron injection layer, lithium fluoride (LiF) was deposited to a thickness of 10 Å, and an Al cathode was employed to a thickness of 1,000 Å, and as a result, an OLED was manufactured.

Meanwhile, all the organic compounds required to manufacture the OLED were vacuum sublimation purified under 10⁻⁶ torr to 10⁻⁸ torr by each material to be used in the OLED manufacture.

Results of measuring driving voltage, light emission efficiency, color coordinate (CIE) and lifetime of the blue organic light emitting devices manufactured according to the present disclosure are as shown in the following Table 17.

TABLE 17

| | | | Light | | |
|---|---|---|---|---|---|
| | | Driving | Emission | | Life- |
| | | Voltage | Efficiency | | time |
| | Compound | (V) | (cd/A) | CIE (x, y) | (T95) |
| Comparative | E1 | 5.58 | 5.93 | (0.134, 0.100) | 28 |
| Example 1-1 | | | | | |
| Comparative | E2 | 5.64 | 5.58 | (0.134, 0.105) | 21 |
| Example 1-2 | | | | | |
| Comparative | E3 | 5.60 | 5.67 | (0.134, 0.104) | 20 |
| Example 1-3 | | | | | |
| Comparative | E4 | 5.67 | 5.66 | (0.134, 0.106) | 21 |
| Example 1-4 | | | | | |
| Comparative | E5 | 5.33 | 6.12 | (0.134, 0.099) | 30 |
| Example 1-5 | | | | | |
| Comparative | E6 | 5.23 | 6.08 | (0.134, 0.098) | 31 |
| Example 1-6 | | | | | |
| Example 1 | 1 | 5.44 | 6.47 | (0.134, 0.102) | 36 |
| Example 2 | 5 | 4.47 | 6.87 | (0.134, 0.100) | 40 |
| Example 3 | 12 | 4.67 | 6.64 | (0.129, 0.100) | 38 |
| Example 4 | 13 | 4.82 | 6.55 | (0.130, 0.099) | 36 |
| Example 5 | 19 | 4.44 | 6.97 | (0.134, 0.101) | 40 |
| Example 6 | 24 | 4.61 | 6.89 | (0.134, 0.103) | 40 |
| Example 7 | 26 | 5.35 | 6.30 | (0.134, 0.102) | 33 |
| Example 8 | 29 | 5.62 | 5.95 | (0.134, 0.103) | 42 |
| Example 9 | 35 | 5.40 | 6.12 | (0.134, 0.101) | 39 |
| Example 10 | 40 | 5.50 | 5.89 | (0.134, 0.100) | 41 |
| Example 11 | 43 | 5.44 | 6.01 | (0.134, 0.101) | 36 |
| Example 12 | 45 | 5.34 | 6.58 | (0.134, 0.100) | 45 |
| Example 13 | 51 | 5.38 | 6.93 | (0.134, 0.100) | 43 |
| Example 14 | 54 | 4.91 | 6.32 | (0.134, 0.100) | 41 |
| Example 15 | 57 | 4.98 | 6.44 | (0.134, 0.100) | 40 |
| Example 16 | 64 | 5.62 | 6.38 | (0.134, 0.100) | 35 |
| Example 17 | 66 | 5.66 | 6.04 | (0.131, 0.102) | 30 |
| Example 18 | 70 | 5.40 | 6.49 | (0.134, 0.101) | 31 |
| Example 19 | 76 | 5.60 | 6.22 | (0.129, 0.100) | 29 |
| Example 20 | 78 | 4.70 | 6.01 | (0.134, 0.101) | 36 |
| Example 21 | 82 | 5.40 | 6.12 | (0.134, 0.103) | 44 |
| Example 22 | 87 | 5.60 | 6.21 | (0.134, 0.102) | 43 |
| Example 23 | 89 | 5.45 | 6.22 | (0.134, 0.101) | 37 |
| Example 24 | 93 | 5.39 | 5.95 | (0.134, 0.102) | 33 |
| Example 25 | 99 | 4.96 | 5.95 | (0.134, 0.101) | 42 |
| Example 26 | 103 | 4.91 | 6.13 | (0.134, 0.101) | 39 |
| Example 27 | 106 | 4.91 | 5.85 | (0.134, 0.100) | 41 |
| Example 28 | 112 | 4.98 | 6.38 | (0.134, 0.101) | 42 |
| Example 29 | 114 | 5.62 | 6.20 | (0.134, 0.100) | 45 |
| Example 30 | 119 | 4.72 | 6.12 | (0.134, 0.100) | 43 |
| Example 31 | 122 | 4.91 | 6.21 | (0.134, 0.101) | 41 |
| Example 32 | 128 | 4.96 | 6.10 | (0.134, 0.100) | 36 |
| Example 33 | 130 | 5.30 | 6.20 | (0.134, 0.101) | 40 |
| Example 34 | 135 | 5.22 | 6.03 | (0.134, 0.101) | 43 |
| Example 35 | 137 | 4.90 | 6.81 | (0.134, 0.101) | 56 |
| Example 36 | 144 | 4.88 | 6.82 | (0.134, 0.102) | 57 |
| Example 37 | 147 | 4.74 | 6.75 | (0.134, 0.101) | 51 |
| Example 38 | 151 | 4.81 | 6.82 | (0.134, 0.102) | 53 |
| Example 39 | 154 | 5.16 | 6.20 | (0.134, 0.101) | 38 |
| Example 40 | 157 | 5.15 | 6.42 | (0.134, 0.102) | 39 |
| Example 41 | 161 | 5.31 | 6.30 | (0.134, 0.103) | 37 |
| Example 42 | 166 | 4.82 | 6.35 | (0.134, 0.100) | 50 |
| Example 43 | 171 | 4.91 | 6.12 | (0.134, 0.101) | 42 |
| Example 44 | 176 | 4.98 | 6.51 | (0.134, 0.101) | 39 |
| Example 45 | 180 | 5.62 | 6.21 | (0.134, 0.100) | 41 |
| Example 46 | 183 | 5.39 | 5.95 | (0.134, 0.101) | 34 |
| Example 47 | 187 | 4.96 | 6.88 | (0.134, 0.100) | 45 |
| Example 48 | 192 | 4.91 | 6.93 | (0.134, 0.102) | 43 |
| Example 49 | 196 | 4.76 | 6.95 | (0.134, 0.102) | 50 |
| Example 50 | 199 | 4.77 | 6.90 | (0.134, 0.102) | 51 |
| Example 51 | 204 | 4.98 | 6.05 | (0.134, 0.101) | 34 |
| Example 52 | 207 | 5.22 | 6.03 | (0.134, 0.101) | 43 |
| Example 53 | 210 | 4.82 | 6.84 | (0.134, 0.101) | 52 |
| Example 54 | 215 | 4.84 | 6.97 | (0.134, 0.102) | 51 |
| Example 55 | 218 | 5.38 | 6.88 | (0.134, 0.100) | 41 |
| Example 56 | 222 | 5.60 | 6.93 | (0.134, 0.101) | 32 |
| Example 57 | 228 | 5.45 | 6.95 | (0.134, 0.100) | 45 |
| Example 58 | 230 | 4.91 | 6.93 | (0.134, 0.100) | 43 |
| Example 59 | 235 | 4.91 | 6.95 | (0.134, 0.100) | 41 |
| Example 60 | 239 | 4.98 | 6.23 | (0.134, 0.100) | 40 |
| Example 61 | 244 | 5.62 | 5.98 | (0.134, 0.100) | 36 |
| Example 62 | 248 | 4.72 | 6.51 | (0.134, 0.102) | 48 |
| Example 63 | 252 | 4.91 | 6.93 | (0.134, 0.100) | 43 |
| Example 64 | 256 | 4.91 | 6.95 | (0.134, 0.100) | 41 |
| Example 65 | 260 | 4.98 | 6.26 | (0.134, 0.100) | 40 |
| Example 66 | 262 | 5.62 | 5.98 | (0.134, 0.100) | 33 |
| Example 67 | 265 | 4.91 | 6.32 | (0.134, 0.100) | 41 |
| Example 68 | 271 | 4.98 | 6.44 | (0.134, 0.100) | 40 |
| Example 69 | 274 | 5.62 | 6.38 | (0.134, 0.100) | 35 |
| Example 70 | 279 | 5.44 | 6.34 | (0.134, 0.102) | 36 |
| Example 71 | 283 | 5.62 | 6.20 | (0.134, 0.101) | 39 |
| Example 72 | 286 | 5.62 | 6.22 | (0.134, 0.100) | 47 |
| Example 73 | 289 | 5.62 | 5.98 | (0.134, 0.100) | 33 |
| Example 74 | 294 | 4.72 | 6.55 | (0.134, 0.102) | 48 |
| Example 75 | 297 | 4.72 | 6.20 | (0.134, 0.102) | 43 |
| Example 76 | 304 | 5.40 | 6.12 | (0.134, 0.101) | 39 |
| Example 77 | 307 | 5.44 | 6.21 | (0.134, 0.100) | 41 |
| Example 78 | 310 | 5.39 | 6.20 | (0.134, 0.101) | 36 |
| Example 79 | 315 | 4.96 | 6.88 | (0.134, 0.100) | 45 |
| Example 80 | 318 | 4.91 | 6.93 | (0.134, 0.100) | 43 |
| Example 81 | 323 | 4.96 | 6.88 | (0.134, 0.100) | 45 |
| Example 82 | 326 | 4.91 | 6.93 | (0.134, 0.102) | 43 |
| Example 83 | 330 | 4.98 | 6.22 | (0.134, 0.100) | 40 |
| Example 84 | 335 | 4.72 | 6.53 | (0.134, 0.102) | 48 |
| Example 85 | 339 | 4.72 | 6.53 | (0.134, 0.102) | 48 |

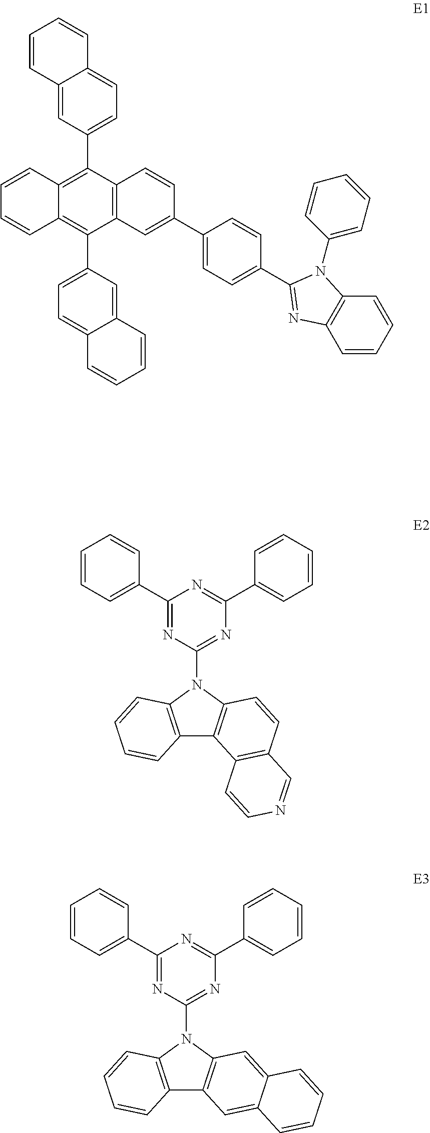 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2nc(-c3ccccc3)nc(-n3c4ccccc4c4c5ccncc5ccc43)n2)cc1 ; c1ccc(-c2nc(-c3ccccc3)nc(-n3c4ccccc4c4cc5ccccc5cc43)n2)cc1]

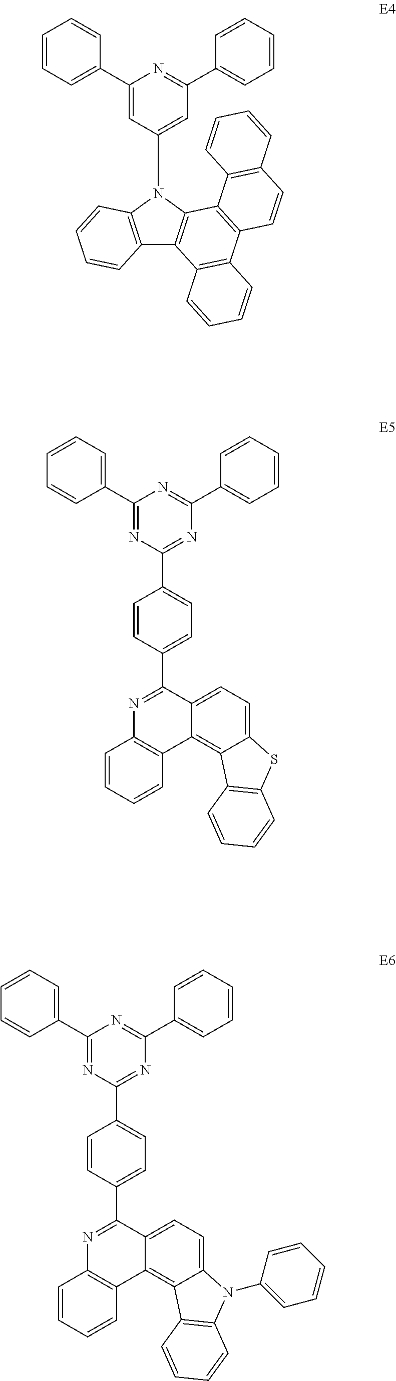 [SMILES: c1ccc(-c2cc(-n3c4ccccc4c4c5ccccc5c5ccc6ccccc6c5c43)cc(-c3ccccc3)n2)cc1 ; c1ccc(-c2nc(-c3ccccc3)nc(-c3ccc(-c4nc5ccccc5c5c4ccc4sc6ccccc6c45)cc3)n2)cc1 ; c1ccc(-c2nc(-c3ccccc3)nc(-c3ccc(-c4nc5ccccc5c5c4ccc4c5c5ccccc5n4-c4ccccc4)cc3)n2)cc1]

As seen from the results of Table 17, the organic light emitting device using the electron transfer layer material of the blue organic light emitting device of the present disclosure had lower driving voltage and significantly improved light emission efficiency and lifetime compared to Comparative Example 1.

### Experimental Example 2

Manufacture of Organic Light Emitting Device

A transparent ITO electrode thin film obtained from glass for an OLED (manufactured by Samsung-Corning Co., Ltd.) was ultrasonic cleaned using trichloroethylene, acetone, ethanol and distilled water consecutively for 5 minutes each, stored in isopropanol, and used.

Next, an ITO substrate was installed in a substrate folder of a vacuum deposition apparatus, and the following 4,4′,4″-tris(N,N-(2-naphthyl)-phenylamino)triphenylamine (2-TNATA) was introduced to a cell in the vacuum deposition apparatus.

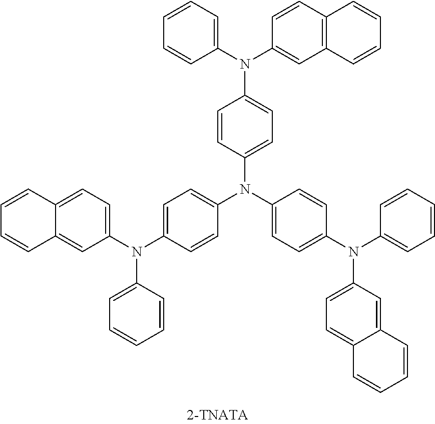 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then 2-TNATA was evaporated by applying a current to the cell to deposit a hole injection layer having a thickness of 600 Å on the ITO substrate.

To another cell of the vacuum deposition apparatus, the following N,N′-bis(α-naphthyl)-N,N′-diphenyl-4,4′-diamine (NPB) was introduced, and evaporated by applying a current to the cell to deposit a hole transfer layer having a thickness of 300 Å on the hole injection layer.

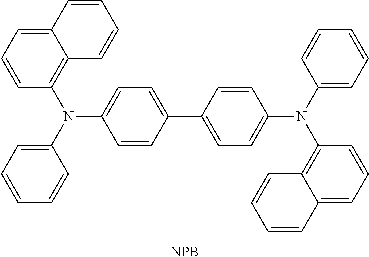 [SMILES: c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transfer layer as above, a blue light emitting material having a structure as below was deposited thereon as a light emitting layer. Specifically, in one side cell in the vacuum deposition apparatus, H1, a blue light emitting host material, was vacuum deposited to a thickness of 200 Å, and D1, a blue light emitting dopant material, was vacuum deposited thereon by 5% with respect to the host material.

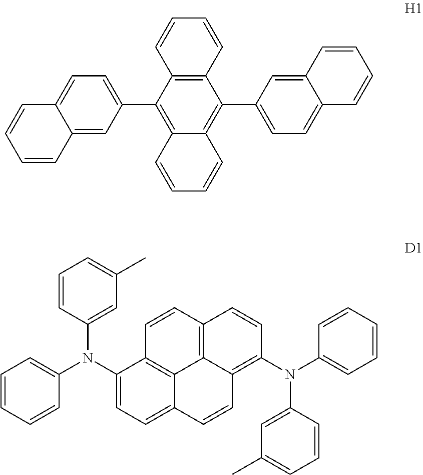

Subsequently, deposition was conducted as follows as an electron transfer layer.

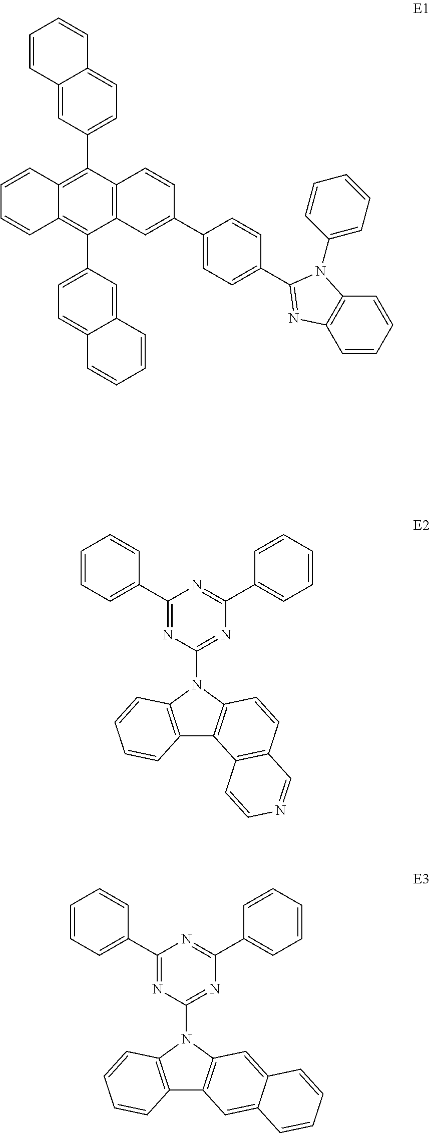 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2nc(-c3ccccc3)nc(-n3c4ccccc4c4c5ccncc5ccc43)n2)cc1 ; c1ccc(-c2nc(-c3ccccc3)nc(-n3c4ccccc4c4cc5ccccc5cc43)n2)cc1]

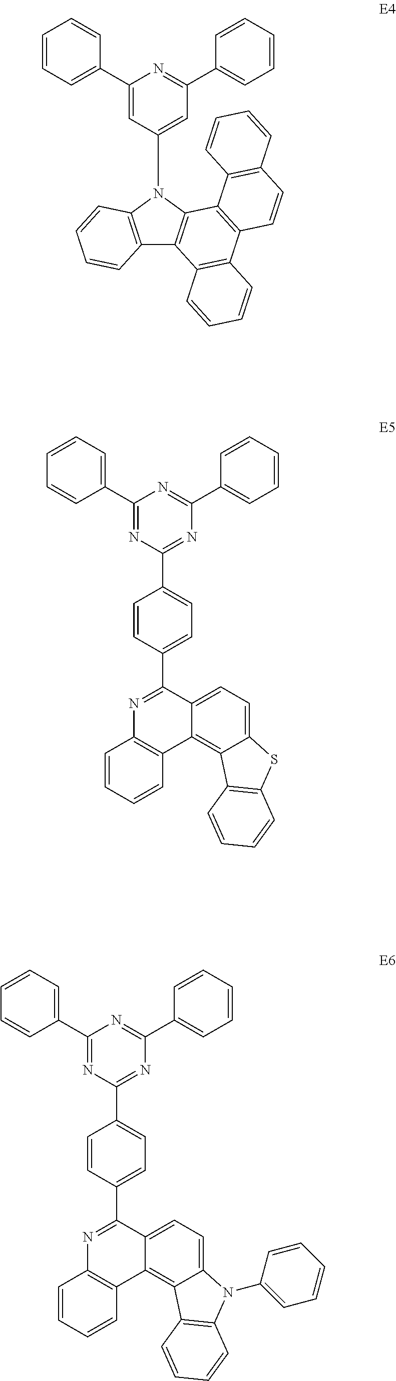 [SMILES: c1ccc(-c2cc(-n3c4ccccc4c4c5ccccc5c5ccc6ccccc6c5c43)cc(-c3ccccc3)n2)cc1 ; c1ccc(-c2nc(-c3ccccc3)nc(-c3ccc(-c4nc5ccccc5c5c4ccc4sc6ccccc6c45)cc3)n2)cc1 ; c1ccc(-c2nc(-c3ccccc3)nc(-c3ccc(-c4nc5ccccc5c5c4ccc4c5c5ccccc5n4-c4ccccc4)cc3)n2)cc1]

An electron transfer layer E1 was formed to a thickness of 250 Å, and then a hole blocking layer was formed on the electron transfer layer using a compound presented in the following Table 18 to a thickness of 50 Å. As an electron injection layer, lithium fluoride (LiF) was deposited to a thickness of 10 Å, and an Al cathode was employed to a thickness of 1,000 Å, and as a result, an OLED was manufactured.

Meanwhile, all the organic compounds required to manufacture the OLED were vacuum sublimation purified under 10⁻⁶ torr to 10⁻⁸ torr by each material to be used in the OLED manufacture.

Results of measuring driving voltage, light emission efficiency, color coordinate (CIE) and lifetime of the blue organic light emitting devices manufactured according to the present disclosure are as shown in the following Table 18.

TABLE 18

| | | | Light | | |
|---|---|---|---|---|---|
| | | Driving | Emission | | Life- |
| | | Voltage | Efficiency | | time |
| | Compound | (V) | (cd/A) | CIE (x, y) | (T95) |
| Comparative | E1 | 5.58 | 5.93 | (0.134, 0.100) | 28 |
| Example 2-1 | | | | | |
| Comparative | E2 | 5.59 | 5.60 | (0.134, 0.104) | 22 |
| Example 2-2 | | | | | |
| Comparative | E3 | 5.63 | 5.67 | (0.134, 0.102) | 21 |
| Example 2-3 | | | | | |
| Comparative | E4 | 5.61 | 5.55 | (0.134, 0.102) | 23 |
| Example 2-4 | | | | | |
| Comparative | E5 | 5.43 | 6.07 | (0.134, 0.100) | 31 |
| Example 2-5 | | | | | |
| Comparative | E6 | 5.35 | 6.11 | (0.134, 0.101) | 30 |
| Example 2-6 | | | | | |
| Example 86 | 1 | 5.44 | 6.47 | (0.134, 0.102) | 36 |
| Example 87 | 5 | 5.41 | 6.44 | (0.134, 0.101) | 43 |
| Example 88 | 12 | 5.34 | 6.38 | (0.134, 0.101) | 39 |
| Example 89 | 13 | 5.28 | 6.28 | (0.134, 0.102) | 32 |
| Example 90 | 19 | 4.57 | 6.74 | (0.128, 0.099) | 36 |
| Example 91 | 24 | 4.62 | 6.72 | (0.133, 0.100) | 37 |
| Example 92 | 26 | 4.55 | 6.85 | (0.134, 0.101) | 39 |
| Example 93 | 29 | 5.62 | 5.95 | (0.134, 0.103) | 42 |
| Example 94 | 35 | 5.40 | 6.12 | (0.134, 0.101) | 39 |
| Example 95 | 40 | 5.50 | 5.89 | (0.134, 0.100) | 41 |
| Example 96 | 43 | 5.44 | 6.01 | (0.134, 0.101) | 36 |
| Example 97 | 45 | 5.34 | 6.58 | (0.134, 0.100) | 45 |
| Example 98 | 51 | 5.38 | 6.93 | (0.134, 0.100) | 43 |
| Example 99 | 54 | 5.63 | 6.11 | (0.131, 0.098) | 28 |
| Example 100 | 57 | 5.66 | 6.04 | (0.131, 0.102) | 30 |
| Example 101 | 64 | 5.40 | 6.49 | (0.134, 0.101) | 31 |
| Example 102 | 66 | 5.60 | 6.22 | (0.129, 0.100) | 29 |
| Example 103 | 70 | 4.70 | 6.01 | (0.134, 0.101) | 36 |
| Example 104 | 76 | 5.44 | 6.34 | (0.134, 0.102) | 36 |
| Example 105 | 78 | 5.62 | 6.20 | (0.134, 0.101) | 39 |
| Example 106 | 82 | 5.40 | 6.12 | (0.134, 0.103) | 44 |
| Example 107 | 87 | 5.60 | 6.21 | (0.134, 0.102) | 43 |
| Example 108 | 89 | 5.45 | 6.22 | (0.134, 0.101) | 37 |
| Example 109 | 93 | 5.39 | 5.95 | (0.134, 0.102) | 33 |
| Example 110 | 99 | 4.96 | 5.95 | (0.134, 0.101) | 42 |
| Example 111 | 103 | 4.91 | 6.13 | (0.134, 0.101) | 39 |
| Example 112 | 106 | 4.91 | 5.85 | (0.134, 0.100) | 41 |
| Example 113 | 112 | 4.98 | 6.38 | (0.134, 0.101) | 42 |
| Example 114 | 114 | 5.62 | 6.20 | (0.134, 0.100) | 45 |
| Example 115 | 119 | 4.72 | 6.12 | (0.134, 0.100) | 43 |
| Example 116 | 122 | 4.96 | 6.10 | (0.134, 0.100) | 36 |
| Example 117 | 128 | 5.30 | 6.20 | (0.134, 0.101) | 40 |
| Example 118 | 130 | 5.22 | 6.03 | (0.134, 0.101) | 43 |
| Example 119 | 135 | 4.90 | 6.81 | (0.134, 0.101) | 56 |
| Example 120 | 137 | 4.88 | 6.82 | (0.134, 0.102) | 57 |
| Example 121 | 144 | 5.44 | 6.41 | (0.134, 0.102) | 47 |
| Example 122 | 147 | 5.34 | 6.44 | (0.134, 0.102) | 36 |
| Example 123 | 151 | 5.38 | 6.38 | (0.134, 0.101) | 39 |
| Example 124 | 154 | 5.38 | 6.20 | (0.134, 0.103) | 40 |
| Example 125 | 157 | 5.39 | 6.62 | (0.134, 0.102) | 43 |
| Example 126 | 161 | 4.96 | 6.21 | (0.134, 0.101) | 37 |
| Example 127 | 166 | 4.91 | 6.22 | (0.134, 0.102) | 33 |
| Example 128 | 171 | 4.91 | 6.12 | (0.134, 0.101) | 42 |
| Example 129 | 176 | 4.98 | 6.51 | (0.134, 0.101) | 39 |
| Example 130 | 180 | 5.62 | 6.21 | (0.134, 0.100) | 41 |
| Example 131 | 183 | 5.39 | 5.95 | (0.134, 0.101) | 34 |
| Example 132 | 187 | 4.96 | 6.88 | (0.134, 0.100) | 45 |
| Example 133 | 192 | 4.91 | 6.93 | (0.134, 0.102) | 43 |
| Example 134 | 196 | 4.90 | 6.71 | (0.134, 0.102) | 50 |
| Example 135 | 199 | 4.72 | 6.98 | (0.134, 0.100) | 51 |
| Example 136 | 204 | 4.80 | 6.89 | (0.134, 0.102) | 58 |
| Example 137 | 207 | 4.76 | 6.95 | (0.134, 0.102) | 50 |
| Example 138 | 210 | 5.44 | 6.63 | (0.134, 0.102) | 48 |
| Example 139 | 215 | 5.34 | 6.13 | (0.134, 0.101) | 39 |
| Example 140 | 218 | 5.38 | 6.88 | (0.134, 0.100) | 41 |
| Example 141 | 222 | 5.60 | 6.93 | (0.134, 0.101) | 32 |
| Example 142 | 228 | 5.45 | 6.95 | (0.134, 0.100) | 45 |
| Example 143 | 230 | 4.91 | 6.93 | (0.134, 0.100) | 43 |
| Example 144 | 235 | 4.91 | 6.95 | (0.134, 0.100) | 41 |
| Example 145 | 239 | 4.98 | 6.23 | (0.134, 0.100) | 40 |
| Example 146 | 244 | 5.62 | 5.98 | (0.134, 0.100) | 36 |
| Example 147 | 248 | 4.72 | 6.51 | (0.134, 0.102) | 48 |
| Example 148 | 252 | 4.91 | 6.93 | (0.134, 0.100) | 43 |
| Example 149 | 256 | 4.91 | 6.95 | (0.134, 0.100) | 41 |
| Example 150 | 260 | 4.98 | 6.26 | (0.134, 0.100) | 40 |
| Example 151 | 262 | 5.62 | 5.98 | (0.134, 0.100) | 33 |
| Example 152 | 265 | 4.98 | 6.05 | (0.134, 0.101) | 34 |
| Example 153 | 271 | 5.22 | 6.03 | (0.134, 0.101) | 43 |
| Example 154 | 274 | 4.82 | 6.84 | (0.134, 0.101) | 52 |
| Example 155 | 279 | 4.91 | 6.93 | (0.134, 0.100) | 43 |
| Example 156 | 283 | 4.98 | 6.95 | (0.134, 0.100) | 41 |
| Example 157 | 286 | 5.62 | 6.22 | (0.134, 0.100) | 47 |
| Example 158 | 289 | 5.62 | 5.98 | (0.134, 0.100) | 33 |
| Example 159 | 294 | 4.72 | 6.55 | (0.134, 0.102) | 48 |
| Example 160 | 297 | 4.72 | 6.20 | (0.134, 0.102) | 43 |
| Example 161 | 304 | 5.40 | 6.12 | (0.134, 0.101) | 39 |
| Example 162 | 307 | 5.44 | 6.21 | (0.134, 0.100) | 41 |
| Example 163 | 310 | 5.39 | 6.20 | (0.134, 0.101) | 36 |
| Example 164 | 315 | 5.45 | 6.21 | (0.134, 0.101) | 37 |
| Example 165 | 318 | 5.44 | 6.22 | (0.134, 0.102) | 34 |
| Example 166 | 323 | 5.50 | 5.89 | (0.134, 0.100) | 41 |
| Example 167 | 326 | 5.44 | 6.01 | (0.134, 0.101) | 36 |
| Example 168 | 330 | 4.91 | 6.32 | (0.134, 0.100) | 41 |
| Example 169 | 335 | 4.72 | 6.53 | (0.134, 0.102) | 48 |
| Example 170 | 339 | 4.72 | 6.53 | (0.134, 0.102) | 48 |

As seen from the results of Table 18, the organic light emitting device using the hole blocking layer material of the blue organic light emitting device of the present disclosure had lower driving voltage and significantly improved light emission efficiency and lifetime compared to Comparative Example 2.

### Experimental Example 3

Manufacture of Organic Light Emitting Device

A glass substrate on which ITO was coated as a thin film to a thickness of 1500 Å was cleaned with distilled water ultrasonic waves. After the cleaning with distilled water was finished, the substrate was ultrasonic cleaned with solvents such as acetone, methanol and isopropyl alcohol, then dried, and ultraviolet ozone (WO) treated for 5 minutes using UV in an ultraviolet (UV) cleaner. After that, the substrate was transferred to a plasma cleaner (PT), and plasma treatment was performed under vacuum for ITO work function and residual film removal, and the substrate was transferred to a thermal deposition apparatus for organic deposition.

On the transparent ITO electrode (anode), organic materials were formed in a 2-stack white organic light emitting device (WOLED) structure. As for the first stack, TAPC was thermal vacuum deposited first to a thickness of 300 Å to form a hole transfer layer. After forming the hole transfer layer, a light emitting layer was thermal vacuum deposited thereon as follows. As the light emitting layer, TCz1, a host, was 8% doped with FIrpic, a blue phosphorescent dopant, and deposited to 300 Å. After forming an electron transfer layer to 400 Å using TmPyPB, the compound described in the following Table 19 was 20% doped with Cs₂CO₃ to form a charge generation layer to 100 Å.

As for the second stack, MoO₃ was thermal vacuum deposited first to a thickness of 50 Å to form a hole injection layer. A hole transfer layer, a common layer, was formed to 100 Å by 20% doping MoO₃ to TAPC and then depositing TAPC to 300 Å. A light emitting layer was formed by 8% doping Ir(ppy)₃, a green phosphorescent dopant, to TCzl, a host, and depositing the result to 300 Å, and then an electron transfer layer was formed to 600 Å using TmPyPB. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured.

Meanwhile, all the organic compounds required to manufacture the OLED were vacuum sublimation purified under 10⁻⁶ torr to 10⁻⁸ torr for each material to be used in the OLED manufacture.

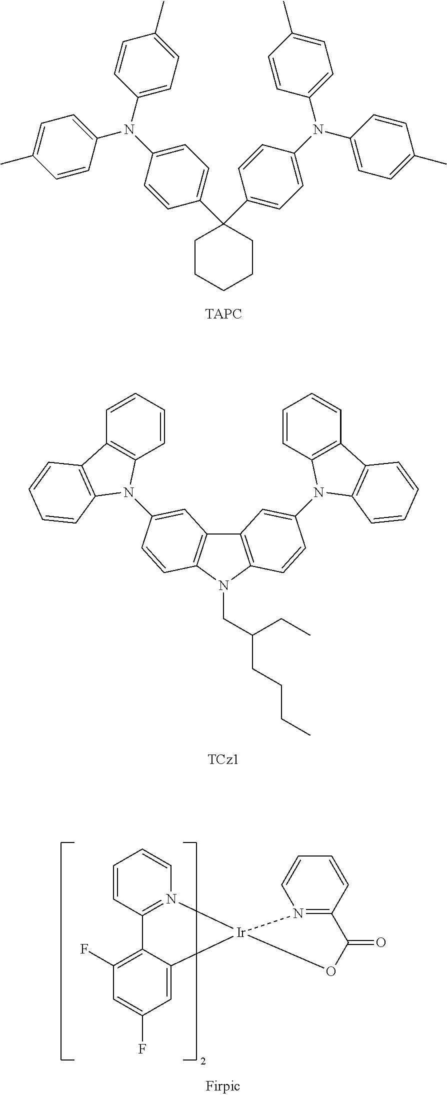

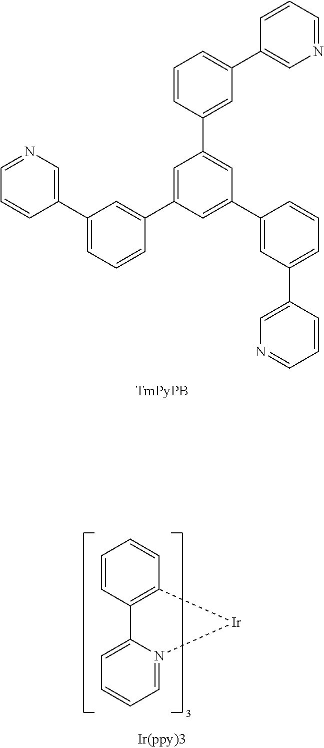

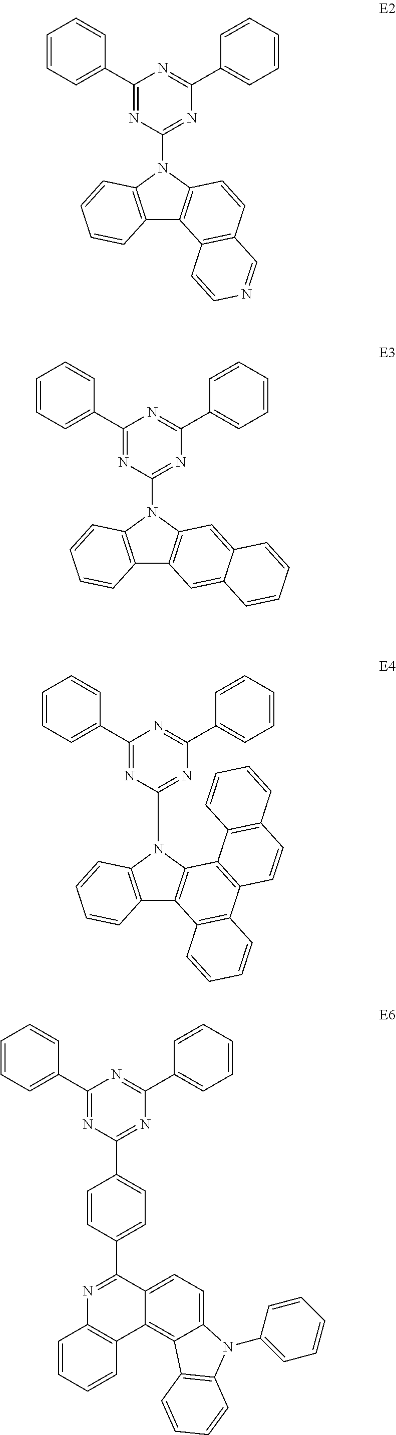

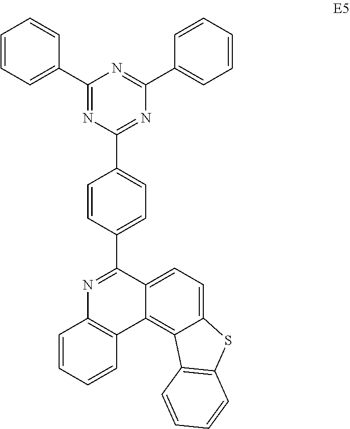 [SMILES: c1ccc(-c2nc(-c3ccccc3)nc(-c3ccc(-c4nc5ccccc5c5c4ccc4sc6ccccc6c45)cc3)n2)cc1]

Results of measuring driving voltage, light emission efficiency, color coordinate (CIE) and lifetime of the white organic light emitting devices manufactured according to the present disclosure are as shown in Table 19.

TABLE 19

| | | | Light | | |
|---|---|---|---|---|---|
| | | Driving | Emission | | Life- |
| | | Voltage | Efficiency | | time |
| | Compound | (V) | (cd/A) | CIE (x, y) | (T95) |
| Comparative | TmPyPB | 8.57 | 43.11 | (0.201, 0.398) | 9 |
| Example 3-1 | | | | | |
| Comparative | E2 | 8.76 | 42.33 | (0.201, 0.412) | 11 |
| Example 3-2 | | | | | |
| Comparative | E3 | 8.63 | 45.61 | (0.201, 0.408) | 8 |
| Example 3-3 | | | | | |
| Comparative | E4 | 8.80 | 44.89 | (0.201, 0.417) | 9 |
| Example 3-4 | | | | | |
| Comparative | E5 | 8.23 | 50.11 | (0.221, 0.428) | 18 |
| Example 3-5 | | | | | |
| Comparative | E6 | 8.33 | 50.23 | (0.223, 0.428) | 19 |
| Example 3-6 | | | | | |
| Example 171 | 20 | 7.29 | 65.55 | (0.220, 0.432) | 53 |
| Example 172 | 40 | 7.31 | 66.32 | (0.221, 0.433) | 51 |
| Example 173 | 60 | 7.05 | 67.93 | (0.221, 0.428) | 50 |
| Example 174 | 80 | 7.06 | 69.82 | (0.221, 0.440) | 42 |
| Example 175 | 100 | 7.01 | 69.45 | (0.220, 0.430) | 40 |
| Example 176 | 120 | 7.12 | 68.55 | (0.215, 0.422) | 44 |
| Example 177 | 140 | 7.08 | 68.21 | (0.214, 0.422) | 45 |
| Example 178 | 160 | 7.07 | 67.44 | (0.212, 0.417) | 40 |
| Example 179 | 180 | 7.09 | 68.01 | (0.211, 0.422) | 42 |
| Example 180 | 200 | 7.11 | 69.44 | (0.223, 0.428) | 41 |
| Example 181 | 220 | 7.02 | 68.08 | (0.222, 0.430) | 42 |
| Example 182 | 240 | 7.04 | 69.11 | (0.231, 0.434) | 40 |
| Example 183 | 260 | 7.08 | 69.45 | (0.220, 0.430) | 40 |
| Example 184 | 280 | 7.12 | 68.58 | (0.216, 0.426) | 35 |
| Example 185 | 300 | 7.09 | 68.01 | (0.211, 0.422) | 42 |
| Example 186 | 320 | 7.11 | 69.44 | (0.223, 0.428) | 41 |
| Example 187 | 340 | 7.02 | 68.08 | (0.222, 0.430) | 42 |

As seen from the results of Table 19, the organic electroluminescent device using the charge generation layer material of the white organic electroluminescent device of the present disclosure had lower driving voltage and significantly improved light emission efficiency compared to Comparative Example 3.

From the results of Tables 17 to 19, it was identified that the compound represented by Chemical Formula 1 had a substituent of -(L)m-(Z)n on one side benzene ring of the carbazole group, and the π-conjugation structure of the compound of Chemical Formula 1 did not continue from the carbazole group to the fused quinoline group. As a result, the π-conjugation structure of the compound represented by Chemical Formula 1 was disconnected widening a bandgap of a HOMO level and a LUMO level, and the T1 value further increased increasing an effect of locking excitons in the light emitting layer. In addition, by decreasing the HOMO level, holes of the light emitting layer were blocked, and the compound was able to be used as a compound of a hole blocking layer.

1. A heterocyclic compound represented by the following Chemical Formula 1: 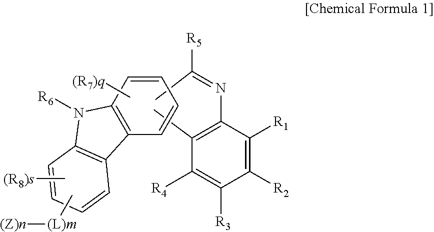
wherein, in Chemical Formula 1,
R₁ to R₄, R₇ and R₈ are the same as or different from each other, and each independently selected from the group consisting of hydrogen; deuterium; a substituted or unsubstituted alkyl group; a substituted or unsubstituted aryl group; a substituted or unsubstituted heteroaryl group; —SiRR′R″; —P(═O)RR′; and an amine group unsubstituted or substituted with a substituted or unsubstituted alkyl group, a substituted or unsubstituted aryl group, or a substituted or unsubstituted heteroaryl group, or two or more groups adjacent to each other bond to each other to form a substituted or unsubstituted aromatic hydrocarbon ring or a substituted or unsubstituted heteroring;
R₅ and R₆ are the same as or different from each other, and each independently selected from the group consisting of a substituted or unsubstituted alkyl group; a substituted or unsubstituted aryl group; a substituted or unsubstituted heteroaryl group; and an amine group unsubstituted or substituted with a substituted or unsubstituted alkyl group, a substituted or unsubstituted aryl group, or a substituted or unsubstituted heteroaryl group;
L is a direct bond; a substituted or unsubstituted arylene group; or a substituted or unsubstituted heteroarylene group;
Z is selected from the group consisting of deuterium; —CN; a substituted or unsubstituted alkyl group; a substituted or unsubstituted aryl group; a substituted or unsubstituted heteroaryl group; —SiRR′R″; —P(═O)RR′; and an amine group unsubstituted or substituted with a substituted or unsubstituted alkyl group, a substituted or unsubstituted aryl group, or a substituted or unsubstituted heteroaryl group;
R, R′ and R″ are the same as or different from each other, and each independently hydrogen; a substituted or unsubstituted alkyl group; a substituted or unsubstituted aryl group; or a substituted or unsubstituted heteroaryl group;
m is an integer of 0 to 5;
n is an integer of 1 to 6;
q is an integer of 0 to 2; and
s is an integer of 0 to 3.

2. The heterocyclic compound of claim 1, wherein the “substituted or unsubstituted” means being substituted with one or more substituents selected from the group consisting of C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine, or being unsubstituted, or being substituted with a substituent linking two or more substituents selected from among the substituents illustrated above, or being unsubstituted; and
R, R′ and R″ have the same definitions as in Chemical Formula 1.

3. The heterocyclic compound of claim 1, wherein Chemical Formula 1 is represented by any one of the following Chemical Formula 2 to Chemical Formula 7: 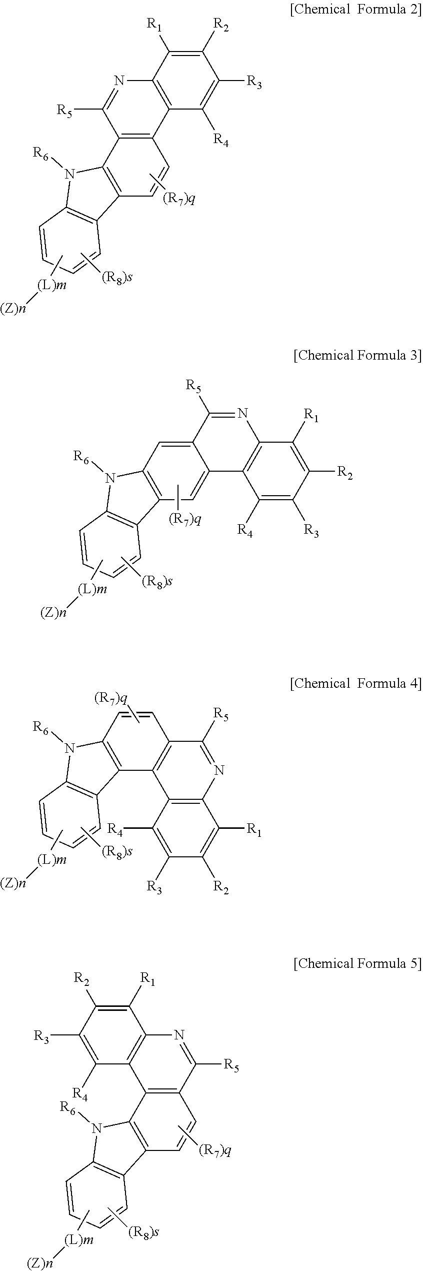 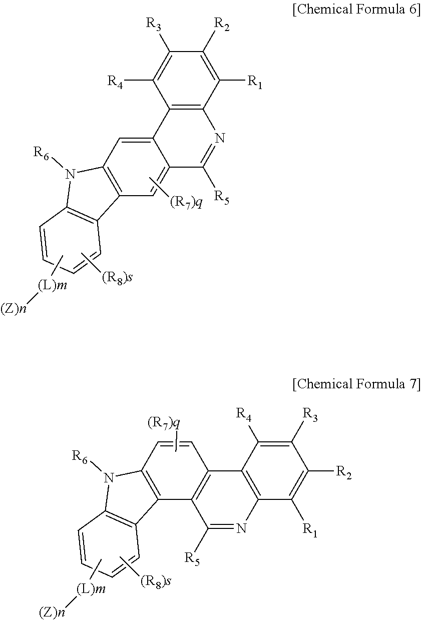
in Chemical Formulae 2 to 7,
R₁ to R₈, L, Z, m, n, s and q have the same definitions as in Chemical Formula 1.

4. The heterocyclic compound of claim 1, wherein Chemical Formula 1 is represented by any one of the following Chemical Formulae 8 to 11: 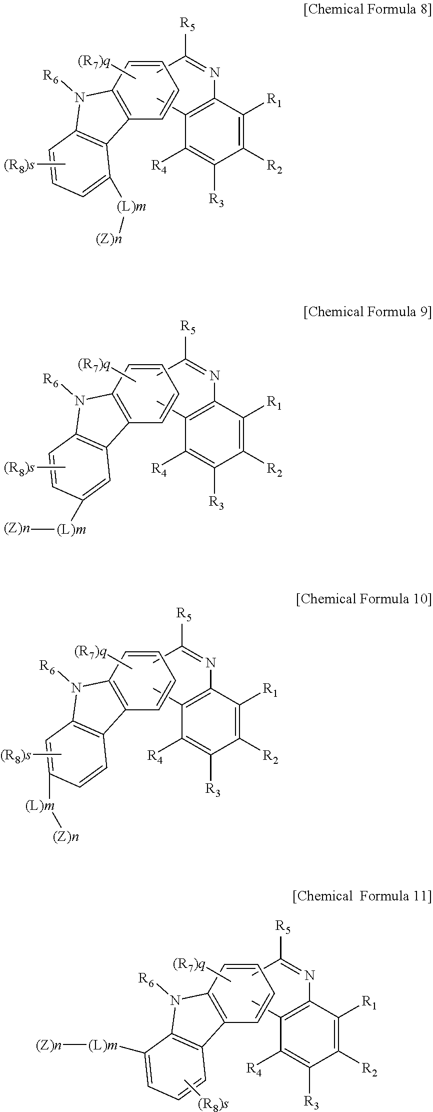
in Chemical Formulae 8 to 11,
R₁ to R₈, L, Z, m, n, s and q have the same definitions as in Chemical Formula 1.

5. The heterocyclic compound of claim 1, wherein R₅ and R₆ are the same as or different from each other, and each independently a substituted or unsubstituted C6 to C40 aryl group.

6. The heterocyclic compound of claim 1, wherein R₁ to R₄, R₇ and R₈ are hydrogen.

7. The heterocyclic compound of claim 1, wherein Chemical Formula 1 is represented by any one of the following compounds:

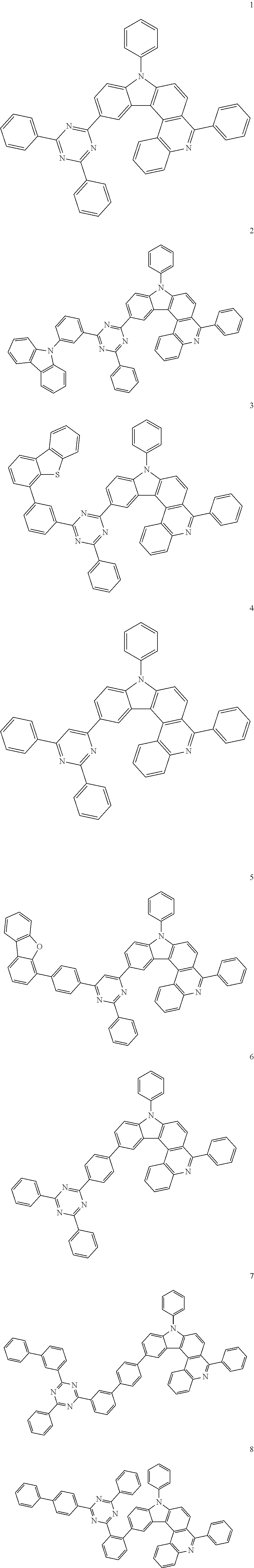

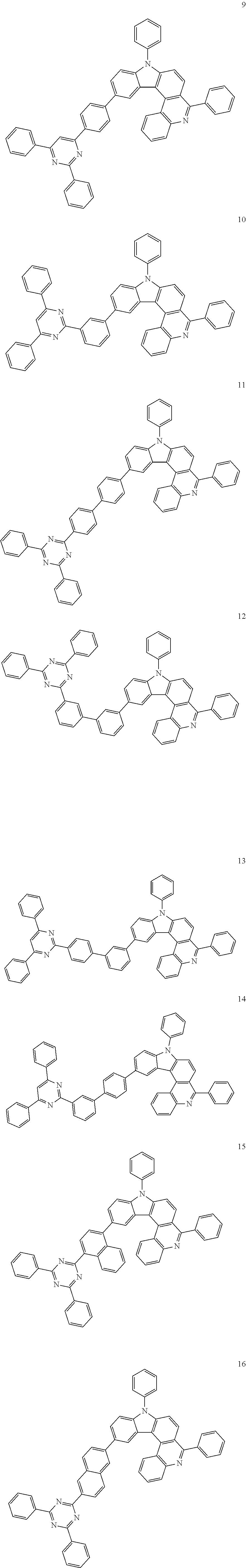

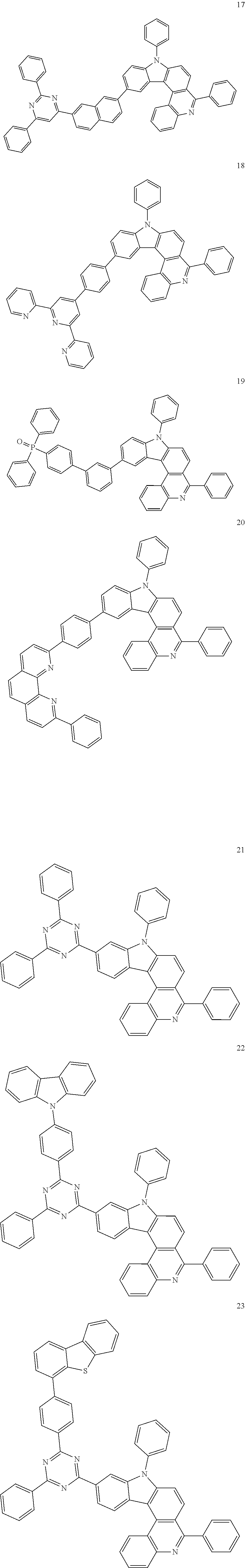

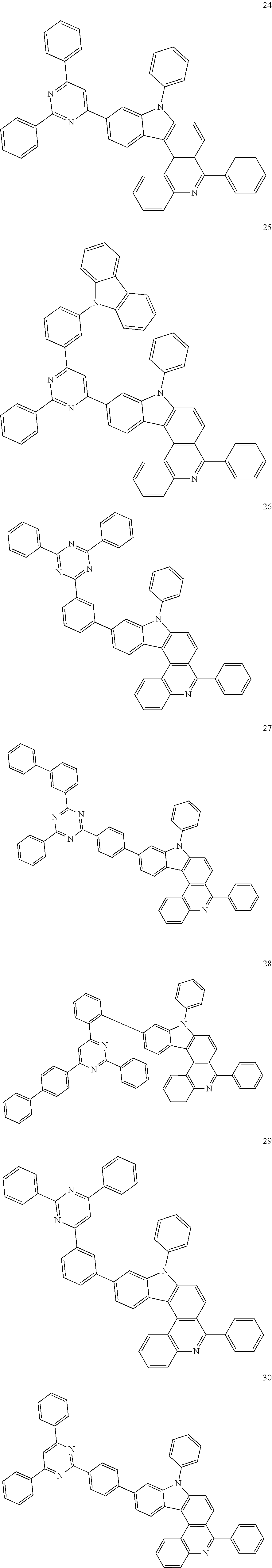

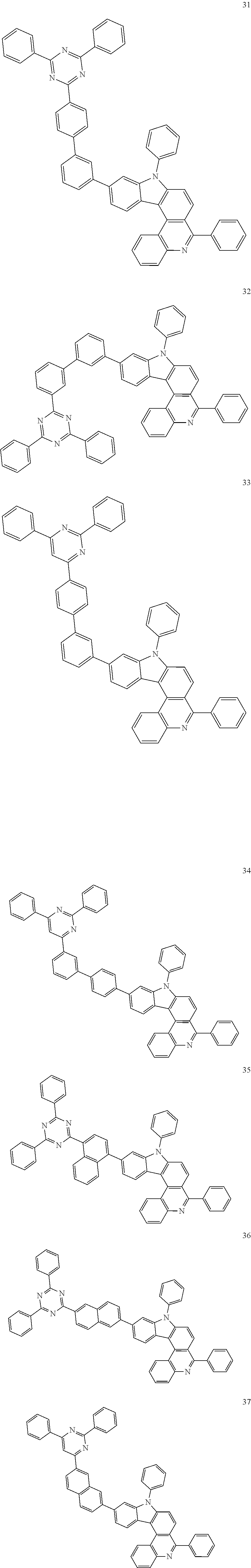

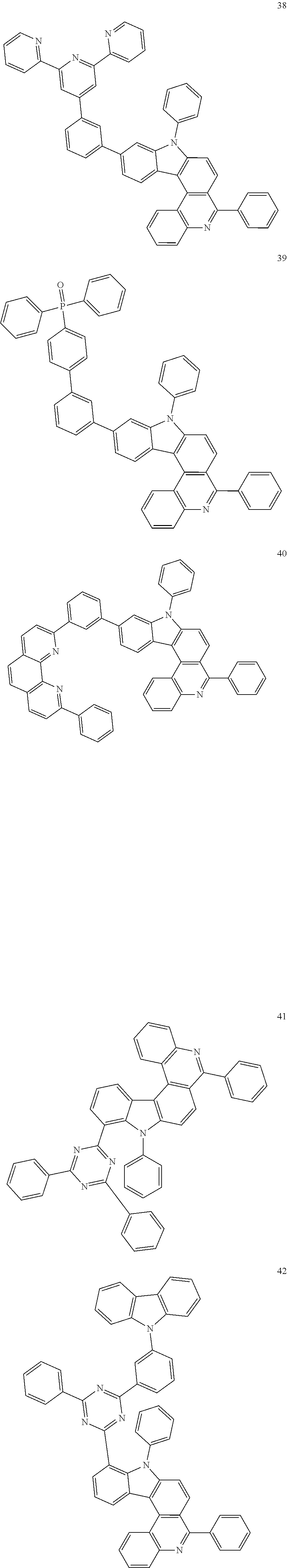

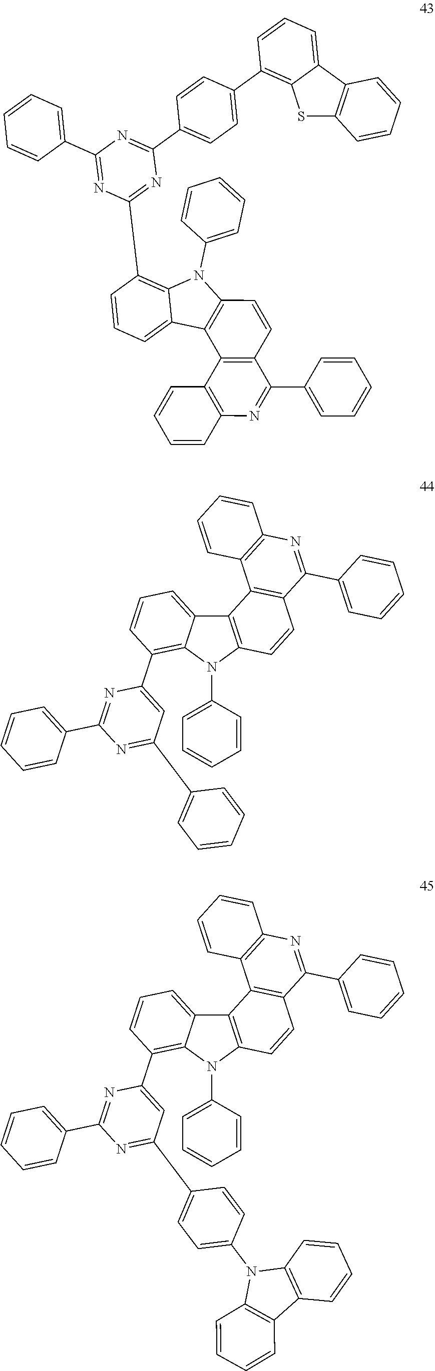 [SMILES: c1ccc(-c2nc(-c3ccc(-c4cccc5c4sc4ccccc45)cc3)nc(-c3cccc4c5c6c(ccc5n(-c5ccccc5)c34)c(-c3ccccc3)nc3ccccc36)n2)cc1 ; c1ccc(-c2cc(-c3cccc4c5c6c(ccc5n(-c5ccccc5)c34)c(-c3ccccc3)nc3ccccc36)nc(-c3ccccc3)n2)cc1 ; c1ccc(-c2nc(-c3ccc(-n4c5ccccc5c5ccccc54)cc3)cc(-c3cccc4c5c6c(ccc5n(-c5ccccc5)c34)c(-c3ccccc3)nc3ccccc36)n2)cc1]

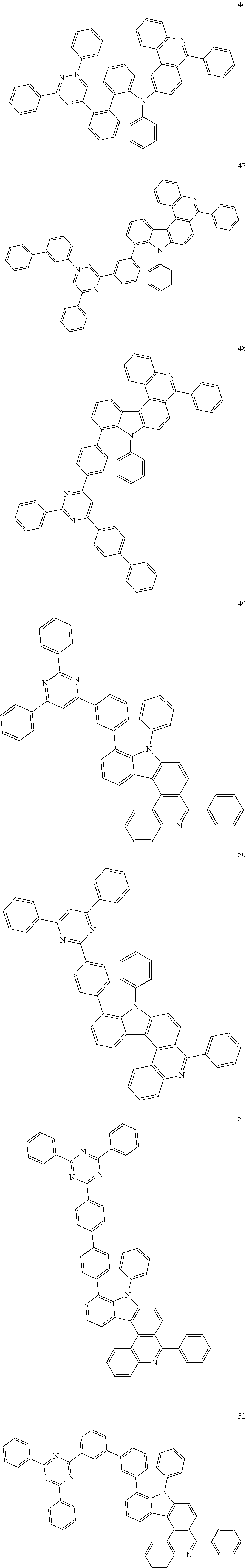

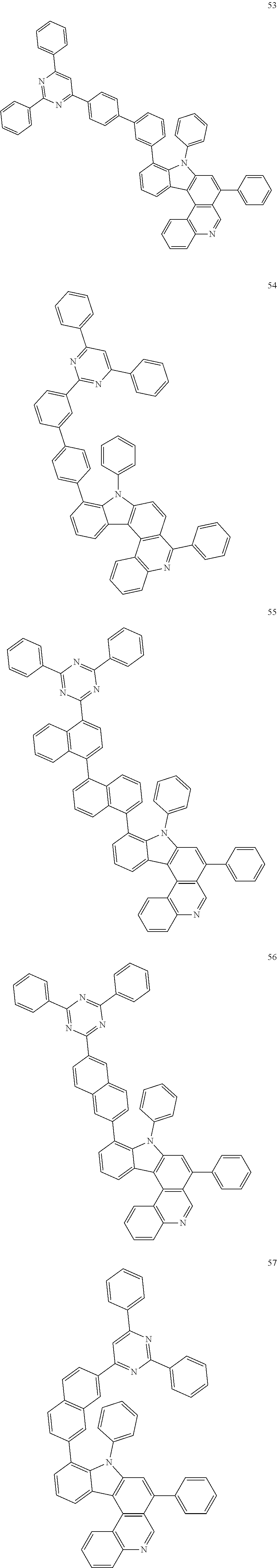

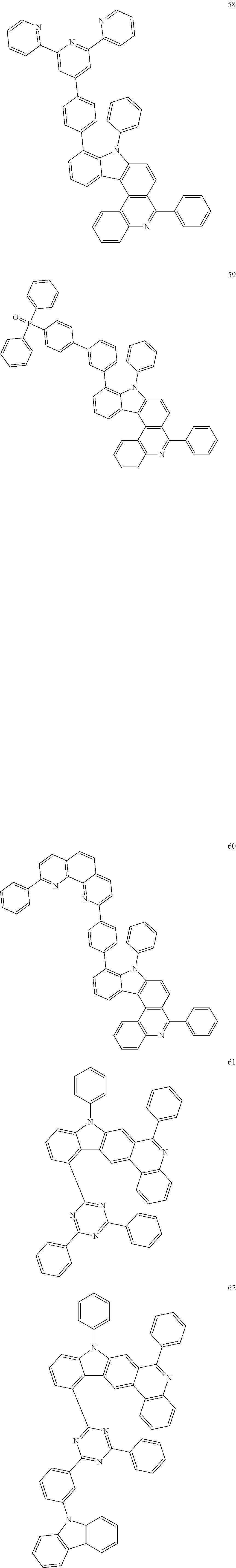

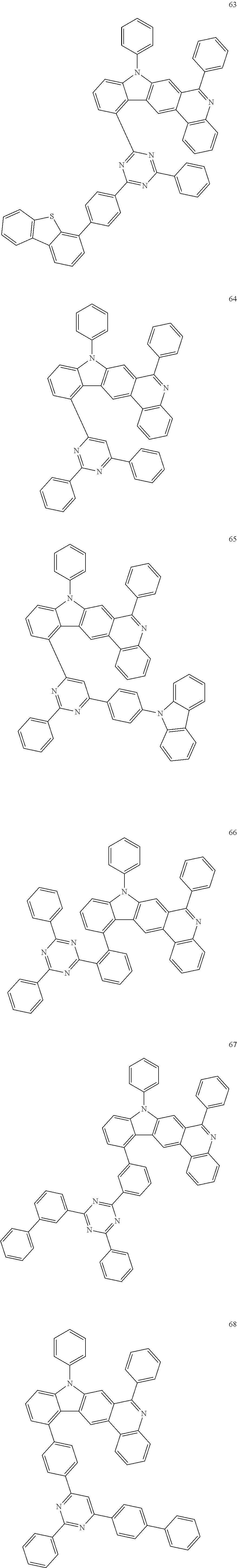

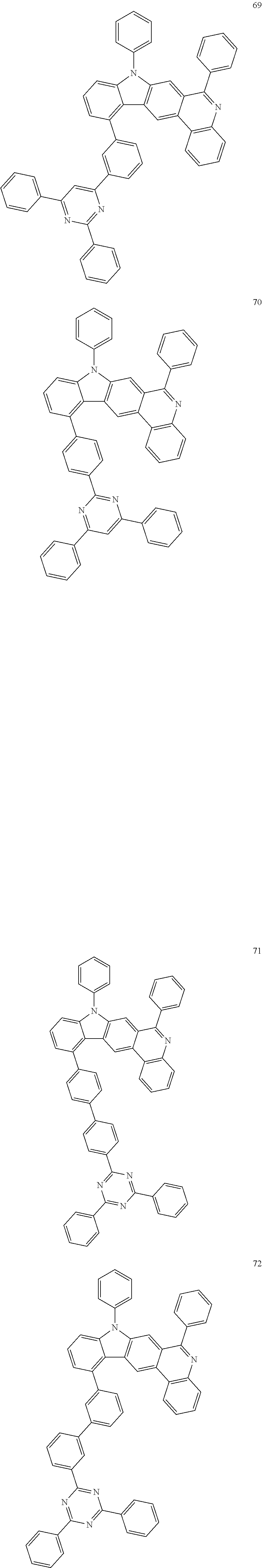 [SMILES: c1ccc(-c2cc(-c3cccc(-c4cccc5c4c4cc6c(cc4n5-c4ccccc4)c(-c4ccccc4)nc4ccccc46)c3)nc(-c3ccccc3)n2)cc1 ; c1ccc(-c2cc(-c3ccccc3)nc(-c3ccc(-c4cccc5c4c4cc6c(cc4n5-c4ccccc4)c(-c4ccccc4)nc4ccccc46)cc3)n2)cc1 ; c1ccc(-c2nc(-c3ccccc3)nc(-c3ccc(-c4ccc(-c5cccc6c5c5cc7c(cc5n6-c5ccccc5)c(-c5ccccc5)nc5ccccc57)cc4)cc3)n2)cc1 ; c1ccc(-c2nc(-c3ccccc3)nc(-c3cccc(-c4cccc(-c5cccc6c5c5cc7c(cc5n6-c5ccccc5)c(-c5ccccc5)nc5ccccc57)c4)c3)n2)cc1]

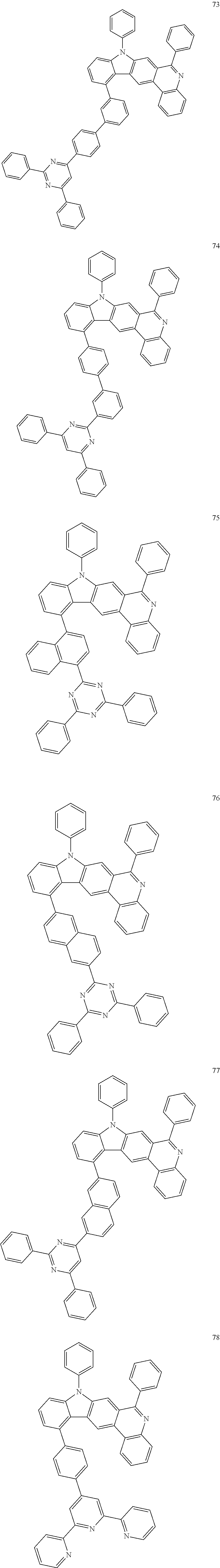

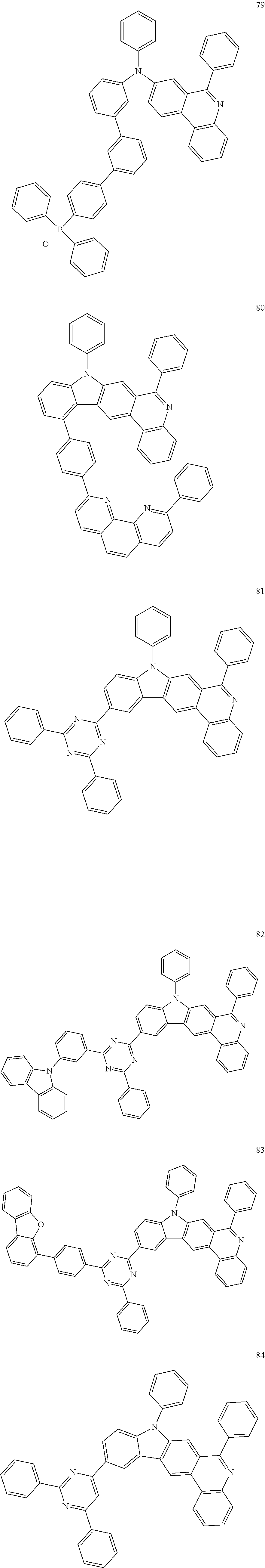

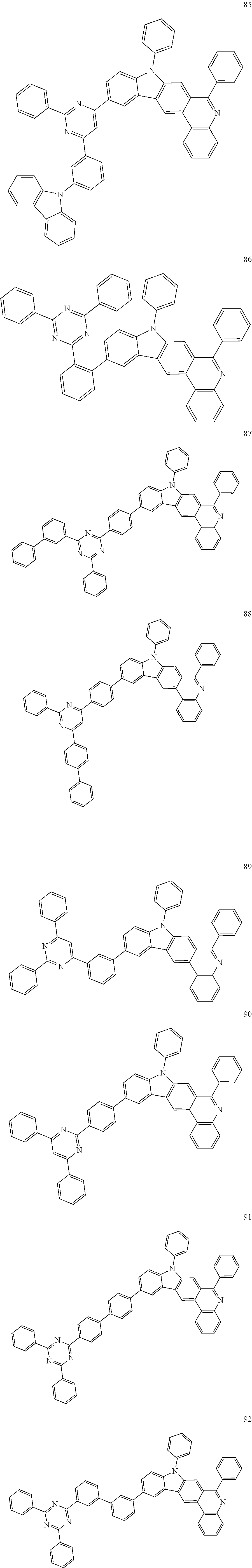

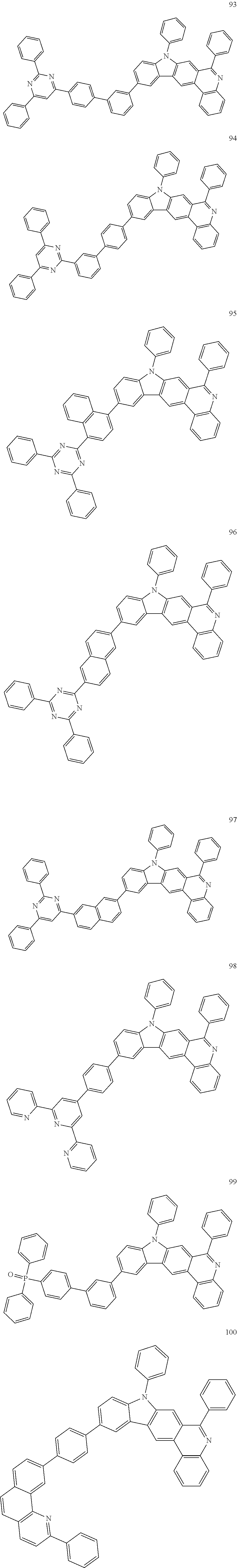

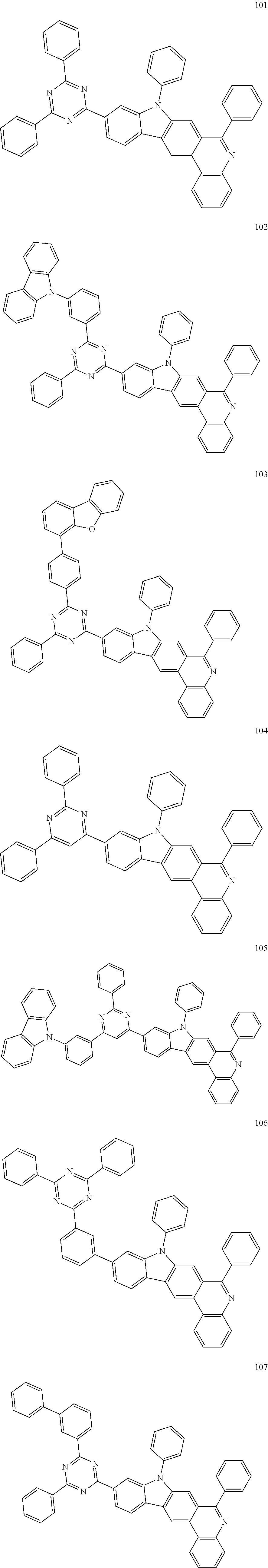

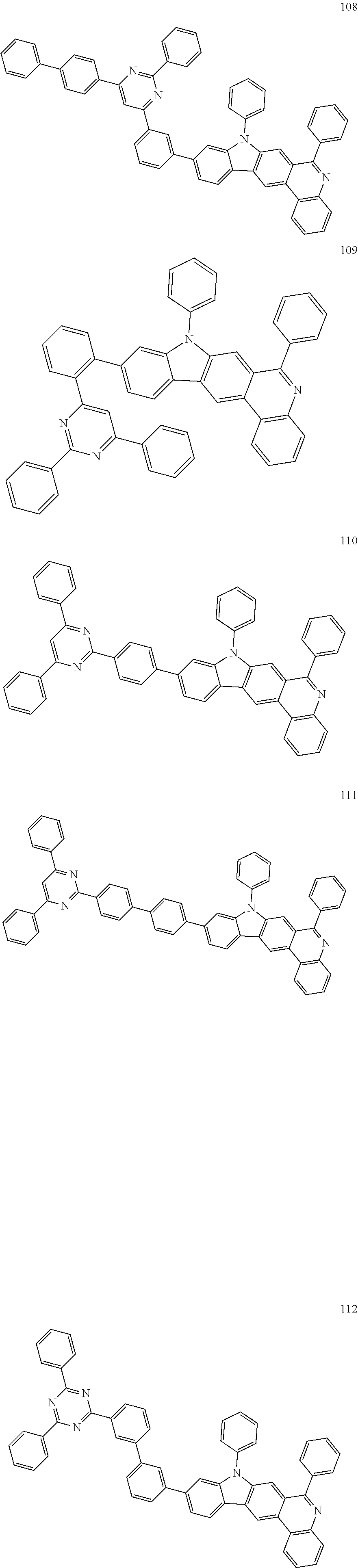

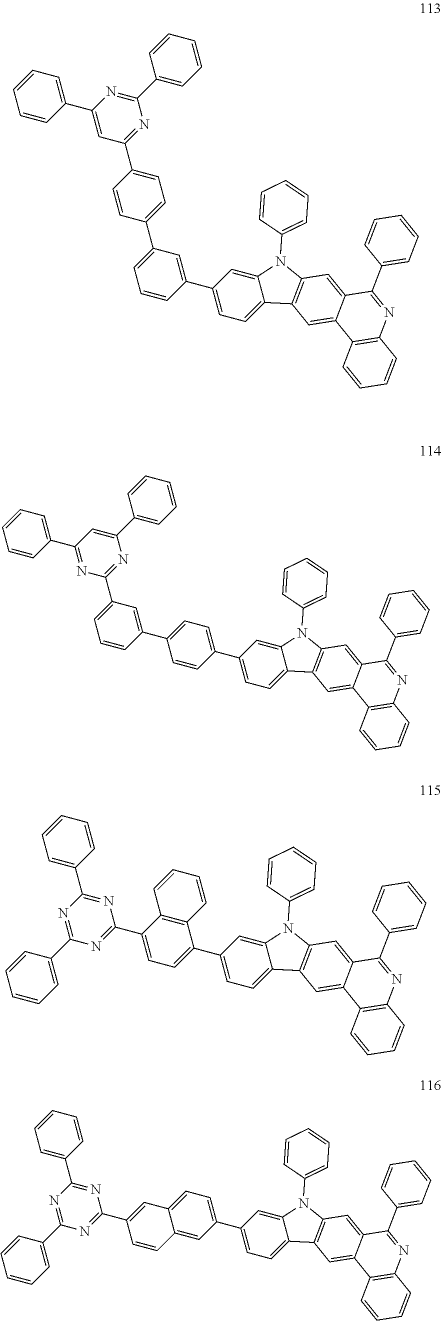 [SMILES: c1ccc(-c2cc(-c3ccc(-c4cccc(-c5ccc6c7cc8c(cc7n(-c7ccccc7)c6c5)c(-c5ccccc5)nc5ccccc58)c4)cc3)nc(-c3ccccc3)n2)cc1 ; c1ccc(-c2cc(-c3ccccc3)nc(-c3cccc(-c4ccc(-c5ccc6c7cc8c(cc7n(-c7ccccc7)c6c5)c(-c5ccccc5)nc5ccccc58)cc4)c3)n2)cc1 ; c1ccc(-c2nc(-c3ccccc3)nc(-c3ccc(-c4ccc5c6cc7c(cc6n(-c6ccccc6)c5c4)c(-c4ccccc4)nc4ccccc47)c4ccccc34)n2)cc1 ; c1ccc(-c2nc(-c3ccccc3)nc(-c3ccc4cc(-c5ccc6c7cc8c(cc7n(-c7ccccc7)c6c5)c(-c5ccccc5)nc5ccccc58)ccc4c3)n2)cc1]

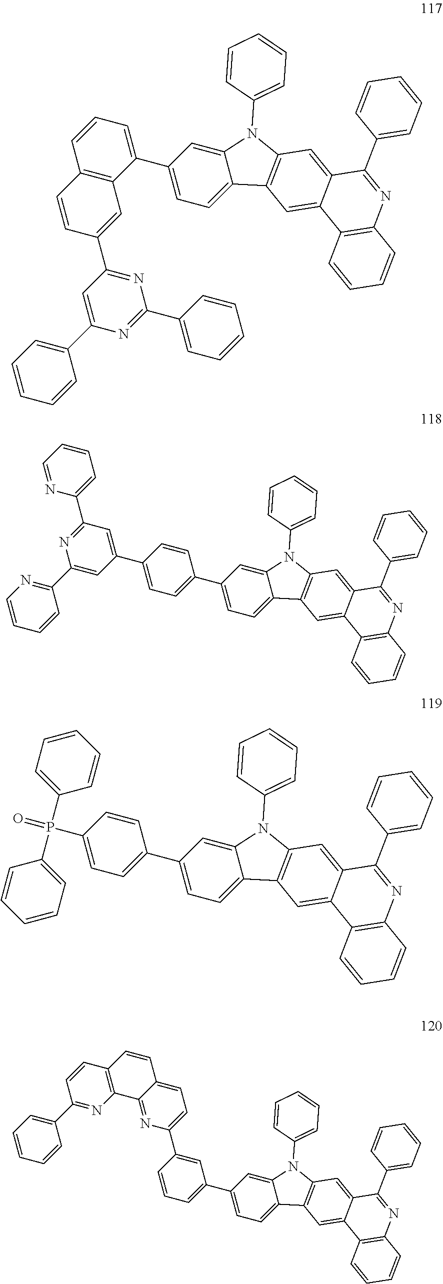 [SMILES: c1ccc(-c2cc(-c3ccc4cccc(-c5ccc6c7cc8c(cc7n(-c7ccccc7)c6c5)c(-c5ccccc5)nc5ccccc58)c4c3)nc(-c3ccccc3)n2)cc1 ; c1ccc(-c2nc3ccccc3c3cc4c5ccc(-c6ccc(-c7cc(-c8ccccn8)nc(-c8ccccn8)c7)cc6)cc5n(-c5ccccc5)c4cc23)cc1 ; O=P(c1ccccc1)(c1ccccc1)c1ccc(-c2ccc3c4cc5c(cc4n(-c4ccccc4)c3c2)c(-c2ccccc2)nc2ccccc25)cc1 ; c1ccc(-c2ccc3ccc4ccc(-c5cccc(-c6ccc7c8cc9c(cc8n(-c8ccccc8)c7c6)c(-c6ccccc6)nc6ccccc69)c5)nc4c3n2)cc1]

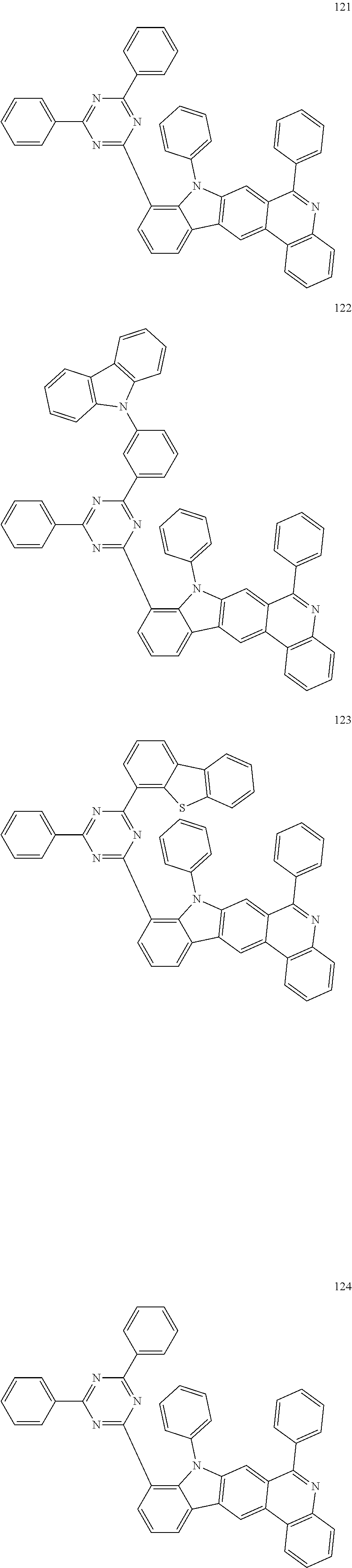 [SMILES: c1ccc(-c2nc(-c3ccccc3)nc(-c3cccc4c5cc6c(cc5n(-c5ccccc5)c34)c(-c3ccccc3)nc3ccccc36)n2)cc1 ; c1ccc(-c2nc(-c3cccc(-n4c5ccccc5c5ccccc54)c3)nc(-c3cccc4c5cc6c(cc5n(-c5ccccc5)c34)c(-c3ccccc3)nc3ccccc36)n2)cc1 ; c1ccc(-c2nc(-c3cccc4c3sc3ccccc34)nc(-c3cccc4c5cc6c(cc5n(-c5ccccc5)c34)c(-c3ccccc3)nc3ccccc36)n2)cc1 ; c1ccc(-c2nc(-c3ccccc3)nc(-c3cccc4c5cc6c(cc5n(-c5ccccc5)c34)c(-c3ccccc3)nc3ccccc36)n2)cc1]

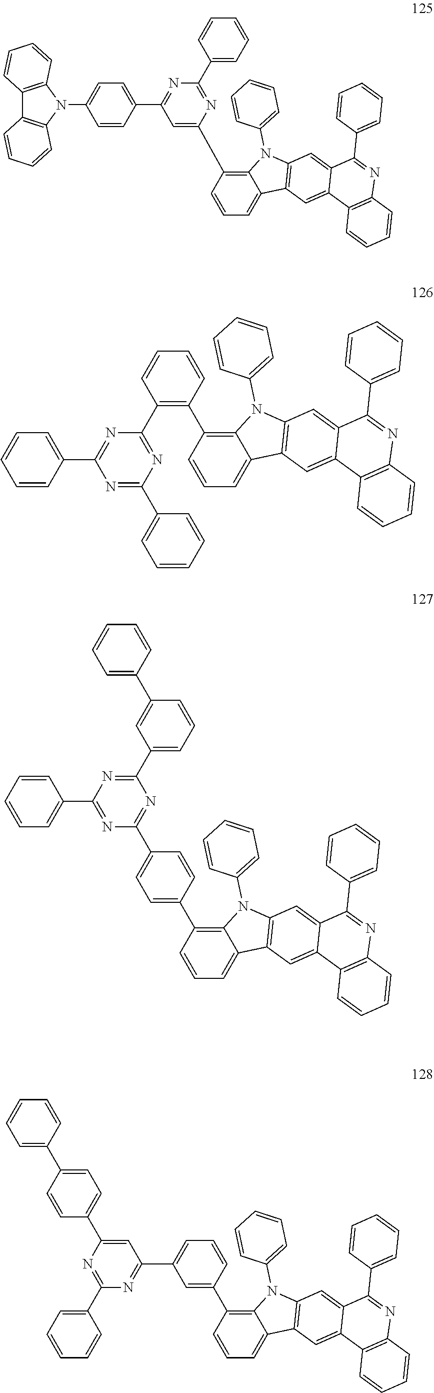 [SMILES: c1ccc(-c2nc(-c3ccc(-n4c5ccccc5c5ccccc54)cc3)cc(-c3cccc4c5cc6c(cc5n(-c5ccccc5)c34)c(-c3ccccc3)nc3ccccc36)n2)cc1 ; c1ccc(-c2nc(-c3ccccc3)nc(-c3ccccc3-c3cccc4c5cc6c(cc5n(-c5ccccc5)c34)c(-c3ccccc3)nc3ccccc36)n2)cc1 ; c1ccc(-c2cccc(-c3nc(-c4ccccc4)nc(-c4ccc(-c5cccc6c7cc8c(cc7n(-c7ccccc7)c56)c(-c5ccccc5)nc5ccccc58)cc4)n3)c2)cc1 ; c1ccc(-c2ccc(-c3cc(-c4cccc(-c5cccc6c7cc8c(cc7n(-c7ccccc7)c56)c(-c5ccccc5)nc5ccccc58)c4)nc(-c4ccccc4)n3)cc2)cc1]

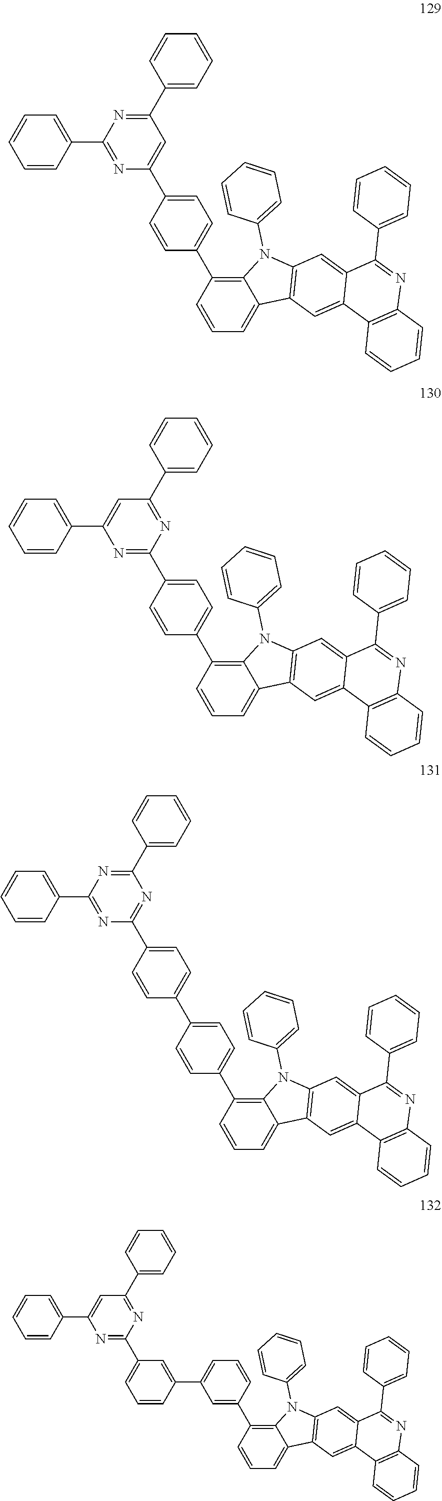 [SMILES: c1ccc(-c2cc(-c3ccc(-c4cccc5c6cc7c(cc6n(-c6ccccc6)c45)c(-c4ccccc4)nc4ccccc47)cc3)nc(-c3ccccc3)n2)cc1 ; c1ccc(-c2cc(-c3ccccc3)nc(-c3ccc(-c4cccc5c6cc7c(cc6n(-c6ccccc6)c45)c(-c4ccccc4)nc4ccccc47)cc3)n2)cc1 ; c1ccc(-c2nc(-c3ccccc3)nc(-c3ccc(-c4ccc(-c5cccc6c7cc8c(cc7n(-c7ccccc7)c56)c(-c5ccccc5)nc5ccccc58)cc4)cc3)n2)cc1 ; c1ccc(-c2cc(-c3ccccc3)nc(-c3cccc(-c4cccc(-c5cccc6c7cc8c(cc7n(-c7ccccc7)c56)c(-c5ccccc5)nc5ccccc58)c4)c3)n2)cc1]

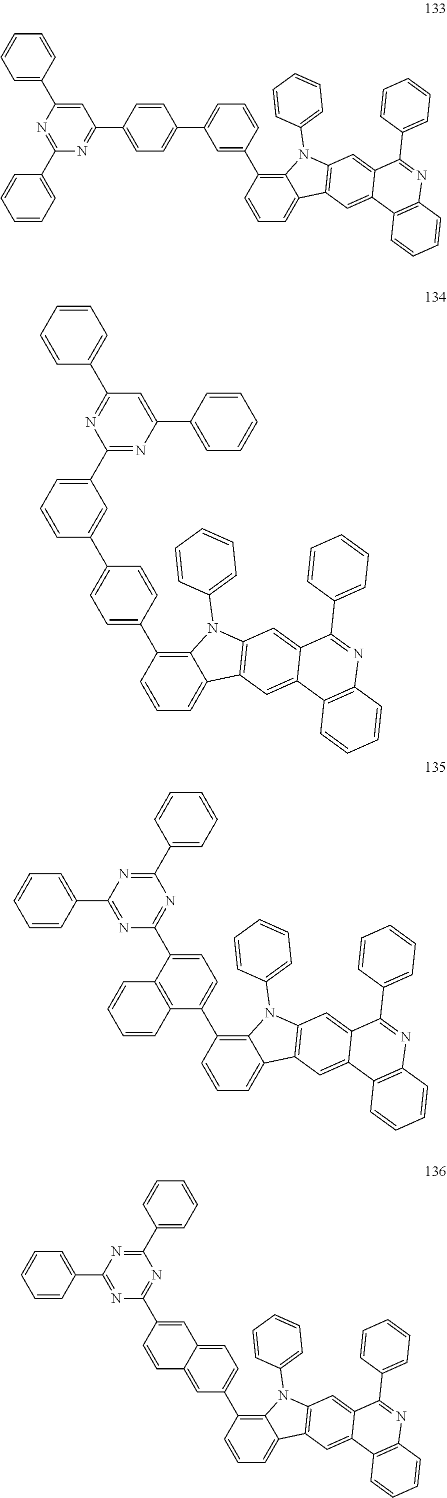 [SMILES: c1ccc(-c2cc(-c3ccc(-c4cccc(-c5cccc6c7cc8c(cc7n(-c7ccccc7)c56)c(-c5ccccc5)nc5ccccc58)c4)cc3)nc(-c3ccccc3)n2)cc1 ; c1ccc(-c2cc(-c3ccccc3)nc(-c3cccc(-c4ccc(-c5cccc6c7cc8c(cc7n(-c7ccccc7)c56)c(-c5ccccc5)nc5ccccc58)cc4)c3)n2)cc1 ; c1ccc(-c2nc(-c3ccccc3)nc(-c3ccc(-c4cccc5c6cc7c(cc6n(-c6ccccc6)c45)c(-c4ccccc4)nc4ccccc47)c4ccccc34)n2)cc1 ; c1ccc(-c2nc(-c3ccccc3)nc(-c3ccc4cc(-c5cccc6c7cc8c(cc7n(-c7ccccc7)c56)c(-c5ccccc5)nc5ccccc58)ccc4c3)n2)cc1]

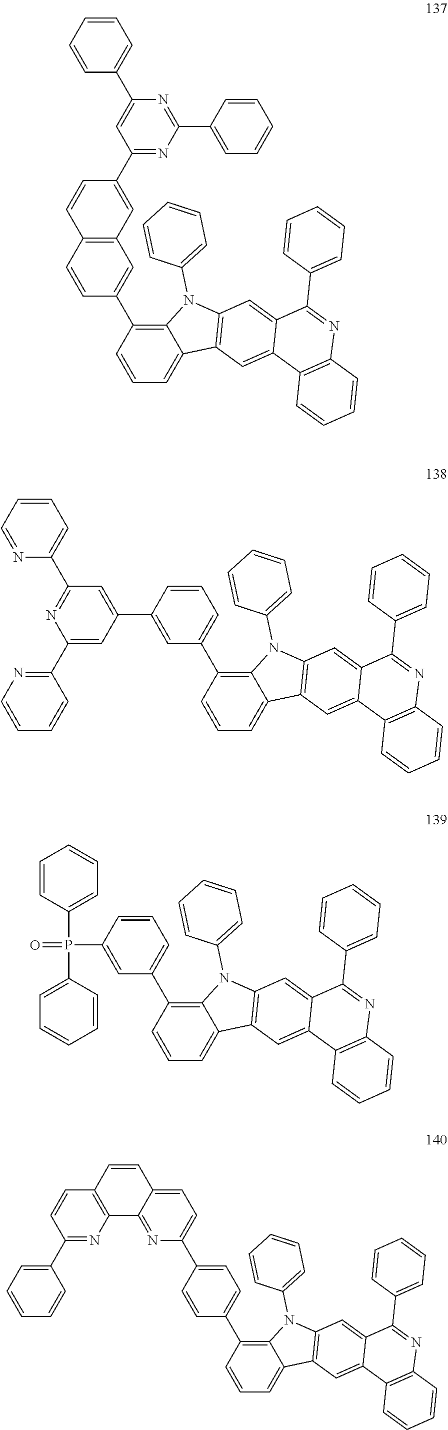 [SMILES: c1ccc(-c2cc(-c3ccc4ccc(-c5cccc6c7cc8c(cc7n(-c7ccccc7)c56)c(-c5ccccc5)nc5ccccc58)cc4c3)nc(-c3ccccc3)n2)cc1 ; c1ccc(-c2nc3ccccc3c3cc4c5cccc(-c6cccc(-c7cc(-c8ccccn8)nc(-c8ccccn8)c7)c6)c5n(-c5ccccc5)c4cc23)cc1 ; O=P(c1ccccc1)(c1ccccc1)c1cccc(-c2cccc3c4cc5c(cc4n(-c4ccccc4)c23)c(-c2ccccc2)nc2ccccc25)c1 ; c1ccc(-c2ccc3ccc4ccc(-c5ccc(-c6cccc7c8cc9c(cc8n(-c8ccccc8)c67)c(-c6ccccc6)nc6ccccc69)cc5)nc4c3n2)cc1]

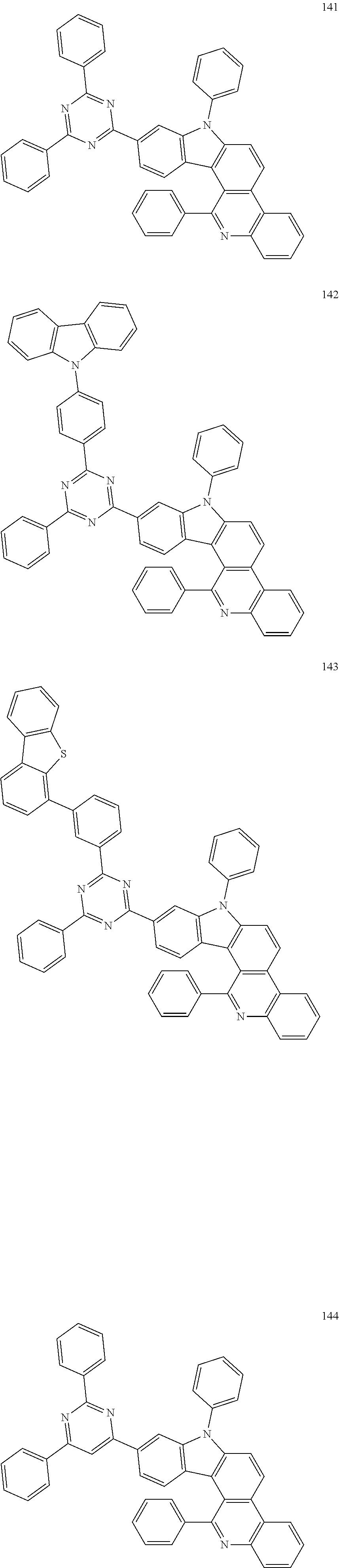 [SMILES: c1ccc(-c2nc(-c3ccccc3)nc(-c3ccc4c5c6c(-c7ccccc7)nc7ccccc7c6ccc5n(-c5ccccc5)c4c3)n2)cc1 ; c1ccc(-c2nc(-c3ccc(-n4c5ccccc5c5ccccc54)cc3)nc(-c3ccc4c5c6c(-c7ccccc7)nc7ccccc7c6ccc5n(-c5ccccc5)c4c3)n2)cc1 ; c1ccc(-c2nc(-c3cccc(-c4cccc5c4sc4ccccc45)c3)nc(-c3ccc4c5c6c(-c7ccccc7)nc7ccccc7c6ccc5n(-c5ccccc5)c4c3)n2)cc1 ; c1ccc(-c2cc(-c3ccc4c5c6c(-c7ccccc7)nc7ccccc7c6ccc5n(-c5ccccc5)c4c3)nc(-c3ccccc3)n2)cc1]

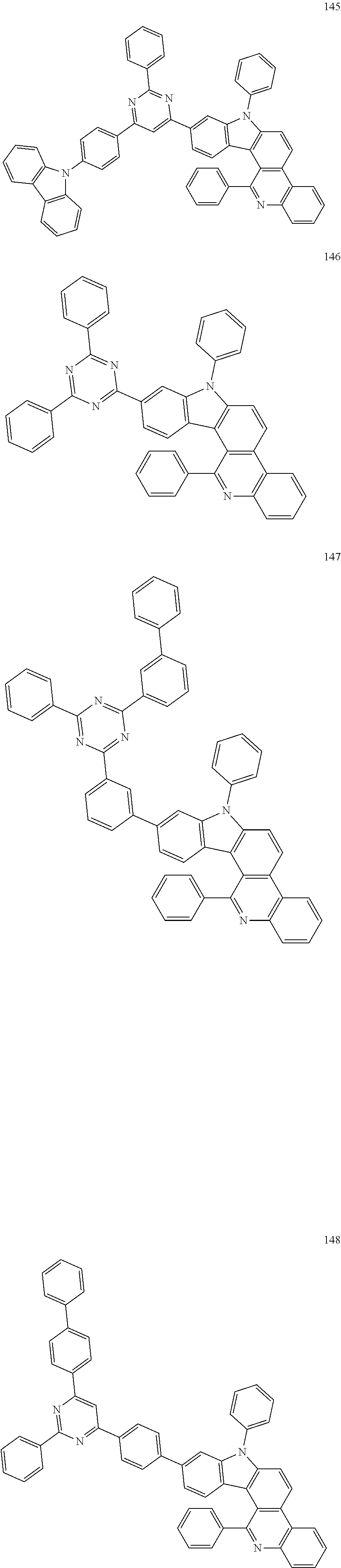 [SMILES: c1ccc(-c2nc(-c3ccc(-n4c5ccccc5c5ccccc54)cc3)cc(-c3ccc4c5c6c(-c7ccccc7)nc7ccccc7c6ccc5n(-c5ccccc5)c4c3)n2)cc1 ; c1ccc(-c2nc(-c3ccccc3)nc(-c3ccc4c5c6c(-c7ccccc7)nc7ccccc7c6ccc5n(-c5ccccc5)c4c3)n2)cc1 ; c1ccc(-c2cccc(-c3nc(-c4ccccc4)nc(-c4cccc(-c5ccc6c7c8c(-c9ccccc9)nc9ccccc9c8ccc7n(-c7ccccc7)c6c5)c4)n3)c2)cc1 ; c1ccc(-c2ccc(-c3cc(-c4ccc(-c5ccc6c7c8c(-c9ccccc9)nc9ccccc9c8ccc7n(-c7ccccc7)c6c5)cc4)nc(-c4ccccc4)n3)cc2)cc1]

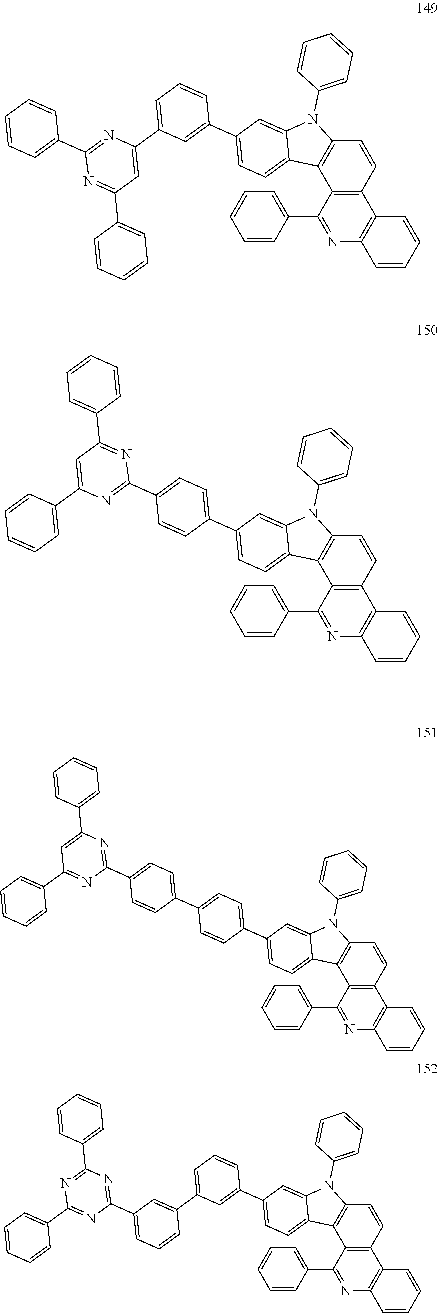 [SMILES: c1ccc(-c2cc(-c3cccc(-c4ccc5c6c7c(-c8ccccc8)nc8ccccc8c7ccc6n(-c6ccccc6)c5c4)c3)nc(-c3ccccc3)n2)cc1 ; c1ccc(-c2cc(-c3ccccc3)nc(-c3ccc(-c4ccc5c6c7c(-c8ccccc8)nc8ccccc8c7ccc6n(-c6ccccc6)c5c4)cc3)n2)cc1 ; c1ccc(-c2cc(-c3ccccc3)nc(-c3ccc(-c4ccc(-c5ccc6c7c8c(-c9ccccc9)nc9ccccc9c8ccc7n(-c7ccccc7)c6c5)cc4)cc3)n2)cc1 ; c1ccc(-c2nc(-c3ccccc3)nc(-c3cccc(-c4cccc(-c5ccc6c7c8c(-c9ccccc9)nc9ccccc9c8ccc7n(-c7ccccc7)c6c5)c4)c3)n2)cc1]

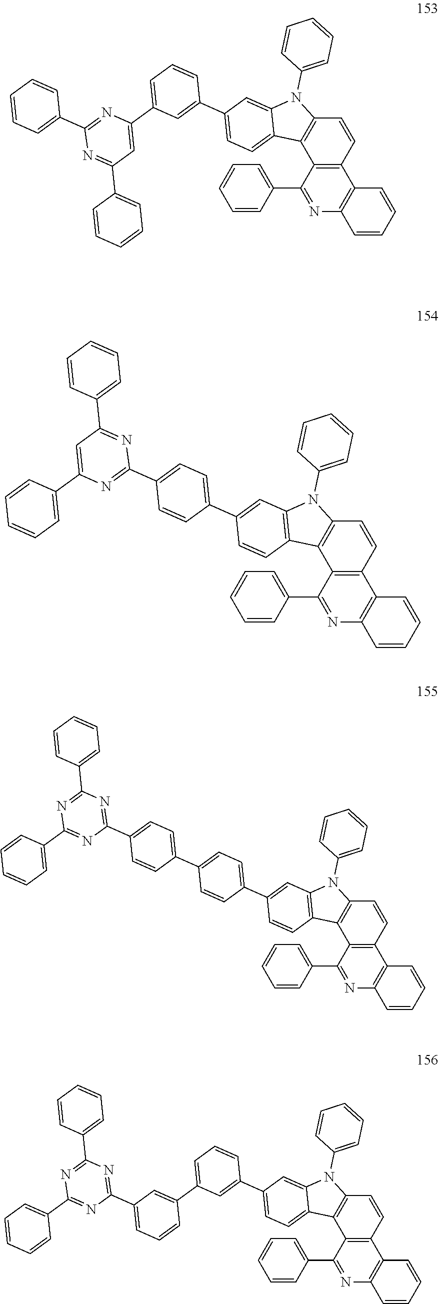

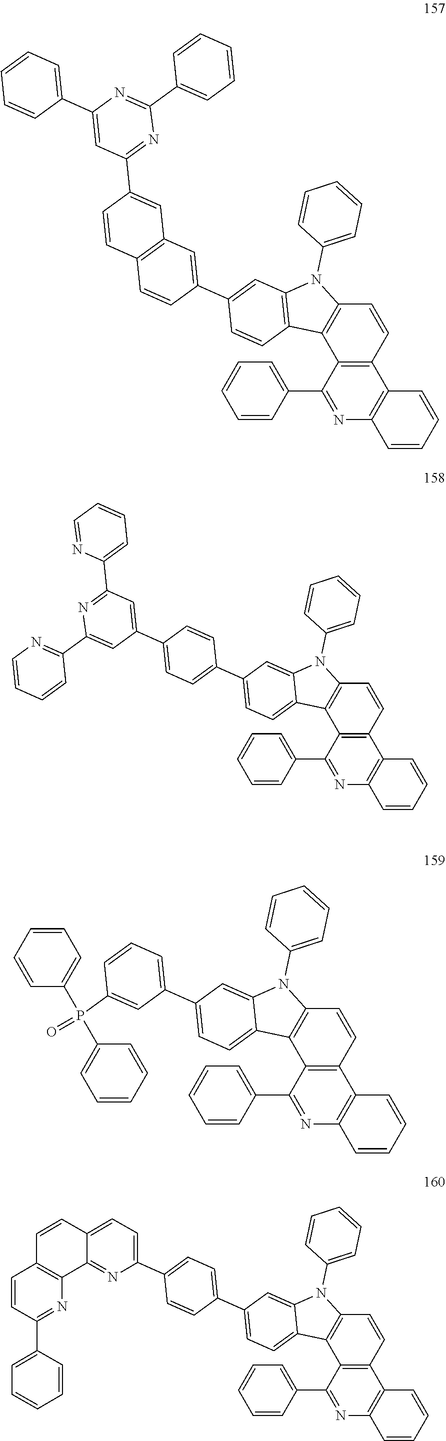 [SMILES: c1ccc(-c2cc(-c3ccc4ccc(-c5ccc6c7c8c(-c9ccccc9)nc9ccccc9c8ccc7n(-c7ccccc7)c6c5)cc4c3)nc(-c3ccccc3)n2)cc1 ; c1ccc(-c2nc3ccccc3c3ccc4c(c5ccc(-c6ccc(-c7cc(-c8ccccn8)nc(-c8ccccn8)c7)cc6)cc5n4-c4ccccc4)c23)cc1 ; O=P(c1ccccc1)(c1ccccc1)c1cccc(-c2ccc3c4c5c(-c6ccccc6)nc6ccccc6c5ccc4n(-c4ccccc4)c3c2)c1 ; c1ccc(-c2ccc3ccc4ccc(-c5ccc(-c6ccc7c8c9c(-c%10ccccc%10)nc%10ccccc%10c9ccc8n(-c8ccccc8)c7c6)cc5)nc4c3n2)cc1]

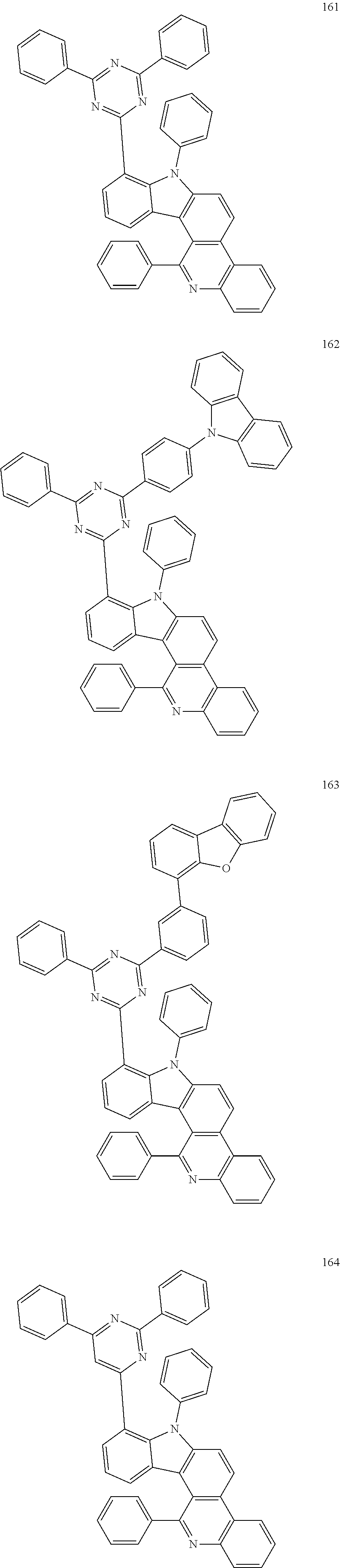 [SMILES: c1ccc(-c2nc(-c3ccccc3)nc(-c3cccc4c5c6c(-c7ccccc7)nc7ccccc7c6ccc5n(-c5ccccc5)c34)n2)cc1 ; c1ccc(-c2nc(-c3ccc(-n4c5ccccc5c5ccccc54)cc3)nc(-c3cccc4c5c6c(-c7ccccc7)nc7ccccc7c6ccc5n(-c5ccccc5)c34)n2)cc1 ; c1ccc(-c2nc(-c3cccc(-c4cccc5c4oc4ccccc45)c3)nc(-c3cccc4c5c6c(-c7ccccc7)nc7ccccc7c6ccc5n(-c5ccccc5)c34)n2)cc1 ; c1ccc(-c2cc(-c3cccc4c5c6c(-c7ccccc7)nc7ccccc7c6ccc5n(-c5ccccc5)c34)nc(-c3ccccc3)n2)cc1]

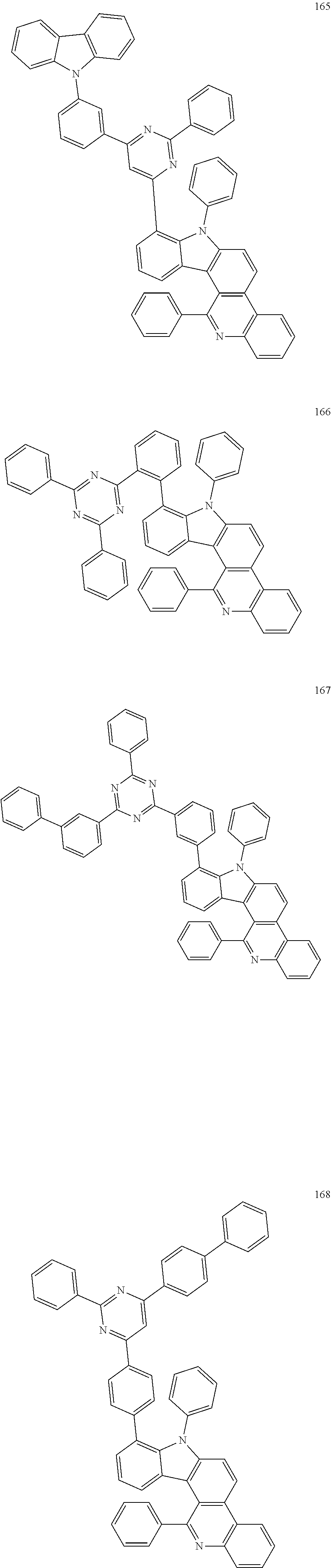 [SMILES: c1ccc(-c2nc(-c3cccc(-n4c5ccccc5c5ccccc54)c3)cc(-c3cccc4c5c6c(-c7ccccc7)nc7ccccc7c6ccc5n(-c5ccccc5)c34)n2)cc1 ; c1ccc(-c2nc(-c3ccccc3)nc(-c3ccccc3-c3cccc4c5c6c(-c7ccccc7)nc7ccccc7c6ccc5n(-c5ccccc5)c34)n2)cc1 ; c1ccc(-c2cccc(-c3nc(-c4ccccc4)nc(-c4cccc(-c5cccc6c7c8c(-c9ccccc9)nc9ccccc9c8ccc7n(-c7ccccc7)c56)c4)n3)c2)cc1 ; c1ccc(-c2ccc(-c3cc(-c4ccc(-c5cccc6c7c8c(-c9ccccc9)nc9ccccc9c8ccc7n(-c7ccccc7)c56)cc4)nc(-c4ccccc4)n3)cc2)cc1]

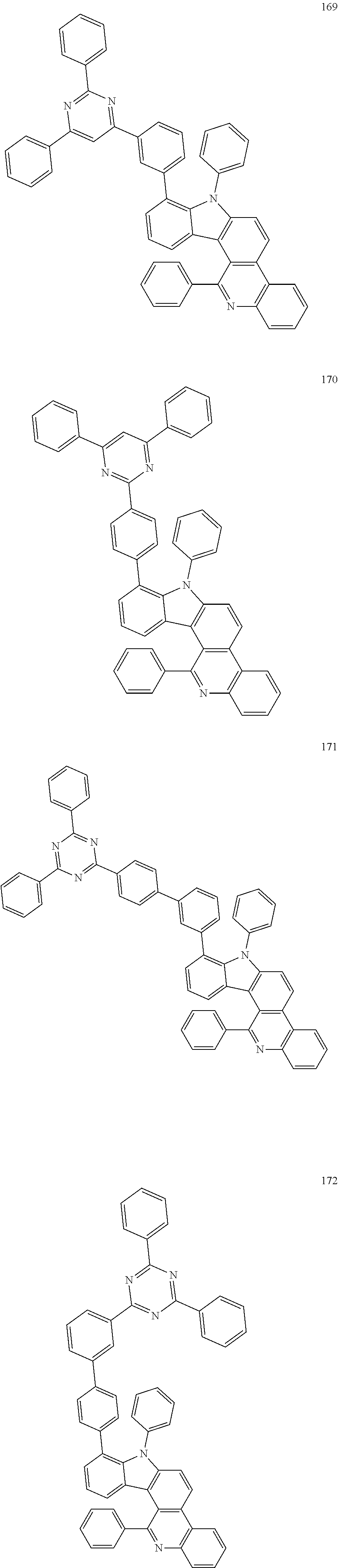 [SMILES: c1ccc(-c2cc(-c3cccc(-c4cccc5c6c7c(-c8ccccc8)nc8ccccc8c7ccc6n(-c6ccccc6)c45)c3)nc(-c3ccccc3)n2)cc1 ; c1ccc(-c2cc(-c3ccccc3)nc(-c3ccc(-c4cccc5c6c7c(-c8ccccc8)nc8ccccc8c7ccc6n(-c6ccccc6)c45)cc3)n2)cc1 ; c1ccc(-c2nc(-c3ccccc3)nc(-c3ccc(-c4cccc(-c5cccc6c7c8c(-c9ccccc9)nc9ccccc9c8ccc7n(-c7ccccc7)c56)c4)cc3)n2)cc1 ; c1ccc(-c2nc(-c3ccccc3)nc(-c3cccc(-c4ccc(-c5cccc6c7c8c(-c9ccccc9)nc9ccccc9c8ccc7n(-c7ccccc7)c56)cc4)c3)n2)cc1]

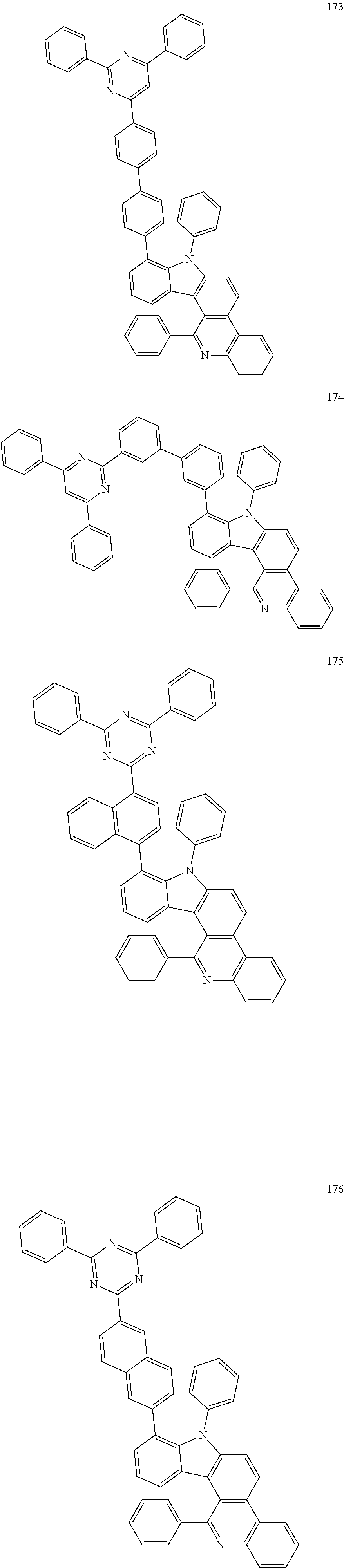 [SMILES: c1ccc(-c2cc(-c3ccc(-c4ccc(-c5cccc6c7c8c(-c9ccccc9)nc9ccccc9c8ccc7n(-c7ccccc7)c56)cc4)cc3)nc(-c3ccccc3)n2)cc1 ; c1ccc(-c2cc(-c3ccccc3)nc(-c3cccc(-c4cccc(-c5cccc6c7c8c(-c9ccccc9)nc9ccccc9c8ccc7n(-c7ccccc7)c56)c4)c3)n2)cc1 ; c1ccc(-c2nc(-c3ccccc3)nc(-c3ccc(-c4cccc5c6c7c(-c8ccccc8)nc8ccccc8c7ccc6n(-c6ccccc6)c45)c4ccccc34)n2)cc1 ; c1ccc(-c2nc(-c3ccccc3)nc(-c3ccc4cc(-c5cccc6c7c8c(-c9ccccc9)nc9ccccc9c8ccc7n(-c7ccccc7)c56)ccc4c3)n2)cc1]

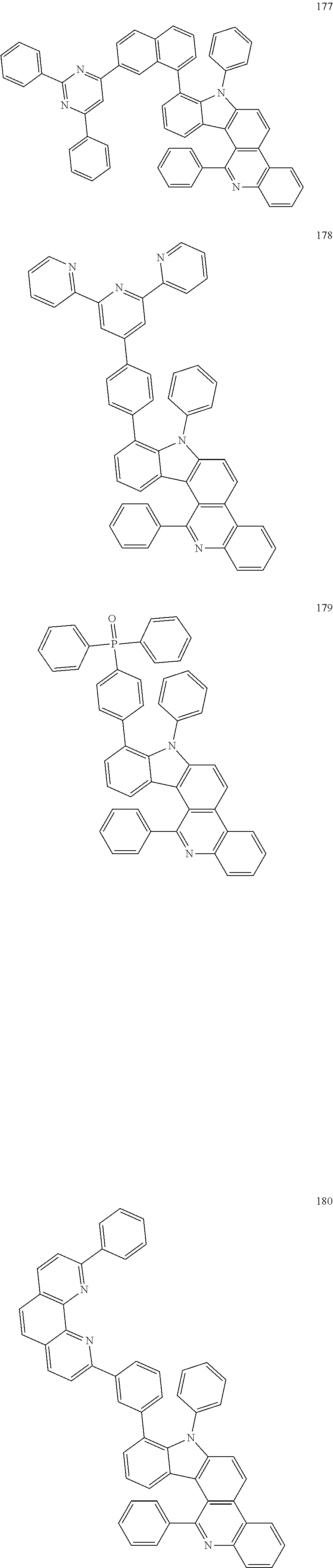 [SMILES: c1ccc(-c2cc(-c3ccc4cccc(-c5cccc6c7c8c(-c9ccccc9)nc9ccccc9c8ccc7n(-c7ccccc7)c56)c4c3)nc(-c3ccccc3)n2)cc1 ; c1ccc(-c2nc3ccccc3c3ccc4c(c5cccc(-c6ccc(-c7cc(-c8ccccn8)nc(-c8ccccn8)c7)cc6)c5n4-c4ccccc4)c23)cc1 ; O=P(c1ccccc1)(c1ccccc1)c1ccc(-c2cccc3c4c5c(-c6ccccc6)nc6ccccc6c5ccc4n(-c4ccccc4)c23)cc1 ; c1ccc(-c2ccc3ccc4ccc(-c5cccc(-c6cccc7c8c9c(-c%10ccccc%10)nc%10ccccc%10c9ccc8n(-c8ccccc8)c67)c5)nc4c3n2)cc1]

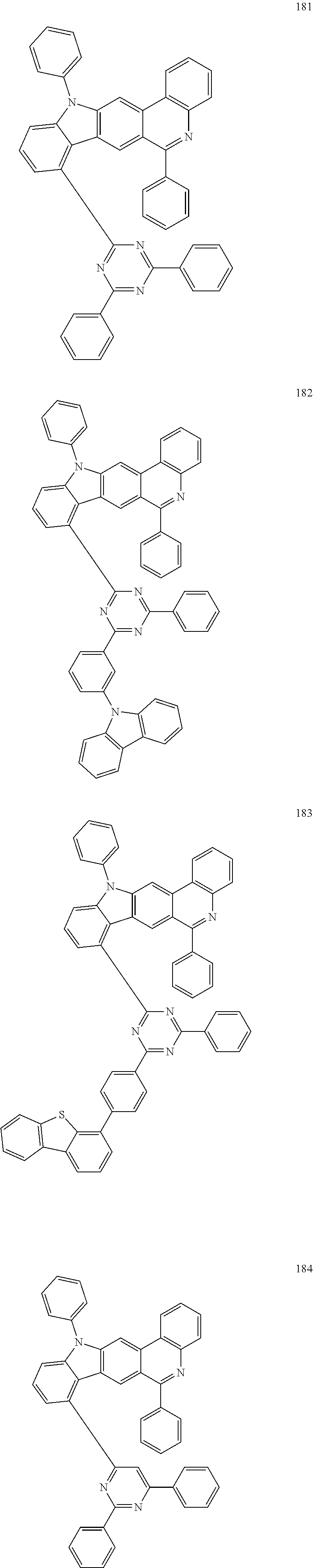 [SMILES: c1ccc(-c2nc(-c3ccccc3)nc(-c3cccc4c3c3cc5c(-c6ccccc6)nc6ccccc6c5cc3n4-c3ccccc3)n2)cc1 ; c1ccc(-c2nc(-c3cccc(-n4c5ccccc5c5ccccc54)c3)nc(-c3cccc4c3c3cc5c(-c6ccccc6)nc6ccccc6c5cc3n4-c3ccccc3)n2)cc1 ; c1ccc(-c2nc(-c3ccc(-c4cccc5c4sc4ccccc45)cc3)nc(-c3cccc4c3c3cc5c(-c6ccccc6)nc6ccccc6c5cc3n4-c3ccccc3)n2)cc1 ; c1ccc(-c2cc(-c3cccc4c3c3cc5c(-c6ccccc6)nc6ccccc6c5cc3n4-c3ccccc3)nc(-c3ccccc3)n2)cc1]

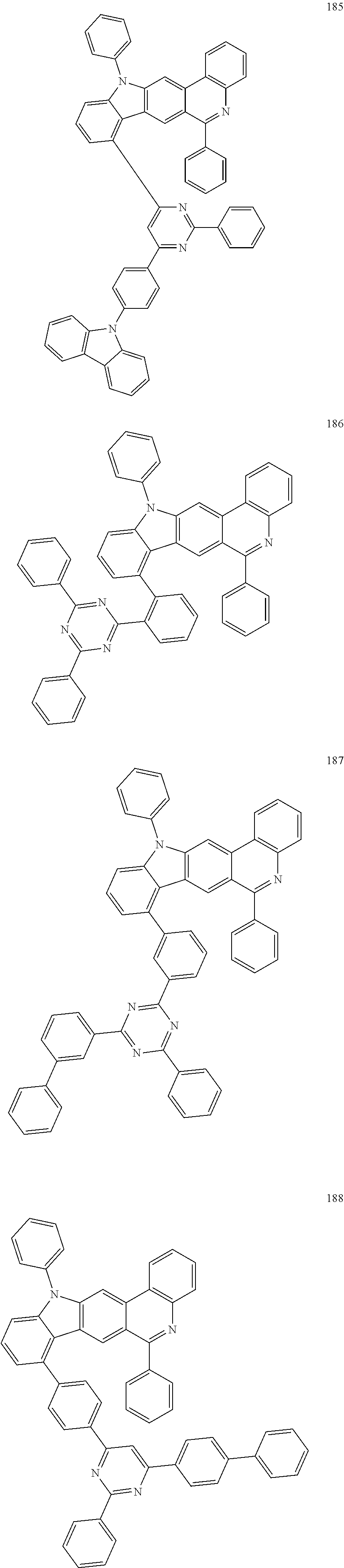

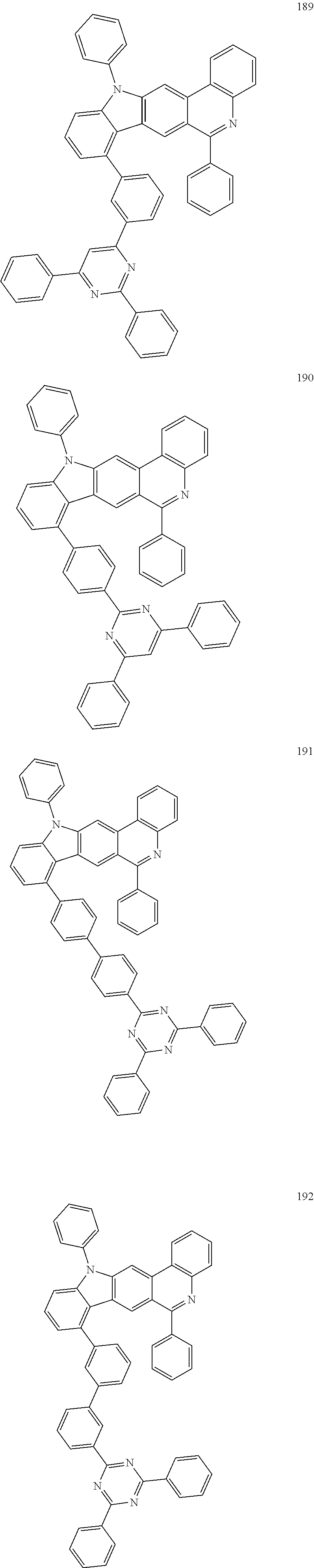 [SMILES: c1ccc(-c2cc(-c3cccc(-c4cccc5c4c4cc6c(-c7ccccc7)nc7ccccc7c6cc4n5-c4ccccc4)c3)nc(-c3ccccc3)n2)cc1 ; c1ccc(-c2cc(-c3ccccc3)nc(-c3ccc(-c4cccc5c4c4cc6c(-c7ccccc7)nc7ccccc7c6cc4n5-c4ccccc4)cc3)n2)cc1 ; c1ccc(-c2nc(-c3ccccc3)nc(-c3ccc(-c4ccc(-c5cccc6c5c5cc7c(-c8ccccc8)nc8ccccc8c7cc5n6-c5ccccc5)cc4)cc3)n2)cc1 ; c1ccc(-c2nc(-c3ccccc3)nc(-c3cccc(-c4cccc(-c5cccc6c5c5cc7c(-c8ccccc8)nc8ccccc8c7cc5n6-c5ccccc5)c4)c3)n2)cc1]

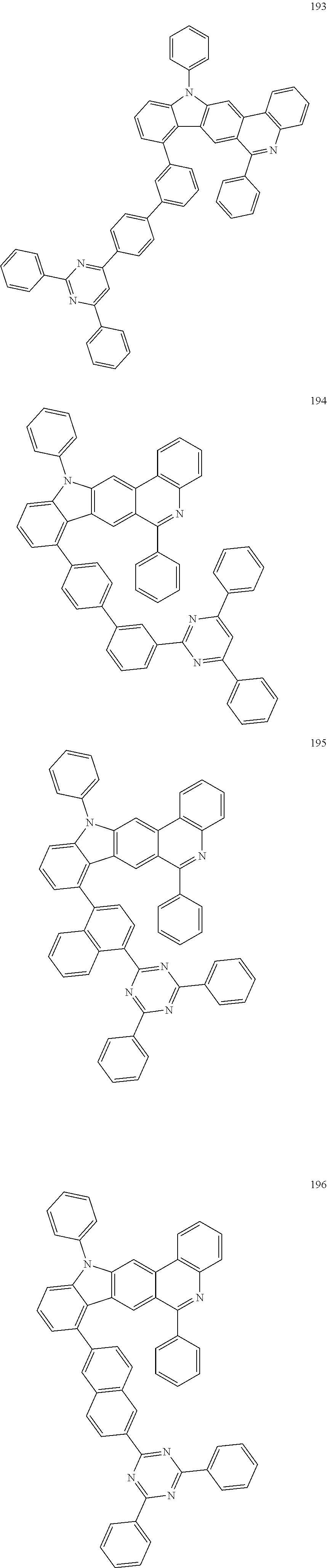 [SMILES: c1ccc(-c2cc(-c3ccc(-c4cccc(-c5cccc6c5c5cc7c(-c8ccccc8)nc8ccccc8c7cc5n6-c5ccccc5)c4)cc3)nc(-c3ccccc3)n2)cc1 ; c1ccc(-c2cc(-c3ccccc3)nc(-c3cccc(-c4ccc(-c5cccc6c5c5cc7c(-c8ccccc8)nc8ccccc8c7cc5n6-c5ccccc5)cc4)c3)n2)cc1 ; c1ccc(-c2nc(-c3ccccc3)nc(-c3ccc(-c4cccc5c4c4cc6c(-c7ccccc7)nc7ccccc7c6cc4n5-c4ccccc4)c4ccccc34)n2)cc1 ; c1ccc(-c2nc(-c3ccccc3)nc(-c3ccc4cc(-c5cccc6c5c5cc7c(-c8ccccc8)nc8ccccc8c7cc5n6-c5ccccc5)ccc4c3)n2)cc1]

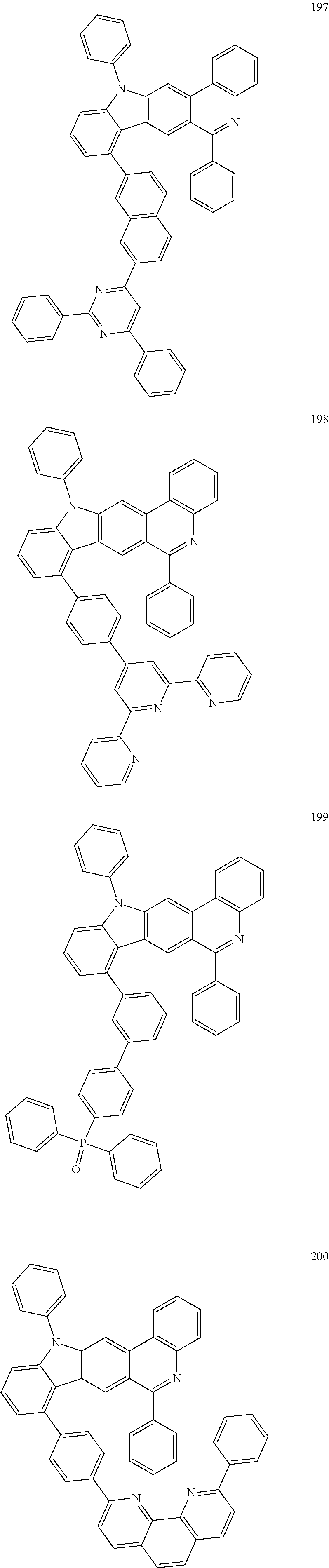

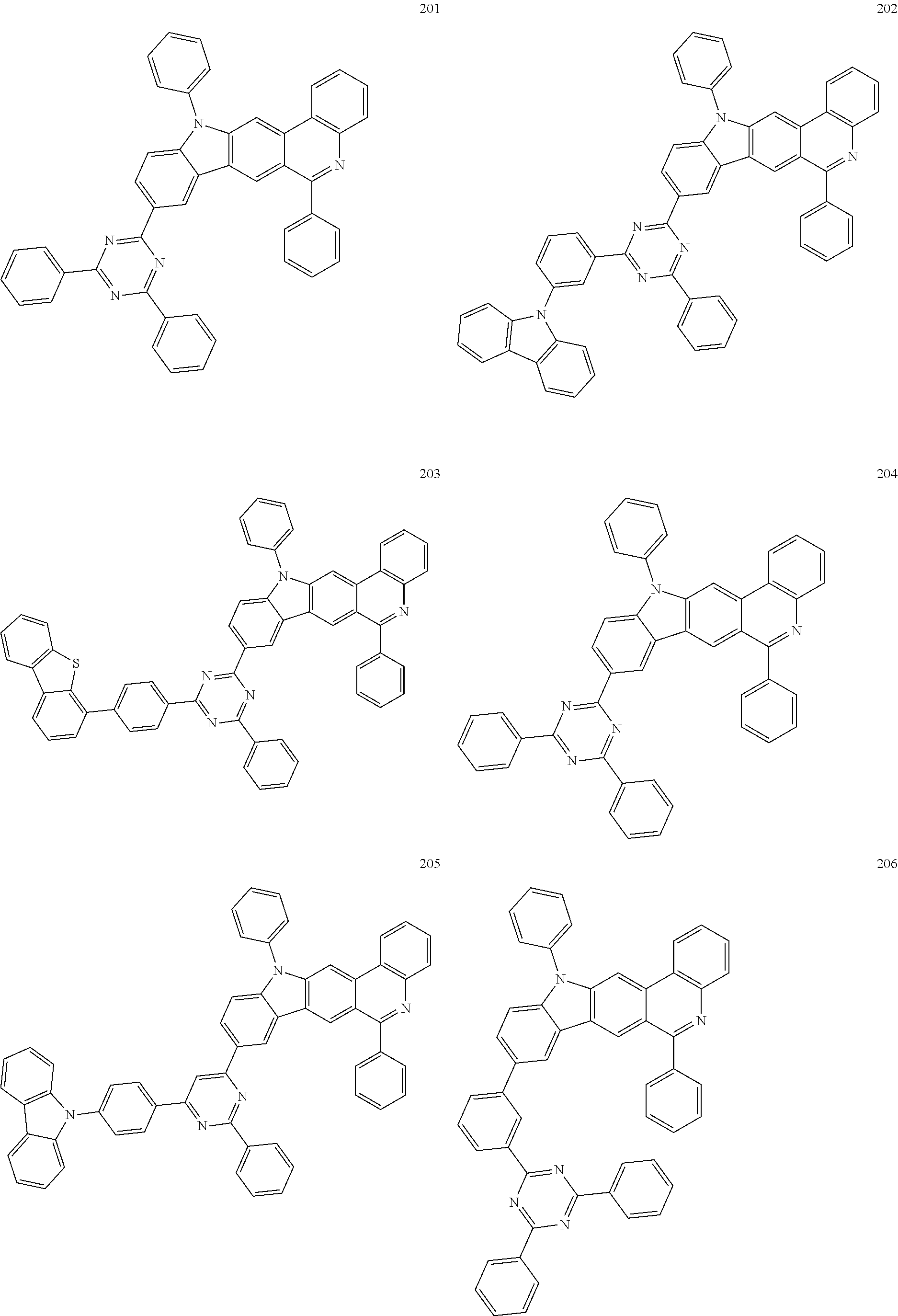

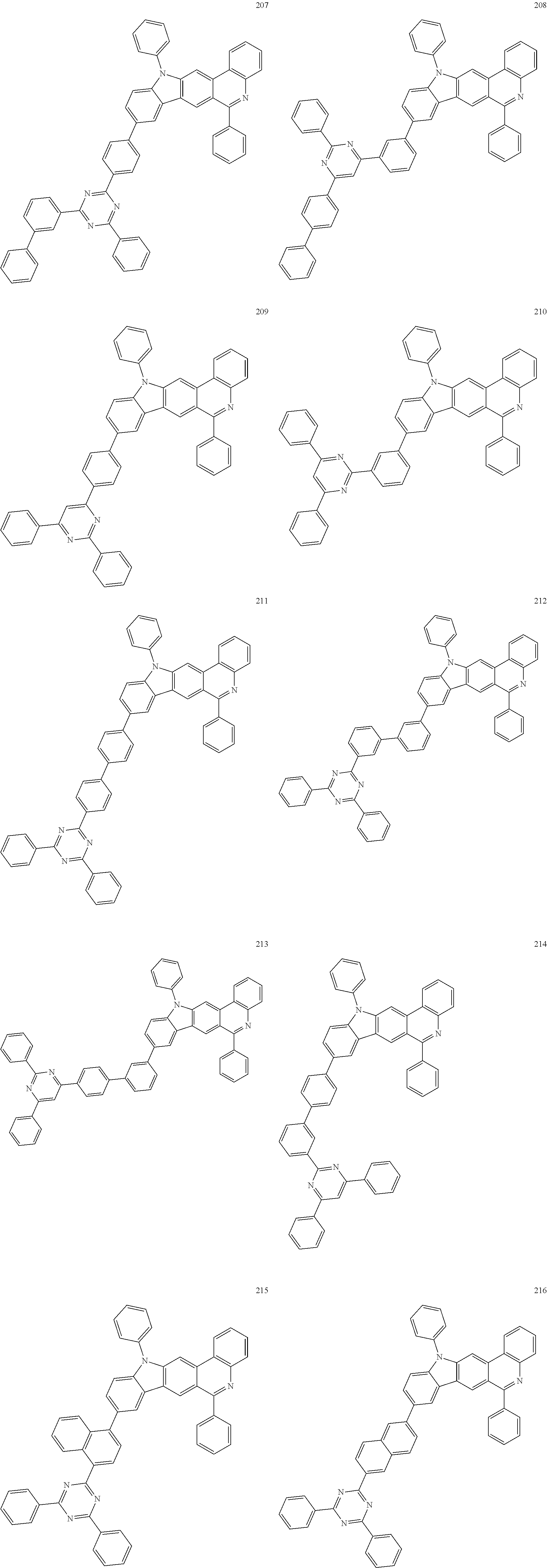

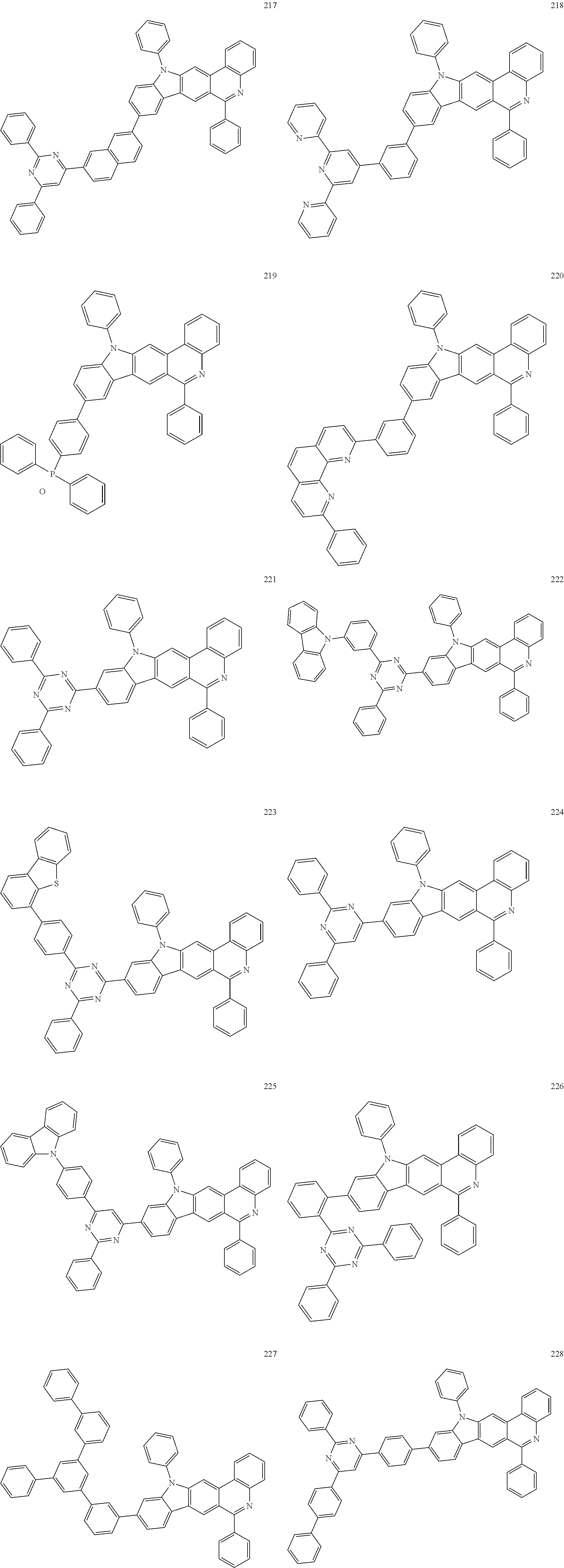

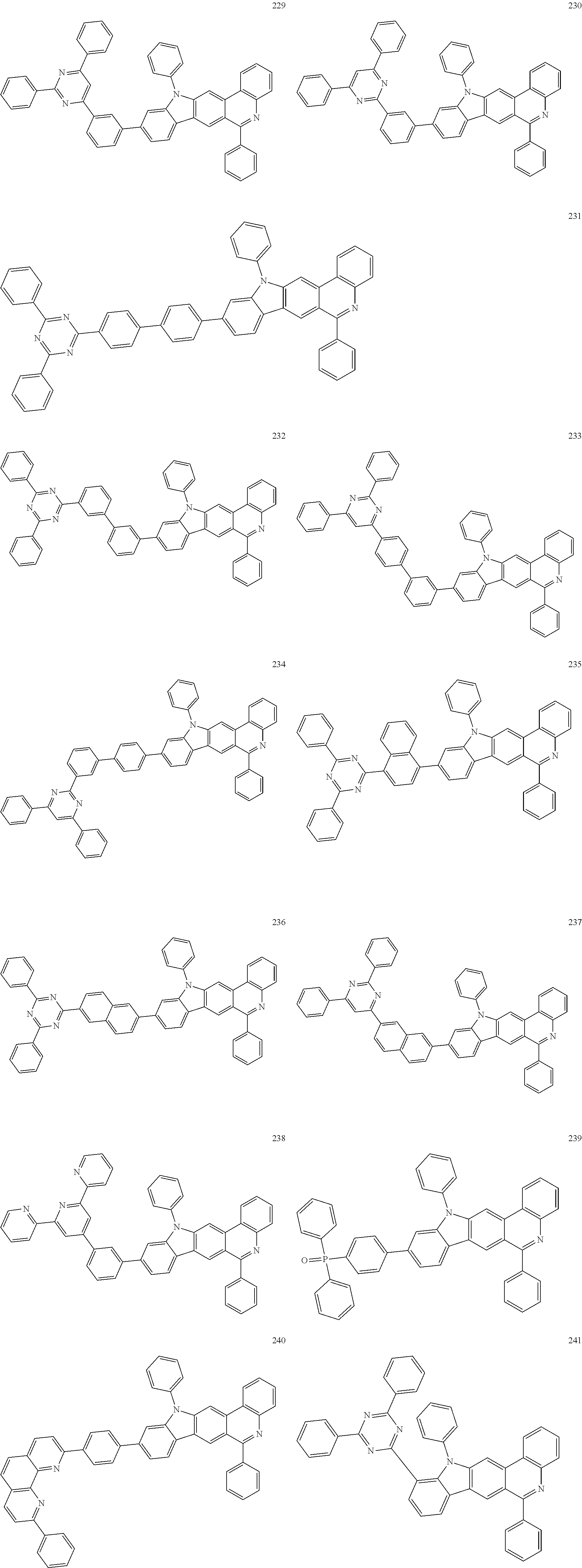

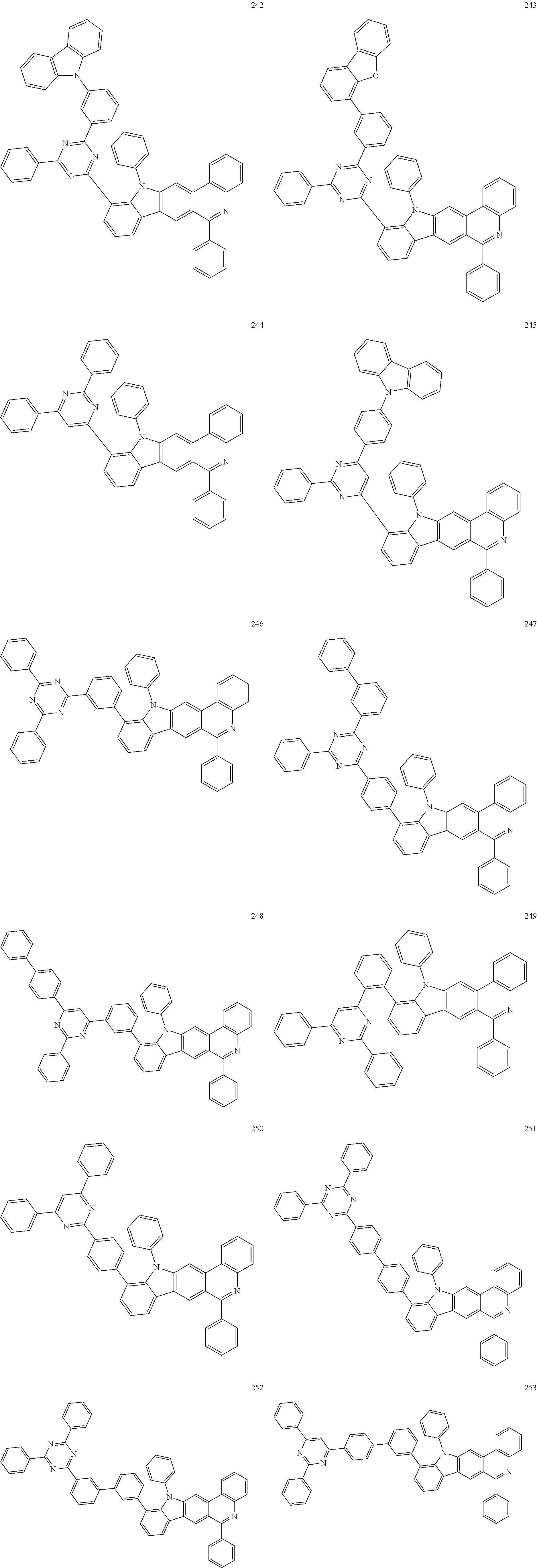

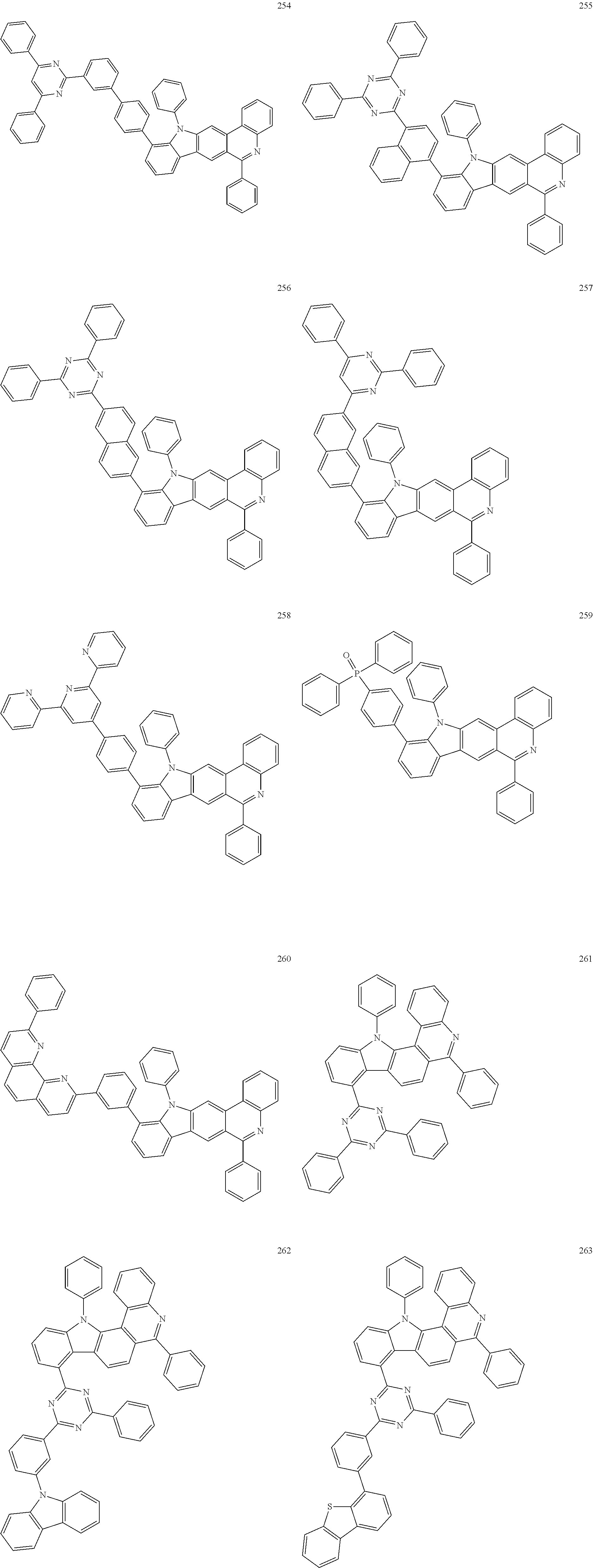

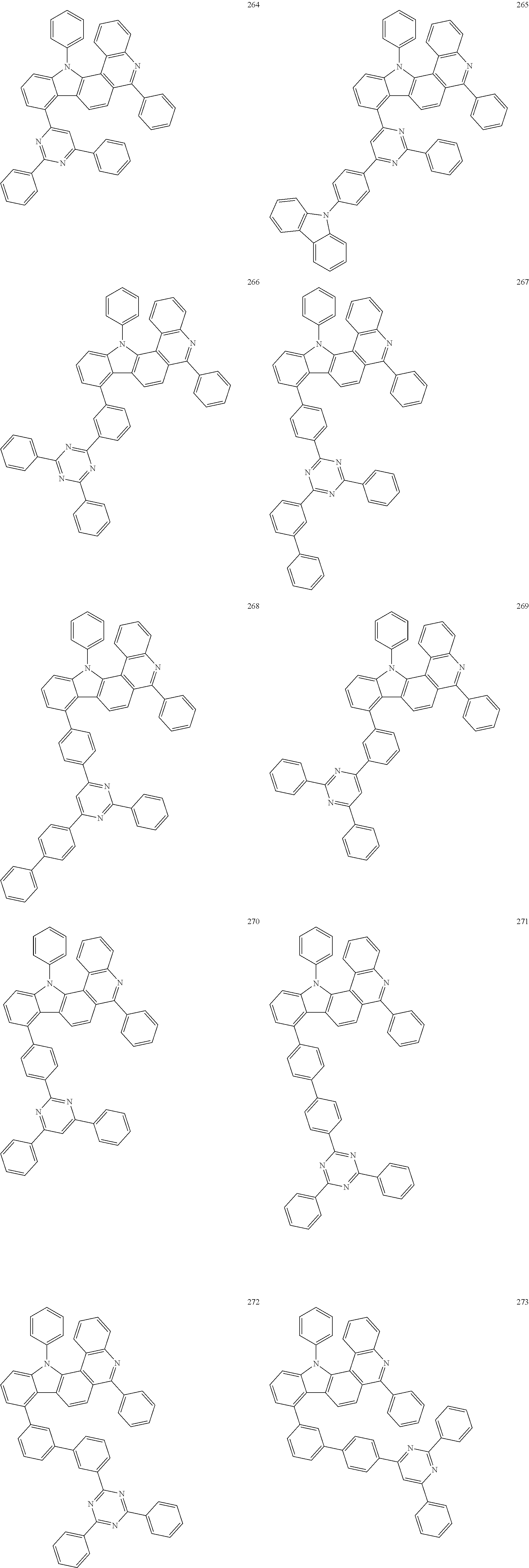

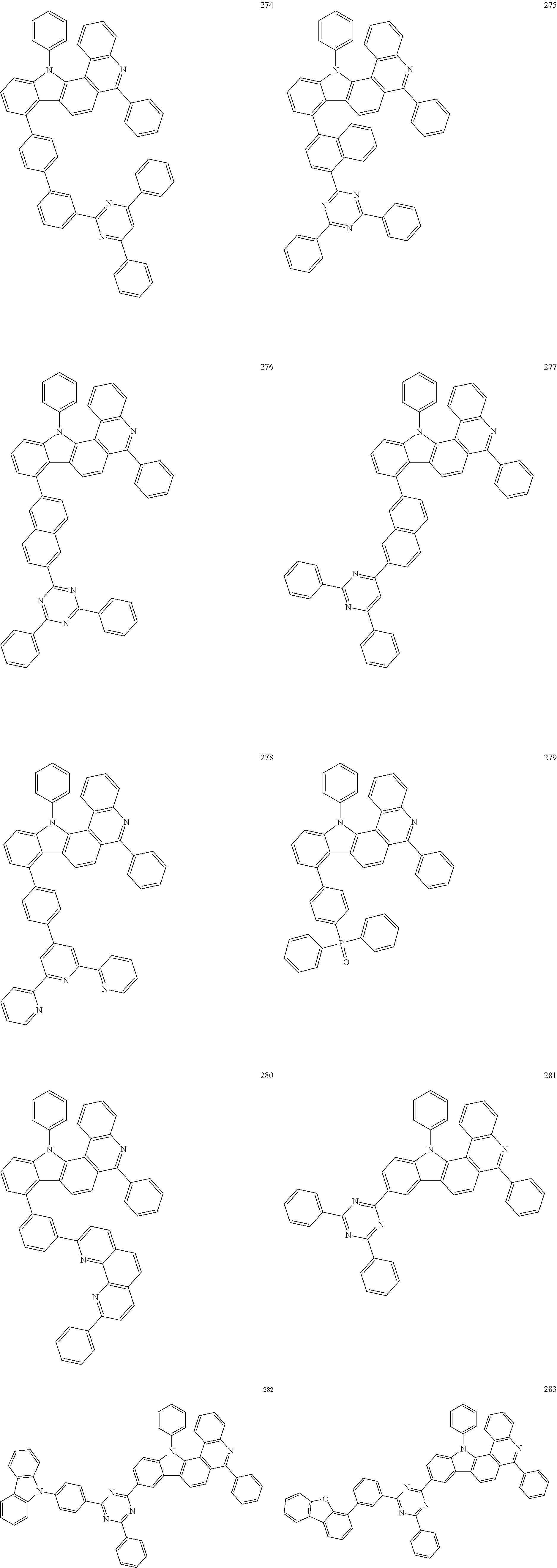

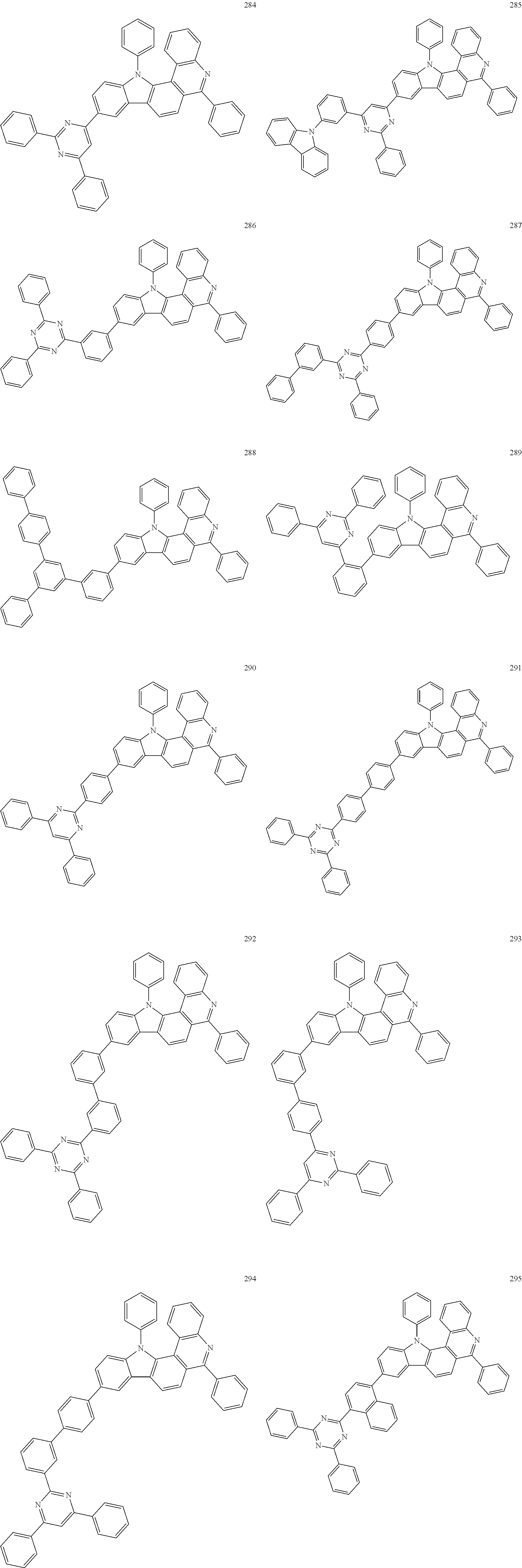

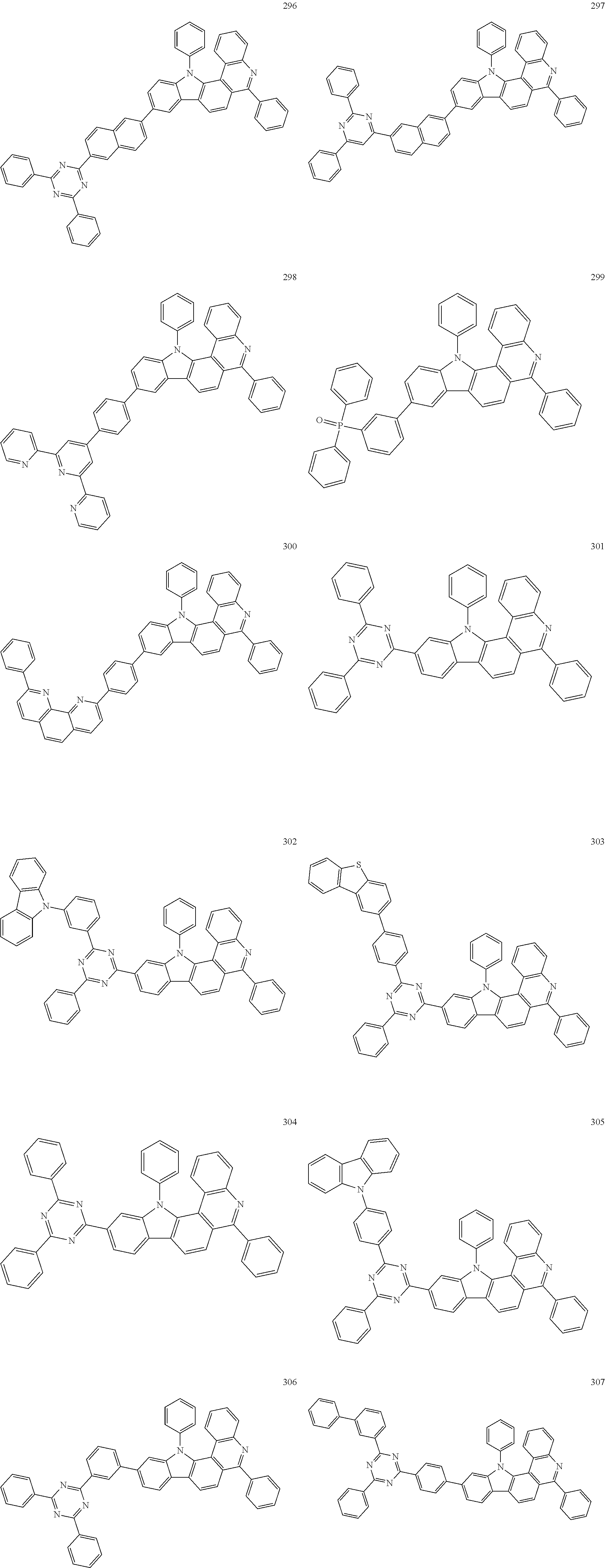

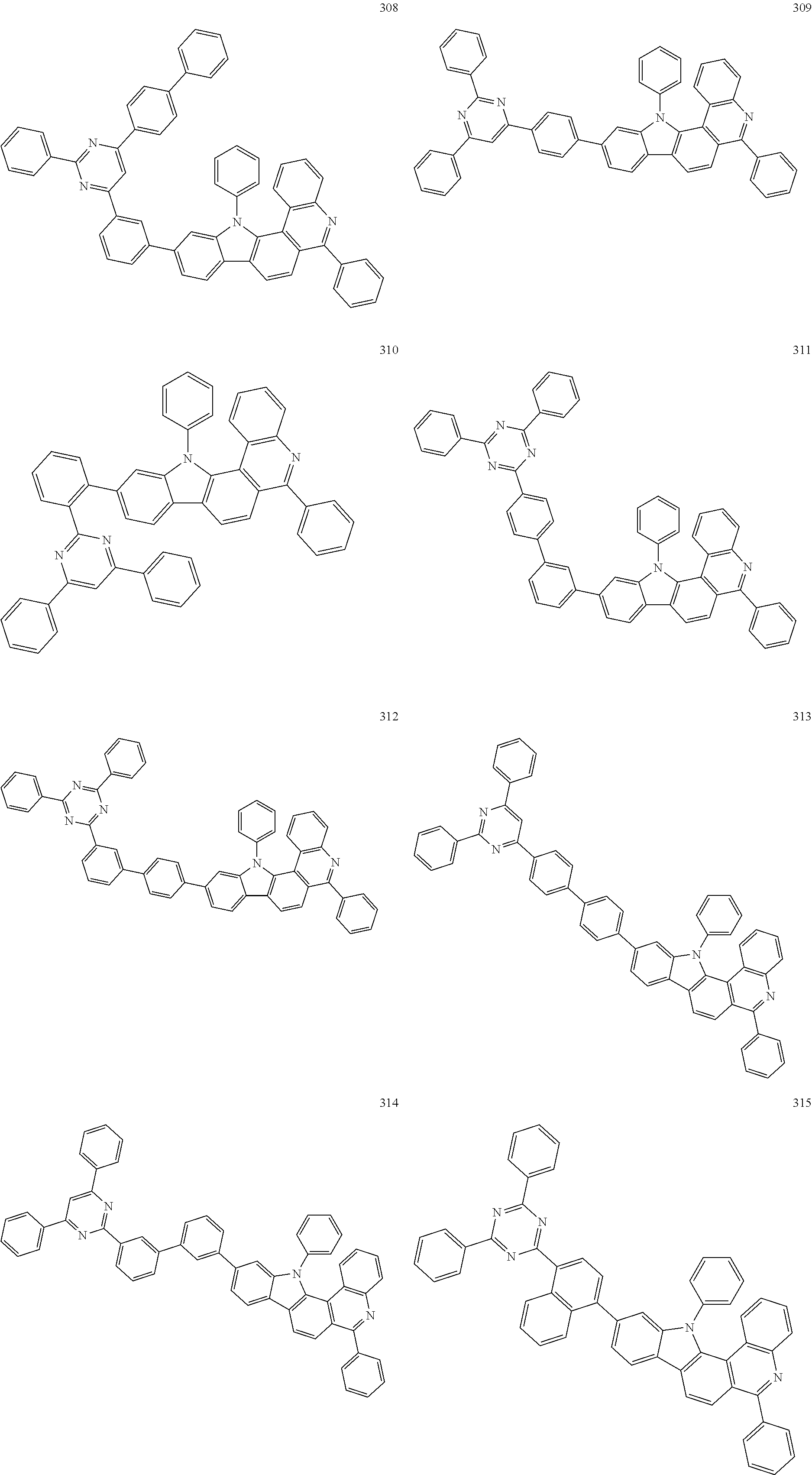

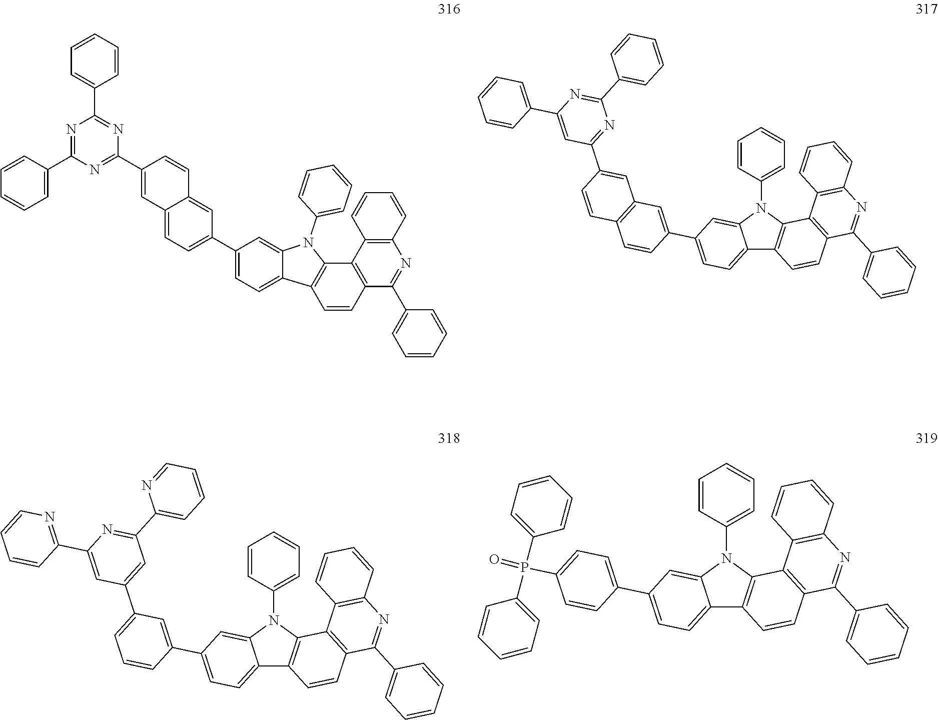 [SMILES: c1ccc(-c2nc(-c3ccccc3)nc(-c3ccc4cc(-c5ccc6c7ccc8c(-c9ccccc9)nc9ccccc9c8c7n(-c7ccccc7)c6c5)ccc4c3)n2)cc1 ; c1ccc(-c2cc(-c3ccc4ccc(-c5ccc6c7ccc8c(-c9ccccc9)nc9ccccc9c8c7n(-c7ccccc7)c6c5)cc4c3)nc(-c3ccccc3)n2)cc1 ; c1ccc(-c2nc3ccccc3c3c2ccc2c4ccc(-c5cccc(-c6cc(-c7ccccn7)nc(-c7ccccn7)c6)c5)cc4n(-c4ccccc4)c23)cc1 ; O=P(c1ccccc1)(c1ccccc1)c1ccc(-c2ccc3c4ccc5c(-c6ccccc6)nc6ccccc6c5c4n(-c4ccccc4)c3c2)cc1]

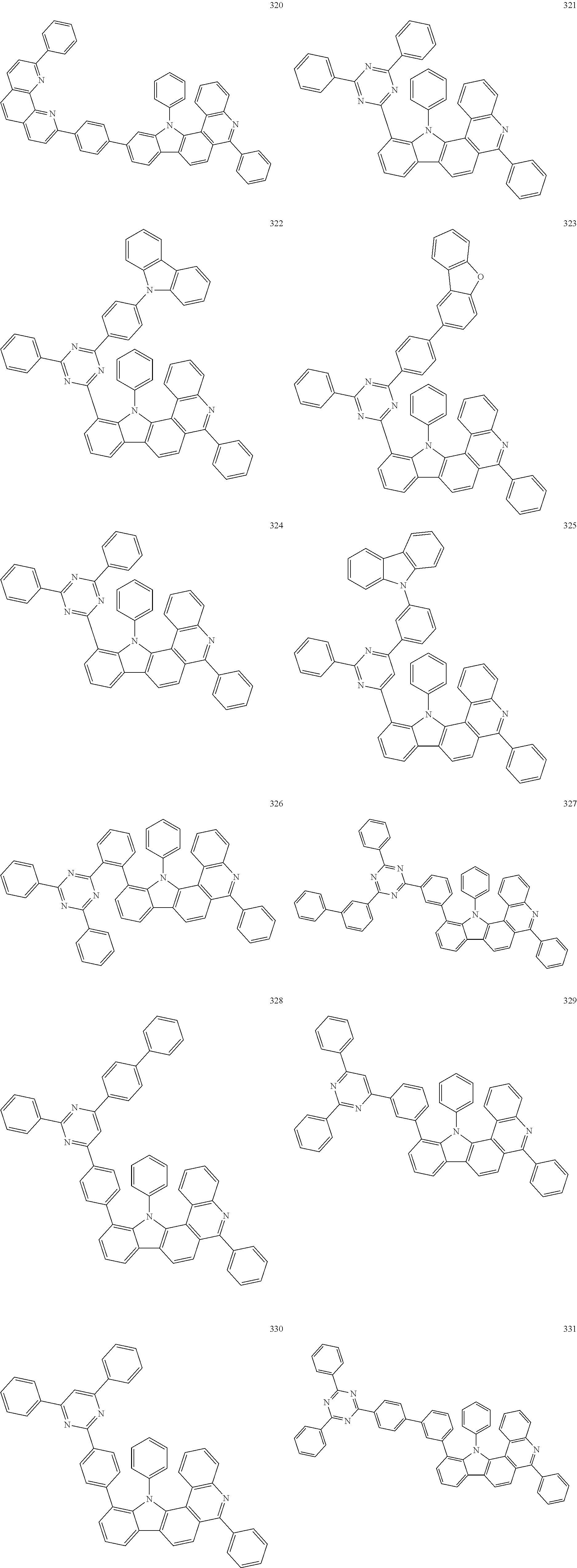

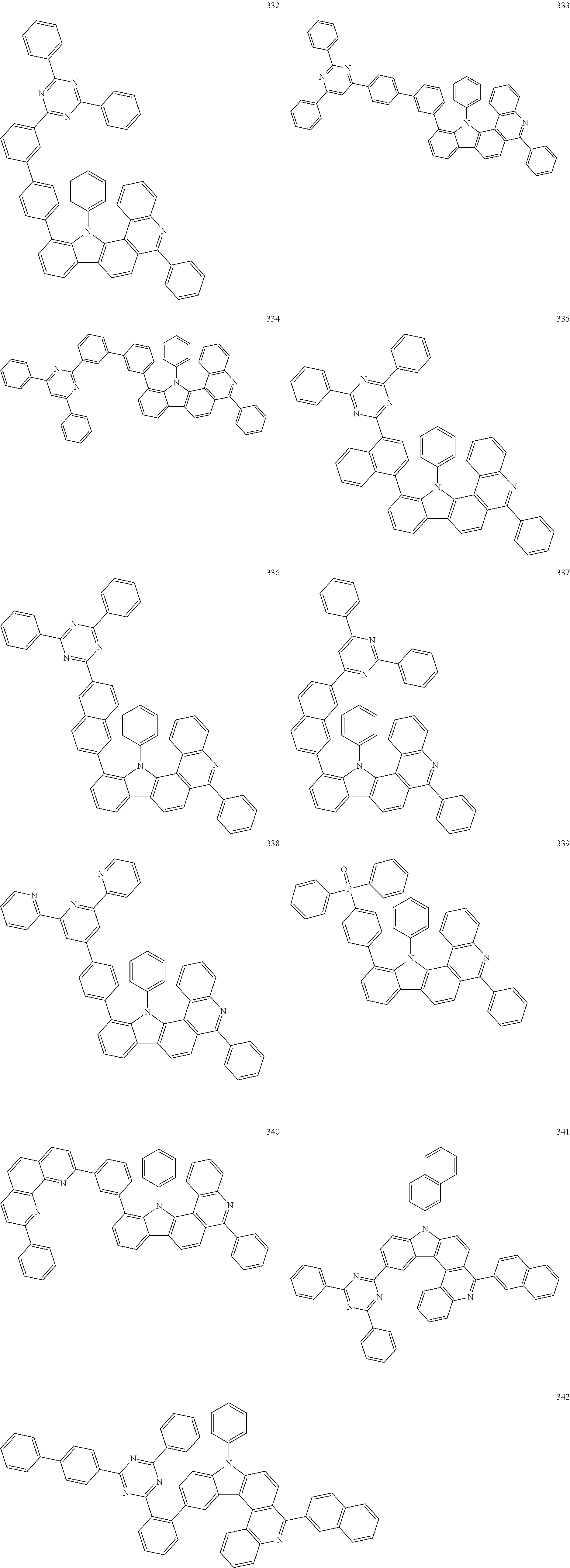

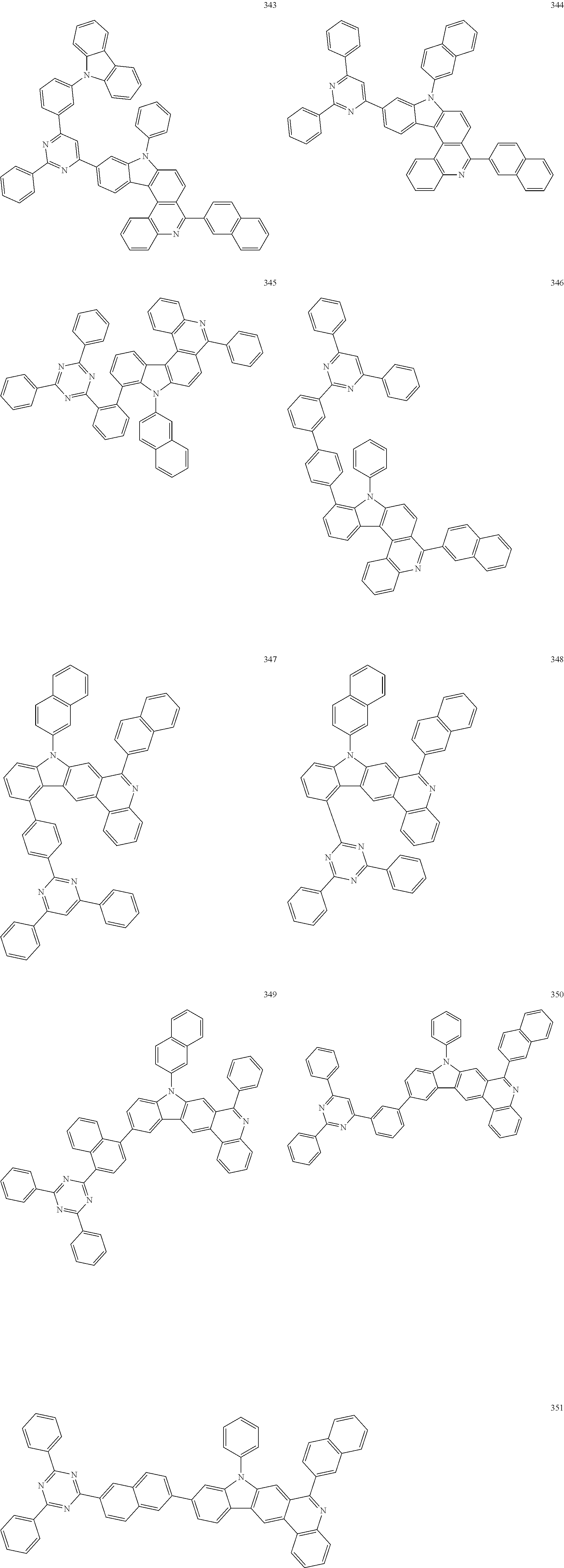

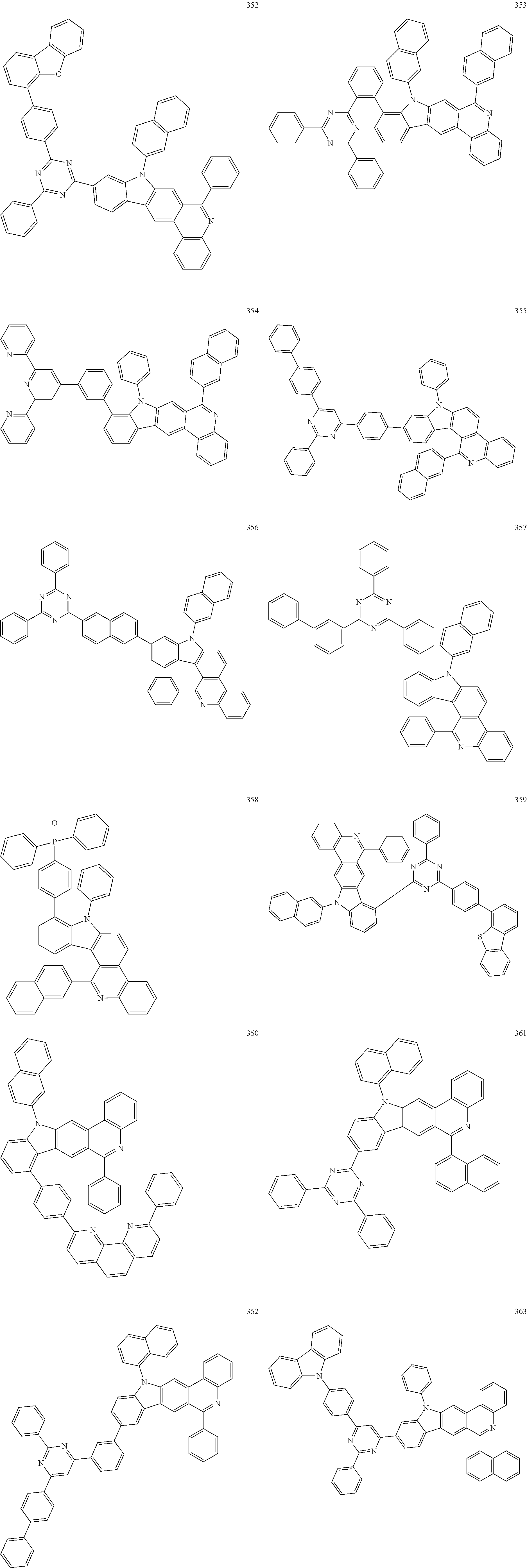

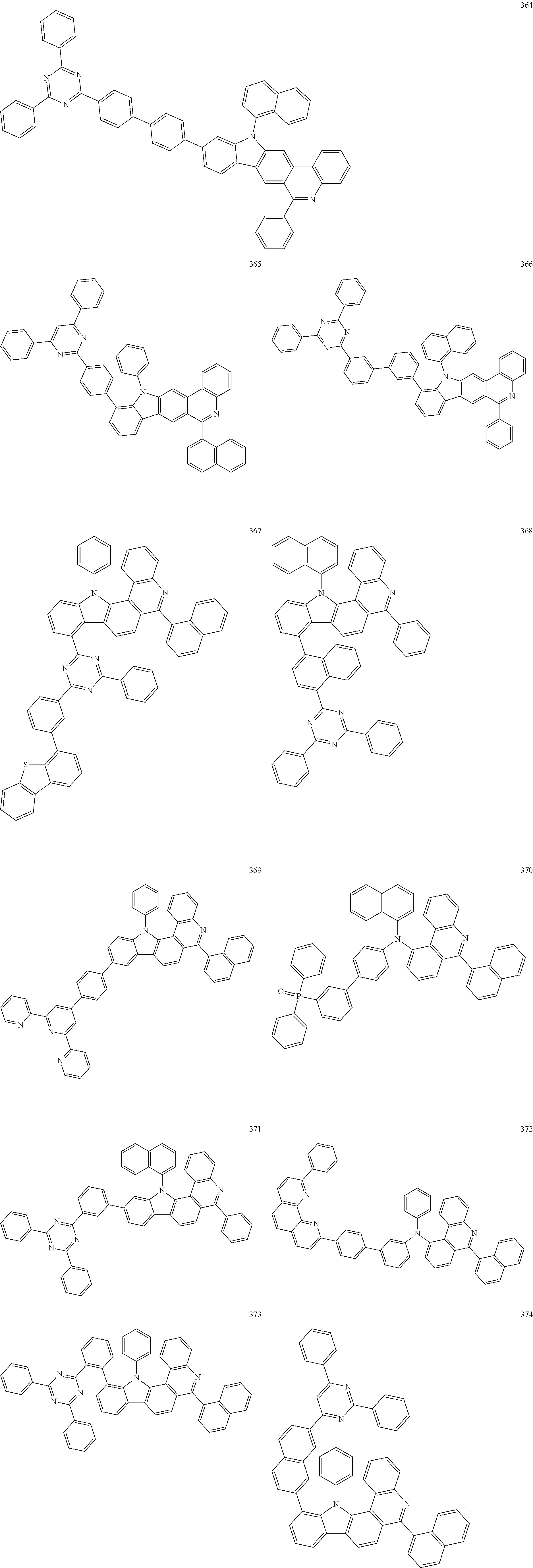

8. An organic light emitting device comprising:
a first electrode;
a second electrode provided opposite to the first electrode; and
one or more organic material layers provided between the first electrode and the second electrode,
wherein one or more layers of the organic material layers comprise the heterocyclic compound of claim 1.

9. The organic light emitting device of claim 8, wherein the organic material layer comprises a light emitting layer, and the light emitting layer comprises the heterocyclic compound.

10. The organic light emitting device of claim 8, wherein the organic material layer comprises an electron injection layer or an electron transfer layer, and the electron injection layer or the electron transfer layer comprises the heterocyclic compound.

11. The organic light emitting device of claim 8, wherein the organic material layer comprises an electron blocking layer or a hole blocking layer, and the electron blocking layer or the hole blocking layer comprises the heterocyclic compound.

12. The organic light emitting device of claim 8, further comprising one, two or more layers selected from the group consisting of a light emitting layer, a hole injection layer, a hole transfer layer, an electron injection layer, an electron transfer layer, an electron blocking layer and a hole blocking layer.

13. The organic light emitting device of claim 8, comprising:
a first electrode;
a first stack provided on the first electrode and comprising a first light emitting layer;
a charge generation layer provided on the first stack;
a second stack provided on the charge generation layer and comprising a second light emitting layer; and
a second electrode provided on the second stack.

14. The organic light emitting device of claim 13, wherein the charge generation layer comprises the heterocyclic compound.

15. The organic light emitting device of claim 14, wherein the charge generation layer is an N-type charge generation layer, and the charge generation layer comprises the heterocyclic compound.
